SEQUENCE LISTING (65 sequences)

SEQ ID NO: 1 - DNA, 41 bp, synthetic construct
  source 1..41: mol_type = other DNA
agcagcaggtgcgggtggcaccctacaggccctctcagccg

SEQ ID NO: 2 - DNA, 43 bp, synthetic construct
  source 1..43: mol_type = other DNA
agcagcaggtgcgggtggcaccctgctggccgncanttagcag

SEQ ID NO: 3 - DNA, 41 bp, synthetic construct
  source 1..41: mol_type = other DNA
ggccgcctgcgtcctcaggccaaggagtaaagtctcctggg

SEQ ID NO: 4 - DNA, 41 bp, synthetic construct
  source 1..41: mol_type = other DNA
ggccgcctgcgccaggggaggtcaggaacgactgggaccca

SEQ ID NO: 5 - DNA, 41 bp, synthetic construct
  source 1..41: mol_type = other DNA
agcactgccattgccagcagtccatcctacctcgcatccag

SEQ ID NO: 6 - DNA, 41 bp, synthetic construct
  source 1..41: mol_type = other DNA
agcactgccattgggcaggctcagcagtttcacaggtctca

SEQ ID NO: 7 - RNA, 30 nt, synthetic construct
  source 1..30: mol_type = other RNA
gagaccagatgctgacttgaacaagctagc

SEQ ID NO: 8 - RNA, 29 nt, synthetic construct
  source 1..29: mol_type = other RNA
aagctgaaggctggctttgtcggaggcca

SEQ ID NO: 9 - DNA, 68 bp, synthetic construct
  source 1..68: mol_type = other DNA
gatgctgacttgaacaagctagcccaaacacacaccaacatccccaagctgaaggctggctttgtcgg

SEQ ID NO: 10 - skipped

SEQ ID NO: 11 - DNA, 52 bp, synthetic construct
  source 1..52: mol_type = other DNA
gatgataacttgaacaagctagcccaaacacacaccaacatccccaagctgg

SEQ ID NO: 12 - DNA, 25 bp, synthetic construct
  source 1..25: mol_type = other DNA
caccgaaacgcctctcccatacggg

SEQ ID NO: 13 - DNA, 25 bp, synthetic construct
  source 1..25: mol_type = other DNA
aaaccccgtatgggagaggcgtttc

SEQ ID NO: 14 - DNA, 25 bp, synthetic construct
  source 1..25: mol_type = other DNA
caccggtcctgaatgcggcctacct

SEQ ID NO: 15 - DNA, 25 bp, synthetic construct
  source 1..25: mol_type = other DNA
aaacaggtaggccgcattcaggacc

SEQ ID NO: 16 - DNA, 25 bp, synthetic construct
  source 1..25: mol_type = other DNA
caccgattcgtagaagtgaacgaat

SEQ ID NO: 17 - DNA, 25 bp, synthetic construct
  source 1..25: mol_type = other DNA
aaacattcgttcacttctacgaatc

SEQ ID NO: 18 - DNA, 25 bp, synthetic construct
  source 1..25: mol_type = other DNA
caccgagtgcaagtttaatgccgcc

SEQ ID NO: 19 - DNA, 25 bp, synthetic construct
  source 1..25: mol_type = other DNA
aaacggcggcattaaacttgcactc

SEQ ID NO: 20 - DNA, 23 bp, synthetic construct
  source 1..23: mol_type = other DNA
gcaaacgagaggaaaagcattaa

SEQ ID NO: 21 - DNA, 22 bp, synthetic construct
  source 1..22: mol_type = other DNA
gcagaccaggaacaacacttca

SEQ ID NO: 22 - DNA, 20 bp, synthetic construct
  source 1..20: mol_type = other DNA
ctgcagaggatgattgctga

SEQ ID NO: 23 - DNA, 20 bp, synthetic construct
  source 1..20: mol_type = other DNA
gaggaagtccagtgtccagc

SEQ ID NO: 24 - DNA, 20 bp, synthetic construct
  source 1..20: mol_type = other DNA
gtcaagtttgcctaccccca

SEQ ID NO: 25 - DNA, 20 bp, synthetic construct
  source 1..20: mol_type = other DNA
gagcccactgctccagaatg

SEQ ID NO: 26 - DNA, 20 bp, synthetic construct
  source 1..20: mol_type = other DNA
tgactggaagagcggagagt

SEQ ID NO: 27 - DNA, 20 bp, synthetic construct
  source 1..20: mol_type = other DNA
gttcgggctgatgtaccagt

SEQ ID NO: 28 - DNA, 20 bp, synthetic construct
  source 1..20: mol_type = other DNA
acagctggtgaacctggaag

SEQ ID NO: 29 - DNA, 20 bp, synthetic construct
  source 1..20: mol_type = other DNA
accaggagatccatctcgac

SEQ ID NO: 30 - DNA, 21 bp, synthetic construct
  source 1..21: mol_type = other DNA
ggccttggaagcatgtagagg

SEQ ID NO: 31 - DNA, 23 bp, synthetic construct
  source 1..23: mol_type = other DNA
ggagaactcgttagagacgactt

SEQ ID NO: 32 - DNA, 20 bp, synthetic construct
  source 1..20: mol_type = other DNA
aagtgctgccgtcattttct

SEQ ID NO: 33 - DNA, 20 bp, synthetic construct
  source 1..20: mol_type = other DNA
gtggcaatgatctcaacacg

SEQ ID NO: 34 - DNA, 20 bp, synthetic construct
  source 1..20: mol_type = other DNA
ccgtgtaagggtcaaagcat

SEQ ID NO: 35 - DNA, 20 bp, synthetic construct
  source 1..20: mol_type = other DNA
acaaggttcgggaagaggtt

SEQ ID NO: 36 - DNA, 20 bp, synthetic construct
  source 1..20: mol_type = other DNA
atgtttgtgatgggtgtgaa

SEQ ID NO: 37 - DNA, 20 bp, synthetic construct
  source 1..20: mol_type = other DNA
atgccaaagttgtcatggat

SEQ ID NO: 38 - DNA, 22 bp, synthetic construct
  source 1..22: mol_type = other DNA
tccacacatgtaccaacatcaa

SEQ ID NO: 39 - DNA, 20 bp, synthetic construct
  source 1..20: mol_type = other DNA
gtcacagtgccattccagtc

SEQ ID NO: 40 - DNA, 20 bp, synthetic construct
  source 1..20: mol_type = other DNA
gcctccggactttcgatctt

SEQ ID NO: 41 - DNA, 20 bp, synthetic construct
  source 1..20: mol_type = other DNA
taggagatgggttcccccag

SEQ ID NO: 42 - DNA, 22 bp, synthetic construct
  source 1..22: mol_type = other DNA
ccctgcagctggagagtgtgga

SEQ ID NO: 43 - DNA, 23 bp, synthetic construct
  source 1..23: mol_type = other DNA
tgtgctctgcttgtgaggtgctg

SEQ ID NO: 44 - DNA, 21 bp, synthetic construct
  source 1..21: mol_type = other DNA
gaagacagacctagacagcgg

SEQ ID NO: 45 - DNA, 21 bp, synthetic construct
  source 1..21: mol_type = other DNA
ccagtagcttcaccactcgac

SEQ ID NO: 46 - DNA, 23 bp, synthetic construct
  source 1..23: mol_type = other DNA
aattgtactctgggaaattgcca

SEQ ID NO: 47 - DNA, 21 bp, synthetic construct
  source 1..21: mol_type = other DNA
tctccaagattccgtccacag

SEQ ID NO: 48 - DNA, 22 bp, synthetic construct
  source 1..22: mol_type = other DNA
ggagttgtgaagtttggatggg

SEQ ID NO: 49 - DNA, 22 bp, synthetic construct
  source 1..22: mol_type = other DNA
attcccgcttctcctacaatcc

SEQ ID NO: 50 - DNA, 20 bp, synthetic construct
  source 1..20: mol_type = other DNA
ctaggcatggtggcagtgct

SEQ ID NO: 51 - DNA, 20 bp, synthetic construct
  source 1..20: mol_type = other DNA
ccgagccgactctgacatcc

SEQ ID NO: 52 - DNA, 20 bp, synthetic construct
  source 1..20: mol_type = other DNA
cgctgacatcgtgggactgg

SEQ ID NO: 53 - DNA, 20 bp, synthetic construct
  source 1..20: mol_type = other DNA
tcgaccctcatgacctggca

SEQ ID NO: 54 - DNA, 20 bp, synthetic construct
  source 1..20: mol_type = other DNA
tgtgaggaaggctgtgcatt

SEQ ID NO: 55 - DNA, 20 bp, synthetic construct
  source 1..20: mol_type = other DNA
ggtcaggttgcctctgtctc

SEQ ID NO: 56 - DNA, 21 bp, synthetic construct
  source 1..21: mol_type = other DNA
gatcgatgtggacgtttccaa

SEQ ID NO: 57 - DNA, 20 bp, synthetic construct
  source 1..20: mol_type = other DNA
atactgctggcgcacatcac

SEQ ID NO: 58 - DNA, 20 bp, synthetic construct
  source 1..20: mol_type = other DNA
gcttctcctcgctgcatttc

SEQ ID NO: 59 - DNA, 20 bp, synthetic construct
  source 1..20: mol_type = other DNA
gcagctggtcccattgaaga

SEQ ID NO: 60 - DNA, 21 bp, synthetic construct
  source 1..21: mol_type = other DNA
ttggtgctttagctgcattca

SEQ ID NO: 61 - DNA, 22 bp, synthetic construct
  source 1..22: mol_type = other DNA
cgccaattcctacacccaattc

SEQ ID NO: 62 - DNA, 21 bp, synthetic construct
  source 1..21: mol_type = other DNA
gcagcaccgtgaagacgaata

SEQ ID NO: 63 - DNA, 19 bp, synthetic construct
  source 1..19: mol_type = other DNA
atgcctgacagttcagcgg

SEQ ID NO: 64 - DNA, 19 bp, synthetic construct
  source 1..19: mol_type = other DNA
ctgggctacactgagcacc

SEQ ID NO: 65 - DNA, 21 bp, synthetic construct
  source 1..21: mol_type = other DNA
aagtggtcgttgagggcaatg

(19) United States
(12) Patent Application Publication
(10) Pub. No.: US 2024/0229043 A1
(43) Pub. Date: Jul. 11, 2024

(54) Methods and Compositions for Treating, Ameliorating, and/or Preventing Chronic Kidney Disease (CKD) and Complications thereof by Regulating DPEP1, CASP9, ACSS2 and/or FASN

(71) Applicant: THE TRUSTEES OF THE UNIVERSITY OF PENNSYLVANIA, Philadelphia, PA (US)

(72) Inventors: Katalin SUSZTAK, Princeton, NJ (US); Dhanunjay MUKHI, Philadelphia, PA (US)

(21) Appl. No.: 18/501,859

(22) Filed: Nov. 3, 2023

Related U.S. Application Data
(60) Provisional application No. 63/382,255, filed on Nov. 3, 2022.
(60) Provisional application No. 63/382,261, filed on Nov. 3, 2022.

(51) Int. Cl. C12N 15/113; A61K 31/502; A61K 45/06; A61P 13/12; G16B 20/20; G16H 50/20

(52) U.S. Cl. CPC C12N 15/1138; A61K 31/502; A61K 45/06; A61P 13/12; G16B 20/20; G16H 50/20; C12N 2310/14

(57) ABSTRACT

The present disclosure includes compositions and methods for treating, ameliorating, and/or preventing a chronic kidney disease and complications thereof. The method for treating CKD comprise administering a therapeutically effective amount at least one selected from the group consisting of DPEP1 inhibitor, a CASP9 inhibitor, a ACSS2 inhibitor, and a FASN inhibitor, or a salt, solvate, tautomer, enantiomer, diastereoisomer, geometric isomer, and/or any combination thereof, wherein administration prevents fibrosis and ferroptosis in the subject.

### CROSS-REFERENCE TO RELATED APPLICATIONS

The present application claims priority under 35 U.S.C. § 119(e) to U.S. Provisional Patent Application No. 63/382,255, filed Nov. 3, 2022, and U.S. Provisional Patent Application No. 63/382,261, file Nov. 3, 2022, both of which are incorporated herein by reference in their entireties.

### STATEMENT REGARDING FEDERALLY SPONSORED RESEARCH OR DEVELOPMENT

This invention was made with government support under DK087635 awarded by the National Institutes of Health. The government has certain rights in the invention.

### SEQUENCE LISTING

The XML text file named “046483-7389US1_Sequence Listing.xml” created on Oct. 29, 2023, comprising 56.6 Kbytes, is hereby incorporated by reference in its entirety.

### BACKGROUND

Chronic kidney disease (CKD) affects 800 million people worldwide and remains the tenth leading cause of deaths. Key molecular pathways that govern CKD pathogenesis remain largely unknown. Disease susceptibility shows substantial heterogeneity, which is thought to be explained by environmental and genetic risk factors that are yet to be fully elucidated. Genome-wide association analyses (GWAS) performed in large populations identified nearly 300 loci for genetic variants associated with kidney function. However, there remains an unmet need for identifying different target genes and developing methods for treating CKD. The present invention addresses this unmet need.

### SUMMARY

In some aspects, the present invention is directed to the following non-limiting embodiments:

In some aspects, the present invention is directed to a method for treating, ameliorating, and/or preventing a chronic kidney disease (CKD), and/or one or more complications thereof, in a subject in need thereof.

In some embodiments, the method comprises administering to the subject a therapeutically effective amount at least one selected from the group consisting of a DPEP1 inhibitor and a CASP9 inhibitor or a salt, solvate, tautomer, enantiomer, diastereoisomer, geometric isomer, and/or any combination thereof.

In some embodiments, the administering prevents fibrosis in a subject.

In some embodiments, the administering prevents ferroptosis in a subject.

In some embodiments, the subject is a mammal.

In some embodiments, the subject is a human subject.

In some aspects, the present invention is directed to a pharmaceutical composition for treating, ameliorating, and/or preventing a chronic kidney disease (CKD), and/or one or more complications thereof, in a subject in need thereof.

In some embodiments, the composition comprises a therapeutically effective amount of at least one selected from the group consisting of a DPEP1 inhibitor and a CASP9 inhibitor or a salt, solvate, tautomer, enantiomer, diastereoisomer, geometric isomer, and/or any combination thereof.

In some embodiments, the composition further comprises a pharmaceutically acceptable carrier or an adjuvant.

In some aspects, the present invention is directed to a kit.

In some embodiments, the kit comprises the pharmaceutical composition herein and instruction material for use thereof.

In some embodiments, the instructional material comprises instructions for treating CKD in a subject.

In some embodiments, the treating includes administering the pharmaceutical composition to the subject.

In some aspects, the present invention is directed to a method for treating, ameliorating, and/or preventing a chronic kidney disease (CKD), and/or one or more complications thereof, in a subject in need thereof.

In some embodiments, the method comprises identifying a target gene responsible for CKD by integrating evidence from omics datasets and analytical tools selected from the group consisting of eQTLs, meQTLs and eQTM, coloc (GWAS and eQTLs), moloc (GWASs, eQTLs and meQTLs), SMR, HEIDI, single-cell; co-accessibility and activity-by-contact (ABC) model.

In some embodiments, the method further comprises administering to the subject a therapeutically effective amount of an agent for modulating expression of the identified target gene.

In some embodiments, the identified target gene is DPEP1 and the agent is a DPEP1 inhibitor.

In some embodiments, the identified target gene is CASP9 and the agent is a CASP9 inhibitor.

In some aspects, the present invention is directed to a method for treating, ameliorating, and/or preventing a chronic kidney disease (CKD), and/or one or more complications thereof, in a subject in need thereof.

In some embodiments, the method comprises administering to the subject a therapeutically effective amount of a ACSS2 inhibitor or a FASN inhibitor.

In some embodiments, the administering prevents fibrosis in the subject.

In some embodiments, the subject is a mammal.

In some embodiments, the subject is a human subject.

In some aspects, the present invention is directed to a pharmaceutical composition for treating, ameliorating, and/or preventing a chronic kidney disease (CKD), and/or one or more complications thereof, in a subject in need thereof.

In some embodiments, the composition comprises a therapeutically effective amount of an ACSS2 inhibitor or a FASN inhibitor.

In some embodiments, the composition further comprises a pharmaceutically acceptable carrier or an adjuvant.

In some aspects, the present invention is directed to a kit.

In some embodiments, the kit comprises the pharmaceutical composition of herein, and instruction material for use thereof.

In some embodiments, the instructional material comprises instructions for treating CKD in a subject.

In some embodiments, the treating includes administering the pharmaceutical composition to the subject.

In some embodiments, the subject is a human subject.

In some aspects, the present invention is directed to a method for treating, ameliorating, and/or preventing a chronic kidney disease (CKD), and/or one or more complications thereof, in a subject in need thereof.

In some embodiments, the method comprises identifying a target gene responsible for CKD by integrating evidence from omics datasets and analytical tools selected from the group consisting of eQTLs, meQTLs and eQTM, coloc (GWAS and eQTLs), moloc (GWASs, eQTLs and meQTLs), SMR, HEIDI, single-cell; co-accessibility and activity-by-contact (ABC) model.

In some embodiments, administering to the subject a therapeutically effective amount of an agent for modulating expression of the identified target gene.

In some embodiments, the identified target gene is ACSS2 and the agent is a an ACSS2 inhibitor.

In some embodiments, the identified target gene is FASN and the agent is a an FASN inhibitor.

### BRIEF DESCRIPTION OF THE DRAWINGS

The following detailed description of specific embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIGS. 1A-1C show shared genetic variants associated with kidney function, human kidney methylation, and DPEP1/CHMP1A expression. FIG. 1A: LocusZoom plots of eGFR GWAS, human kidney mQTL analysis (genotype-methylation, n=188), eQTLs (genotype-expression of DPEP1) in kidney compartments (tubule n=121 or glomerulus n=119). FIG. 1B: LocusZoom plots of eGFR GWAS, human kidney mQTL analysis (genotype-methylation, n=188), eQTLs (genotype-expression of CHMP1A) in kidney compartments (tubule n=121 or glomerulus n=119). The x-axis indicates the genomic location on chromosome 16. The arrow indicates the transcriptional direction for specific genes. Each dot represents one SNP. The dots are colored according to their correlation to the index SNP (rs164748). Some dots indicate strong correlation (r²>0.8) (LD) with the index SNP. The left y-axis indicates −log₁₀ (P value). The right y-axis indicates recombination rate (cM/Mb). FIG. 1C: Genotype (rs164748) and gene expression (DPEP1 and CHMP1A) association in human tubules (n=121) and glomeruli (n=119) from database. The effect size estimate (Beta) and standard error (SE) are as below: DPEP1 tubule Beta=0.811 and SE=0.11; DPEP1 glom Beta=0.889 and SE=0.11; CHMP1A tubule Beta=−0.766 and SE=0.113; CHMP1A glom Beta=−0.587 and SE=0.127. Centerlines show the medians; box limits indicate the 25th and 75th percentiles; whiskers extend to the 5th and 95th percentiles. P value was calculated.

FIGS. 2A-2C show fine mapping of the GWAS locus via human kidney single nuclei ATAC-seq analysis coupled with genome editing. FIG. 2A: From top to bottom: locuszoom plots of eGFR GWAS; Gene browser view of the single nucleotide polymorphisms within the regions; genome browser view of chromatin accessibility for proximal tubules (PT), loop of Henle (LOH), distal convoluted tubule (DCT), collecting duct principal cell types (PC), collecting duct intercalated cells (IC), podocytes (Podo), endothelial cells (Endo), immune cells (Immune); genome browser view of whole kidney H3K27ac, H3K4me1, and H3K4me3 histone ChIP-seq; ChomHMM annotation human adult and fetal kidneys. Proximal tubule-specific open chromatin region across this region was circled and numbered. FIG. 2B Schematic of CRISPR/Cas9 mediated open chromatin region deletion. FIG. 2C: Relative transcript levels of DPEP1 and CHMP1A following open chromatin region deletion (n=4). All data are represented as mean±SEM. P value was calculated by one-way ANOVA with post hoc Tukey test. P<0.05 is statistically significant.

FIGS. 3A-3L show Dpep1 deficiency ameliorated but Chmp1a haploinsufficiency exacerbated renal injury in mice. FIG. 3A: Serum blood urea nitrogen (BUN) and creatinine measurement of control and Dpep1+/− mice following sham or cisplatin injection. Sham-treated group: WT (n=4), Dpep1+/− (n=4); cisplatin-treated group: WT (n=7), Dpep1+/− (n=6). FIG. 3B: Representative images of HE-stained kidney sections from control and Dpep1+/− mice following sham or cisplatin injection. Scale bar: 20 μm. FIG. 3C: Relative mRNA level of injury markers Kim1 and Lcn2 in the kidneys of control and Dpep1+/− mice following sham or cisplatin injection. Sham-treated group: WT (n=4), Dpep1+/− (n=4); cisplatin-treated group: WT (n=7), Dpep1+/− (n=6). FIG. 3D: Serum BUN and creatinine measurement of control and Dpep1+/− mice following sham or folic acid (FA) injection. Sham-treated group: WT (n=3), Dpep1+/− (n=3); cisplatin-treated group: WT (n=5), Dpep1+/− (n=7). FIG. 3E: Representative images of HE- and Sirius Red-stained kidney sections from control and Dpep1+/− mice following sham or FA injection. Scale bar: 20 μm. FIG. 3F: Western blots of fibrosis markers aSMA, Collagen, and Fibronectin in kidneys of control and Dpep1+/− mice following sham or FA injection. FIG. 3G: Serum BUN and creatinine measurement of control and Chmp1a^{+/-} mice following sham or cisplatin injection. Sham-treated group: WT (n=3), Dpep1+/− (n=3); cisplatin-treated group: WT (n=4), Dpep1+/−(n=5). FIG. 3H: Representative images of HE-stained kidney sections from control and Chmp1a^{+/-} mice following sham or cisplatin injection. Scale bar: 20 μm. FIG. 3I: Relative transcript level of injury markers Kim1 and Lcn2 in control and Chmp1a^{+/-} mice following sham or cisplatin injection. Sham-treated group: WT (n=3), Dpep1+/−(n=3); cisplatin-treated group: WT (n=4), Dpep1+/−(n=5). FIG. 3J: Serum BUN and creatinine levels of control and Chmp1a^{+/-} mice following sham or FA injection (n=3 per group). FIG. 3K: Representative images of HE- and Sirius Red-stained kidney sections from control and Chmp1a^{+/-} mice following sham or FA injection. Scale bar: 20 μm. FIG. 3L: Western blots of fibrosis markers aSMA, Collagen3, and Fibronectin in control and Chmp1a^{+/-} mice following sham or FA injection. All data are represented as mean±SEM. P value was calculated by two-way ANOVA with post hoc Tukey test. P<0.05 is statistically significant.

FIG. 4A-4N show Dpep1 deficiency protects from cisplatin-induced injury without affecting necroptosis or pyroptosis. FIG. 4A: Relative mRNA level of Dpep1 in scramble siRNA (siControl) and Dpep1 siRNA (siDpep1) transfected tubule cell (n=3). FIG. 4B Western blots of DPEP1 and CHMP1A in scramble and Dpep1 siRNA transfected tubule cell. FIG. 4C LDH level of NRK52E cell treated with varying dose of cisplatin for varying degree of time (n=3). FIG. 4D The percentage of viable cells following siControl and siDpep1 transfection and in the presence and absence of cisplatin treatment (n=3). FIG. 4E LDH level of following siControl and siDpep1 transfection and sham or cisplatin treatment (n=3). FIG. 4F The ratio of cell-impermeable peptide substrate bis-AAF-R110 (dead cell indicator) to cell-permeable GF-AFC substrate (live cell indicator) from siControl and siDpep1 transfected cell following sham or cisplatin treatment (n=3). FIG. 4G: Relative transcript level of Ripk1 and Mk of siControl and siDpep1 transfected cell following sham or cisplatin treatment (n=3). FIG. 4H: Relative transcript level of Ripk1 in kidneys of folic acid and cisplatin-treated wild-type and Dpep1^{+/-} mice. Sham-treated group: WT (n=3), Dpep1^{+/-} (n=3); FA-treated group: WT (n=5), Dpep1^{+/-} (n=7); cisplatin-treated group: WT (n=7), Dpep1^{+/-} (n=6). FIG. 4I: Relative transcript level of Mk in kidneys of folic acid and cisplatin-treated wild-type and Dpep1^{+/-} mice. Sham-treated group: WT (n=3), Dpep1^{+/-} (n=3); FA-treated group: WT (n=5), Dpep1^{+/-} (n=7); cisplatin-treated group: WT (n=7), Dpep1^{+/-} (n=6). FIG. 4J: Relative transcript level of Nlrp3 and Il1beta from siControl and siDpep1 transfected cell following sham or cisplatin treatment (n=3). FIG. 4K: Relative transcript level of Nlrp3 and Il1beta in kidneys of folic acid and cisplatin-treated wild-type and Dpep1^{+/-} mice. Sham-treated group: WT (n=3), Dpep1^{+/-} (n=3); FA-treated group: WT (n=5), Dpep1^{+/-} (n=7); cisplatin-treated group: WT (n=7), Dpep1^{+/-} (n=6). FIG. 4L Western blots of RIPK3 and cleaved caspase 1 in kidneys of wild-type and Dpep1^{+/-} mice following sham or cisplatin treatment. FIG. 4M LDH level of siControl and siDpep1 transfected cell following sham or Nigericin treatment (n=3). FIG. 4N Relative mRNA level of Ly6G in kidneys of control, folic acid and cisplatin-treated wild-type and Dpep1^{+/-} mice. Sham-treated group: WT (n=3), Dpep1^{+/-} (n=3); FA-treated group: WT (n=5), Dpep1^{+/-} (n=7); cisplatin-treated group: WT (n=7), Dpep1^{+/-} (n=6). All data are represented as mean±SEM. P value was calculated by two-way ANOVA with post hoc Tukey test for FIGS. 4C-4N. P value was calculated by two-tailed t-test for FIG. 4A. P<0.05 is statistically significant.

FIGS. 5A-5L show Chmp1a sensitizes for cisplatin-induced cell death without altering necroptosis, pyroptosis, and apoptosis. FIG. 5A Relative mRNA level of Chmp1a in scramble siRNA (siControl) and Chmp1a siRNA (siChmp1a) transfected kidney tubule cell (n=3). FIG. 5B Western blots of CHMP1A and DPEP1 from siControl and siChmp1a transfected cells. FIG. 5C Viability of siControl and siChmp1a transfected tubule cell following sham or cisplatin treatment (n=3). FIG. 5D LDH level of siControl and siChmp1 transfected tubule cell following sham or cisplatin treatment (n=3). FIG. 5E The ratio of cell-impermeable peptide substrate bis-AAF-R110 (dead cell indicator) to cell-permeable GF-AFC substrate (live cell indicator) from siControl siChmp1a transfected cell following sham or cisplatin treatment (n=3). FIG. 5F Relative mRNA level of Ripk1 and Mlkl of siControl and siChmp1a transfected cell following sham or cisplatin treatment (n=3). FIG. 5G Relative mRNA level of Ripk1 and Mlkl in kidneys of folic acid and cisplatin-treated wild-type and Chmp1a^{+/-} mice. Sham-treated group: WT (n=3), Chmp1a^{+/-} (n=3); FA-treated group: WT (n=5), Chmp1a^{+/-} (n=5); cisplatin-treated group: WT (n=4), Chmp1a^{+/-} (n=5). FIG. 5H Relative mRNA level of Nlrp3 and Il1beta of siControl and siChmp1a transfected cell following sham or cisplatin treatment (n=3). FIG. 5I Relative mRNA level of Nlrp3 and Il1beta in kidneys of folic acid and cisplatin-treated wild-type and Chmp1a^{+/-} mice. Sham-treated group: WT (n=3), Chmp1a^{+/-} (n=3); FA-treated group: WT (n=5), Chmp1a^{+/-} (n=5); cisplatin-treated group: WT (n=4), Chmp1a^{+/-} (n=5). FIG. 5J (left) Representative cleaved Caspase 3 staining of siControl and siChmp1a transfected cell following sham or cisplatin treatment. (right) Quantification of cleaved Caspase 3 positive cell (n=5). Scale bar: 20 μm. FIG. 5K Relative mRNA transcript of Bak and Bax in kidneys of folic acid and cisplatin-treated wild-type and Chmp1a^{+/-} mice. Sham-treated group: WT (n=3), Chmp1a^{+/-} (n=3); FA-treated group: WT (n=5), Chmp1a^{+/-} (n=5); cisplatin-treated group: WT (n=4), Chmp1a^{+/-} (n=5). FIG. 5L: LDH level of siControl and siChmp1a transfected cell with or without cisplatin and necroptosis (Nec1), pyroptosis (Vx765), and apoptosis (Z-VAD-FMK) inhibitors (n=3). All data are represented as mean±SEM. P value was calculated by two-way ANOVA with post hoc Tukey test for FIG. 5C-5L. P value was calculated by two-tailed t-test for a. P<0.05 is statistically significant.

FIGS. 6A-6J show Dpep1 knockdown ameliorated cisplatin-induced apoptosis and ferroptosis. FIG. 6A: (Left) Representative cleaved caspase 3 staining of siControl and siDpep1 cell following sham or cisplatin treatment. (Right) Quantification of positive cleaved caspase 3 cell (n=7). Scale bar: 20 μm. FIG. 6B: Relative mRNA transcript of Bax and Bak in kidneys of folic acid and cisplatin-treated wild-type and Dpep1^{+/-} mice. Sham-treated g group: WT (n=3), Dpep1^{+/-} (n=3); FA-treated group: WT (n=5), Dpep1^{+/-} (n=7); cisplatin-treated group: WT (n=7), Dpep1^{+/-} (n=6). FIG. 6C: (Left) LDH level of siControl and siDpep1 transfected cell with or without apoptosis activator camptothecin (CPT) treatment. (Right) The ratio of cell-impermeable peptide substrate bis-AAF-R110 (dead cell indicator) to cell-permeable GF-AFC substrate (live cell indicator) of siControl and siDpep1 transfected cell with or without CPT treatment (n=3). FIG. 6D: (left) Representative BODIPY 581/591 C11 fluorescence of siControl and siDpep1 transfected cell following sham or cisplatin treatment. (right) Quantification of the oxidized vs. reduced probe (n=5). Scale bar: 50 μm. FIG. 6E: Ferrous iron concentration (normalized to kidney weight) in kidneys of cisplatin-treated wild-type and Dpep1^{+/-} mice. Sham-treated group: WT (n=3), Dpep1^{+/-} (n=3); cisplatin-treated group: WT (n=7), Dpep1^{+/-} (n=6). FIG. 6F: Relative mRNA level of Acsl4 in kidneys of folic acid and cisplatin-treated wild-type and Dpep1^{+/-} mice. Sham-treated group: WT (n=3), Dpep1^{+/-} (n=3); FA-treated group: WT (n=5), Dpep1^{+/-} (n=7); cisplatin-treated group: WT (n=7), Dpep1^{+/-} (n=6). FIG. 6G: Representative images of ACSL4 immunostaining in kidney sections of folic acid-treated wild-type and Dpep1^{+/-} mice. Scale bar: 10 μm. FIG. 6H: Western blots of ACSL4 kidneys of folic acid-treated wild-type and Dpep1^{+/-} mice. FIG. 6I: LDH level of scramble siControl and siDpep1 transfected cell following sham or ferroptosis activator Erastin, FIN56, FINO2, and RSL3 treatment (n=3). FIG. 6J: (Left) Representative images of transferrin uptake of siControl and siDpep1 transfected cells. (Right) Quantification of arbitrary fluorescence unit of transferrin in siControl and siDpep1 transfected cell (n=10). Scale bar: 50 μm. All data are represented as mean±SEM. P value was calculated by two-way ANOVA with post hoc Tukey test for FIG. 6A-6I was calculated by two-tailed t-test for FIG. 6J: P<0.05 is statistically significant.

FIGS. 7A-7I show Chmp1a knockdown enhanced ferroptosis through iron accumulation. FIG. 7A: (left) Representative BODIPY 581/591 C11 staining of siControl and siChmp1a transfected cell following sham or cisplatin treatment. Scale bar: 50 μm. (right) Quantification of the oxidized vs. reduced probe (n=5). FIG. 7B: Relative mRNA level of Acsl4 in the kidneys of folic acid and cisplatin-treated wild-type and Chmp1a^{+/-} mice. Sham-treated group: WT (n=3), Chmp1a^{+/-} (n=3); FA-treated group: WT (n=5), Chmp1a^{+/-} (n=5); cisplatin-treated group: WT (n=4), Chmp1a^{+/-} (n=5). FIG. 7C: Western blots of ACSL4 in kidneys of folic acid-treated wild-type and Chmp1a^{+/-} mice. FIG. 7D: Representative images of ACSL4 immunostaining of kidney sections from folic acid-treated wild-type and Chmp1a^{+/-} mice. Scale bar: 10 μm. FIG. 7E: LDH level of siControl and siChmp1a transfected cell with or without cisplatin and ferroptosis inhibitor liproxstatin1 (n=3). FIG. 7F: (upper) Western blots of CD63 of kidney exosomes of wild-type and Chmp1a^{+/-} mice. (bottom) Quantification of three independent experiments (n=3). FIG. 7G: Ferrous iron concentrations (normalized to kidney weight) of kidneys of cisplatin-treated wild-type and Chmp1a^{+/-} mice. Sham-treated group: WT (n=3), Chmp1a^{+/-} (n=3); cisplatin-treated group: WT (n=4), Chmp1a^{+/-} (n=5). FIG. 7H: Serum creatinine level of control and Chmp1a^{+/-} mice with or without cisplatin and liproxstatin injection. Sham-treated group: WT (n=3), Chmp1a^{+/-} (n=3); cisplatin-treated group: WT (n=5), Chmp1a^{+/-} (n=5); cisplatin and liproxstatin-treated group: WT (n=4), Chmp1a^{+/-} (n=4). FIG. 7I: Relative transcript level of injury marker Kim1 in kidneys of control and Chmp1a^{+/-} mice with or without cisplatin and liproxstatin injection. Sham-treated group: WT (n=3), Chmp1a^{+/-} (n=3); cisplatin-treated group: WT (n=5), Chmp1a^{+/-} (n=5); cisplatin and liproxstatin-treated group: WT (n=4), Chmp1a^{+/-} (n=4). All data are represented as mean±SEM. P value was calculated by two-way ANOVA with post hoc Tukey test for FIGS.: A, B, E, G, H, I. P value was calculated by two-tailed t-test for FIG. F: P<0.05 is statistically significant.

FIGS. 8A-8E show DPEP1 and CHMP1A levels correlate and associated with ferroptosis gene expression in human kidney samples. FIG. 8A: Relative expression of DPEP1 (y-axis) and CHMP1A (x-axis) in 432 microdissected human kidney tubule samples. Pearson correlation is shown. Student t-test based on the Pearson correlation coefficient was used to calculate the statistical significance of the association. FIG. 8B: Genes showing significant correlation with DPEP1 and CHMP1A in microdissected human kidney tubule samples. y-axis represents the Pearson correlation P value. FIG. 8C: Relative transcript levels of ACSL4, ACSL3, and SLC3A2 (FPKM, y-axis), and kidney function (eGFR, x-axis) or kidney fibrosis (x-axis) as analyzed in 432 microdissected human kidney samples. Pearson correlation is shown. Student's t-test based on the Pearson correlation coefficient was used to calculate the statistical significance of the association. FIG. 8D: Representative images of ACSL4 immunostaining in healthy control and CKD kidney samples. Scale bar: 20 μm. FIG. 8E: Western blots of ACSL4 in healthy control and CKD kidney samples.

FIGS. 9A-9D show genotype-phenotype (GWAS) and genotype-gene expression (eQTL) association analysis at the chromosome 16 eGFR GWAS locus. FIG. 9A: LocusZoom plots of chromosome 16 regions of eGFR GWAS, using the CKDGen 2016 release (upper), the CKDGen 2019 release (middle), and the Million Veteran Program (bottom). FIG. 9B: The association of genotype (rs164748) and CHMP1A, DPEP1, CDK10, SPATA33 expression in human tubules (n=166) and glomeruli (n=136) in the NephQTL database1 (http://nephqtl.org). Centerlines show the medians; box limits indicate the 25th and 75th percentiles; whiskers extend to the 5th and 95th percentiles; outliers are represented by dots. FIG. 9C: LocusZoom plots of eGFR GWAS (top), CDK10 eQTL (middle) and SPATA33 eQTL (bottom) analysis in tubules (n=121) around the region of rs164748. The x-axis shows the chromosomal location. The y axis shows −log 10(P) of association tests (by linear regression). FIG. 9D: The association of genotype (rs164748) and gene expression (CDK10 and SPATA33) in human tubules (n=121) eQTL database (www.susztaklab.com). Centerlines show the medians; box limits indicate the 25th and 75th percentiles; whiskers extend to the 5th and 95th percentiles. P-value was calculated by linear regression.

FIGS. 10A-10D show Cicero-inferred co-accessibility of open chromatin regions in mouse Dpep1 and Chmp1a locus and conditional eGFR GWAS analysis. FIG. 10A: From top to bottom: (top) Cicero-inferred co-accessibility of open chromatin regions in adult mouse kidney. (bottom) Genome browser view of chromatin accessibility of nephron progenitors (NP), podocytes (Podo), proximal tubules segment 1 and 2 (PT S1 and PT S2), the loop of Henle (LOH), distal convoluted tubule (DCT), collecting duct principal cell types (PC), collecting duct intercalated cells (IC), endothelial cells (Endo), immune cells (Immune) and stromal cells (stroma). FIGS. 10B-10D: LocusZoom plots of conditional eGFR GWAS analysis of snATAC-seq harbored multiple SNPs. Peak locations of 1-12 are shown in FIG. 2A.

FIGS. 11A-11C show Genome editing-based fine mapping of the kidney function GWAS region. FIG. 11A: CRISPR/Cas9 mediated deletion of the genomic region. FIGS. 11B-11C: Genome Brower view of transcription factor binding in the genomic regions of 8, 9 and 12. The sequences shown in FIG. 11A are listed in the following table:

Peak 8-WT
AGCAGCAGGTGCGGGTGGCACCCTACAGGCCCTCTCAGCCG (SEQ ID NO: 1)
Peal 8-deletion
AGCAGCAGGTGCGGGTGGCACCCTGCTGGCCGNCANTTAGCAG (SEQ ID NO: 2)
Peak 9-WT
GGCCGCCTGCGTCCTCAGGCCAAGGAGTAAAGTCTCCTGGG (SEQ ID NO: 3)
Peak 9-deletion
GGCCGCCTGCGCCAGGGGAGGTCAGGAACGACTGGGACCCA (SEQ ID NO: 4)
Peak 12-WT
AGCACTGCCATTGCCAGCAGTCCATCCTACCTCGCATCCAG (SEQ ID NO: 5)
Peak 12-deletion
AGCACTGCCATTGGGCAGGCTCAGCAGTTTCACAGGTCTCA (SEQ ID NO: 6)

FIGS. 12A-12J show generation of Dpep1 knock-out animals. FIG. 12A: The guide RNA targeting region at the mouse Dpep1 locus. FIG. 12B: Electrophoresis image of Dpep1 founder lines genotyping. FIG. 12C: Nucleotide deletion information for the #5 founder line. FIG. 12D: Relative mRNA level of Dpep1 in kidneys of wildtype, Dpep1 heterozygous and homozygous mice (n=4 per group). FIG. 12E: Western blots of DPEP1 from kidneys of wildtype, Dpep1 heterozygous and homozygous mice. FIG. 12F: Tubular injury scores in kidneys of control and Dpep+/− mice following sham or cisplatin injection. Sham-treated group: WT (n=4), Dpep1+/− (n=4); cisplatin-treated group: WT (n=7), Dpep1+/− (n=6). FIG. 12G: Relative mRNA level of immune cell markers Cd68 and Ccl2 in the kidneys of control and Dpep+/− mice following sham or cisplatin injection. Sham-treated group: WT (n=4), Dpep+/−(n=4); cisplatin-treated group: WT (n=7), Dpep+/−(n=6). FIG. 12H: Quantification of Sirius Red-stained kidney sections from control and Dpep+/− mice following sham or folic acid injection (n=7 per group). FIG. 12I: Relative transcript levels of fibrosis markers in kidneys of control and Dpep+/− mice following sham or folic acid injection. Sham-treated group: WT (n=3), Dpep1+/−(n=3); FA-treated group: WT (n=5), Dpep1^{+/-} (n=7). FIG. 12J: Relative mRNA levels of fibrosis markers in kidneys of control and Dpep+/− mice following sham or UUO procedure. Sham-treated group: WT (n=5), Dpep1^{+/-} (n=5); UUO-treated group: WT (n=3), Dpep+/−(n=3). All data are represented as mean±SEM. P-value was calculated by one-way or two-way ANOVA with post hoc Tukey test. P<0.05 is statistically significant. The sequences shown in FIGS. 12A and 12C are listed in the following table:

sgRNA1
GAGACCAGATGCTGACTTGAACAAGCTAGC (SEQ ID NO: 7)
sgRNA2
AAGCTGAAGGCTGGCTTTGTCGGAGGCCA (SEQ ID NO: 8)
#5 Founder WT
GATGCTGACTTGAACAAGCTAGCCCAAACACACACCAACATCCCCAAGCTGAA
GGCTGGCTTTGTCGG (SEQ ID NO: 9)
#5 Founder −62 bp
GATCGG (SEQ ID NO: 10)
#5 Founder −16 bp
GATGATAACTTGAACAAGCTAGCCCAAACACACACCAACATCCCCAAGCTGG
(SEQ ID NO: 11)

FIGS. 13A-13D show Dpep1 knock-out mice are protected from cisplatin-induced renal injury. FIG. 13A: Serum BUN and creatinine measurement of control and Dpep1^{-/-} mice following sham or cisplatin injection. Sham-treated group: WT (n=4), Dpep1^{-/-} (n=4); cisplatin-treated group: WT (n=7), Dpep1^{-/-} (n=7). FIG. 13B: Representative images of HE-stained kidney sections from control and Dpep1^{-/-} mice following sham or cisplatin injection. Scale bar: 20 μm. FIG. 13C: Relative mRNA level of injury markers Kim1 and Lcn2, and immune cell markers Cd68 and Ccl2 in kidneys of control and Dpep1^{-/-} mice following sham or cisplatin injection. Sham treated group: WT (n=4), Dpep1^{-/-} (n=4); cisplatin-treated group: WT (n=7), Dpep1^{-/-} (n=7). FIG. 13D: TUNEL staining of kidney sections of control and Dpep1^{-/-} mice following sham or cisplatin injection. Quantification of TUNEL positive cells per field in cortex and medulla (n=3 per group). Scale bar: 20 μm. All data are represented as mean±SEM. P-value was calculated two-way ANOVA with post hoc Tukey test. P<0.05 is statistically significant.

FIGS. 14A-14G show Chmp1a haploinsufficiency exacerbates kidney diseases.

FIG. 14A: Relative mRNA level of Chmp1a in kidney tissue of wildtype (n=3) and Chmp1a+/− mice (n=5). FIG. 14B: Western blots of CHMP1A of kidney tissue of wildtype and Chmp1a+/− mice. FIG. 14C: Tubular injury scores of kidney sections of control and Chmp1a+/− mice following sham or cisplatin injection. Sham-treated group: WT (n=3), Chmp1a+/− (n=3); cisplatin-treated group: WT (n=4), Chmp1a+/− (n=5). FIG. 14D: Relative transcript levels of immune cell markers Cd68 and Ccl2 in kidneys of control and Chmp1a+/− mice following sham or cisplatin injection. Sham-treated group: WT (n=3), Chmp1a+/− (n=3); cisplatin-treated group: WT (n=4), Chmp1a+/− (n=5). FIG. 14E: Quantification of Sirius Red-stained kidney sections of control and Chmp1a+/− mice following sham or folic acid injection. Sham-treated group: WT (n=5), Chmp1a+/− (n=6); FA-treated group: WT (n=9), Chmp1a+/− (n=9). FIG. 14F: Relative transcript levels of fibrosis markers in kidneys of control and Chmp1a+/− mice following sham or folic acid injection. Sham-treated group: WT (n=3), Chmp1a+/− (n=3); FAtreated group: WT (n=5), Chmp1a+/− (n=5). FIG. 14G: Relative transcript levels of fibrosis markers in kidneys of control and Chmp1a+/− mice following sham or UUO procedure (n=3 per group). All data are represented as mean±SEM. P-value was calculated by two-tailed t-test or two-way ANOVA with post hoc Tukey test. P<0.05 is statistically significant.

FIGS. 15A-15C show characterization of DPEP1 expression in mouse kidney tissue and cells. FIG. 15A: Representative images of double staining of DPEP1 and kidney segment markers LTL, AQP2, or DBA in healthy mouse kidney samples. Scale bar: 50 μm. FIG. 15B: Relative Dpep1 expression in mouse kidney single-cell RNA sequencing dataset. FIG. 15C: Representative images of double staining of DPEP1 and early endosome marker RAB5, late endosome marker RAB7, recycling endosome marker RAB11, Golgi marker GM130, and coated vesicles marker clathrin in rat epithelial NRK52E cell. Scale bar: 10 μm.

FIGS. 16A-16B show characterization of CHMP1A expression in kidney tissue samples and cells. FIG. 16A: Representative images of double staining of CHMP1A and kidney segment markers LTL, PNA, AQP2, or DBA in healthy mouse kidney samples. Scale bar: 20 μm. FIG. 16B: Representative images of double staining of CHMP1A and early endosome marker EEA1, RAB5, late endosome marker RAB7 and VAMP7, and recycling endosome marker RAB11 in rat epithelial NRK52E cell. Scale bar: 10 μm.

FIGS. 17A-17E show Dpep1 knockdown is associated with lower lipid peroxidation. FIG. 17A: Representative mitoSOX staining of siControl and siDpep1 transfected cells following sham or cisplatin treatment. Scale bar: 10 μm. FIG. 17B: Relative mRNA level of Acsl4 from siControl and siDpep1 transfected cell following sham or cisplatin treatment (n=3). FIG. 17C: Representative BODIPY 581/591 C11 staining of wildtype and wildtype and Dpep1+/− primary kidney tubule cell following sham or cisplatin treatment. Scale bar: 50 μm. FIG. 17D:LDH level of wildtype and Dpep1+/− primary kidney tubule cell with or without cisplatin, erastin, camptothecin, and nigericin treatment (n=3). FIG. 17E: Representative images of dextran uptake from siControl and siDpep1 transfected cells. Scale bar: 50 μm. All data are represented as mean±SEM. P-value was calculated by two-way ANOVA with post hoc Tukey test. P<0.05 is statistically significant.

FIGS. 18A-18C show the effect of Dpep1 loss on GSH and GPX4 levels. FIG. 18A: The total and free GSH concentration in kidneys of wildtype and Dpep1^{-/-} mice treated with or without cisplatin (n=5 per group). FIG. 18B: Relative mRNA level of Gpx4 in kidneys of control and Dpep1−/− mice following sham or cisplatin injection. Sham-treated group: WT (n=4), Dpep1−/− (n=4); cisplatin-treated group: WT (n=6), Dpep1−/− (n=6). FIG. 18C: Western blots of GPX4 in kidneys of control and Dpep−/− mice following sham or cisplatin injection (n=6 per group). All data are represented as mean±SEM. P-value was calculated by two-way ANOVA with a post hoc Tukey test. P<0.05 is statistically significant.

FIGS. 19A-19B show overexpression of DPEP1 is cytotoxic to kidney tubule cells.

FIG. 19A: LDH level of vector-transfected and Dpep1-overexpressing vector-transfected cell with or without cisplatin treatment (n=6). FIG. 19B: Representative BODIPY 581/591 C11 labeling of vector-transfected and Dpep1-overexpressing vector-transfected cell with or without cisplatin treatment. Scale bar: 20 μm. All data are represented as mean±SEM. P-value was calculated by two-way ANOVA with post hoc Tukey test. P<0.05 is statistically significant.

FIGS. 20A-20E show haploinsufficiency of Chmp1a increases cytotoxicity of kidney tubule cells. FIG. 20A: Relative mRNA level of Acsl4 in siControl and siChmp1a transfected tubule cell following sham or cisplatin treatment (n=3). FIG. 20B: Representative mitoSOX staining of siControl and siChmp1a transfected tubule cell following sham or cisplatin treatment. Scale bar: 10 μm. FIG. 20C: LDH level of primary kidney tubule cells isolated from wildtype and Chmp1a^{+/-} mice treated with or without Mito-TEMPO in the presence or absence of cisplatin (n=3). FIG. 20D: Representative BODIPY 581/591 C11 labeling of primary tubule cells isolated from wildtype and Chmp1a^{+/-} mice following sham or cisplatin treatment with or without liproxstatin1 or Mito-TEMPO. Scale bar: 20 μm. FIG. 20E: LDH level of primary kidney tubule cells isolated from wildtype and Chmp1a^{+/-} mice treated with or without cisplatin, erastin, camptothecin, and nigericin (n=3). All data are represented as mean±SEM. P-value was calculated by two-way ANOVA with a post hoc Tukey test. P<0.05 is statistically significant.

FIGS. 21A-21B show the expression of DPEP1 and CHMP1A in mouse kidney samples. FIG. 21A: Relative expression of Dpep1 (x-axis) and Chmp1a (y-axis) in kidneys of control and UUO, FA, APOL1, and PGC1a mouse kidney disease models (as analyzed by RNAseq). Pearson correlation is shown. Student t-test based on the Pearson correlation coefficient was used to calculate the statistical significance of the association. FIG. 21B: Relative transcript levels of CHMP1A and DPEP1 (y-axis), and kidney function (eGFR, xaxis) kidney fibrosis (x-axis) as analyzed in 432 microdissected human kidney samples. Pearson correlation is shown. Student t-test based on the Pearson correlation coefficient was used to calculate the statistical significance of the association.

FIG. 22 shows gene ontology analysis (DAVID) of genes whose levels correlated with CHMP1A and DPEP1 in human kidney samples. Genes that were correlated with CHMP1A and DPEP1 expression level in the RNA-seq data of 432 microdissected human kidney samples (Correlation coefficient>0.6) were subjected to Gene ontology analysis (DAVID).

FIGS. 23A-23E show regional plots of genotype and eGFR (GWAS) and kidney CASP9 expression (eQTL). FIG. 23A: Locus zoom plots of chromosome 1 region of eGFR GWAS, and CASP9 eQTL in kidney tubules, glomeruli, and whole kidney. X axis shows the chromosomal location of SNPs. Y axis shows the strength of association [−log 10(P)]. The data are centered at rs12736181; colors indicate linkage disequilibrium (LD) association.

FIG. 23B: Genotype (x axis) and relative CASP9 expression (y axis) association in human kidney tubules. FIG. 23C: The effect sizes of SNPs from eGFR GWAS (y axis) and eQTL studies (x axis). The dashed line represents the SMR estimate, and the red triangle is the rs6690758 that shows strong cis-eQTL effect size. Error bars are the SEs of SNP effects. FIG. 23D: Genome browser view of human kidney snATAC-seq data. The top row shows the gene, followed by the eGFR risk SNPs, and cell type-specific open chromatin information, including proximal tubule (PT), loop of Henle (LOH), distal convoluted tubule (DCT), principal cells of the collecting duct (CDPC), intercalated cells of the collecting tubule (CDIC), podocytes (Podo), endothelial (Endo), and immune cell. The zoom-out version of the region can be viewed in FIG. 12. FIG. 23E: The relative mRNA expression levels of CASP9, DNAJC16, and CELA2B in human embryonic kidney (HEK) 293 cells stably expressing Cas9 with or without deletion of the open chromatin region harboring rs12741552 (last bar) and rs12736181 (middle bar) using CRISPR-Cas9 gene editing. The glyceraldehyde-3-phosphate dehydrogenase (GAPDH) was used for internal control. The deleted region was shown on (FIG. 23D) by arrows. n=3, technical experimental triplicate. *P<0.05. N.S., not significant.

FIGS. 24A-24H show Casp9 expression and localization in mouse kidney disease models. FIG. 24A: The intrinsic apoptosis pathway. FIGS. 24B-24F: Relative mRNA levels of Casp9 (FIG. 24B), CASP9 activity (FIG. 24C), CASP3 activity (FIG. 24D), relative mRNA levels of Apaf1 (FIG. 24E), and Bax (FIG. 24F) in kidneys from matched controls (CTRL) and injury models. FA, folic acid; UNx-STZ, uninephrectomy-streptozotocin; UUO, unilateral ureteral obstruction; ApoL1, APOL1 transgenic mice; Cis, cisplatin. FIG. 24G: Representative images of double immunostaining with CASP9 and proximal tubule marker, LTL (Lotus tetragonolobus lectin) in FA, and UUO kidneys. Scale bar, 10 μm. The right panels are enlarged images of the highlighted areas. FIG. 24H: Left: Representative images of Sirius red staining of CTRL, FA, UUO, UNx-STZ, and aging mice. Right: The correlation of Casp9 expression with % area of Sirius red staining in the kidney from CTRL, FA, UUO, UNx-STZ, and aging mice. (FIGS. 24B-24F) FA CTRL (n=6), FA injury (n=6), UNx-STZ CTRL (n=8), UNx-STZ injury (n=12), aging CTRL (n=6), aging injury (n=4), UUO CTRL (n=6), UUO injury (n=6), ApoL1 CTRL (n=6), ApoL1 transgenic (n=6), Cis CTRL (n=6), and Cis injury (n=6). Data are presented as means±SEM. *P<0.05 and **P<0.01.

FIGS. 25A-25G show Casp9 HZ mice are protected from Cis-induced AKI, apoptosis, and inflammation. FIG. 25A: Experiment setup. Wild-type (WT) and Casp9 HZ (HZ) mice were euthanized 3 days after Cis. CTRL mice were injected with phosphate-buffered saline (PBS). FIGS. 25B-25D: The levels of renal Casp9 mRNA (FIG. 25B), BUN (FIG. 25C), and renal Havcr1 mRNA (FIG. 25D) in CTRL and Cis-treated mice. (FIG. 25E) Representative images and quantification of tubulointerstitial damage in hematoxylin and eosin sections in CTRL and Cis-injected kidneys of WT and Casp9 HZ (HZ) mice. Scale bar, 20 μm. FIG. 25F: Western blot image and quantification of pro-CASP9 (CASP9), cleaved CASP9 (cCASP9), BAX, BCL-2, and cleaved CASP3 (cCASP3) in CTRL and Cis-injected kidneys of WT and Casp9 HZ (HZ) mice. GAPDH was used for loading CTRL. FIG. 25G: Relative expression [quantitative reverse transcription polymerase chain reaction (qRT-PCR)] of Il1b, Csf2, Tnfa, Cxcl10, and Icam1 in CTRL and Cis-injected Cis kidneys of WT and Casp9 HZ (HZ) mice. CTRL WT (n=6), CTRL HZ (n=6), Cis WT (n=6), and Cis HZ (n=6). Data are presented as means±SEM. *P<0.05 and **P<0.01.

FIGS. 26A-26E show pharmacological inhibition of CASP9 protected from Cis-induced kidney injury and inflammation. FIG. 26A: Experimental design. (FIGS. 26B-26D) The levels of serum creatinine (Cr) (FIG. 26B), BUN (FIG. 26C), and renal Havcr1 mRNA (FIG. 26D) in PBS-injected (PBS) or Cis-injected mice with CASP9 inhibitor (inhibitor) or vehicle CTRL. FIG. 26E: Relative renal expression (qRT-PCR) of Il1b, Csf2, Tnfa, Cxcl10, and Icam1. PBS with vehicle CTRL (n=3), PBS with CASP9-specific inhibitor (n=3), Cis with vehicle CTRL (n=6), and Cis with CASP9-specific inhibitor (n=6). Data are presented as means±SEM. *P<0.05.

FIGS. 27A-27F show improved autophagy flux in Cis-treated Casp9 HZ renal tubule cells. FIG. 27A: Western blot image and quantification of RIPK3, GSDMD, NLRP3, ACSL4, and LC3-II in CTRL and Cis-treated kidneys from WT and Casp9 HZ (HZ) mice. GAPDH was used for loading CTRL. FIG. 27B: Isolation of renal tubule cells from WT and Casp9 HZ (HZ) mice. FIG. 27C: Representative Western blot and quantification of LC3-II in cultured renal tubule cells from WT and Casp9 HZ (HZ) mice in indicated conditions (Cis+BafA1, Cis with bafilomycin A1). Cells were exposed to Cis for 8 hours. GAPDH was used for loading CTRL. FIG. 27D: Autophagy flux in WT and Casp9 HZ (HZ) renal tubule cells. Flux was calculated as a difference in LC3-II levels (corrected by GAPDH) between Cis and Cis+BafA1 group. Three independent experiments were performed. FIG. 27E: Representative confocal image of the renal tubule cell transfected with EGFP-LC3 plasmid or mRFP-EGFP-LC3 plasmid in indicated conditions. Scale bar, 10 μm. FIG. 27F: Quantification of autophagosome (left) and autolysosome (right) in the renal tubule cell in indicated conditions. Experiments were performed three times, and in each experiment, n=10 cells were counted, and mean value was plotted in the graph. Data are presented as means SEM. *P<0.05.

FIGS. 28A-28F show improved mitophagy and lower cGAS-STING activation in Cis-treated Casp9 HZ renal tubule cells. FIG. 28A: Representative confocal images of renal tubule cells transfected with COX8-EGFP-mCherry plasmid. Quantification of mCherry dots (mitolysosome) in indicated conditions. Scale bar, 10 μm. Three independent experiments were performed. In each experiment, n=10 cells were counted, and mean value was plotted. FIG. 28B: Representative confocal image of JC-1-stained Cis or PBS-treated (CTRL) WT and Casp9 HZ (HZ) renal tubule cells. Scale bar, 10 μm. Green fluorescence intensity indicates mitochondrial depolarization. FIG. 28C: Extraction and quantification of cytosolic mitochondrial DNA (mtDNA) of Cis or PBS-treated (CTRL) WT and Casp9 HZ (HZ) renal tubule cells. FIG. 28D: Representative Western blot image of cGAS, STING, pTBK1, TBK1, pp65, p65, pIRF3, and IRF3 in Cis- or PBS-treated (CTRL) WT and Casp9 HZ (HZ) renal tubule cells. FIG. 28E:Relative expression (qRT-PCR) of Il1b, Csf2, Tnfa, Cxcl10, and Icam1 in Cis- or PBS-treated (CTRL) WT and Casp9 HZ (HZ) renal tubule cells. FIG. 28F: Western blot image and quantification of cGAS and STING in CTRL and Cis kidneys of WT and Casp9 HZ (HZ) mice. GAPDH was used for loading CTRL. Data are presented as means SEM. *P<0.05.

FIGS. 29A-29I show Casp9 HZ mice are protected from FA-induced kidney fibrosis. FIG. 29A: WT and Casp9 HZ (HZ) mice were euthanized 7 days after FA injection (intraperitoneal injection of 250 mg/kg FA). FIGS. 29B-29D: The levels of renal Casp9 mRNA (FIG. 29B), BUN (FIG. 29C), and renal Havecr1 mRNA (FIG. 29D) in CTRL and FA-induced mice. FIG. 29E: Western blot image and quantification of pro-CASP9 (CASP9), cleaved CASP9 (cCASP9) in CTRL, and FA-induced kidneys from WT and Casp9 HZ (HZ) mice. GAPDH was used for loading CTRL. FIGS. 29F-29G: Relative expression (qRT-PCR) of Slc22a30, Slc27a2, Slc12a2 (FIG. 29F), Il1b, Csf2, Tnfa, Cxcl10, and Icam1 (FIG. 29G), in CTRL and Cis-induced kidneys from WT and Casp9 HZ (HZ) mice. FIG. 29H: Representative images and quantification of tubulointerstitial fibrosis by Sirius red staining in CTRL and FA-induced kidneys from WT and Casp9 HZ (HZ) mice. Scale bar, 20 μm. FIG. 29I: Relative expression (qRT-PCR) of Col1a1, Col1a3, Fn1, and Vim in CTRL and FA-induced kidneys from WT and Casp9 HZ (HZ) mice. CTRL WT (n=6), CTRL HZ (n=6), FA WT (n=6), and FA HZ (n=6). Data are presented as means±SEM. *P<0.05 and **P<0.01.

FIGS. 30A-30F show lower inflammation and kidney fibrosis in Casp9 HZ mice subjected to UUO injury. FIG. 30A: Experimental design. FIG. 30B: Western blot image and quantification of pro-CASP9 (CASP9), cleaved CASP9 (cCASP9), BAX, BCL2, and cleaved CASP3 (cCASP3) in CTRL and UUO kidney from WT and Casp9 HZ (HZ) mice. GAPDH was used for loading CTRL. FIG. 30C: Relative expression (qRT-PCR) of Il1b, Csf2, Tnfa, and Cxcl10 in CTRL and UUO kidney from WT and Casp9 HZ (HZ) mice. FIG. 30D: Western blot image and quantification of LC3-II, cGAS, and STING in CTRL and UUO kidney from WT and Casp9 HZ (HZ) mice. GAPDH was used for loading CTRL. FIG. 30E: Representative images and quantification of tubulointerstitial fibrosis by Sirius red staining in CTRL and UUO kidney from WT and Casp9 HZ (HZ) mice. Scale bar, 20 μm. FIG. 30F: Relative transcript expression (qRT-PCR) of Col1a1, Col1a3, Fn1, and Vim in CTRL and UUO kidney from WT and Casp9 HZ (HZ) mice. CTRL WT (n=6), CTRL HZ (n=6), UUO WT (n=6), and UUO HZ (n=7). Data are presented as means±SEM. *P<0.05 and **P<0.01.

FIGS. 31A-31C are regional plots of genotype and eGFR (GWAS) in eGFR genome wide association studies (FIGS. 31A-31C) Locuszoom plots of chromosome 1 region of different eGFR GWAS. X-axis shows the chromosomal location of SNPs. Y-axis shows the strength of association (−log 10(p)).

FIG. 32 shows multi-tissue eQTL analysis (rs12736181) and CASP9 expression in GTEx database (Left) The effect size of single tissue eQTL with 95% confidence interval. The organs were arranged according to the observed effect sizes. (Right) Single-tissue eQTL p-value vs. multi-tissue posterior probability. Y axis; −log 10 (singletissue eQTL p-value). X axis; m-value (0-1), indicating posterior probability of eQTL effect in each tissue. M-value<0.1 indicates no significant eQTL effect in tissue, while m-value>0.9 indicates significant eQTL effect in tissue.

FIGS. 33A-33B show genotype-driven expression changes in CELA2B and DNAJC16. FIG. 33A: LocusZoom plots of chromosome 1 region of CKD eGFR GWAS, CELA2B eQTL in kidney tubules and glomeruli. FIG. 33B: LocusZoom plots of chromosome 1 region of CKD eGFR GWAS, DNAJ16 eQTL in kidney tubules and glomeruli. X-axis; chromosomal location of SNPs. Y-axis; the strength of association (−log 10(p)).

FIG. 34 shows human kidney single nuclei open chromatin data and fine mapping of eGFR-associated GWAS variants Human kidney single cell open chromatin atlas of eGFR GWAS chromosome 1 region. The 2 genetic variants (rs12741552 and rs12736181) with eGFR GWAS and tubule eQTL effects on CASP9 overlapped with open chromatin area in human kidney cells were highlighted. The top row shows genome organization, followed by eGFR GWAS risk SNPs, open chromatin tracks in PT; proximal tubule, LOH; loop of Henle, DCT; distal convoluted tubule, CDPC; principal cells of the collecting duct, CDIC; intercalated cells of the collecting tubule, Podo; podocytes, Endo; endothelial, immune cell.

FIG. 35 shows representative images of CASP9 and LTL staining in control mouse kidneys. Representative images of double immunostaining with CASP9 and proximal tubule marker, LTL (Lotus tetragonolobus lectin) in control kidneys. Scale bar:10 μm. The right panels are enlarged images of the boxed area.

FIG. 36 shows representative images of CASP9 and LTL staining in control and CKD human kidneys. Representative images of double immunostaining with CASP9 and proximal tubule marker, LTL (Lotus tetragonolobus lectin) in healthy (upper panel) and CKD (lower panel) human kidneys. Scale bar:20 μm. The right panels are enlarged images of the boxed areas. White arrow indicates CASP9 positive renal proximal tubules.

FIGS. 37A-37B show cleaved caspase-3 expression in mouse kidney disease models. FIGS. 37A-37B: Representative cleaved caspase-3 staining and quantification of cleaved caspase-3 positive cells in kidneys of wild type (WT) and Casp9 HZ (HZ) mice in control (CTRL) and cisplatin (Cis) (FIG. 37A) or folic acid (FA) (FIG. 37B) Bar; 20 μm. Data are presented as the mean±SEM. *P<0.05.

FIGS. 38A-38C show reduced apoptosis in Casp9 HZ renal tubule cells. FIG. 38A: Experimental design. The renal tubule cells were isolated form wild type (WT) and Casp9 HZ (HZ) mice. FIG. 38A: Relative fluorescence unit (RFU)-based live cell analysis of cisplatin-(Cis) or PBS-treated (CTRL) renal tubule cells from wild type (WT) and Casp9 HZ (HZ) mice. (n=3 independent experiments). FIG. 38C: Representative western blot image and quantification of pro-caspase-9 (CASP9), cleaved caspase-9 (cCSP9), cleaved caspase-3 (cCASP3) of cisplatin-(Cis) or PBS-treated (CTRL) renal tubule cells from wild type (WT) and Casp9 HZ (HZ) mice. GAPDH was used for loading control. (n=3 independent experiment). Data are presented as the mean±SEM. *P<0.05, **P<0.01.

FIG. 39 show reduced protein expression of cytosolic nucleotide sensing pathways in cisplatin-treated Casp9 HZ mice. Quantification of cGAS, STING, pTBK1, pp65, and pIRF3 kidney western blots in control (CTRL) or cisplatintreated (Cis) wild type (WT) and Casp9 HZ (HZ) renal tubule cells. GAPDH was used for loading control. 3 independent experiments were performed. Data are presented as the mean±SEM. *P<0.05.

FIG. 40 show reduced tubulointerstitial injury in Casp9 HZ mice subjected to UUO injury. Representative Hematoxylin and Eosin stains of kidneys and quantification of renal tubule injury score in control (CTRL) and UUO-subjected kidney from WT (WT) and Casp9 HZ (HZ) mice. Bar; 20 μm. CTRL WT (n=6), CTRL HZ (n=6), UUO WT (n=6), UUO HZ (n=7). Data are presented as the mean±SEM. *P<0.05.

FIG. 41 show alteration in cell death pathway in WT and Casp9 HZ mice subjected to UUO injury. Western blot image and quantification of RIPK3, gasdermin D (GSDMD), NLRP3, ACSL4 in control (CTRL) and UUO kidney from wild type (WT) and Casp9 HZ (HZ) mice. GAPDH was used for loading control. Data are presented as the mean±SEM. N.S. not significant.

FIG. 42 show the result of the multi tissue eQTL analysis.

FIG. 43 is a table showing characteristics of control and aging mice of wild type (WT) and Casp9 HZ (HZ) mice.

FIG. 44 is a table showing guide RNA sequences and target deleted region. The sequences of the guide RNAs are also listed in the following Table:

Oligo 1 Forward for SNP rs12736181:
CACCGAAACGCCTCTCCCATACGGG (SEQ ID NO: 12)
Oligo 1 Reverse for SNP rs12736181:
AAACCCCGTATGGGAGAGGCGTTTC (SEQ ID NO: 13)
Oligo 2 Forward for SNP rs12736181:
CACCGGTCCTGAATGCGGCCTACCT (SEQ ID NO: 14)
Oligo 2 Reverse for SNP rs12736181:
AAACAGGTAGGCCGCATTCAGGACC (SEQ ID NO: 15)
Oligo 1 Forward for SNP rs12741552:
CACCGATTCGTAGAAGTGAACGAAT (SEQ ID NO: 16)
Oligo 1 Reverse for SNP rs12741552:
AAACATTCGTTCACTTCTACGAATC (SEQ ID NO: 17)
Oligo 2 Forward for SNP rs12741552:
CACCGAGTGCAAGTTTAATGCCGCC (SEQ ID NO: 18)
Oligo 2 Reverse for SNP rs12741552:
AAACGGCGGCATTAAACTTGCACTC (SEQ ID NO: 19)

FIG. 45 is a table showing primer information for QRT-PCR. The sequences of the primers are also listed in the following table:

Apaf1 forward:
GCAAACGAGAGGAAAAGCATTAA (SEQ ID NO: 20)
Apaf1 reverse:
GCAGACCAGGAACAACACTTCA (SEQ ID NO: 21)
Bax forward:
CTGCAGAGGATGATTGCTGA (SEQ ID NO: 22)
Bax reverse:
GAGGAAGTCCAGTGTCCAGC (SEQ ID NO: 23)
Casp9 forward:
GTCAAGTTTGCCTACCCCCA (SEQ ID NO: 24)
Casp9 reverse:
GAGCCCACTGCTCCAGAATG (SEQ ID NO: 25)
Col1a1 forward:
TGACTGGAAGAGCGGAGAGT (SEQ ID NO: 26)
Col1a1 reverse:
GTTCGGGCTGATGTACCAGT (SEQ ID NO: 27)
Col3a1 forward:
ACAGCTGGTGAACCTGGAAG (SEQ ID NO: 28)
Col3a1 reverse:
ACCAGGAGATCCATCTCGAC (SEQ ID NO: 29)
Csf2 forward:
GGCCTTGGAAGCATGTAGAGG (SEQ ID NO: 30)
Csf2 reverse:
GGAGAACTCGTTAGAGACGACTT (SEQ ID NO: 31)
Cxcl10 forward:
AAGTGCTGCCGTCATTTTCT (SEQ ID NO: 32)
Cxcl10 reverse:
GTGGCAATGATCTCAACACG (SEQ ID NO: 33)
Fn1 forward:
CCGTGTAAGGGTCAAAGCAT (SEQ ID NO: 34)
Fn1 reverse:
ACAAGGTTCGGGAAGAGGTT (SEQ ID NO: 35)
Gapdh forward:
ATGTTTGTGATGGGTGTGAA (SEQ ID NO: 36)
Gapdh reverse:
ATGCCAAAGTTGTCATGGAT (SEQ ID NO: 37)
Havcr1 forward:
TCCACACATGTACCAACATCAA (SEQ ID NO: 38)
Havcr1 reverse:
GTCACAGTGCCATTCCAGTC (SEQ ID NO: 39)
Icam1 forward:
GCCTCCGGACTTTCGATCTT (SEQ ID NO: 40)
Icam1 reverse:
TAGGAGATGGGTTCCCCCAG (SEQ ID NO: 41)
Il1b forward:
CCCTGCAGCTGGAGAGTGTGGA (SEQ ID NO: 42)
Il1b reverse:
TGTGCTCTGCTTGTGAGGTGCTG (SEQ ID NO: 43)
Mlk1 forward:
GAAGACAGACCTAGACAGCGG (SEQ ID NO: 44)
Mlk1 reverse:
CCAGTAGCTTCACCACTCGAC (SEQ ID NO: 45)
Ripk3 forward:
AATTGTACTCTGGGAAATTGCCA (SEQ ID NO: 46)
Ripk3 reverse:
TCTCCAAGATTCCGTCCACAG (SEQ ID NO: 47)
Slc12a1 forward:
GGAGTTGTGAAGTTTGGATGGG (SEQ ID NO: 48)
Slc12a1 reverse:
ATTCCCGCTTCTCCTACAATCC (SEQ ID NO: 49)
Slc22a30 forward:
CTAGGCATGGTGGCAGTGCT (SEQ ID NO: 50)
Slc22a30 reverse:
CCGAGCCGACTCTGACATCC (SEQ ID NO: 51)
Slc27a2 forward:
CGCTGACATCGTGGGACTGG (SEQ ID NO: 52)
Slc27a2 reverse:
TCGACCCTCATGACCTGGCA (SEQ ID NO: 53)
Tnfa forward:
TGTGAGGAAGGCTGTGCATT (SEQ ID NO: 54)
Tnfa reverse:
GGTCAGGTTGCCTCTGTCTC (SEQ ID NO: 55)
Vim forward:
GATCGATGTGGACGTTTCCAA (SEQ ID NO: 56)
Vim reverse:
ATACTGCTGGCGCACATCAC (SEQ ID NO: 57)
hCASP9 forward:
GCTTCTCCTCGCTGCATTTC (SEQ ID NO: 58)
hCASP9 reverse:
GCAGCTGGTCCCATTGAAGA (SEQ ID NO: 59)
hDNAJC16 forward:
TTGGTGCTTTAGCTGCATTCA (SEQ ID NO: 60)
hDNAJC16 reverse:
CGCCAATTCCTACACCCAATTC (SEQ ID NO: 61)
hCELA2B forward:
GCAGCACCGTGAAGACGAATA (SEQ ID NO: 62)
hCELA2B reverse:
ATGCCTGACAGTTCAGCGG (SEQ ID NO: 63)
hGAPDH forward:
CTGGGCTACACTGAGCACC (SEQ ID NO: 64)
hGAPDH reverse:
AAGTGGTCGTTGAGGGCAATG (SEQ ID NO: 65)

FIGS. 46A-46G show gene prioritization analysis identified Acetyl CoA synthetase 2 (ACSS2) as a kidney risk gene.

FIG. 47A-47K show that (ACSS2) genetic deletion attenuates chronic kidney disease.

FIG. 48A-48K show that ACSS2 drives fatty acid synthesis during kidney injury.

FIGS. 49A-49L show that inhibition of fatty acid synthesis and ACSS2 in mice prevents kidney fibrosis.

FIGS. 50A-50V show elevated ROS and depleted antioxidant system in cells with elevated DNL. MitoROS depletion prevents pyroptosis.

FIGS. 51A-51L: prioritization of ACSS2 as a kidney disease gene from GWAS studies.

FIG. 51A: Regional plot showing single nucleotide variants associated with kidney eGFR GWAS dataset (n=1,508,569 individuals). X-axis chromosomal location y-axis strength of association (−log(p)) on chromosome 20. The locus top variants (rs11698977) tagging the independent signal closest to ACSS2 gene was selected as index variant to calculate LD (r2) with other variants in the locus shown in blue dots (lower r2) to red dots (higher r2). FIG. 51B: Genetic analyses used and prioritization scoring strategy used in gene prioritization (top left). Genes with 3-6 gene priority scores at the chromosome 20 eGFR GWAS locus (right). Color indicates priority score. FIG. 51C: Regional plot of genetic variants associated with kidney tubule cytosine methylation levels (mQTL) (n=443). X-axis chromosomal location y-axis strength of association (−log(p)) on chromosome 20. FIG. 51D: Regional plot for SNPs associated with kidney glomeruli ACSS2 expression (n=303). X axis chromosomal location y-axis strength of association (−log(p)) on chromosome 20. FIG. 51E: Regional plot for SNPs associated with kidney tubule ACSS2 expression (n=356). X-axis chromosomal location y-axis strength of association (−log(p)) on chromosome 20. FIG. 51F: Human kidney ACSS2 gene expression in tubules (n=356) in microdissected samples. Y-axis shows normalized ACSS2 expression and X-axis shows genotype information. FIG. 51G: Upper panel is the locus zoom plot of eGFRcrea GWAS associations (n=1,508,659 individuals) in ACSS2 locus. Lower panel epigenetic information of ACSS2 locus in human kidney samples including mQTL SNPs, all eGFR GWAS SNPs (Blue) followed by eGFR GWAS SNPs with priority score>2 (dark green), eGFRcrea GWAS SNPs with priority score>4 (magenta), adult human kidney snATAC-seq chromatin accessibility information in each cell type, histone modification information (H34me3, H3K27ac and H3K4me1) from human kidney ChIP-seq and chromatin states predicted by ChromHMM. Cell type annotation; Proximal tubules (PT) segment (S1-3), loop of Henle (LOH), distal convoluted tubule (DCT), collecting duct principal cell types (PC), collecting duct intercalated cells (IC), podocytes (Podo), endothelial cells (Endo), immune cells (Immune); stromal fraction (stroma). FIG. 51H: Scheme of CRISPR-mediated genetic deletion of prioritized variants. FIG. 51I: Transcript levels of ACSS2 following deletion of the genetic risk locus containing SNPs 1, 3, 4, 5, and 6. Data are represented as mean±SEM. P values determined by one-way ANOVA in Graphpad Prism 9 software. *p<0.05, **p<0.01, ***p<0.001 and ****p<0.0001. See also FIGS. 58A-58L.

FIGS. 52A-52N demonstrate that genetic deletion of ACSS2 protects from kidney disease, in accordance with some embodiments. FIG. 52A: Experimental outline. FIG. 52B: Daily body weights of vehicle (n=7) or adenine-gavage WT (n=7) (wild type) and Acss2−/− (n=6) (knock-out) mice. FIG. 52C: Final body weights of WT (n=7) and Acss2−/− (n=6) mice gavaged with adenine or vehicle (n=7). FIG. 52D: Immunoblots of ACSS2 and GAPDH in vehicle-treated (n=4), or adenine-treated WT (n=5) and Acss2−/− (n=5) mice. FIG. 52E: Transcript levels of alpha smooth muscle actin (Acta2), Collagen 1a1 (Col1a1), collagen type 3a (Col3a) and fibronectin (Fn1) in kidneys of vehicle (n=7) or adenine-treated WT (n=7), and Acss2−/− (n=6) mice. FIG. 52F: Immunoblots of Fibronectin (FN1), αSMA and GAPDH in whole kidney lysates of vehicle or adenine-treated WT (n=5) and Acss2−/− (n=5) mice (n=4). FIG. 52G: Representative H&E and Sirius Red staining in kidney sections of vehicle or adenine treated WT and Acss2−/− mice. Scale bars 20 μm. FIG. 52H: Biochemical indicators of kidney function serum creatinine (sCr), and blood urea nitrogen (BUN) in vehicle- or adenine-treated WT and Acss2−/− mice gavage. FIG. 52I: Experimental outline. FIG. 52J: Relative transcript levels of ACSS2 in WT (n=5) and Acss2−/− (n=7) mice following unilateral obstruction surgery (UUO). FIG. 52K: Immunoblots of ACSS2 and GAPDH of UUO or SHAM operated kidneys of WT and Acss2−/− mice. FIG. 52L: FN1, αSMA and GAPDH Western blots of whole kidney lysates of WT and Acss2−/− mice following UUO or SHAM operation. FIG. 52M: Quantification of immunoblots of FN1, and αSMA by image J. FIG. 52N: Representative H&E and Sirius Red staining images of kidneys from WT and Acss2−/− mice with UUO. Scale bars 20 μm. Data are represented as mean±SEM. P values determined by one-way ANOVA in Graphpad Prism 9 software. *p<0.05, **p<0.01, ***p<0.001 and ****p<0.0001. See also FIGS. 59A-59Q.

FIG. 53A-53O demonstrate that kidney ACSS2 correlates with changes in genes in de novo lipogenesis, in accordance with some embodiments. FIG. 53A: Biochemical functions associated with ACSS2. FIG. 52B: Immunoblots of total H3K27ac and H3 protein levels in histone extracted from kidneys of WT and Acss2−/− mice with UUO surgery (left). Bar graph showing quantification of H3K27ac levels by image J (right). FIG. 53C: Relative transcript levels of acyl coA oxidase 1 (Acox1), Acox2, carnitine palmitoyl transferase 1 (Cpt1) and Cpt2 in whole kidney lysates of WT (n=6) and Acss2−/− (n=7) mice with and without UUO surgery. FIG. 53D: Scheme of measuring FAO using labeled palmitic acid. FIG. 53E: FAO rate in WT (n=6) and ACSS2−/− (n=7) mice with and without UUO surgery. FIG. 53F: Relative transcript levels of hydroxy methyl glutaryl coA synthase 1 (Hmgcs1), reductase (Hmgcr) and farnesyl diphosphate synthase (Fdps) in whole kidney lysates of WT (n=6) and Acss2−/− (n=7) mice with and without UUO surgery. FIG. 53G: Total cholesterol levels in the whole kidney lysates of WT (n=6) and Acss2−/− (n=7) mice subjected to UUO surgery. FIG. 53H: Relative transcript levels of sterol regulatory binding protein (Srebp) cleavage protein (Scap) and Srebp1 in whole kidney lysates of WT (n=6) and ACSS2−/− (n=7) mice subjected to UUO surgery. FIG. 53I: Relative transcript levels of fatty acid synthase (Fasn) and acetyl Co-A carboxylase (Acaca) in whole kidney lysates of WT (n=6) and Acss2−/− (n=7) mice subjected to UUO surgery. FIG. 53J: Immunoblotting of FASN and GAPDH protein levels in total kidneys lysates of WT and Acss2−/− mice. FIG. 53K: Scheme of measuring de novo lipogenesis (DNL) rate using deuterated water in vivo. FIG. 53L: DNL rate in WT (n=3) and ACSS2−/− (n=4) mice with and without UUO surgery. FIG. 53M Relative transcript levels perilipin2 (Plin2) in whole kidney extracts of WT (n=6) and Acss2−/− (n=7) mice with UUO surgery. FIG. 53N: Total kidney triglyceride levels were measured in WT (n=6) and Acss2−/− (n=7) mice with UUO surgery. FIG. 53O: Representative images of Oil Red 0 staining of kidneys of WT and Acss2−/− mice with UUO. Scale bars 10 μm. Data are represented as mean±SEM. P values determined by one-way ANOVA in Graphpad Prism 9 software. *p<0.05, **p<0.01, ***p<0.001 and ****p<0.0001. See also FIGS. 60A-60R.

FIG. 54A-54L demonstrate that tubule cell specific deletion of FASN or pharmacological inhibition of ACSS2 protects from kidney disease, in accordance with some embodiments. FIG. 54A: Experimental design. FIG. 54B: Transcript levels of fatty acid synthase (Fasn) (n=4, WT; n=6, Fasn f/f; Ksp Cre). FIG. 54C: Immunoblotting of FASN and GAPDH in the Fasn f/f Ksp Cre and WT mice. FIG. 54D: Transcript levels of alpha smooth muscle actin (Acta2), collagen1a1 (Col1a1), collagen 3a (Col3a) and fibronectin (Fn1) in whole kidney extracts of Fasn f/f Ksp Cre (n=6) and WT (n=4) mice. FIG. 54E: Fibronectin (FN1), alpha smooth muscle actin (α-SMA) and GAPDH immunoblots of whole kidney lysates of Fasn f/f; Ksp Cre and WT mice. FIG. 54F: Quantification of FN1 and αSMA immunoblots by image J. FIG. 54G: Representative images H&E and Sirius red stained kidney sections from Fasn f/f; Ksp Cre and WT mice (left). Relative percentage of fibrosis quantified by Image J (right). Scale bars 20 μm. FIG. 54H: Experimental design with ACSS2i. FIG. 54I: Representative images of H&E and Sirius Red-stained kidney sections of mice injected with ACSS2i followed by UUO injury. Scale bars 20 μm·j. FN1, αSMA and GAPDH immunoblotting of whole kidney lysates of mice injected with ACSS2i followed by UUO surgery. FIG. 54K: Quantification of FN1 and αSMA immunoblots by image J. FIG. 54L: Transcript levels of alpha smooth muscle actin (Acta2), Collagen 1a1 (Col1a1), collagen type 3a (Col3a) and fibronectin (Fn1) were measured in whole kidney extracts of mice injected with ACSS2i (n=3) or PBS (n=3) with and without UUO injury. Data are represented as mean±SEM. P values determined by one-way ANOVA in Graphpad Prism 9 software. *p<0.05, **p<0.01, ***p<0.001 and ****p<0.0001. See also FIGS. 61A-61Z.

FIGS. 55A-55V demonstrate that de novo lipogenesis in kidney tubules is associated with higher mitochondrial ROS, in accordance with some embodiments. FIG. 55A: Fatty acid synthesis requires large amount of NADPH, leading to elevated ROS levels in kidney. FIG. 55B: Experimental scheme showing simultaneous measurements of NADPH & NAD+ and GSH & GSSH in various treatment conditions in cultured primary kidney tubule cells. FIG. 55C: NADPH/NDP+ ratio measured in Acss2−/− primary kidney tubule cells treated with TGF-β1. FIG. 55D: Total NADPH levels measured in Acss2−/− cells treated with TGF-β1. FIG. 55E: GSH/GSSH ratio measured inAcss2−/− cells treated with TGF-β1. FIG. 55F: Total GSH levels measured inAcss2−/− cells treated with TGF-β1. FIG. 55G: NADPH/NDP+ ratio measured in cells treated with TGF-β1 in the presence of FASNall. FIG. 55H: Total NADPH levels measured in cells treated with TGF-β1 in the presence of FASNall. FIG. 55I: GSH/GSSH ratio measured in cells treated with TGF-β1 in the presence of FASNall. FIG. 55J: Total GSH levels measured in cells treated with TGF-β1 in the presence of FASNall.

FIG. 55K: Representative images of MitoSox staining in WT, Acss2−/−, and FASNall treated primary kidney tubule cells with TGF-β1. Scale bars 10 μm. FIG. 55L: Mitochondrial ROS levels quantified by MitoSox in WT, Acss2−/−, and FASNall treated cells with TGF-β1. FIG. 55M: Representative images of JC-1 staining to quantify mitochondrial membrane potential in WT, Acss2−/−, and FASNall primary kidney tubule cells treated with TGF-β1. Scale bars 10 μm. FIG. 55N: Quantification of JC-1 (red to green ratio) relative fluorescence units in WT, Acss2−/, and FASNall treated cells following treatment with TGF-β1. FIG. 55O: Experimental scheme of cells treated with FASNall or TVB-3664 and TGF-β1. FIG. 55P: Transcript levels of Nlrp3 were measured in primary tubular cells treated with FASNall and TGF-β1. FIG. 55Q: Transcript levels of IL-1β were measured in primary tubular cells treated with FASNall and TGF-β1. FIG. 55R: Transcript levels of caspase1 were measured in primary tubular cells treated with FASNall and TGF-β1. FIG. 55S: Experimental scheme. FIG. 55T: Transcript levels of caspase 1, IL-1β, IL18, and GSDMD were measured in Acss2−/− primary tubular cells treated with TGF-β1. FIG. 55U: Representative images of MitoSox staining in WT and Acss2−/− primary tubule cells treated with TGF-β1 in the presence of MitoTempo (100 μM). Scale bars 10 μm. FIG. 55V: Transcript levels of IL-1β, IL18 and caspase1 were measured inAcss2−/− primary tubular cells treated with TGF-β1 in the presence of MitoTempo. Data are represented as mean±SEM. P values determined by one-way ANOVA in Graphpad Prism 9 software. *p<0.05, **p<0.01, ***p<0.001 and ****p<0.0001. See also FIGS. 62A-62K.

FIGS. 56A-56J demonstrate that genetic deletion or pharmacological inhibition of FASN or ACSS2 attenuates inflammasome activation, in accordance with some embodiments. FIG. 56A: Transcript levels of Nlrp3, IL-1β, IL18 and Gasdermin D (GSDMD) were measured in control and UUO kidneys of Fasn f/f Ksp Cre (n=6) and WT (n=4) mice. FIG. 56B: NLRP3, Caspase1, p20 (cleaved Caspase1), GSDMD full length (GSDMD-F), N-terminal GSDMD (GSDMD-N) and GAPDH immunoblots of kidneys of mice with control and UUO surgery from WT and Fasn f/f Ksp Cre mice. FIG. 56C: Experimental design of ACSS2i administration followed by UUO surgery. FIG. 56D: Transcript levels of Nlrp3, IL-1β, IL18 and Gsdmd were measured in control and UUO kidneys of mice injected with ACSS2i (n=3) or vehicle (n=3). FIG. 56E: NLRP3, Caspase1, p20, GSDMD-F, GSDMD-N and GAPDH immunoblots of UUO kidneys of mice injected with vehicle (n=3) or ACSS2i (n=3). FIG. 56F: Experimental design. FIG. 56G: Transcript levels of Nlrp3, IL-1β, IL18 and Gsdmd were measured in WT (n=6) and Acss2−/− (n=7) mice subjected to UUO. FIG. 56H: NLRP3, Caspase1, p20, GSDMD-F, GSDMD-N and GAPDH immunoblots of UUO kidneys of WT and Acss2−/− mice. FIG. 56I: Transcript levels of Nlrp3, IL-1β, IL18 and Gsdmd were measured in WT (n=7) and Acss2−/− (n=6) gavage with adenine for 30 days. FIG. 56J: NLRP3, Caspase1, p20, GSDMD-F, GSDMD-N and GAPDH immunoblots of adenine-CKD kidneys of WT and Acss2−/− mice. Data are represented as mean±SEM. P values determined by one-way ANOVA in Graphpad Prism 9 software. *p<0.05, **p<0.01, ***p<0.001 and ****p<0.0001. See also FIGS. 63A-63K.

FIG. 57A-57I: Changes in gene expression of de novo lipogenesis in kidneys of patients with CKD, in accordance with some embodiments. FIG. 57A: Relative gene expression (z-score) of ACSS2, NR1H3, NR1H4, SREBF1, SCAP, PLIN2, PLIN5, ACACAB, ACLY, and FASN in healthy human kidney single nuclear RNA sequencing. This expression data identifies all cell types named as endothelial cells (Endo), fibroblasts (Fibro), podocytes (Podo), parietal epithelial cells (PEC), mesangial (Mes), proximal tubule (PT), loo of Henle (LOH), distal convoluted tubule (DCT), convoluted tubule (CNT), principal cells (PC), intercalated cells A (IC_A) and intercalated cells B (IC_B). FIG. 57B: Relative gene expression (z-score) of ACSS2, NR1H3, NR1H4, SREBF1, SCAP, PLIN2, PLIN5, ACACAB, ACLY, and FASN in human CKD kidney single nuclear RNA sequencing. This expression data identifies all cell types named as endothelial cells (Endo), fibroblasts (Fibro), podocytes (Podo), parietal epithelial cells (PEC), mesangial (Mes), proximal tubule (PT), loo of Henle (LOH), distal convoluted tubule (DCT), convoluted tubule (CNT), principal cells (PC), intercalated cells A (IC_A) and intercalated cells B (IC_B). FIG. 57C: In situ hybridization of human ACSS2 and LRP2 (PT marker) in healthy and CKD human kidney samples. (Scale bar 20 μm) and 60× (scale bar 10 μm) magnification. FIG. 57D: RNA-in situ hybridization quantification in healthy and CKD kidneys. FIG. 57E: In situ hybridization of mouse Acss2 and Lrp2 (PT marker) in healthy mouse kidney samples at 20× (scale bar 20 μm) and 60× (scale bar 10 μm) magnification. FIG. 57F: Immunofluorescence staining of ACSS2 in healthy human kidneys. LTL was used to identify PT segments of the kidney. Scale bars 20 μm and inset scale bar is 10 μm. FIG. 57G: Immunofluorescence staining of FASN in healthy human kidneys. LTL was used to identify PT segments of the kidney. Scale bars 20 μm and inset scale bar is 10 m. FIG. 57H: Immunoblotting of SCAP, FASN, PLIN2, NLRP3 and GSDMD in healthy and CKD human kidney lysates (n=6). FIG. 57I: Quantification of immunoblots of SCAP, FASN, PLIN2, NLRP3 and full length GSDMD (GSDMD-F) proteins in whole kidney lysates of healthy and CKD humans. Normalized to relative intensity of GAPDH blot. Data are represented as mean±SEM. P values determined by one-way ANOVA in Graphpad Prism 9 software. *p<0.05, **p<0.01, ***p<0.001 and ****p<0.0001. See also FIGS. 64A-64C.

FIGS. 58A-58L: Prioritization of ACSS2 as a kidney disease risk gene, in accordance with some embodiments. FIG. 58A: Fine mapping regional plot showing single nucleotide variants associated with kidney eGFR GWAS dataset. X-axis chromosomal location with gene position and y-axis shows the strength of association (−log(p)) on chromosome 20. Variants shown in red color shows strongest correlation (extreme right r2=0.0-1.0) with underlying genes. Color key indicates LD r2 which indicates linkage disequilibrium. FIG. 58B: Human kidney ACSS2 gene expression in glomeruli (n=303) in microdissected samples. Y-axis shows normalized ACSS2 expression and X-axis shows genotype information. FIG. 58C: GWAS and eQTL effect sizes (upper plot tubule; lower plot glom) plotted for ACSS2 gene in 1.5M human samples. FIG. 58D: Transcript levels of CEP250 measured in Cas9 expressing HEK293 cells transfected with guide RNAs which target identified risk regions in the human snATAC-seq data. Risk SNPs 1, 3, 4, 5, and 6 deleted using Crispr-based gene editing technology. FIG. 58E: Transcript levels of SPAG4 measured in Cas9 expressing HEK293 cells transfected with guide RNAs which target identified risk regions in the human snATAC-seq data. Risk SNPs 1, 3, 4, 5, and 6 deleted using Crispr-based gene editing technology. FIG. 58F: Immunoblots of ACSS2, KIM-1 and GAPDH in whole kidney lysates of WT (n=5) and Acss2−/− mice (n=6). FIG. 58G: Transcript levels of Acss2 measured in whole kidneys of WT (n=5) and Acss2−/− mice (n=6). FIG. 58H: Weekly body weight changes recorded in WT (n=5) and Acss2−/− mice (n=6) at baseline. FIG. 58I: Blood urea nitrogen (BUN) measured in 10 weeks old WT (n=5) and Acss2−/− (n=6) mice at baseline. FIG. 58J: Serum creatinine (sCr) measured in 10 weeks old WT (n=5) and Acss2−/− (n=6) mice at baseline. FIG. 58K: Ki67 immunofluorescence in WT and Acss2−/− mice at 10 weeks of age. Scale bars 10 μm. FIG. 58L: Transcript levels of mKi67, Havcr1 and Lcn2 measured in 10 weeks old WT (n=5) and Acss2−/− (n=6) mice at baseline. Data are represented as mean±SEM. P values determined by one-way ANOVA. *p<0.05, **p<0.01, ***p<0.001 and ****p<0.0001.

FIGS. 59A-59Q demonstrate that the loss of ACSS2 protects from kidney disease, in accordance with some embodiments. FIG. 59A: Kidney weights in WT (n=7) and Acss2−/− (n=6) gavage with adenine. FIG. 59B: Quantification of ACSS2 immunoblot by image J. FIG. 59C: Quantification of FN1, αSMA and GAPDH immunoblots by image J. FIG. 59D. Quantification of Sirius red images by image J and plotted as % relative fibrosis area in WT (n=7) and Acss2−/− (n=6) mice gavage with adenine. FIG. 59E: Quantification of ACSS2 immunoblot in UUO kidneys by image J. FIG. 59F: Transcript levels of Acss2 measured in WT (n=5) and Acss2−/− (n=7) mice injected with folic acid nephropathy (FAN). FIG. 59G: (Left) Immunoblotting performed for ACSS2 in whole kidney lysates from WT and Acss2−/− mice injected with folic acid. (Right) Quantification of ACSS2 protein levels by image J. FIG. 59H: Immunoblots of FN1 and αSMA in whole kidney lysates of WT and Acss2−/− mice injected with folic acid. FIG. 59I: Quantification of FN1, αSMA and GAPDH immunoblots by image J. FIG. 59J: Collagen 1a1 (Col1a1), collagen type 3a (Col3a) and fibronectin (Fn1) mRNA levels were measured in kidneys of WT (n=5) and Acss2−/− (n=7) mice injected with folic acid. FIG. 59K: Quantification of fibrosis by image J and plotted as % relative fibrosis area in UUO of WT (n=6) and Acss2−/− (n=7) mice kidney sections. FIG. 59L: H&E and Sirius red staining images of WT and Acss2−/− mice injected with folic acid. Scale bars 20 μm. FIG. 59M: Quantification of Sirius red images by image J and plotted as % relative fibrosis area in WT (n=5) and Acss2−/− (n=7) mice injected with folic acid. FIG. 59N: Creatinine (sCr) levels estimated in serum samples collected from WT (n=5) and Acss2−/− (n=7) mice injected with folic acid. FIG. 59O: Blood urea nitrogen (BUN) levels estimated in serum samples collected from WT (n=5) and Acss2−/− (n=7) mice injected with folic acid. FIG. 59P: Alpha smooth muscle actin (Acta2), collagen 1a1 (Col1a1), collagen type 3a (Col3a) and fibronectin (Fn1) mRNA levels were measured in tubular epithelial cells (TECs) of WT and Acss2−/− mice treated with TGF-β1. FIG. 59Q: Western blotting performed in whole TECs lysates of WT and Acss2−/− mice for FN1, aSMA and GAPDH. Data are represented as mean±SEM. P values determined by one-way ANOVA in Graphpad Prism 9 software. *p<0.05, **p<0.01, ***p<0.001 and ****p<0.0001.

FIGS. 60A-60R demonstrate that TGF-β1 induces de novo lipogenesis in primary tubule cells, in accordance with some embodiments. FIG. 60A: Quantification of immunoblots of FN1, aSMA and GAPDH protein by image J. FIG. 60B: Seahorse based oxygen consumption rate (OCR) analysis in WT and Acss2−/− primary cells treated with palmitic acid. FIG. 60C: Seahorse based extracellular acidification rate (ECAR) analysis in WT and Acss2−/− primary cells treated with palmitic acid. FIG. 60D: Spare respiration and ATP production by WT and Acss2−/− cells treated with palmitic acid. FIG. 60E: Relative gene expression of Acox1, Acox2, Cpt1, and Ppara in WT and Acss2−/− primary TECs treated with TGF-β1. FIG. 60F: Quantification of FASN and GAPDH immunoblots in whole kidney lysates of WT UUO and ACSS2−/− UUO by image J. FIG. 60G: Experimental scheme. FIG. 60H: Cell viability was measured in FASNall or TVB-3664 condition at various concentrations for 48 h. FIG. 60I: Relative Fasn gene expression was measured in primary TECs treated with TGF-β1 in presence or absence of FASNall. FIG. 60J: Relative perilipin 2 (Plin2) gene expression was measured in primary TECs treated with TGF-β1 in presence or absence of FASNall. FIG. 60K: Oil Red 0 staining of primary TECs treated with TGF-β1 in presence or absence of FASNall (5 μM). Scale bars 20 μM. FIG. 60L: Quantification of Oil Red 0 staining. FIG. 60M: Triglycerides measured in TECs treated with TGF-β1 in the presence or absence of FASNall or TVB-3664. FIG. 60N: Relative gene expression levels of alpha smooth muscle actin (Acta2), Collagen 1a1 (Col1a1), collagen type 3a (Col3a) and fibronectin (Fn1) were measured in primary TECs cultured in the presence or absence of FASNall and TGF-β1. FIG. 60O: Oil Red O staining of primary TECs treated with TGF-β1 in presence or absence of TVB-3664. Scale bars 20 μM. FIG. 60P: Relative Fasn gene expression was measured in primary TECs treated with TGF-β1 in presence or absence of TVB-3664. FIG. 60Q: Relative perilipin 2 (Plin2) gene expression was measured in primary TECs treated with TGF-β1 in presence or absence of TVB-3664. FIG. 60R: Relative gene expression of alpha smooth muscle actin (Acta2), Collagen 1a1 (Col1a1), collagen type 3a (Col3a) and fibronectin (Fn1) measured in TECs treated with TGF-β1 or TVB-3664. All graphs present means of ±SEM. P values determined by one-way Anova in Graphpad Prism 9 software. *p<0.05, **p<0.01, ***p<0.001 and ****p<0.0001.

FIGS. 61A-61Z demonstrate that the inhibition of de novo lipogenesis prevents kidney fibrosis, in accordance with some embodiments. FIG. 61A: Gene expression levels of fatty acid synthase (Fasn) and perilipin2 (Plin2) measured in TECs transfected with siFasn and treated with TGF-β1 for 48 h. FIG. 61B: Gene expression levels of alpha smooth muscle actin (Acta2), Collagen 1a1 (Col1a1), collagen type 3a (Col3a) and fibronectin (Fn1) measured TECs transfected with siFasn and treated with TGF-β1 for 48 h. FIG. 61C: Gene expression levels of SREBP cleavage activating protein (Scap), fatty acid synthase (Fasn), and perilipin 2 (Plin2) were measured in WT and Scapf/f TECs treated with Adeno-Cre virus (Ad Cre) for 24 h and treated with TGF-β1 for 48 h. FIG. 61D: Gene expression levels of alpha smooth muscle actin (Acta2), Collagen 1a1 (Col1a1), collagen type 3a (Col3a) and fibronectin (Fn1) measured in WT and Scapf/f TECs treated with Adeno-Cre virus (Ad Cre) for 24 h and treated with TGF-β1 for 48 h. FIG. 61E: Experimental design. FIG. 61F: Gene expression levels of fatty acid synthase (Fasn) and perilipin 2 (Plin2) in whole kidney lysates of mice injected with FASNall or PBS followed by UUO injury. FIG. 61G: Total triglyceride levels were measured in kidneys of mice injected with FASNall or PBS and subjected to UUO injury. FIG. 61H: Representative images of Oil Red O-stained kidney sections of mice injected with FASNall, or PBS followed by UUO injury. Scale bars 10 μm. FIG. 61I: Fibronectin (FN1), alpha smooth muscle actin (α-SMA) and GAPDH immunoblots of whole kidney lysates of mice injected with FASNall or PBS in followed by UUO injury. FIG. 61J; Quantification of immunoblots of α-SMA and FN1 proteins in whole kidney lysates of UUO and FASNall treated UUO mice. Normalized to relative intensity of GAPDH blot. FIG. 61K: (Left) Representative images H&E and Sirius Red stained the kidney sections of mice injected with FASNall or PBS in followed by UUO injury. Scale bars 20 μm. (Right) Relative percentage of fibrosis was quantified in kidneys of UUO and FASNall treated UUO mice by image J. FIG. 61L: Gene expression levels of alpha smooth muscle actin (Acta2), Collagen 1a1 (Col1a1), collagen type 3a (Col3a) and fibronectin (Fn1) in whole kidney extracts of mice injected with FASNall or PBS in followed by UUO injury. FIG. 61M: Quantification of FASN and GAPDH immunoblots of Fasn f/f Ksp Cre mice with or without UUO. FIG. 61N: Fasn gene expression levels measured in mice Fasn f/f Ksp Cre (n=7) and WT (n=7) mice treated with adenine. FIG. 61O: Daily body weight changes recorded in adenine-CKD model of Fasn f/f Ksp Cre and WT mice. FIG. 61P: Final body weights of Fasn f/f Ksp Cre (n=7) and WT (n=7) mice treated with adenine. FIG. 61Q: Gene expression levels of alpha smooth muscle actin (Acta2), Collagen 1a1 (Col1a1), collagen type 3a (Col3a) and fibronectin (Fn1) measured in WT (n=7) and Fasn f/f Ksp Cre (n=7) mice gavaged with adenine. FIG. 61R: Fibronectin (FN1), alpha smooth muscle actin (α-SMA) and GAPDH immunoblots of whole kidney lysates of mice gavaged with adenine in WT (n=5) and Fasn f/f Ksp Cre (n=5). FIG. 61S: Quantification of FN1, α-SMA and GAPDH immunoblots of Fasn f/f Ksp Cre and WT mice gavaged with adenine. FIG. 61T: Representative images H&E and Sirius Red stained the kidney sections of mice treated with adenine or vehicle. Scale bars 20 μm. FIG. 61U: Quantification of Sirius red staining in image J in kidneys of mice gavaged with adenine in WT and Fasn f/f Ksp Cre mice. FIG. 61V: Serum creatinine (sCr) and blood urea nitrogen (BUN) estimated in WT (n=7) and Fasn f/f Ksp Cre (n=7) mice gavaged with adenine. FIG. 61W: Quantification of Sirius red staining in image J in kidneys mice with UUO following sham or ACSS2i treatment. FIG. 61X: Gene expression levels of Fasn and Plin2 were measured in WT (n=3) and ACSS2i (n=3) injected control and UUO kidneys. FIG. 61Y: Triglycerides measured in kidneys of WT (n=3) and ACSS2i (n=3) injected control and UUO kidneys. FIG. 61Z: Oil Red O staining for lipid droplets in fresh kidney sections of WT and ACSS2i injected control and UUO mice. Scale bars 10 μm. Data are represented as mean±SEM. P values determined by one-way ANOVA in Graphpad Prism 9 software. *p<0.05, **p<0.01, ***p<0.001 and ****p<0.0001.

FIGS. 62A-62K: Suppression of mitochondrial ROS suppresses NLRP3-inflammasome activation in primary tubular cells. FIG. 62A: Mitophagy was assessed with mitoCox8 eGFP-mCherry plasmid. Plasmid was transfected into primary TECs for 48 h and subjected to various mitophagy inducers for 2 h. Scale bars 10 μm. FIG. 62B: Mitolysosomes quantified using Image J in Fed, Starve and CCCP treatment conditions. FIG. 62C: Immunoblotting of LC3B and Parkin1 proteins in primary TEC cell lysates. FIG. 62D: Quantification of immunoblots of LC3B in Image J. Normalized to relative intensity of GAPDH blot. FIG. 61E: Relative gene expression of Nlrp3, IL-1B, and caspase1 in TECs treated with TVB-3664 or TGF-β1 for 48 hr. FIG. 62F: Relative gene expression of IL-1B, IL18 and caspase1 were measured in WT and Scapf/f TECs transfected with Adeno-Cre virus (Ad Cre) for 24 h and treated with TGF-β1. FIG. 62G: Relative gene expression of Fasn was measured in WT and Fasnf/-TECs transfected with Adeno-Cre virus (Ad Cre) for 24 h and treated with TGF-β1. FIG. 62H: Relative gene expression of IL-1B, IL18 and caspase1 were measured WT and Fasnf/-TECs transfected with Adeno-Cre virus (Ad Cre) for 24 h and treated with TGF-β1. FIG. 62I: Relative fluorescence of MitoSox quantified in WT and Acss2−/− cells treated with vehicle or TGF-β1 or mitoTempo (MT). FIG. 62J: Relative fluorescence of MitoSox quantified in WT and Fasnf/− cells transfected with Adeno-Cre virus (Ad Cre) for 24 h and treated with vehicle or TGF-β1 or MT. FIG. 62K: Relative gene expression of alpha smooth muscle actin (Acta2), Collagen 1a1 (Col1a1), collagen type 3a (Col3a) and fibronectin (Fn1) measured in primary TECs treated with TGF-β1 or mitoTempo (MT). Data are represented as mean±SEM. P values determined by one-way ANOVA in Graphpad Prism 9 software. *p<0.05, **p<0.01, ***p<0.001 and ****p<0.0001.

FIGS. 63A-63L demonstrate that the inhibition of de novo lipogenesis suppresses ROS-induced NLRP3 inflammasome activation, in accordance with some embodiments. FIG. 63A: Gene expression levels of Nlrp3, Caspase 1 (Casp1), Gasdemin D (Gsdmd), IL-1B, and IL-18 measured in kidneys of mice injected with vehicle (n=6) or FASNall (n=4). FIG. 63B: In situ hybridization of mouse GSDMD in kidneys of WT and Acss2−/− mice with UUO injury (upper panel) and its quantification (lower panel). HNF-4A was used to detect proximal tubule (PT) cells. Scale bars 10 μm. FIG. 63C: In situ hybridization of mouse GSDMD in kidneys of mice injected with ACSS2i or UUO injury (upper panel) and its quantification (lower panel). HNF-4A was used to detect proximal tubule (PT) cells. Scale bars 10 μm. FIG. 63D: Quantification of immunoblots of NLRP3, CASPASE1, P20 (Cleaved-CASPASE1), GSDMD-F (GSDMD-Full length), GSDMD-N(Cleaved-GSDMD) and GAPDH in control and UUO kidneys of WT and Fasn f/f; Ksp Cre mice. FIG. 63E: Quantification of immunoblots performed for NLRP3, total and p20 forms of Caspase1 and full length GSDMD and N-GSDMD in WT UUO and ACSS2i injected UUO mice. FIG. 63F: Immunoblots of NLRP3, CASPASE1, P20 (Cleaved-CASPASE1), GSDMD-F (GSDMD-Full length), GSDMD-N(Cleaved-GSDMD) and GAPDH in kidney lysates of mice injected with vehicle (n=6) or FASNall (n=4). FIG. 63G: Quantification of immunoblots of NLRP3, CASPASE1, P20 (Cleaved-CASPASE1), GSDMD-F (GSDMD-Full length), GSDMD-N (Cleaved-GSDMD) and GAPDH in vehicle or FASNall injected mice with UUO. FIG. 63H: Quantification of immunoblots performed for NLRP3, total and P20 forms of Caspase1 and full length GSDMD (GSDMD-F) and N-terminal GSDMD (GSDMD-N) in WT UUO and Acss2−/− mice subjected to UUO. FIG. 63I: Quantification of immunoblots of NLRP3, CASPASE1, P20 (Cleaved-CASPASE1), GSDMD-F (GSDMD-Full length), GSDMD-N (Cleaved-GSDMD) and GAPDH in vehicle (n=4), WT (n=5) and Acss2−/− (n=5) mice gavage with adenine. FIG. 63J: Immunoblots of NLRP3, CASPASE1, P20 (Cleaved-CASPASE1), GSDMD-F (GSDMD-Full length), GSDMD-N(Cleaved-GSDMD) and GAPDH in vehicle (n=4), WT (n=5) and Fasn f/f; Ksp Cre (n=5) mice gavage with adenine. FIG. 63K: Quantification of immunoblots of NLRP3, CASPASE1, P20 (Cleaved-CASPASE1), GSDMD-F (GSDMD-Full length), GSDMD-N(Cleaved-GSDMD) and GAPDH in vehicle (n=4), WT (n=5) and Fasn f/f; Ksp Cre (n=5) mice gavage with adenine. FIG. 63L: Gene expression of levels of Nlrp3, Caspase 1 (Casp1), Gasdemin D (Gsdmd), IL-1β, and IL-18 measured in kidneys of mice injected with vehicle (n=7), or adenine gavage in WT (n=7) and Fasn f/f; Ksp Cre (n=7) mice. Data are represented as mean±SEM. P values determined by one-way ANOVA in Graphpad Prism 9 software. *p<0.05, **p<0.01, ***p<0.001 and ****p<0.0001.

FIGS. 64A-64C demonstrate that the fatty acid synthesis correlated with fibrosis in CKD patients, in accordance with some embodiments. FIG. 64A: ACSS2 gene expression in human snRNA-seq data of 39,176 cells from 6 controls and 5 DKD samples. The size of the bubble correlates with the percent positive cells, and the color indicates the level of the expression (darker higher). Cell types: proximal tubule (PT), VCAM1 positive PT cells (PT-VCAM1+), parietal epithelial cells (PEC), ascending Loop of Henle (ATL), thick ascending Loop of Henle (TAL 1 and 2), distal convoluted tubule (DCT 1 and 2), principal cells (PC), intercalated cells A (IC_A) and intercalated cells B (IC_B), podocytes (Podo), endothelial cells (Endo), mesangial (Mes), fibroblasts (Fibro), leukocytes (LEUK). FIG. 64B: ACSS2 gene expression in mouse kidney snRNA-seq atlas. The size of the dot correlates with the percent positive cells, and the color indicates the level of the expression (darker higher). Cell types: podocytes (Podo), parietal epithelial cells (PEC), proximal convoluted tubule (PCT), proximal strait tubule (PST), injured PT (Inj. PT), descending limb of Loop of Henle (DTL), thick ascending Loop of Henle (TAL 1 and 2), macula densa (MD), distal convoluted tubule (DCT), principal cells (PC), intercalated cells A (IC_A) and intercalated cells B (IC_B), endothelial cells (EC), fibroblasts (Fib), juxtaglomerular apparatus (JGA) and immune cells. FIG. 64C: Immunofluorescence of Plin2 in healthy (upper panel) and CKD (lower panel) kidneys of human subjects. LTL was used to stain PT segments of the kidney. Scale bars 20 μm and enlarge scale is 10 μm.

FIG. 65: ACSS2 was prioritized as the top likely causal gene by integrating multiple genetic evidence and statistical methods with GWAS eGFRcrea. snATAC-seq identified open chromatin accessibility in proximal tubule (PT-S1-3) segments of adult human kidneys. Experimental studies identified the mechanism of ACSS2 in kidney disease. Either inhibition of ACSS2 or inhibition of FASN attenuates kidney fibrosis by disrupting proposed mechanism.

### DETAILED DESCRIPTION

Genome-wide association studies (GWAS) have identified more than 300 loci where genetic variants are associated with CKD development. However, these loci remain mostly unexplored. In one aspect, the present invention provides a method to explore the genetic loci of DPEP1 and CASP9, determine the causal variant regulating DPEP1 and CASP9 gene expression in a cell type specific manner, and its association with kidney disease. In another aspect the present invention is related to targeting DPEP1 and CASP9 genes for treating, ameliorating, and/or preventing chronic kidney disease (CKD) and complications thereof.

### Definitions

Unless defined otherwise, all technical and scientific terms used herein have the same meaning as commonly understood by one of ordinary skill in the art to which the disclosure pertains. Although any methods and materials similar or equivalent to those described herein can be used in the practice for testing of the present disclosure, illustrative materials and methods are described herein. In describing and claiming the present disclosure, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “chromosomal region” or “chromosomal segment”, as used herein, denotes a contiguous length of nucleotides in a genome of an organism. A chromosomal region may be in the range of 1000 nucleotides in length to an entire chromosome, e.g., 100 kb to 10 MB for example.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

As used herein, an “instructional material” includes a publication, a recording, a diagram, or any other medium of expression which can be used to communicate the usefulness of the compositions and methods of the disclosure. The instructional material of the kit of the disclosure may, for example, be affixed to a container which contains the nucleic acid, peptide, and/or composition of the disclosure or be shipped together with a container which contains the nucleic acid, peptide, and/or composition. Alternatively, the instructional material may be shipped separately from the container with the intention that the instructional material and the compound be used cooperatively by the recipient.

The term “limited toxicity” as used herein, refers to the peptides, polynucleotides, cells and/or antibodies of the disclosure manifesting a lack of substantially negative biological effects, anti-tumor effects, or substantially negative physiological symptoms toward a healthy cell, non-tumor cell, non-diseased cell, non-target cell or population of such cells either in vitro or in vivo.

By the term “modified” as used herein, is meant a changed state or structure of a molecule or cell of the disclosure. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g. subcutaneous (s.c.), intravenous (i.v.), intramuscular (i.m.), or intrastemal injection, or infusion techniques.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The terms “sequence alteration” or “sequence variation”, as used herein, refer to a difference in nucleic acid sequence between a test sample and a reference sample that may vary over a range of 1 to 10 bases, 10 to 100 bases, 100 to 100 kb, or 100 kb to 10 MB. Sequence alteration may include single nucleotide polymorphism and genetic mutations relative to wild-type. In certain embodiments, sequence alteration results from one or more parts of a chromosome being rearranged within a single chromosome or between chromosomes relative to a reference. In certain cases, a sequence alteration may reflect a difference, e.g. abnormality, in chromosome structure, such as an inversion, a deletion, an insertion or a translocation relative to a reference chromosome, for example.

As used herein, the term “single nucleotide polymorphism”, or “SNP” for short, refers to single nucleotide position in a genomic sequence for which two or more alternative alleles are present at appreciable frequency (e.g., at least 1%) in a population.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

A “target site” or “target sequence” refers to a genomic nucleic acid sequence that defines a portion of a nucleic acid to which a binding molecule may specifically bind under conditions sufficient for binding to occur.

The term “therapeutic” as used herein means a treatment and/or prophylaxis. A therapeutic effect is obtained by suppression, remission, or eradication of a disease state.

As used herein, the term “treatment” or “treating” encompasses prophylaxis and/or therapy. Accordingly, the compositions and methods of the present invention are not limited to therapeutic applications and can be used in prophylaxis ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Abbreviations:

GWAS: Genome-wide association studies; eGFRcrea: glomerular filtration rate estimated by serum creatinine; eQTLs: expression quantitative trait loci; meQTLs/mQTLs: methylation quantitative trait loci; SMR: summary-based Mendelian randomization; HEIDI: heterogeneity in dependent instruments; ABC model: activity-by-contact model; CKD: chronic kidney disease.

Methods Involving DPEP1 Inhibition and/or CASP9 Inhibition

In one aspect, the present invention includes a method for treating, ameliorating, and/or preventing a chronic kidney disease (CKD) and/or one or more complications thereof, in a subject (e.g. a human) in need thereof, wherein the method comprises administering to the subject a therapeutically effective amount at least one selected from the group consisting of DPEP1 inhibitor and a CASP9 inhibitor or a salt, solvate, tautomer, enantiomer, diastereoisomer, geometric isomer, and/or any combination thereof.

The inhibitors used in the disclosed methods can be any type of inhibitor known to one of ordinary skill in the art. The inhibitor can inhibit the function of any form of molecule including proteins or nucleic acids (DNA or RNA). As contemplated herein, an inhibitor is a chemical and/or biological agent that decreases and/or nullifies the biological role of a target molecule by decreasing the expression, concentration, and/or biological activity of the target molecule. In certain embodiments, the inhibitor decreases expression and/or concentration of the target molecule by decreasing expression of and/or increasing degradation of the target molecule. In certain embodiments, the inhibitor decreases biological activity of the target molecule by binding to the target molecule and interfering with biological process(es) in which the target molecule takes part.

In certain embodiments, the administering protects against fibrosis. In certain embodiments, the administering protects against ferroptosis.

In certain embodiments, the subject is a mammal. In certain embodiments, the subject is a human subject.

In another aspect, the invention provides a method for prioritizing and identifying target genes for kidney function GWAS loci. The method comprises integrating evidence from any or all of omics datasets or analytical tools such as, for example, eQTLs, meQTLs and eQTM, coloc (GWAS and eQTLs), moloc (GWASs, eQTLs and meQTLs), SMR, HEIDI, single-cell; co-accessibility and activity-by-contact (ABC) model.

Pharmaceutical Compositions that Inhibit DPEP1 and/or CASP9

In another aspect, the invention provides a pharmaceutical composition that comprises a therapeutically effective amount of DPEP1 and/or CASP9 inhibitor(s), as described elsewhere herein, in combination with one or more pharmaceutically or physiologically acceptable carriers, diluents, adjuvants or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Pharmaceutical compositions of the present disclosure may be administered in a manner appropriate for prevention and treatment of a CKD. The quantity and frequency of administration will be determined by such factors as the condition of the patient, and the type and severity of the patient's disease, although appropriate dosages may be determined by clinical trials.

Pharmaceutical compositions may be administered multiple times or in a single administration. Administration of the pharmaceutical composition may be combined with other methods useful to treat the disease or condition as determined by those of skill in the art.

The administration of the composition of the disclosure may be carried out in any convenient manner known to those of skill in the art. For example, the composition may be administered to a subject by aerosol inhalation, injection, ingestion, transfusion, implantation, transplantation, transarterially, subcutaneously, intradermally, intranodally, intramedullary, intramuscularly, by intravenous (i.v.) injection, or intraperitoneally.

It should be understood that the method and compositions that would be useful in the present disclosure are not limited to the particular formulations set forth in the examples. The following examples are put forth so as to provide those of ordinary skill in the art with a complete disclosure and description of how to make and use the disclosure and are not intended to limit the scope of what the inventors regard as their disclosure.

Kits that Inhibit DPEP1 and/or CASP9

In yet another aspect, the invention provides a kit including pharmaceutical composition comprising a therapeutically effective amount of DPEP1 and/or CASP9 inhibitor(s), inhibitors as described elsewhere herein and an instructions regarding using the composition for treating, ameliorating, and/or preventing a chronic kidney disease (CKD) and/or one or more complications thereof, in a subject in need thereof.

Methods Involving ACSS2 and/or FASN Inhibition

In one aspect, the present invention includes a method for treating, ameliorating, and/or preventing a chronic kidney disease (CKD) and/or one or more complications thereof, in a subject (e.g. a human) in need thereof, wherein the method comprises administering to the subject a therapeutically effective amount of ACSS2 inhibitor and/or FASN inhibitor.

The inhibitors used in the disclosed methods can be any type of inhibitor known to one of ordinary skill in the art. The inhibitor can inhibit any form of molecule including proteins or nucleic acids (DNA or RNA). As contemplated herein, an inhibitor is a chemical and/or biological agent that decreases and/or nullifies the biological role of a target molecule by decreasing the expression, concentration, and/or biological activity of the target molecule. In certain embodiments, the inhibitor decreases expression and/or concentration of the target molecule by decreasing expression of and/or increasing degradation of the target molecule. In certain embodiments, the inhibitor decreases biological activity of the target molecule by binding to the target molecule and interfering with biological process(es) in which the target molecule takes part.

In certain embodiments, reducing ACSS2 and/or FASN expression levels inhibits/protects against fibrosis.

In certain embodiments, the subject is a mammal. In certain embodiments, the subject is a human subject.

In another aspect, the invention provides a method for identifying target genes for treating CKD by integrating GWAS, eQTL and mQTL data.

In another aspect, the invention provides a method for prioritizing and identifying target genes for kidney function GWAS loci. The method comprises integrating evidence from any or all of omics datasets or analytical tools such as, for example, eQTLs, meQTLs and eQTM, coloc (GWAS and eQTLs), moloc (GWASs, eQTLs and meQTLs), SMR, HEIDI, single-cell; co-accessibility and activity-by-contact (ABC) model.

In certain embodiments, the genetic locus containing ACSS2 and/or FASN was identified by expression quantitative trait loci (eQTL and mQTL) analysis. eQTL and mQTL analysis using computational integration (coloc and moloc) to combine genomic data with transcriptomic data. Further, to see which kidney cell types express the genetic variants associated with ACSS2 and/or FASN expression, kidney single cell RNA and ATAC-seq data were integrated.

Pharmaceutical Compositions that Inhibit ACSS2 and/or FASN

In another aspect, the invention provides a pharmaceutical composition that comprises therapeutically effective amount of ACSS2 inhibitor(s) and/or FASN inhibitor(s), as described elsewhere herein, in combination with one or more pharmaceutically or physiologically acceptable carriers, diluents, adjuvants or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Pharmaceutical compositions of the present disclosure may be administered in a manner appropriate for prevention and treatment of a CKD. The quantity and frequency of administration will be determined by such factors as the condition of the patient, and the type and severity of the patient's disease, although appropriate dosages may be determined by clinical trials.

Pharmaceutical compositions may be administered multiple times or in a single administration. Administration of the pharmaceutical composition may be combined with other methods useful to treat the disease or condition as determined by those of skill in the art.

The administration of the composition of the disclosure may be carried out in any convenient manner known to those of skill in the art. For example, the composition may be administered to a subject by aerosol inhalation, injection, ingestion, transfusion, implantation, transplantation, transarterially, subcutaneously, intradermally, intranodally, intramedullary, intramuscularly, by intravenous (i.v.) injection, or intraperitoneally.

It should be understood that the method and compositions that would be useful in the present disclosure are not limited to the particular formulations set forth in the examples. The following examples are put forth so as to provide those of ordinary skill in the art with a complete disclosure and description of how to make and use the disclosure and are not intended to limit the scope of what the inventors regard as their disclosure.

Kits that Inhibit ACSS2 and/or FASN

In yet another aspect, the invention provides a kit including pharmaceutical composition comprising a therapeutically effective amount of ACSS2 and/or FASN inhibitor(s) as described elsewhere herein and an instruction regarding using the composition for treating, ameliorating, and/or preventing a chronic kidney disease (CKD) and/or one or more complications thereof, in a subject in need thereof.

### Down-Regulating DPEP1/CASP9/ACSS2/FASN by Small Molecule Inhibitors

In some embodiments, the compound that downregulates the expression level or the activity of DPEP1/CASP9/ACSS2/FASN includes a small molecule that inhibits the activity of DPEP1/CASP9/ACSS2/FASN. As used herein, the term “small molecule” refers to a molecule having a size of about 2,000 daltons or less, such as about 1,500 daltons or less, about 1,000 daltons or less, about 900 daltons or less, about 800 daltons or less, about 700 daltons or less, about 600 daltons or less, or about 500 daltons or less.

Non-limiting examples of small molecule inhibitors of DPEP1 include the LSALT peptide, cilastatin, and the like.

Non-limiting examples of small molecule inhibitors of CASP9 include Caspase-9 Inhibitor I

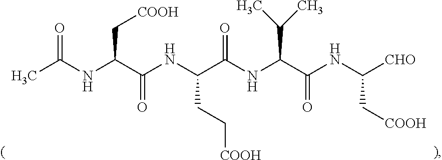 [SMILES: CC(=O)N[C@@H](CC(=O)O)C(=O)N[C@@H](CCC(=O)O)C(=O)N[C@H](C(=O)N[C@H](C=O)CC(=O)O)C(C)C]

### Caspase-9 Inhibitor II, Caspase-9 Inhibitor III

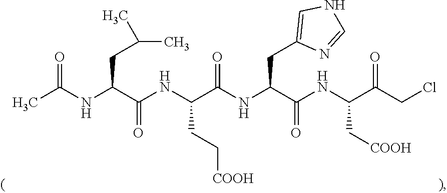 [SMILES: CC(=O)N[C@@H](CC(C)C)C(=O)N[C@@H](CCC(=O)O)C(=O)N[C@@H](Cc1c[nH]cn1)C(=O)N[C@@H](CC(=O)O)C(=O)CCl]

and the like.

Non-limiting examples of small molecule inhibitors of ACSS2 include MTB-9655, and the like.

Non-limiting examples of small molecule inhibitors of FASN include FASNall, TVB-3664, and the like.

### Downregulating DPEP1/CASP9/ACSS2/FASN by Protein Inhibitors

In some embodiments, the compound that downregulates the expression level or the activity of DPEP1/CASP9/ACSS2/FASN includes a protein that downregulates the expression level or the activity of DPEP1/CASP9/ACSS2/FASN.

In some embodiments, the protein that downregulates the expression level or the activity of DPEP1/CASP9/ACSS2/FASN is an antibody (or an antigen binding fragment thereof) that specifically target DPEP1/CASP9/ACSS2/FASN.

Non-limiting examples of antibodies that specifically target DPEP1 include PA5-52984, PA5-52670, MA5-43849 and PA5-32723 from Invitrogen (Waltham, Massachusetts, USA); ab230977, ab230978, and ab242083 from abcam (Cambridge, United Kingdom); and the like.

Non-limiting examples of antibodies that specifically target CASP9 include LAP6 96-2-22/MA1-16842, PA5-19904, and MA1-12562 from Invitrogen (Waltham, Massachusetts, USA); ab32539, ab202068, ab185719, and ab32068 from abcam (Cambridge, United Kingdom); and the like.

Non-limiting examples of antibodies that specifically target ACSS2 include T.407.4/MA5-14810, OTI3H4/MA5-25697, and PA5-26612 from Invitrogen (Waltham, Massachusetts, USA); ab133664, ab314490, and ab66038 from abcam (Cambridge, United Kingdom); and the like.

Non-limiting examples of antibodies that specifically target FASN include EPR7466, EPR7465 and ab99359 from abcam (Cambridge, United Kingdom); antibody #3180 from Cell Signaling Technology (Danvers, Massachusetts, USA); and the like.

### Downregulating DPEP1/CASP9/ACSS2/FASN by RNA Interference

In some embodiments, the compound that downregulates the activity or expression level of DPEP1/CASP9/ACSS2/FASN includes a nucleic acid that downregulates the activity and/or expression level of DPEP1/CASP9/ACSS2/FASN by the means of RNA interreference.

In some embodiments, the nucleic acid that downregulates the expression level of DPEP1/CASP9/ACSS2/FASN by the means of RNA interreference includes an isolated nucleic acid. In other embodiments, the modulator is an RNAi molecule (such as but not limited to siRNA and/or shRNA and/or miRNAs) or antisense molecule, which inhibits DPEP1/CASP9/ACSS2/FASN expression and/or activity. In yet other embodiments, the nucleic acid comprises a promoter/regulatory sequence, such that the nucleic acid is preferably capable of directing expression of the nucleic acid. Thus, the instant specification provides expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In certain embodiments, siRNA is used to decrease the level of DPEP1/CASP9/ACSS2/FASN. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; Engelke, Ed., RNA Interference (RNAi) Nuts & Bolts of RNAi Technology, DNA Press, Eagleville, P A (2003); and Hannon, Ed., RNAi A Guide to Gene Silencing, Cold Spring Harbor Laboratory Press, Cold Spring Harbor, NY (2003). Soutschek et al. (2004, Nature 432:173-178) describes a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the instant specification also includes methods of decreasing levels of DPEP1/CASP9/ACSS2/FASN using RNAi technology.

In certain embodiments, the instant specification provides a vector comprising an siRNA or antisense polynucleotide. In other embodiments, the siRNA or antisense polynucleotide inhibits the expression of DPEP1/CASP9/ACSS2/FASN. The incorporation of a desired polynucleotide into a vector and the choice of vectors is well-known in the art.

In certain embodiments, the expression vectors described herein encode a short hairpin RNA (shRNA) inhibitor. shRNA inhibitors are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleaves the shRNA to form siRNA.

The siRNA, shRNA, or antisense polynucleotide can be cloned into a number of types of vectors as described elsewhere herein. For expression of the siRNA or antisense polynucleotide, at least one module in each promoter functions to position the start site for RNA synthesis.

In order to assess the expression of the siRNA, shRNA, or antisense polynucleotide, the expression vector to be introduced into a cell can also contain either a selectable marker gene or a reporter gene or both to facilitate identification and selection of expressing cells from the population of cells sought to be transfected or infected using a viral vector. In certain embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

Following the generation of the siRNA polynucleotide, a skilled artisan will understand that the siRNA polynucleotide has certain characteristics that can be modified to improve the siRNA as a therapeutic compound. Therefore, in some embodiments, the siRNA polynucleotide is further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl- methyl-, thio- and other modified forms of adenine, cytidine, guanine, thymine, and uridine.

In certain embodiments, an antisense nucleic acid sequence expressed by a plasmid vector is used to inhibit DPEP1/CASP9/ACSS2/FASN protein expression. The antisense expressing vector is used to transfect a mammalian cell or the mammal itself, thereby causing reduced endogenous expression of DPEP1/CASP9/ACSS2/FASN.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the instant specification may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the instant specification include oligonucleotide derivatives known in the art which have improved biological activity compared to unmodified oligonucleotides (see U.S. Pat. No. 5,023,243).

### Downregulating DPEP1/CASP9/ACSS2/FASN by Ribozyme

In some embodiments, the compound that down regulates the activity or expression level of DPEP1/CASP9/ACSS2/FASN includes a ribosome that inhibits DPEP1/CASP9/ACSS2/FASN protein expression.

A ribozyme is used to inhibit DPEP1/CASP9/ACSS2/FASN protein expression. Ribozymes useful for inhibiting the expression of a target molecule may be designed by incorporating target sequences into the basic ribozyme structure which are complementary, for example, to the mRNA sequence encoding DPEP1/CASP9/ACSS2/FASN. Ribozymes are antisense RNAs which have a catalytic site capable of specifically cleaving complementary RNAs. Therefore, ribozymes having sequence complementary to DPEP1/CASP9/ACSS2/FASN mRNA sequences are capable of downregulating the expression of DPEP1/CASP9/ACSS2/FASN by reduces the level of DPEP1/CASP9/ACSS2/FASN mRNA. Ribozymes targeting DPEP1/CASP9/ACSS2/FASN, may be synthesized using commercially available reagents (Applied Biosystems, Inc., Foster City, CA) or they may be genetically expressed from DNA encoding them. In some embodiments, the DNA encoding the ribozymes are incorporated in a vector, which is described in the “Vector” section elsewhere in the instant specification.

### Downregulating DPEP1/CASP9/ACSS2/FASN by CRISPR Knockout and Other Knockouts/Knockdown Techniques

In some embodiments, the compound that down regulates the activity or expression level of DPEP1/CASP9/ACSS2/FASN comprises a nucleic acid that down regulates the expression level of DPEP1/CASP9/ACSS2/FASN by the means of CRISPR knockout.

The CRISPR/Cas9 system is a facile and efficient system for inducing targeted genetic alterations. Target recognition by the Cas9 protein requires a “seed” sequence within the guide RNA (gRNA) and a conserved di-nucleotide containing protospacer adjacent motif (PAM) sequence upstream of the gRNA-binding region. The CRISPR/Cas9 system can thereby be engineered to cleave virtually any DNA sequence by redesigning the gRNA in cell lines (such as 293T cells), primary cells, and CAR T cells. The CRISPR/Cas9 system can simultaneously target multiple genomic loci by co-expressing a single Cas9 protein with two or more gRNAs, making this system uniquely suited for multiple gene editing or synergistic activation of target genes.

The Cas9 protein and guide RNA form a complex that identifies and cleaves target sequences. Cas9 is comprised of six domains: REC I, REC II, Bridge Helix, PAM interacting, HNH, and RuvC. The RecI domain binds the guide RNA, while the Bridge helix binds to target DNA. The HNH and RuvC domains are nuclease domains. Guide RNA is engineered to have a 5′ end that is complementary to the target DNA sequence. Upon binding of the guide RNA to the Cas9 protein, a conformational change occurs activating the protein. Once activated, Cas9 searches for target DNA by binding to sequences that match its protospacer adjacent motif (PAM) sequence. A PAM is a two or three nucleotide base sequence within one nucleotide downstream of the region complementary to the guide RNA. In one non-limiting example, the PAM sequence is 5′-NGG-3′. When the Cas9 protein finds its target sequence with the appropriate PAM, it melts the bases upstream of the PAM and pairs them with the complementary region on the guide RNA. Then the RuvC and HNH nuclease domains cut the target DNA after the third nucleotide base upstream of the PAM.

One non-limiting example of a CRISPR/Cas system used to inhibit gene expression, CRISPRi, is described in U.S. Patent Appl. Publ. No. US2014/0068797. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

CRISPR/Cas gene disruption occurs when a guide nucleic acid sequence specific for a target gene and a Cas endonuclease are introduced into a cell and form a complex that enables the Cas endonuclease to introduce a double strand break at the target gene. In certain embodiments, the CRISPR/Cas system comprises an expression vector, such as, but not limited to, an pAd5F35-CRISPR vector. In other embodiments, the Cas expression vector induces expression of Cas9 endonuclease. Other endonucleases may also be used, including but not limited to, T7, Cas3, Cas8a, Cas8b, Cas10d, Cse1, Csy1, Csn2, Cas4, Cas10, Csm2, Cmr5, Fok1, other nucleases known in the art, and any combinations thereof.

In certain embodiments, inducing the Cas expression vector comprises exposing the cell to an agent that activates an inducible promoter in the Cas expression vector. In such embodiments, the Cas expression vector includes an inducible promoter, such as one that is inducible by exposure to an antibiotic (e.g., by tetracycline or a derivative of tetracycline, for example doxycycline). However, it should be appreciated that other inducible promoters can be used. The inducing agent can be a selective condition (e.g., exposure to an agent, for example an antibiotic) that results in induction of the inducible promoter. This results in expression of the Cas expression vector.

In certain embodiments, guide RNA(s) and Cas9 can be delivered to a cell as a ribonucleoprotein (RNP) complex. RNPs are comprised of purified Cas9 protein complexed with gRNA and are well known in the art to be efficiently delivered to multiple types of cells, including but not limited to stem cells and immune cells (Addgene, Cambridge, MA, Mirus Bio LLC, Madison, WI).

The guide RNA is specific for a genomic region of interest and targets that region for Cas endonuclease-induced double strand breaks. The target sequence of the guide RNA sequence may be within a loci of a gene or within a non-coding region of the genome. In certain embodiments, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length.

Guide RNA (gRNA), also referred to as “short guide RNA” or “sgRNA”, provides both targeting specificity and scaffolding/binding ability for the Cas9 nuclease. The gRNA can be a synthetic RNA composed of a targeting sequence and scaffold sequence derived from endogenous bacterial crRNA and tracrRNA. gRNA is used to target Cas9 to a specific genomic locus in genome engineering experiments. Guide RNAs can be designed using standard tools well known in the art.

In the context of formation of a CRISPR complex, “target sequence” refers to a sequence to which a guide sequence is designed to have some complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In certain embodiments, a target sequence is located in the nucleus or cytoplasm of a cell. In other embodiments, the target sequence may be within an organelle of a eukaryotic cell, for example, mitochondrion or nucleus. Typically, in the context of an endogenous CRISPR system, formation of a CRISPR complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) the target sequence. As with the target sequence, it is believed that complete complementarity is not needed, provided this is sufficient to be functional.

In certain embodiments, one or more vectors driving expression of one or more elements of a CRISPR system are introduced into a host cell, such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In certain embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron).

In certain embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Additional domains that may form part of a fusion protein comprising a CRISPR enzyme are described in U.S. Patent Appl. Publ. No. US20110059502, incorporated herein by reference. In certain embodiments, a tagged CRISPR enzyme is used to identify the location of a target sequence.

Conventional viral and non-viral based gene transfer methods can be used to introduce nucleic acids in mammalian and non-mammalian cells or target tissues. Such methods can be used to administer nucleic acids encoding components of a CRISPR system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell (Anderson, 1992, Science 256:808-813; and Yu, et al., 1994, Gene Therapy 1:13-26).

In certain embodiments, the CRISPR/Cas is derived from a type II CRISPR/Cas system. In other embodiments, the CRISPR/Cas sytem is derived from a Cas9 protein. The Cas9 protein can be from Streptococcus pyogenes, Streptococcus thermophilus, or other species.

In general, Cas proteins comprise at least one RNA recognition and/or RNA binding domain. RNA recognition and/or RNA binding domains interact with the guiding RNA. Cas proteins can also comprise nuclease domains (i.e., DNase or RNase domains), DNA binding domains, helicase domains, RNAse domains, protein-protein interaction domains, dimerization domains, as well as other domains. The Cas proteins can be modified to increase nucleic acid binding affinity and/or specificity, alter an enzymatic activity, and/or change another property of the protein. In certain embodiments, the Cas-like protein of the fusion protein can be derived from a wild type Cas9 protein or fragment thereof. In other embodiments, the Cas can be derived from modified Cas9 protein. For example, the amino acid sequence of the Cas9 protein can be modified to alter one or more properties (e.g., nuclease activity, affinity, stability, and so forth) of the protein. Alternatively, domains of the Cas9 protein not involved in RNA-guided cleavage can be eliminated from the protein such that the modified Cas9 protein is smaller than the wild type Cas9 protein. In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-stranded break in DNA. (Jinek, et al., 2012, Science, 337:816-821). In certain embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

In one non-limiting embodiment, a vector drives the expression of the CRISPR system. The art is replete with suitable vectors that are useful in the instant specification. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the instant specification may also be used for nucleic acid standard gene delivery protocols. Methods for gene delivery are known in the art (U.S. Pat. Nos. 5,399,346, 5,580,859 & 5,589,466, incorporated by reference herein in their entireties).

Further, the vector may be provided to a cell in the form of a viral vector. Viral vector technology is well known in the art and is described, for example, in Sambrook et al. (4^{th} Edition, Molecular Cloning: A Laboratory Manual, Cold Spring Harbor Laboratory, New York, 2012), and in other virology and molecular biology manuals. Viruses, which are useful as vectors include, but are not limited to, retroviruses, adenoviruses, adeno-associated viruses, herpes viruses, Sindbis virus, gammaretrovirus and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

In some embodiments, the present invention includes any other methods for effecting gene knockdown and/editing, which allow for deletion and/or inactivation of PDL3, such as but not limited to those described in WO 2018/236840 (which is incorporated herein in its entirety by reference).

Downregulating DPEP1/CASP9/ACSS2/FASN by Inactivating and/or Sequestering

In some embodiments, the compound that downregulates the activity or expression level of DPEP1/CASP9/ACSS2/FASN includes a protein that downregulates the activity of DPEP1/CASP9/ACSS2/FASN by inactivating and/or sequestering PDL3. In some embodiment, the compound includes a nucleic acid that express the protein that downregulates the activity of DPEP1/CASP9/ACSS2/FASN by inactivating and/or sequestering DPEP1/CASP9/ACSS2/FASN. In some embodiments, the compound includes an expression vector that express the protein that downregulates the activity of DPEP1/CASP9/ACSS2/FASN by inactivating and/or sequestering DPEP1/CASP9/ACSS2/FASN (see “Vector” section for descriptions on vectors).

In some embodiments, the compound that downregulates the expression level of DPEP1/CASP9/ACSS2/FASN is a trans-dominant negative mutant of DPEP1/CASP9/ACSS2/FASN, and/or a nucleic acid or a vector expressing the trans-dominant negative mutant of DPEP1/CASP9/ACSS2/FASN.

### Vectors

Vectors can increase the stability of the nucleic acids, make the delivery easier, or allow the expression of the nucleic acids or protein products thereof in the cells.

Therefore, in some embodiments, the protein inhibitors or the nucleic acids that that down regulates the activity or expression level of DPEP1/CASP9/ACSS2/FASN is incorporated into a vector.

In some embodiments, the instant specification relates to a vector, including the nucleic acid sequence of the instant specification or the construct of the instant specification. The choice of the vector will depend on the host cell in which it is to be subsequently introduced. In certain embodiments, the vector of the instant specification is an expression vector. Suitable host cells include a wide variety of prokaryotic and eukaryotic host cells. In certain embodiments, the expression vector is selected from the group consisting of a viral vector, a bacterial vector and a mammalian cell vector. Prokaryote- and/or eukaryote-vector based systems can be employed for use with the instant specification to produce polynucleotide, or their cognate polypeptides. Many such systems are commercially and widely available.

In some embodiments, the vector is a viral vector. Viral vector technology is well known in the art and is described, for example, in virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

In some embodiments, the viral vector is a suitable adeno-associated virus (AAV), such as the AAV1-AAV8 family of adeno-associated viruses. In some embodiments, the viral vector is a viral vector that can infect a human. The desired nucleic acid sequence, such as the nucleic acids that downregulates DPEP1/CASP9/ACSS2/FASN described above, can be inserted between the inverted terminal repeats (ITRs) in the AAV. In various embodiments, the viral vector is an AAV2 or an AAV8. The promoter can be a thyroxine binding globulin (TBG) promoter. In various embodiments, the promoter is a human promoter sequence that enables the desired nucleic acid expression in the liver. The AAV can be a recombinant AAV, in which the capsid comes from one AAV serotype and the ITRs come from another AAV serotype. In various embodiments, the AAV capsid is selected from the group consisting of a AAV1, AAV2, AAV3, AAV4, AAV5, AAV6, AAV7, and a AAV8 capsid. In various embodiments, the ITR in the AAV is at least one ITR selected from the group consisting of a AAV1, AAV2, AAV3, AAV4, AAV5, AAV6, AAV7, and an AAV8 ITR. In various embodiments, the instant specification contemplates an AAV8 viral vector (recombinant or non-recombinant) containing a desired nucleic acid expression sequence and at least one promoter sequence that, when administered to a subject, causes elevated systemic expression of the desired nucleic acid. In some embodiments, the viral vector is a recombinant or non-recombinant AAV2 or AAV5 containing any of the desired nucleic acid expression sequences described herein.

In some embodiments, the vector in which the nucleic acid sequence is introduced is a plasmid that is or is not integrated in the genome of a host cell when it is introduced in the cell. Illustrative, non-limiting examples of vectors in which the nucleotide sequence of the instant specification or the gene construct of the instant specification can be inserted include a tet-on inducible vector for expression in eukaryote cells.

The vector may be obtained by conventional methods known by persons skilled in the art (Sambrook et al., 2012). In certain embodiments, the vector is a vector useful for transforming animal cells.

In certain embodiments, the recombinant expression vectors may also contain nucleic acid molecules which encode a peptide or peptidomimetic inhibitor of the instant specification, described elsewhere herein.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

It will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression. The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

The recombinant expression vectors may also contain a selectable marker gene which facilitates the selection of transformed or transfected host cells. Suitable selectable marker genes are genes encoding proteins such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

### Combination Therapies

In some embodiments, the method of treating, ameliorating, and/or preventing the chronic kidney disease includes administering to the subject the effective amount of at least one compound and/or composition contemplated within the disclosure.

In some embodiments, the composition for treating chronic kidney disease includes at least one compound and/or composition contemplated within the disclosure.

In some embodiments, the subject is further administered at least one additional agent that treats, ameliorates, and/or prevents a disease and/or disorder contemplated herein. In other embodiments, the compound and the at least one additional agent are co-administered to the subject. In yet other embodiments, the compound and the at least one additional agent are co-formulated.

The compounds contemplated within the disclosure are intended to be useful in combination with one or more additional compounds. These additional compounds may comprise compounds of the present disclosure and/or at least one additional agent for treating chronic kidney disease, and/or at least one additional agent that treats one or more diseases or disorders contemplated herein.

A synergistic effect may be calculated, for example, using suitable methods such as, for example, the Sigmoid-Eₘₐₓ equation (Holford & Schemer, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations contemplated within the disclosure may be administered to the subject either prior to or after the onset of a disease and/or disorder contemplated herein. Further, several divided dosages, as well as staggered dosages may be administered daily or sequentially, or the dose may be continuously infused, or may be a bolus injection. Further, the dosages of the therapeutic formulations contemplated within the disclosure may be proportionally increased or decreased as indicated by the exigencies of the therapeutic or prophylactic situation.

Administration of the compositions contemplated within the disclosure to a patient, preferably a mammal, more preferably a human, may be carried out using known procedures, at dosages and for periods of time effective to treat a disease and/or disorder contemplated herein in the patient. An effective amount of the therapeutic compound necessary to achieve a therapeutic effect may vary according to factors such as the state of the disease or disorder in the patient; the age, sex, and weight of the patient; and the ability of the therapeutic compound contemplated within the disclosure to treat a disease and/or disorder contemplated herein in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound contemplated within the disclosure is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions contemplated within the disclosure may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds contemplated within the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms contemplated within the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease and/or disorder contemplated herein.

In certain embodiments, the compositions of the disclosure are formulated using one or more pharmaceutically acceptable excipients or carriers. In certain embodiments, the pharmaceutical compositions of the disclosure comprise a therapeutically effective amount of a compound of the disclosure and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the disclosure are administered to the patient in dosages that range from one to five times per day or more. In another embodiment, the compositions of the disclosure are administered to the patient in range of dosages that include, but are not limited to, once every day, every two, days, every three days to once a week, and once every two weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

Compounds of the disclosure for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the disclosure is from about 1 mg and about 2,500 mg. In some embodiments, a dose of a compound of the disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of chronic kidney disease in a patient.

Formulations may be employed in admixtures with conventional excipients, i.e., pharmaceutically acceptable organic or inorganic carrier substances suitable for intracranially, oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the disclosure include oral, nasal, rectal, intravaginal, parenteral, buccal, sublingual or topical. The compounds for use in the disclosure may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the disclosure may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

The present disclosure also includes a multi-layer tablet comprising a layer providing for the delayed release of one or more compounds of the disclosure, and a further layer providing for the immediate release of another medication. Using a wax/pH-sensitive polymer mix, a gastric insoluble composition may be obtained in which the active ingredient is entrapped, ensuring its delayed release.

### Parenteral Administration

For parenteral administration, the compounds of the disclosure may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this disclosure include dosage forms as described in U.S. Pat. Nos. 6,340,475; 6,488,962; 6,451,808; 5,972,389; 5,582,837; and 5,007,790. Additional dosage forms of this disclosure also include dosage forms as described in U.S. Patent Applications Nos. 20030147952; 20030104062; 20030104053; 20030044466; 20030039688; and 20020051820. Additional dosage forms of this disclosure also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In certain embodiments of the disclosure, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present disclosure depends on the age, sex and weight of the patient, the current medical condition of the patient and the progression of the chronic kidney disease in the patient being treated. The skilled artisan is able to determine appropriate dosages depending on these and other factors.

A suitable dose of a compound of the present disclosure may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the modulator of the disclosure is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the patient's condition, to a level at which the improved disease is retained. In certain embodiments, patients require intermittent treatment on a long-term basis upon any recurrence of symptoms and/or infection.

The compounds for use in the method of the disclosure may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. Capsid assembly modulators exhibiting high therapeutic indices are preferred. The data obtained from cell culture assays and animal studies are optionally used in formulating a range of dosage for use in human. The dosage of such capsid assembly modulators lies preferably within a range of circulating concentrations that include the ED₅₀ with minimal toxicity. The dosage optionally varies within this range depending upon the dosage form employed and the route of administration utilized.

Those skilled in the art recognizes, or is able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in assay and/or reaction conditions, with art-recognized alternatives and using no more than routine experimentation, are within the scope of the present application.

The practice of the present disclosure employs, unless otherwise indicated, conventional techniques of molecular biology (including recombinant techniques), microbiology, cell biology, biochemistry and chemistry, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the disclosure, and, as such, may be considered in making and practicing the disclosure. Particularly useful techniques for particular embodiments will be discussed in the sections that follow.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Identification of Target Genes, Cell Types and Target Mechanisms for Chronic Kidney Disease Using Human Genetic Information

### Example 1-1: Material and Methods

### Prioritization of Kidney Disease Genes

The method for prioritizing and identifying target genes for kidney function GWAS loci comprises a priority scoring strategy by integrating evidence from any and/or all eight different datasets or analytical tools including (1) significant SNP-gene associations by kidney eQTLs (FDR<0.05); (2) significant SNP-CpG-gene associations by kidney meQTLs (FDR<0.05) and eQTM (CpG-level FDR<0.05); (3) SNP-gene pairs by coloc analysis between eGFRcrea GWAS and eQTLs (H4>0.8); (4) SNP-gene pairs by moloc analysis among eGFRcrea GWAS, eQTLs and meQTLs (PPA.abc>0.8); (5) significant SNP-gene pairs by Mendelian randomization analysis between eGFRcrea GWASs and eQTLs (PSMR<1.38×10-4); (6) SNP-gene pairs passing the HEIDI test between eGFRcrea GWAS and eQTLs (PHEIDI>0.01); (7) co-accessibility (Cicero connections) identified using 57,229 snATAC-seq cells (co-accessibility score>0.2); and (8) enhancer-promoter contacts identified by an ABC model that predicts enhancers regulating genes based on estimating enhancer activity and enhancer-promoter contact frequency from epigenomic datasets (ABC scores≥0.015). Promoters were defined as ±2,000 bp from the TSS of protein-coding transcripts from GENCODE v.351ift37 to annotate Cicero connections or ABC connections between gene promoters and eGFRcrea GWAS variants.

For each significant eGFRcrea GWAS variant, protein-coding genes were extracted within 1 Mb of the SNP as potential targets. For each SNP-gene pair, a priority score was defined by counting the number of datasets supporting the association. For each variant, the gene with the highest priority score was assigned as its target gene. If multiple genes shared the highest priority score, the closest gene with the most significant eQTL was assigned as the target gene. For each independent locus, the top target gene was determined according to the highest priority score from all variant gene pairs in the same locus. If multiple genes shared the highest priority score, the gene targeted by the variant with the most significant GWAS association was assigned as the top target gene for the locus. Newly prioritized loci were defined if they did not overlap with 309 independent signals (using gene prioritization score≥1) prioritized in eGFRcrea GWAS by Stanzick et al. (Stanzick, K. J. et al.; Nat. Commun. 12, 4350 (2021)) or 53 creatinine-associated exome rare variants identified in exome association studies by Backman et al. (Backman, J. D. et al. Nature 599, 628-634 (2021). or Barton et al. (Barton, A. R., Nat. Genet. 53, 1260-1269 (2021).

Furthermore, 328 GWAS loci with 559 target genes with a priority score of at least 3. First, 110 loci were inspected with 2 or more target genes by counting the number of independent signals (fine-mapped in 1 million European ancestry individuals) and co-expression gene pairs (FDR<0.05 accounting for all correlation tests) for each locus. To explore the function of prioritized genes, gene-set enrichment was performed for tissue specificity and GWAS catalog genes using GENE2FUNC of FUMA (Watanabe, K. et al., Nat. Commun. 8, 1826 (2017)). with protein-coding genes as a background gene set. Functional enrichment analysis for these genes was performed using DAVID Bioinformatics Resources (v.6.8) (Huang da, W., Nat. Protoc. 4, 44-57 (2009)). For enrichment to the cell type-specific genes, the present study obtained their mouse orthologues and overlapped with cell type-specific expressed genes identified using mouse single-cell RNA-seq (Park, J. et al. Science 360, 758-763 (2018)). The cell-type enrichment significance was determined using a hypergeometric test.

Further details of epigenomic and transcriptomic analyses that define core cell types, genes and targetable mechanisms for kidney disease are presented in Liu, Hongbo, et al. Nature Genetics 54.7 (2022): 950-962 and incorporated herein in their entireties. A list of genes that were identified as the genes that cause kidney disease is as shown in Table 1.

In the present study, various genes were identified as genes that involved in kidney disease. These genes are listed below in Table 1.

TABLE 1
List of genes that were identified as the genes that cause kidney disease.

| | | | | |
|---|---|---|---|---|
| SPATA5L1 | GPN1 | LARP4B | NFATC3 | CDC14A |
| UMOD | CHAC1 | PPM1G | TNRC6B | CYP17A1 |
| SHROOM3 | CHP1 | SLC28A2 | NIPSNAP1 | DNAJB4 |
| CCDC158 | NDUFAF1 | PIP5K1B | CLEC18A | GAB2 |
| PRKAG2 | RPP25 | FAM122A | LACTB | USP38 |
| F12 | ZNF585A | DAB2 | RPRD2 | NTN5 |
| PFN3 | ZNF781 | SLC47A1 | PRDX1 | NARS2 |
| SLC34A1 | UQCC1 | PHTF2 | TESK2 | ARNT |
| UNCX | SPIRE2 | PTPN12 | CRIP3 | CYP2D6 |
| GP2 | DPEP1 | CTSS | ETNPPL | SH3YL1 |
| MPPED2 | INO80 | TFDP2 | SIPA1L3 | ALKAL2 |
| RGS14 | SPATA2L | TMEM60 | MMP24OS | SPATA7 |
| STBD1 | SUPT7L | PPM1J | PSMC3 | PPP2R3A |
| TBX2 | SHF | ACSS2 | CA12 | QSOX2 |
| NAT8 | EXOG | PIGU | MMACHC | AP3B2 |
| TPRKB | XYLB | MYH7B | ZNF641 | CCDC163P |
| NRBP1 | SPATA33 | MAP3K11 | MUC1 | CLDN23 |
| PGAP3 | NRG4 | OVOL1 | BCL2L14 | TUB |
| FBXL20 | DACH1 | ATP1B3 | ATP6V1FNB | CCDC77 |
| MED1 | A1CF | PDCD5 | SLC5A6 | NEIL2 |
| LARP4B | SPATA5L1 | GPN1 | BORCS7 | NAGA |
| PPM1G | UMOD | CHAC1 | NT5C2 | SORD |
| SLC28A2 | SHROOM3 | CHP1 | THBS3 | DUS2 |
| PIP5K1B | CCDC158 | NDUFAF1 | STYXL1 | UBA7 |
| FAM122A | PRKAG2 | RPP25 | SLC7A9 | APEH |
| DAB2 | F12 | ZNF585A | MUTYH | RAI1 |
| SLC47A1 | PFN3 | ZNF781 | MIEN1 | KDM1A |
| PHTF2 | SLC34A1 | UQCC1 | KLHDC7A | NIPSNAP2 |
| PTPN12 | UNCX | SPIRE2 | SMDT1 | GRB10 |
| CTSS | GP2 | DPEP1 | TPPP | PCK1 |
| TFDP2 | MPPED2 | INO80 | CASP9 | RTF1 |
| TMEM60 | RGS14 | SPATA2L | NRIP1 | RRAGD |
| PPM1J | STBD1 | SUPT7L | PPDPF | USP12 |
| ACSS2 | TBX2 | SHF | EBNA1BP2 | CPXM1 |
| PIGU | NAT8 | EXOG | TMEM125 | WHAMM |
| MYH7B | TPRKB | XYLB | C1orf210 | THOC7 |
| MAP3K11 | NRBP1 | SPATA33 | MED4 | PRXL2A |
| OVOL1 | PGAP3 | NRG4 | CPS1 | MAT1A |
| ATP1B3 | FBXL20 | DACH1 | LANCL1 | LEAP2 |
| PDCD5 | MED1 | A1CF | NUDT15 | C8orf58 |
| BIN3 | WDR6 | NDUFA6 | PPM1E | SLC22A4 |
| OGDHL | IHO1 | MARCHF5 | ECH1 | PDLIM4 |
| PARG | CPEB1 | DCAF4 | LGALS7 | NAAA |
| TRAIP | WBP2NL | PCCB | LGALS4 | SYCE2 |
| NSUN4 | ACOT1 | UGT8 | AL138847.1 | ITPK1 |
| FAAH | PPP1R3B | CALR | ANGPTL3 | USP37 |
| IQCN | ALPK3 | GCDH | FBXO42 | VIL1 |
| CCAR2 | NMB | ARL16 | BANF1 | CYP27A1 |
| JUND | PPFIA2 | SLC25A10 | IST1 | CD164L2 |
| DHRS11 | CDK7 | DNA2 | CRCP | DHX36 |
| FIBP | GAS8 | GSTM4 | ERV3-1 | MFSD4A |
| SNX32 | HYAL3 | ALDH1L1 | GUSB | LASP1 |
| SFMBT1 | ZNF19 | RPL13 | VKORC1L1 | ACSM3 |
| ITIH4 | KCNJ13 | GNL3 | NUP54 | NPLOC4 |
| AC006254.1 | ARSB | PSMD12 | ACOT4 | SBK1 |
| TREX1 | XPO5 | C1QTNF4 | HEATR4 | PRR15L |
| TMA7 | PIP5K1A | B4GALT1 | TIMM10 | DOK7 |
| HSPA4 | PIGV | SRR | NUPR1 | ORMDL3 |
| WIPI1 | MIPOL1 | ARHGEF19 | NFATC2IP | NUDT13 |
| RGS19 | NEK4 | FDFT1 | ST13 | CFAP70 |
| MAST2 | ALDH3A2 | ACSM1 | TRIM37 | FAM149B1 |
| GGNBP2 | PPM1M | NXPH4 | FAM102A | FAM177A1 |
| ZGPAT | MLLT3 | TSPAN10 | DPM2 | DENND5B |
| SLC2A4RG | NCKIPSD | TUFM | PRR13 | DNAJC8 |
| AL121845.2 | ZNF589 | SULT1A2 | CTSB | PTAFR |
| LIME1 | WDR73 | EIF3C | IL12RB1 | SESN2 |
| ARFRP1 | CYB561D1 | NPIPB6 | RFT1 | ATP5IF1 |
| AL121845.3 | AP1G1 | RGS9BP | ASIC1 | KCNMA1 |
| TSPAN14 | GOLGA6L4 | PAX8 | DIP2B | TSPO |
| STRA6 | PHETA2 | ZBTB46 | SMARCD1 | MRPS26 |
| BCAR3 | CEP89 | TM6SF2 | PPP1R3C | CXCL10 |
| FSD2 | AGAP6 | SLC47A2 | METTL21A | SH2B2 |
| SYN2 | SEC14L6 | CPLANE2 | RABGEF1 | ALKBH5 |
| FAM167A | BHMT | CDK10 | IQCH | HSPB7 |
| MTCH2 | DBNDD1 | SYNGR3 | EIF6 | CPNE1 |
| PMF1 | PBLD | STOX1 | FAAP20 | CEP250 |
| SLC25A44 | TCEA3 | ZFAND2A | ATOH7 | GNRH2 |
| USP24 | GDF9 | ADK | RIF1 | COLCA2 |
| ESPL1 | GOLGA6L10 | THUMPD1 | SFXN2 | CAND2 |
| EXOC3 | NINJ1 | REXO5 | AL139353.1 | HSBP1L1 |
| TXNL4A | LCAT | HOXB8 | FADS2 | WFIKKN1 |
| CYB5A | ANGPT2 | FLRT3 | FADS1 | WDR90 |
| ASAH2B | SLC39A4 | DIXDC1 | ABO | FBXL16 |
| ZNF529 | UBASH3B | ARHGEF26 | PPP6C | JMJD8 |
| OXT | IDUA | DNPH1 | EFL1 | BRD3 |
| ODR4 | BTBD1 | CUL7 | PRKCZ | LYRM1 |
| TYW1 | SPINT1 | ZNF713 | HYDIN | SLC2A2 |
| POMGNT1 | CWF19L1 | NME6 | RBM6 | GCLM |
| MRPL12 | NPHP3 | ATP12A | DLK1 | LY75 |
| ERGIC3 | FLCN | SEC24C | DHRS7 | TMEM116 |
| CDC25A | FCGRT | BBS1 | FUT11 | DPP8 |
| P4HTM | TRMT61A | TRIM65 | NOM1 | PPCDC |
| PRMT7 | SDCCAG8 | SGK223 | MESD | ANXA11 |
| MST1 | RDH12 | LRRCC1 | AMPD3 | ACOX1 |
| TMEM82 | RDH11 | NCOA7 | PIM1 | CDC42SE2 |
| DEPTOR | TMEM163 | ANKRD27 | CRTAC1 | LPA |
| CLCNKB | AS3MT | AMBP | GRK4 | N6AMT1 |
| MTMR3 | EBPL | AP2A2 | ALDH2 | HOXB7 |
| TBC1D17 | FBXL17 | WASHC2A | ATMIN | PANK1 |
| GSDMB | MIER1 | RAD54L2 | FLT3 | ATRN |
| ELMO3 | WDR78 | DOCK3 | PAN3 | ANKRD6 |
| PAPLN | WDR5B | CA3 | CHCHD2 | TTLL13P |
| YEATS4 | FAM162A | SUCLA2 | NUPR2 | CCDC32 |
| KY | VTI1B | ZDHHC11 | TRPM4 | RCBTB2 |
| ANAPC13 | MRPS7 | CCDC17 | TTYH3 | ITPA |
| CEP63 | FABP3 | CCDC163 | RAPGEF3 | CA13 |
| TET2 | ADGRV1 | IPP | SLC48A1 | ZNF664 |
| SPAG4 | HAPLN4 | LURAP1 | HOXB2 | |
| NRBF2 | LTBP3 | DEF8 | WARS1 | |
| CWH43 | MGMT | UVSSA | WDR25 | |
| ACP2 | TRAPPC4 | SLC66A3 | PLAU | |
| DDB2 | CARD19 | ELP3 | DPP3 | |
| SKI | HTR2B | WDR1 | CLN3 | |
| NUDT2 | C2orf72 | UNC79 | CAPN12 | |
| EYA4 | ASAP3 | MFSD13A | RNF123 | |
| ROCK2 | MYOZ1 | DZIP1 | NSFL1C | |
| XPO7 | TARS2 | SLC9A4 | SRSF9 | |
| LRPAP1 | ANXA9 | IL18R1 | ARPC5L | |
| UBE2Q2 | ARMC9 | SLC9A2 | GMPPB | |
| COX14 | CENPH | SLC22A18AS | SKA2 | |

### I. For DPEP1/CHMP1A Experiments

GWAS, eQTL, mQTL, and Moloc Analysis

A eGFR GWAS summary dataset was downloaded from CKDGen Consortium website (www.ckdgen.imbi.uni-freiburg.de/), and significant associations were defined by P value<5.0E-8. Human kidney and glomerular eQTL datasets was generated. The DNA mQTL was performed on 188 human healthy kidney samples with genotyping and CpG methylation data. Briefly human kidney samples were genotyped using Affymetrix Axiom arrays. Illumina EPIC arrays were used for methylation analysis. Cis-mQTL (referred to as mQTL) association analysis was conducted using 188 samples with imputed genotyping data and methylation data by EPIC array. Beta values of each CpG were transformed by an inverse-normal transformation (INT). Missing values were imputed based on nearest neighbor averaging implemented by R package impute (v1.64.0). For each SNP-CpG pair within a cis window of ±1 Mb from the queried CpG site, the association between INT transformed methylation and genotype dosage was quantified using MatrixQTL (v2.1.0). R package using an additive linear model. This model was fitted with covariates including general variables (sample collection site, age, sex, top five genotype PCs, degree of bisulfite conversion, sample plate, and sentrix position) and PEER factors. Multiple trait colocalization among GWAS, eQTL, and mQTL was performed by moloc. In short, SNPs+/−100 kb of each leading GWAS SNPs were used to calculate the posterior probability. In the moloc results, PP_abc represents the posterior probability of three traits are associated and share a single causal variant. PP_abc>0.8 was use as the threshold of multiple trait colocalization.

### Human Kidney RNA-Seq Data

Gene expression changes were examined in the microdissected human kidney RNA sequencing data (n=432). The clinical information was shown.

### Single-Cell ATAC Sequencing

Mouse snATAC-seq data (three healthy mouse kidneys; 16,887 nuclei) and human snATAC-seq data (two healthy human kidneys; 12,720 nuclei), was reanalyzed as described earlier

### Conditional Analysis

Each of the 12 open-chromatin peaks was evaluated for conditional analysis. SNPs within each peak were identified and combined into peak-specific lists. Peak-specific conditional analyses used the-cojo-cond command in GCTA, with the list of peak SNPs being input as the conditional SNP-list, with eGFR GWAS analysis results used as the input summary data, and BioVU imputed genetic data as the reference dataset for evaluating linkage disequilibrium. Output for each peak provides the conditional analysis results of all SNPs within the locus region after conditioning on all available SNPs within the open chromatin peak. When peak SNPs were in strong LD introducing collinearity, one SNP from each group was excluded from the analysis. Final included SNPs were assigned to peaks as follows (Peaks 2 and 7 did not contain any available SNPs):
Peak 1: rs8059821; rs80089054; rs4785697
Peak 3: rs111857923; rs187720
Peak 4: rs62068712; rs192325916
Peak 5: rs154665
Peak 6: rs11641525; rs258340; rs12930346; rs7197490; rs146442848
Peak 8: rs908951; rs11649482
Peak 9: rs142099578; rs12920969; rs4785581; rs1657380
Peak 10: rs164751; rs58290281; rs201976326; rs12921177
Peak 11: rs35415928; rs151272435; rs13329897
Peak 12: rs16965913; rs5818725; rs80164364; rs117418297; rs59863025
CRISPR/Cas9 mediated peak deletion

HEK293 cell stably expressing Cas9 was a gift from Dr. Liling Wan from University of Pennsylvania. The sgRNA expression plasmids were generated. Briefly, Annealed sgRNA oligos were subcloned into pLKO5.sgRNA.EFS.GFP with the Bsmb1 site. All constructs were verified by Sanger sequencing. sgRNA expression plasmids were transfected to Cas9 stable HEK293 cell using lipofectamine 3000. After 72 h, cells were harvested, and Dpep1 and Chmp1a expression were determined by QRT-PCR. At the same time, DNA was isolated, and sgRNAs target regions were determined by Sanger sequencing.

### Mice

Eight- to ten-week-old male mice were used in this study. All mice were maintained under SPF conditions with ambient temperature 20-22, humidity 50-70%, and a 12/12 h light/dark cycle. All animal experiments were reviewed and approved and were performed in accordance with the institutional guidelines. Dpep1 mutant mice were generated by co-injection of Cas9 mRNA (100 ng/μl; ThermoFisher, A29378), sgRNA (50 ng/μl) in CRISPR Cas9 Mouse Targeting Core of University of Pennsylvania. Two sgRNAs were generated with Guide-it™ sgRNA In Vitro Transcription Kit (Takara #632635). Chmp1a mutant mice were imported from Mutant Mouse Regional Resource Center of UC Davis (#031089-UCD). For FA-induced nephropathy mouse models, 8-week-old male wild-type and Dpep1^{+/-} or Chmp1a^{+/-} mice were injected with FA (250 or 200 mg/kg, dissolved in 300 mM sodium bicarbonate) intraperitoneally and euthanized on day 7. For the cisplatin-induced injury model, 8-week-old male wild-type, Dpep1^{+/-} or Chmp1a^{+/-} mice were injected with cisplatin (25 or 20 mg/kg) intraperitoneally. Mice were euthanized on day 3. For the UUO model, mice underwent ligation of the left ureter and were euthanized on day 7, and sham-operated mice were used as controls.

### BUN and Creatinine Level

Serum creatinine was measured by Creatinine Enzymatic and Creatinine Standard (DIAZYME #DZ072B-KY1). Serum BUN was measured by Infinity™ Urea Liquid Stable Reagent (Pointe Scientific #B7552150). Both measurements were performed according to the manufacturers' instructions.

### Histopathology Analysis

Kidneys were harvested from mice, rinsed in PBS, fixed in 10% formalin, and embedded in paraffin. Histological analysis was examined by H&E and Picrosirius red (Polyscience #24901). The acute tubular injury was scored. In brief, semi-quantitation was evaluated, including tubular dilation, tubular atrophy, tubular cast formation, vacuolization, degeneration, using the following scoring system, Score 0: no tubular injury; Score 1: <10% of tubules injury; Score 2: 10-25% of tubules injury; Score 3: 25-50% of tubules injury; Score 4: 50-74% of tubules injury; Score 5: >75% of tubules injury.

### Real-Time RT-PCR

RNA was isolated from mouse kidneys or cultured cells using Trizol reagent (Invitrogen) and was reverse transcribed into cDNA using cDNA Archival Kit (Life Technologies). Real-time RT-PCR was performed using SYBR Green Master Mix (Applied Biosystems).

### Cell Culture

Rat epithelial cells (NRK-52E; ATCC (CRL-1571)) were cultured in DMEM with 5% fetal bovine serum (FBS). Dpep1 and Chmp1a siRNA were purchased from Dharmacon. Dpep1-overexpressing vector pCMV6-Entry was purchased from OriGene Technologies, Inc. Transfection of gene-targeting siRNA, negative control siRNA, and overexpressing plasmid were performed using Lipofectamine 3000.

For transfection, cells were seeded in six-well plates, grown overnight until 60-70% confluency, and then transfected with 50 nM (final concentration) siRNA or 5 μg Dpep1-overexpression plasmids. Transfection efficiency was determined under a fluorescence microscope by the presence of Cy3 transfection control. After 24 h of Chmp1a siRNA transfection, cells were pretreated with the following inhibitors: 20 μM Nec-1 (Cayman #11658); 1 μM Liproxstatin1 (Cayman #17730); 10 μM VX-765 (Cayman #28825); 20 μM Z-VAD(OMe)-FMK for 18 h and then cells were treated with 20 μM cisplatin (Cayman #15663-27-1) for 24 h. After 24 h of Dpep1 siRNA transfection, cells were treated with cell death inducers for 18-20 h, respectively: 5 μM Nigericin (Sigma #N7143); 5 μM CPT (Cayman #11694); 5 μM erastin (Cayman #17754), 20 μM FIN56 (Cayman #25180), 5 μM FINO2 (Cayman #25096) and 5 μM RSL3 (Cayman #19288).

For primary culture of renal tubule cells, kidneys were collected from 2- to 4-week-old male mice. Cells were isolated by 2 mg/ml collagenase I (Gibco #17018-029) digestion for 30 min at 37° C. with gentle stirring and filtered through a 100-μm mesh. The cell suspension was cultured in RPMI 1640 (Corning #10-040-CM) supplement with 10% FBS (Atlanta Biologicals #S11950), 20 ng/ml EGF (Peprotech #AF-100-15), 1×ITS (Gibco #51500-056), and 1% penicillin-streptomycin (Corning #30-002-CI) at 5% CO₂ and 37° C. Primary renal tubule cells were pretreated with 50 μM Mito-TEMPO (Sigma #SML0737) for 1 h and treated with 20 μM cisplatin for 24 h.

### Western Blot

Kidney tissue or cultured cell lysates were prepared with ice-cold lysis buffer (CST #9806) containing protease inhibitor cocktail (cOmplete Mini, Roche #11836153001) and phosphatase inhibitor (PhosSTOP, Roche #4906837001), resolved on 8-12% gradient gels, transferred on to polyvinylidene difluoride membranes, and probed with the following antibodies: DPEP1 (Proteintech #12222-1-AP 1:500), CHMP1A (Proteintech #15761-1-AP 1:500), RIPK3 (Sigma #PRS2283 1:1000), Cleaved Caspase 1 (Santa cruz #sc-56036 1:500), Collagen III (Abcam #ab7778 1:1000), Fibronectin (Abcam #ab2413 1:1000), aSMA (Sigma #A5228 1:1000), ACSL4 (Abcam #ab155282 1:1000), CD63 (Abcam #ab193349 1:1000), GPX4 (Abcam #ab125066 1:1000), Actin (Sigma #A3854 1:20000), GAPDH (Proteintech #60004-1-Ig 1:1000), and Tubulin (BioLegend #801202 1:1000). Anti-rabbit IgG (H+L) (DyLight™ 800 4×PEG Conjugate) (CST #5151 1:10000) and Anti-mouse IgG (H+L) (DyLight™ 680 Conjugate 1:10000) (CST #5470) was used as a secondary antibody.

### Immunofluorescence

Cells were washed with PBS, fixed with 4% paraformaldehyde, permeabilized with PBS-0.2% Triton×100, and blocked with 5% FBS. Immunostaining were performed using the following primary antibodies: Cleaved Caspase 3 (CST #9664 1:500), Fluorescein labeled LTL (Vector #FL-1321 1:500), AQP2 (Santa Cruz #sc-9882 1:200), Fluorescein labeled DBA (Vector #FL-1031-5 1:500), Fluorescein labeled PNA (Vector #FL-1071-5 1:500), DPEP1 (Invitrogen #PA5-52984 1:200), CHMP1A (Proteintech #15761-1-AP 1:200), EEA1 (BD #610456 1:200), RAB5 (CST #3547 1:200), RAB7 (Sigma #R8779 1:200), RAB11 (BD #610658 1:200), VAMP7 (NOVUS #NBP1-07118 1:200), and GM130 (BD #610822 1:200).

### Live Cell Imaging

Primary cultured renal tubule cells or NRK52E cells were incubated with 1 μM transferrin (Thermo Fisher #T13342) for 4 h, or 5 μM mitoSOX (ThermoFisher #M36008) for 10 min, or 2 μM BODIPY™ 581/591 C11 (Thermo Fisher #D3861) for 0.5 h, or 20 ng/ml dextran conjugated to 488 (Thermo Fisher #D22910) for 2 h, at 5% CO₂ and 37° C., respectively. After washing with PBS, cells were imaged directly under the confocal or regular fluorescence microscope.

### Cell Count and Cytotoxicity Assays

Mouse primary tubular epithelial cells from wild-type, mutant mice or NRK52E cells were plated in 96-well plate. Transfection of NRK52E cells with siRNA was carried out as described above. After 24 h of transfection, cisplatin was added for 24 h. Cells were harvested and stained with trypan blue (Thermo Fisher #T10282) to visualize dead cells. Cell counts were analyzed in Countess Auto Counter (Invitrogen, C10227). LDH release was quantified using CytoTox 96 Non-Radioactive Cytotoxicity Assay (Promega #G1780). Medium was collected in triplicates, spun down, and incubated in a 96-well plate with the CytoTox reagent for 20-30 min. After adding the stop solution, the absorbance signal was measured at 490 nm in a plate reader. The ratio between dead cell and live cell was calculated through the measurement using MultiTox-Fluor Multiplex Cytotoxicity Assay (Promega #9201). Cells were incubated in assay buffer containing live cell substrate GF-AFC and dead cell substrate bis-AAF-R110 for 30 min at 37° C. The plate fluorescence was measured using a plate reader as following: viability: excitation ˜400 nm; emission ˜505 nm. cytotoxicity: excitation ˜485 nm; emission ˜520 nm.

### TUNEL Assay

Apoptotic cells in the kidney were detected by Click-iT™ Plus TUNEL Assay for In Situ Apoptosis Detection, Alexa Fluor™ 594 dye (ThermoFisher Scientific, #C10618) according to the manufacturer's instruction. Images were obtained under fluorescence microscope. Apoptotic cells were quantified and presented as the number of TUNEL positive cells per field.

### Glutathione Assay

The GSH and oxidized glutathione (GSSG) content in the kidney was measured by Glutathione Colorimetric Detection Kit (Invitrogen #EIAGSHC) according to the instruction of the manufacturer.

### Iron Assay

Kidney tissue was weighed, cut, and lysed, and iron content was measured by Iron Colorimetric Assay Kit according to the manufacturers' instructions (BioVision #K390-100). The iron content was normalized to the weight of the tissue (microgram of iron per gram of tissue).

### Statistics and Reproducibility

Statistical analyses were performed using GraphPad Prism software (GraphPad Software Inc., La Jolla, CA). A two-tailed t-test was used to compare two groups. One-way or two-way ANOVA was used to compare multiple groups with post hoc Tukey test.

### II. For CASP9 Experiments.

Visualization of SNPs in GWAS and eQTL

LocusZoom was used to view genomic regions of interest in chromosome 1 for kidney function GWAS) and eQTL of CASP9, CELA2B, and DNAJC16 in tubules, glomeruli, and whole kidneys. eQTL box plots were used to show the associations between reference and alternative alleles of given genotypes and CASP9 expression in tubule samples.

### Colocalization Analysis

The eQTL dataset of tubules, glomeruli, and whole kidney were generated. To estimate the posterior probabilities of sharing the same genetic variants between kidney function and gene expression in tubuli and glomeruli, colocalization analysis was performed using coloc, GWAS, and eQTL summary data. A 100-kilobase region around the index SNP was used to calculate posterior probability. In the coloc results, H3 represents the posterior probability that both traits (kidney function and gene expression) are associated, but with different causal variants; H4 represents the posterior probability that both traits are associated and share a single causal variant. PP_H4>0.8 was used for the threshold of colocalization.

### TWAS Fusion

TWAS FUSION pipeline was performed using eGFR GWAS and tubule eQTL data. Gene expression weights were generated using human kidney tubule RNA sequencing (n=121) data following the FUSION pipeline. Genotypes were imputed to the 1000 Genomes Phase 1 v3, and restricted to well-imputed (information score, >0.9) sites. Reads per kilobase of transcript per million mapped reads and log-adjusted gene expression levels were estimated in a generalized linear model controlling for three gene expression principal components and rank-normalized. The genes that did not exhibit cis-genetic regulation at current samples sizes were filtered by keeping only genes with nominally significant estimates of cis-SNP heritability. Due to the complicated LD patterns genes in the human leukocyte antigen region were refrained from being reported. To generate predictive models, FUSION defines gene expression for samples as a linear function of SNPs in a 1-megabase region flanking the gene as where are the SNP weights, are covariates (e.g., sex, age, genotype principal components, genotyping platform, PEER factors) and their effects, and is random environmental noise. FUSION estimated weights for expression of a gene in a tissue using multiple penalized linear models (here, LASSO was used).

### Summary-Based Mendelian Randomization

SMR was used to test potential causal effects of a gene on complex trait, given an SNP as an instrumental variable, using summary-level data from eGFR GWAS and eQTL studies. SMR reports the association for visualization of SMR analysis, and SMR effect plot was depicted with SMREffectPlot function in plot_SMR.r using multiple variants in the cis-eQTL region of genes.

Multiple Tissue eQTL Mapping

The eQTL summary results of 44 other human tissues were downloaded from GTEx. METASOFT, a meta-analysis method, was performed on all variant-gene pairs that were significant (false discovery rate<5%) in at least 1 of the 46 tissues (two kidney compartments; tubules and glomeruli and 44 GTEx tissues). A random-effects model in METASOFT (called RE2) was used, and the posterior probability (m value) was calculated for each SNP-gene pair and tissue tested. The significance cutoff of m>0.9 was used to discover high-confidence eQTLs.

Human Kidney snATAC Sequencing

Human kidneys were homogenized and single-nuclear suspension for snATAC sequencing (snATACseq) was prepared according to the manufacturer's protocol (10× Genomics). Quality control for constructed library was perform by an Agilent Bioanalyzer High Sensitivity DNA kit. The libraries were sequenced on an Illumina HiSeq. The data processing and analysis were performed.

### CRISPR-Cas9 Genomic Deletions

Human embryonic kidney (HEK) 293 cells stable expressing Cas9 was a gift of L. Wang and L. Song (University of Pennsylvania). Guide RNAs were designed by CRISPOR and cloned into pLKO.sgRNA plasmid. After confirming insert sequences, plasmids were transfected into Cas9-expressing HEK293 using Lipofectamine 3000 (Thermo Fisher Scientific, #L3000015) at 70 to 80% confluent according to the manufacturer's instruction. Cultured cells were collected 48 hours after transfection, and RNA was extracted using TRIzol. Gene expression was quantified by quantitative reverse transcription polymerase chain reaction (qRT-PCR). Target genomic region deletion was confirmed by Sanger sequencing.

### SNP Prioritization

The likely causal SNP was prioritized by the following method: (i) reached genome-wide significance in eGFRcrea GWAS, (ii) reached genome-wide significance in human kidney eQTL for CASP9, (iii) located in an open chromatin area in human kidney snATACseq data, and (iv) altered the expression of the target gene in CRISPR-Cas9 locus deletion experiments.

### Sample Procurement

Human kidney samples were obtained from surgical nephrectomies. Nephrectomies were deidentified, and the corresponding clinical information was collected through an honest broker; therefore, no consent was obtained from the individuals.

### Mouse Studies

Eight- to 10-week-old male WT mice and littermate male Casp9 HZ mice were used for the cisplatin, FA, and UUO models. Casp9 HZ mice were obtained. Mice were injected intraperitoneally with FA (#10752485 ACROS Organics, 250 mg/kg, dissolved in 300 nM sodium bicarbonate) or sodium bicarbonate (#56014, Sigma-Aldrich) and euthanized on day 7. Mice were euthanized on day 7 after left ureteral ligation or sham surgery. Mice were euthanized on day 3 after administration of a single dose of cisplatin (20 mg/kg body weight) (Cayman, #Cay13119) or phosphate-buffered saline (PBS). Mice were euthanized 18 weeks after uninephrectomy and following low-dose STZ (50 mg/kg, i.p. for 5 days) (streptozotocin, Santa Cruz Biotechnology, #U-9889) or PBS injection. Aging mice were euthanized at 2 years of age. Transgenic mice (Nphs1-rtTA/TRE-APOL1-G2) were placed on a doxycycline diet to induce transgene expression and euthanized.

### Quantitative Reverse Transcription Polymerase Chain Reaction

RNA was isolated from kidney tissue or cells using TRIzol (Invitrogen, #15596018). RNA was reverse-transcribed using the High-Capacity cDNA Reverse Transcription Kit (Applied Biosystems, #4368813), and qRT-PCR was run in the ViiA 7 System (Life Technologies) instrument using SYBR Green Master Mix (Applied Biosystems, #4367659) and gene-specific primers. For quantitative analysis, samples were normalized to glyceraldehyde-3-phosphate dehydrogenase (GAPDH) with the ΔΔCₜ method. Primer sequences are listed in FIG. 45.

### Western Blot

Tissue lysates were homogenized in SDS lysis buffer [Cell Signaling Technology (CST), #7722] and transferred onto polyvinylidene difluoride membranes. After blocking in 5% milk in tris-buffered saline and 0.1% Tween 20, membranes were incubated overnight with the following antibodies at 4° C.: CASP9 (Abcam, #ab202068), cleaved CASP3 (CST, #9664), RIP3 (Millipore Sigma, #PRS2283), BAX (CST, #2772), BCL2 (CST, #3498), GSDMD (Santa Cruz Biotechnology, sc-393656), NLRP3 (CST, #15101), ACSL4 (Abcam, #ab155282), LC-3 (CST, #2775S), cGAS (CST, #31659), STING (CST, #13647S), TBK1 (CST, #38066), pTBK1 (CST, #5483), IRF3 (CST, #4302), pIRF3 (CST, #4947), p65 (CST, #8242), pp65 (CST, #3033), and GAPDH (CST, #5174). Membranes were incubated with horseradish peroxidase-conjugated secondary anti-rabbit or anti-mouse antibody (CST, #7074 and #7076) for 1 hour at room temperature. Signal was detected by enhanced chemiluminescence (SuperSignal West Femto Maximum Sensitivity Substrate, #34094; Thermo Fisher Scientific). Western blots were quantified using the Fiji software.

### Renal Function and Histology

BUN was determined by a TRACE DMA Urea kit (Thermo Electron Corporation, #TR12003), according to the manufacturer's instructions. Electrolytes (Na, K, and Cl) and hemoglobin were measured with the iSTAT Portable Clinical Analyzer using i-STAT CHEM8+ cartridge (Abaxis, #10023291). Kidney tissue was stained with hematoxylin and eosin to investigate renal injury. The degree of tubulointerstitial damage was scored semiquantitatively on a 0 to 5+ scale, according to the percentage of the area affected by hydropic degeneration, hyaline casts, cytoplasmic vacuolization, loss of the brush border, and tubular lumen dilation (0=normal, 1=<10%, 2=10 to 25%, 3=26 to 50%, 4=51 to 75%, and 5=>75%). Sirius red staining was performed according to the manufacturer's protocol (Polysciences, #24901) to determine the degree of fibrosis. For each analysis, five to eight fields were randomly selected and quantified using the Fiji software. Each dot represents the mean score of a single animal.

### Primary Culture of Kidney Tubule Cells

Primary kidney tubule cells were isolated from 3- to 4-week-old mice. Kidneys were minced and incubated for 30 min at 37° C. with collagenase I (Worthington Biochemical Product, #CLS-1). Digested kidney cells were filtered through the 100-, 70-, and 40-μm mesh to isolate single cells. Cell suspensions were cultured in RPMI 1640 (Corning, #10-040-CM) supplement with 10% fetal bovine serum (Atlanta Biologicals, #S11950), EGF (20 ng/ml; PeproTech #AF-100-15), insulin-transferrin-selenium (ITS) (Gibco, #51500-056), and 1% penicillin-streptomycin (Corning, #30-002-CI) at 5% CO₂ and 37° C. Approximately 1×10⁶ cells were seeded per well in six-well plates and allowed to adhere overnight. Kidney tubule cells were treated with cisplatin (20 μM) for 8 hours in the presence and absence of BafA1 (100 nM; Sigma-Aldrich, #B1793).

### Cytotoxicity Assay

Renal tubule cells from WT and Casp9 HZ mice were seeded on 96-well plates (10,000 cells per well). After reaching 80% confluency, cells were treated with cisplatin (20 μM) for 8 hours. Live cells were monitored according to the manufacturer's protocol (Promega, #G9200). Fluorescent signal at 400/505 nm (excitation/emission) was detected in a microplate reader (BioTek, Synergy H1).

### Cytosolic Fraction Extraction

Cytosolic, mitochondrial, and nuclear fractions from kidney tubules were extracted by lysing cells with digitonin buffer (Thermo Fisher Scientific, #BN2006). Cytosolic and mitochondrial DNAs were purified with the DNeasy Blood and Tissue Kit according to the manufacturer's instructions (Qiagen, #69506). Quantitative PCR was performed using the ViiA 7 System (Life Technologies). The data were analyzed using the ΔΔCₜ method and shown as fold change.

### Plasmid Transfection

EGFP-LC3 (#11546), ptfLC3 (#21074), and Cox8-EGFP-mCherry (#78520) were purchased from Addgene. Plasmids were transfected with Lipofectamine 3000 according to the manufacturer's protocol (Thermo Fisher Scientific, #L3000008). After transfection, tubule cells were incubated for 24 hours. The fluorescent signal was detected by a confocal microscope (Leica TCS SP8 Confocal). The number of puncta was counted in 10 cells, and mean puncta number were plotted.

### Immunostaining

Kidneys were fixed in 10% neutral formalin and 5-μm-thick sections were prepared. Sections were deparaffinized, followed by antigen retrieval in citrate buffer at 95° C. for 10 min. Sections were allowed to cool slowly, washed in distilled water, and incubated in 3% H₂O₂ for 10 min. Sections were incubated in 5% goat serum (blocking buffer) at room temperature for 1 hour. In addition, avidin/biotin blocking was performed using the avidin/biotin blocking kit (Vector Laboratories, #004303). Sections were incubated overnight at 4° C. with primary antibody for cleaved CASP3 (1:500; CST, #9664). After washing, sections were incubated with goat anti-rabbit secondary antibody for 1 hour at room temperature. ABC ready-to-use reagent (Vector Laboratories, #PK-6101) and ImmPACT DAB Peroxidase (HRP) Substrate (Vector Laboratories, #SK-4105) were used for visualization.

For immunofluorescence studies, sections were incubated with CASP9 (Novus, NB100-56118) antibody, washed with PBS, and incubated with donkey anti-mouse IgG (H+L) highly cross-adsorbed secondary antibody, Alexa Fluor 594 (Thermo Fisher Scientific, #A-21203) and fluorescein isothiocyanate-labeled LTL (Vector Laboratories, FL-1321-2), coverslip-mounted, and examined under a fluorescence microscope (OLYMPUS DP73).

### Caspase Activity Assay

Kidney tissue was homogenized in lysis buffer according to the manufacturer's protocol (Biovision, #K118). Protein concentrations were measured using the Pierce BCA Protein Assay Kit (Thermo Fisher Scientific, #23225). Fifty micrograms of total protein was incubated at 37° C. for 1 hour with reaction buffer (Biovision, #K118) and CASP3 or CASP9 substrate (Ac-DEVD-AFC: Biovision, #1007 and Ac-LEHD-AFC, Biovision #1075). The enzyme-catalyzed release of AFC was quantified in a fluorimeter (excitation of 400 nm and emission of 505 nm).

### Statistics

Data are presented as means±SEM. Unpaired two-tailed Student's t test was used for comparisons between two groups. One-way analysis of variance (ANOVA) with Tukey's post hoc test was used to compare multiple groups. P<0.05 was considered significant.

### Example 1-2: Shared Causal Variants for Kidney Function and CHMP1A/DPEP1 Expression

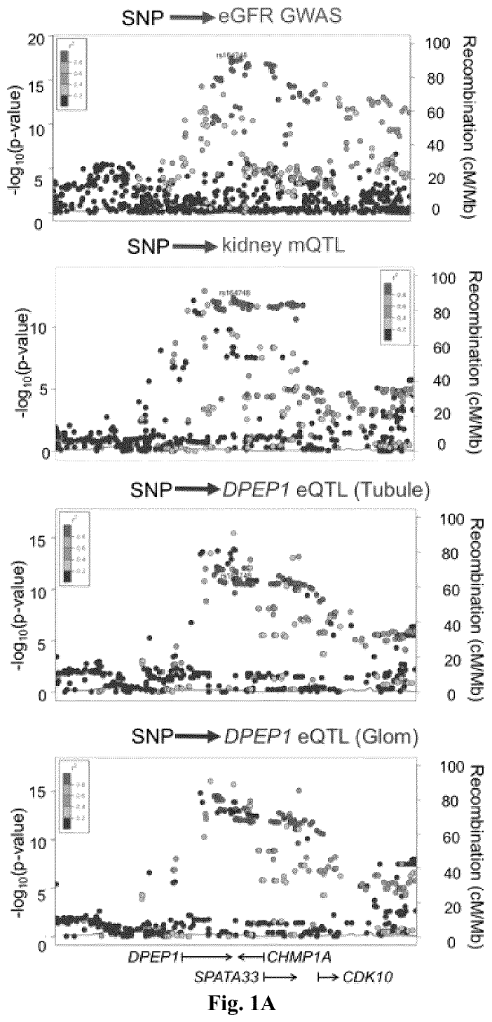

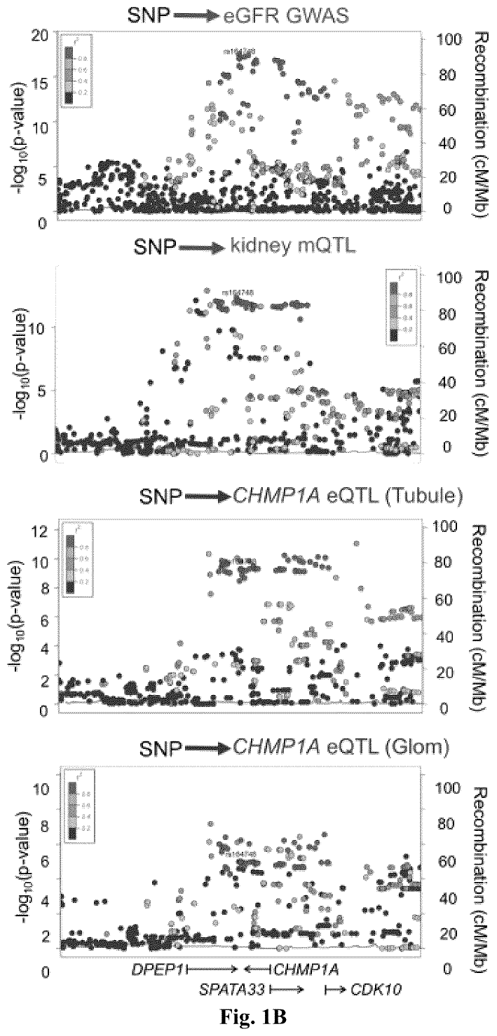

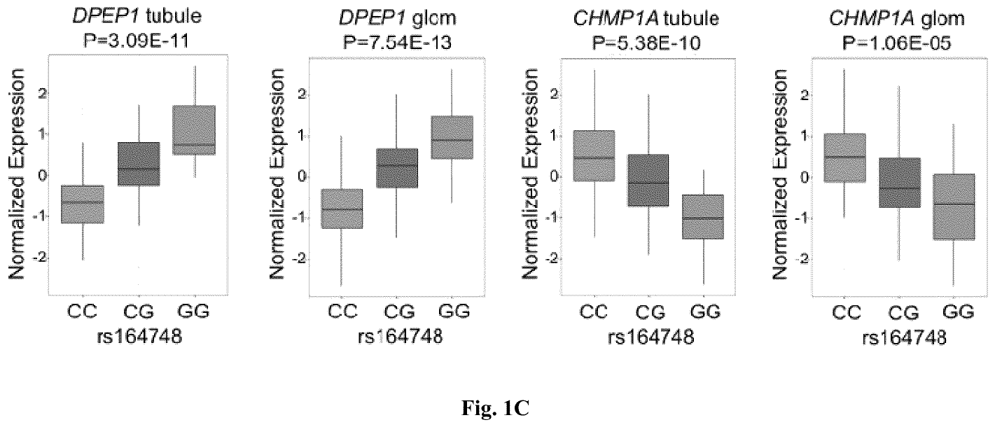

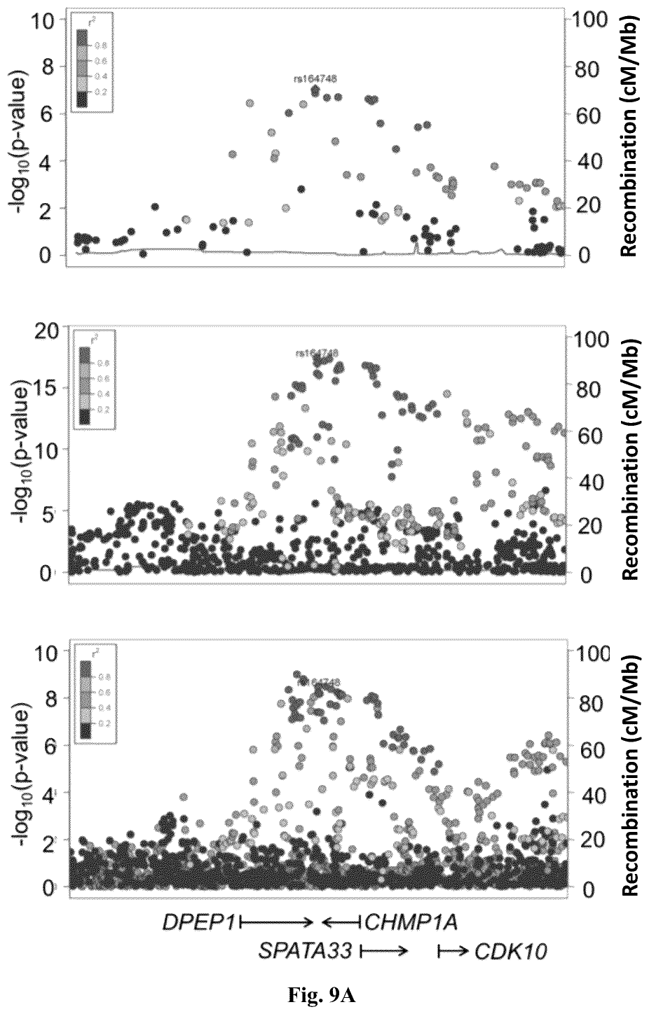

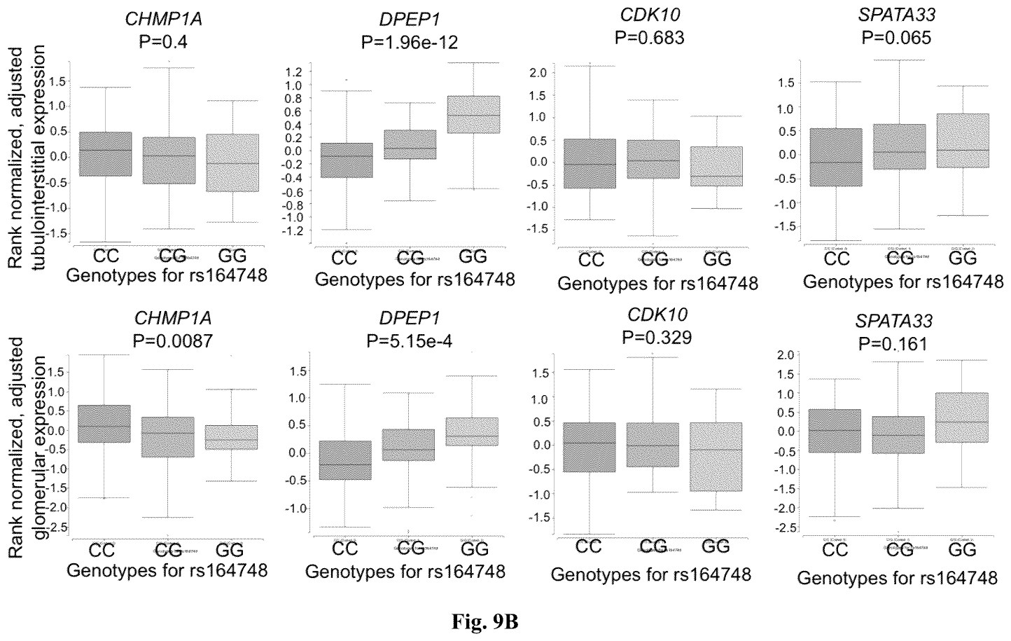

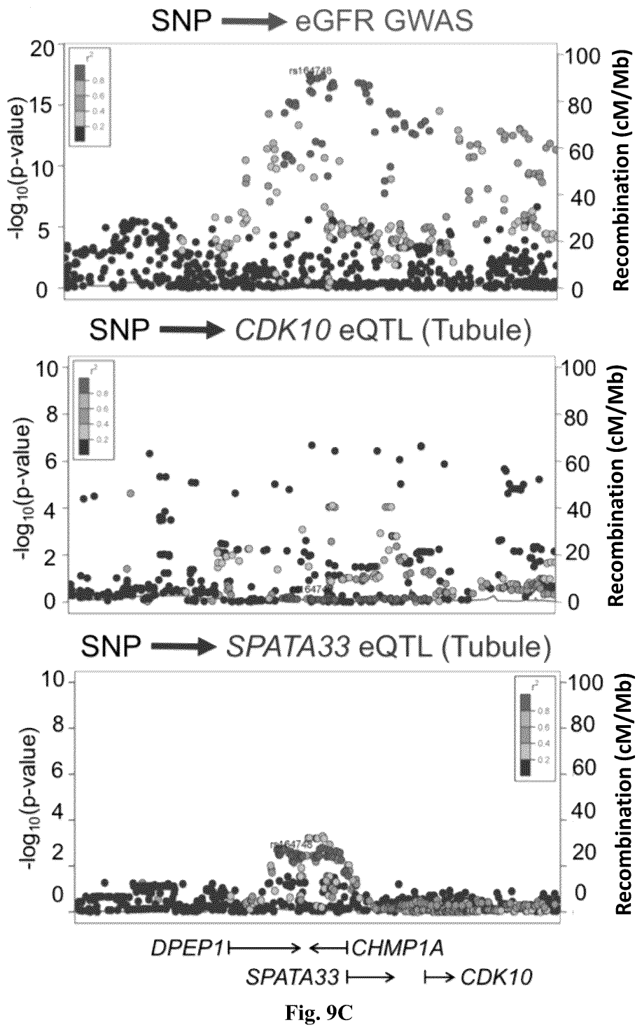

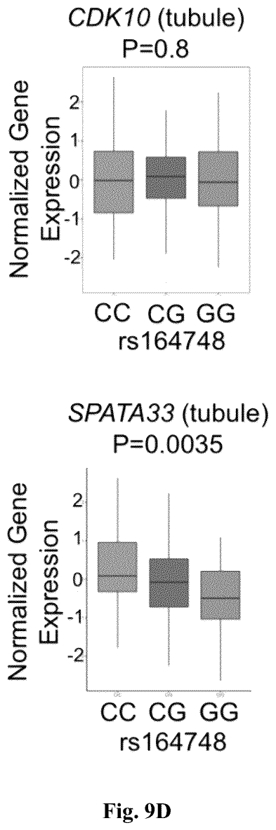

GWAS have demonstrated robust and reproducible association of the chromosome 16 region tagged by rs164748 with kidney function (eGFR) (FIGS. 1A and 9A). This locus spans multiple genes, which include DPEP1, CHMP1A, SPATA33, and CDK10. To dissect this region, the GWAS data was integrated with kidney methylation quantitative trait loci and (mQTL) and eQTL, respectively. First, an association between nucleotide variants at this region and cytosine methylation level was observed in healthy human kidney samples (n=188) (FIGS. 1A,1B). Furthermore, human kidney eQTL information demonstrated an association between variants at this region and the expression of DPEP1 and CHMP1A (FIGS. 1A,1B). This eQTL effect was detected both in the tubule (n=121) and glomerular (n=119) compartments.

As both mQTL and eQTLs appeared to be tagged by rs164748, statistical colocalization was next performed to quantify the extent to which causal genetic variants for kidney function, methylation, and gene expression were shared. Moloc analysis was performed with a+/−100 kb window, covering most cis-regulatory interactions. Molocalization analyses suggested a high posterior probability that the eGFR GWAS, mQTL, and eQTL could be explained by common variants (PP_abc=0.92, “Methods” section). The moloc identified CpGs (cg21038338, cg11907426, cg07716468, cg01510696, and cg00487989) were kidney-specific mQTLs (when compared to whole blood and skeletal muscle). By examining the association between GWAS variants and gene expression in a collection of microdissected human kidney tubule and glomerulus samples, it was found that the genotype of the top GWAS eGFR SNP (rs164748) strongly influenced the level of DPEP1 both in glomeruli and tubuli. Specifically, the GWAS risk allele G was associated with increased DPEP1 expression. Moreover, it was found that the genotype of this top GWAS eGFR SNP (rs164748) strongly influenced the expression level of CHMP1A in tubuli. The GWAS risk allele G was associated with lower CHMP1A expression (FIG. 1C). It was noted that the tubule and glomerular eQTL effects at rs164748 on DPEP1 and the glomerular eQTL effect on CHMP1A replicated in the publicly available NephQTL database, which contained only 136 samples (FIG. 9B). In the GWAS, the C allele of rs164748 was associated with high eGFR, while in the eQTL, the C allele was associated with lower DPEP1 and higher CHMP1A expression.

Next examined were the potential eQTL effects of rs164748 in the GTEx project. While no association was noted between the CKD risk genotype and CHMP1A expression in skin samples, no association was observed between genetic variation of rs164748 and expression of DPEP1 in any GTEx tissue. Also, no association between rs164748 genotype and expression of CDK10 and SPATA33 in kidney samples (FIG. 9B-9D). Collectively, these results suggest a kidney-specific mQTL/eQTL effect of rs164748, which prioritized both DPEP1 and CHMP1A as kidney disease risk genes.

### Example 1-3: Human Kidney-Specific Single Nuclei Chromatin Accessibility Analysis Highlights Likely Causal Variants in Kidney Proximal Tubules

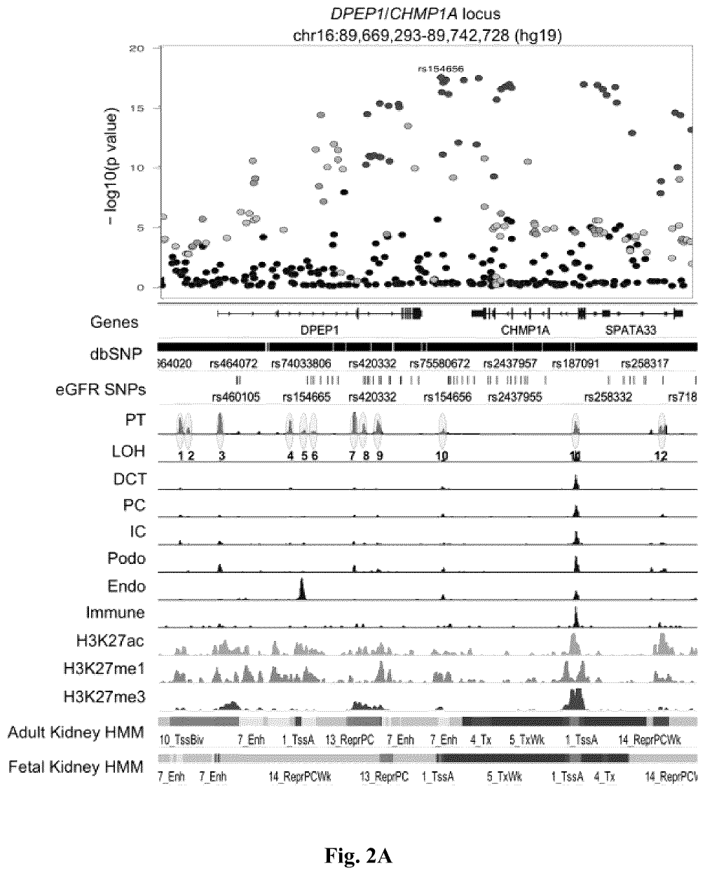

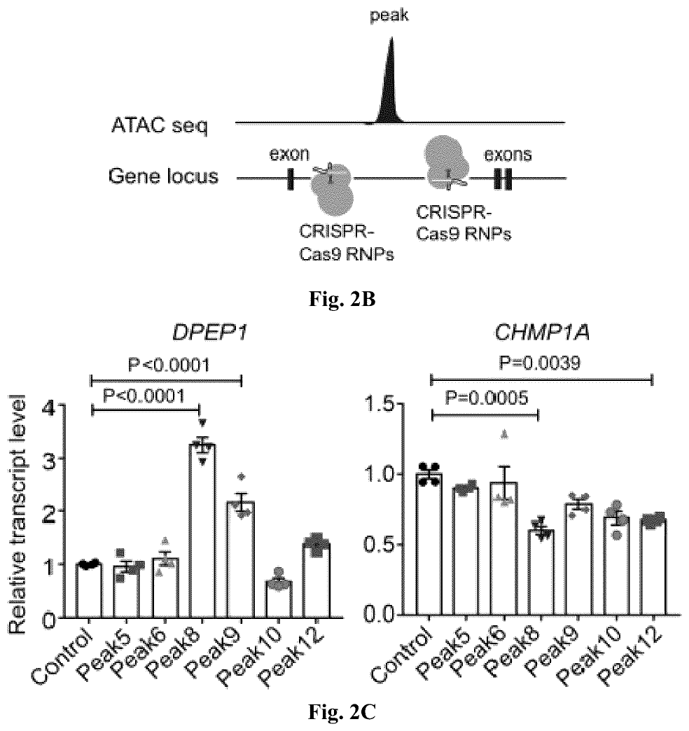

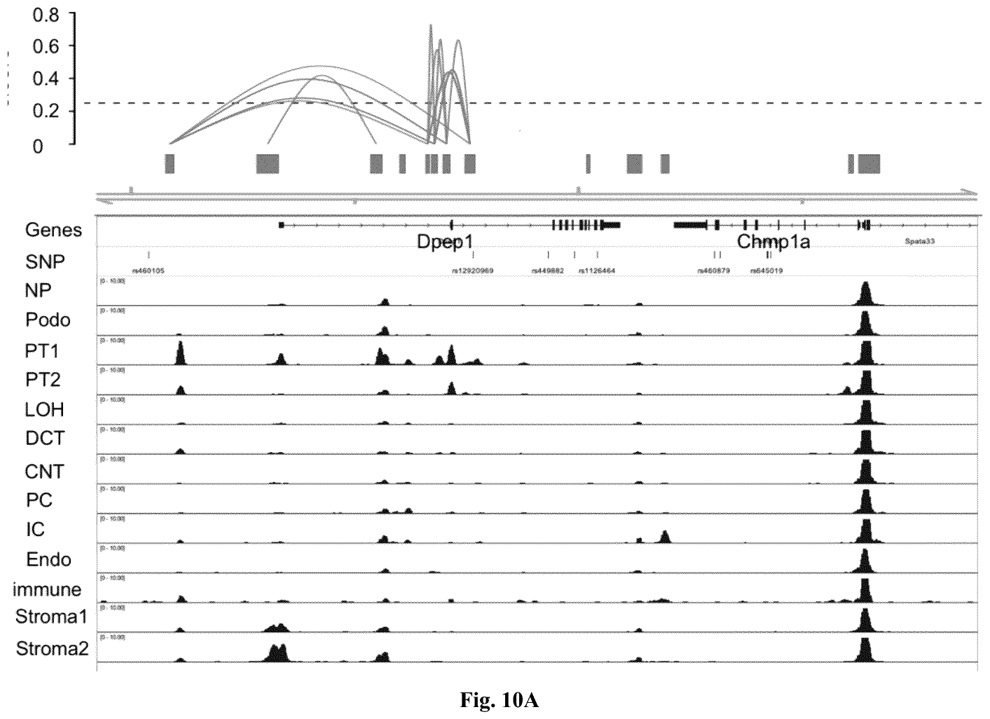

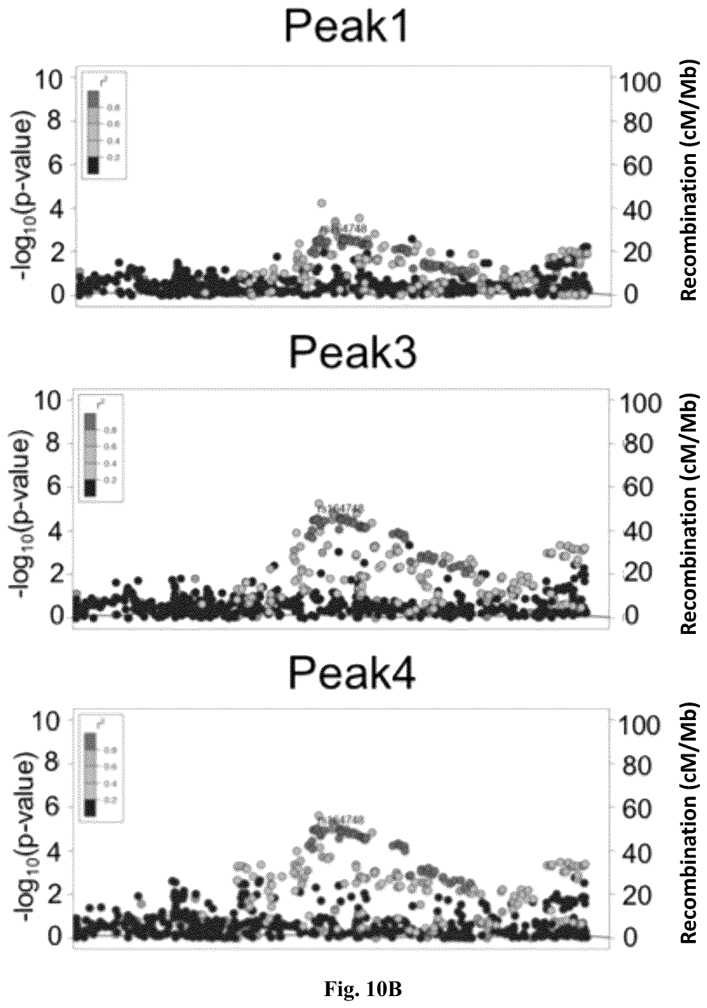

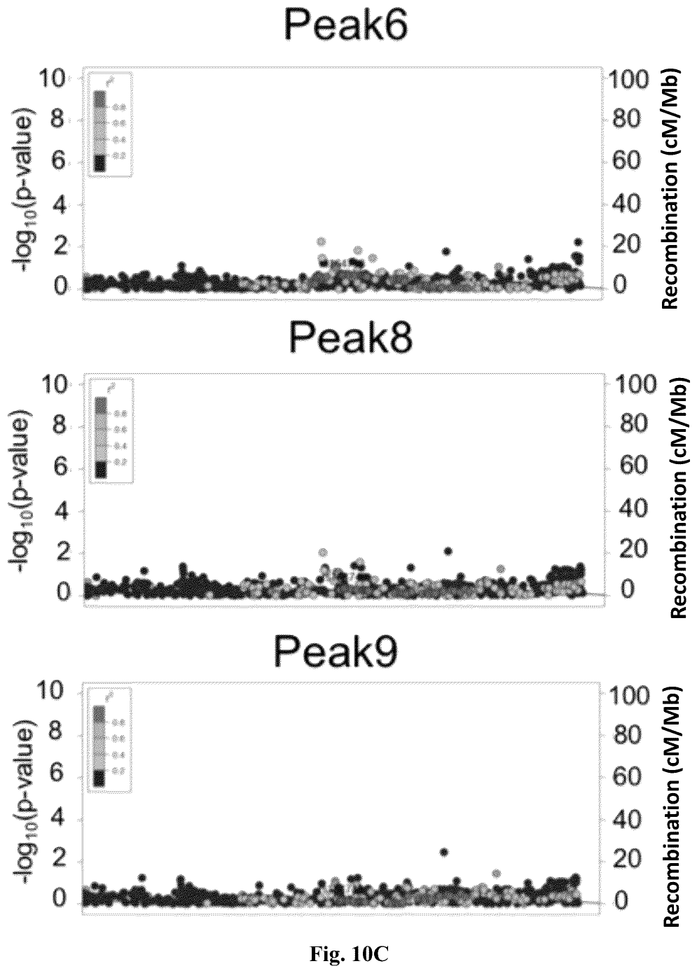

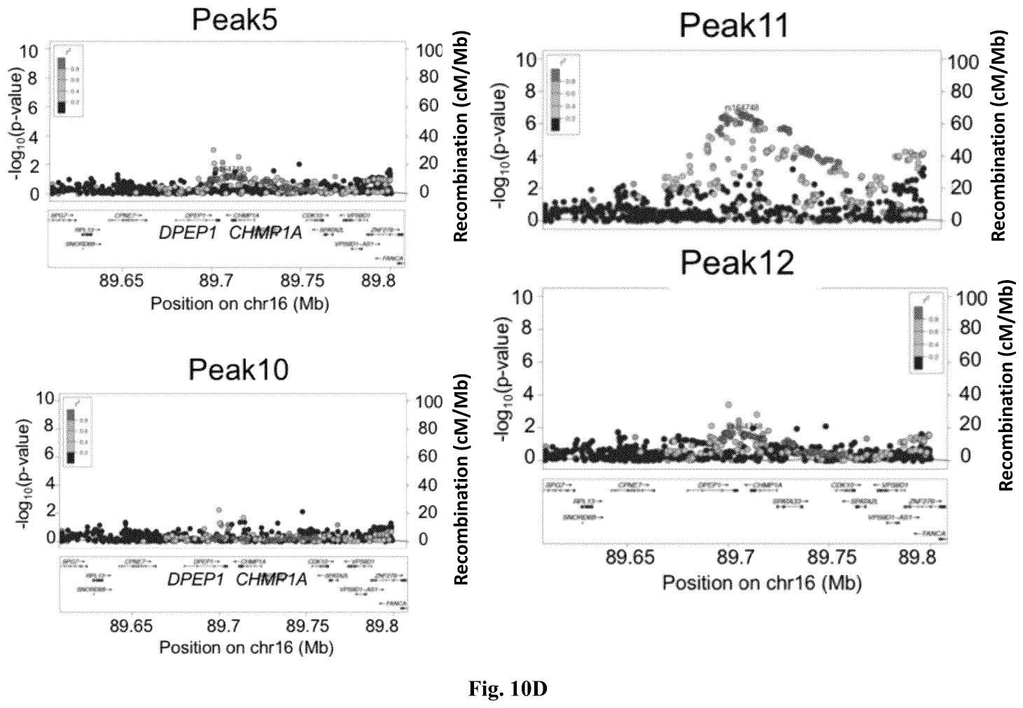

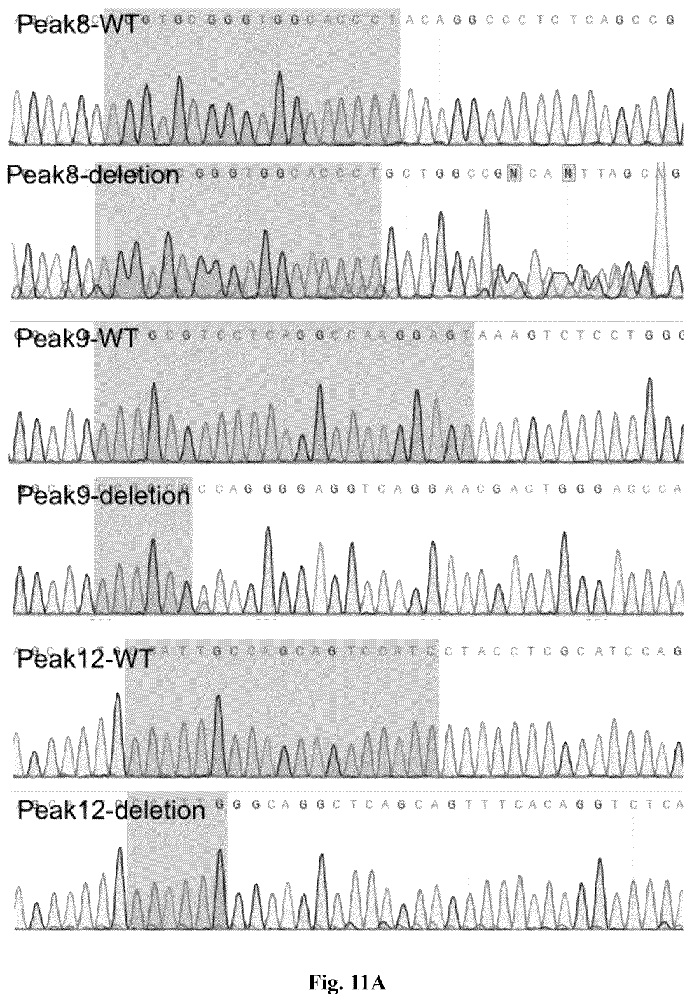

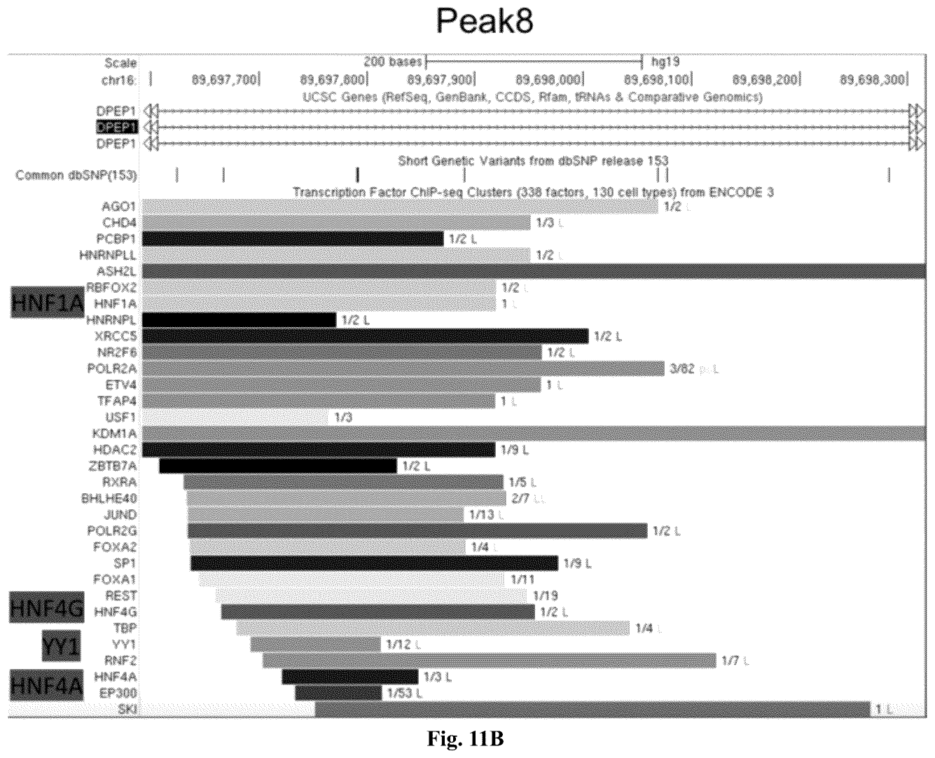

To narrow likely causal variants, human kidney single nuclei sequencing assay was utilized for transposase-accessible chromatin (snATAC-seq). Interrogation of the snATAC-Seq data at this locus identified 12 accessible chromatin regions. One accessible chromatin region, nearby the 5′ end of CHMP1A, was present in all analyzed cells, likely corresponding to the promoter region. The remaining 11 regions were only present in proximal tubule cells of the human kidney (FIG. 2A). Mouse kidney single-cell accessible chromatin analysis showed important conservation of the locus (FIG. 10A), including the genomic organization, the open chromatin at the promoter region, and the proximal tubule-specific open chromatin pattern. Human kidney bulk H3K27ac, H3K4me1, and H3K4me3 chromatin immunoprecipitation (ChIP-seq) information further supported the single-cell data (FIG. 2A). By direct overlapping the kidney disease-associated SNPs with peaks observed in the snATAC-seq data, it was found that the 5th, 6th, 8th, 9th, 10th, and 12th peaks harbored eGFR GWAS significant SNPs.

To narrow down the set of variants within peaks of accessible chromatin, conditional analysis was performed for eGFR GWAS to explain the observed pattern of association. It was found that SNPs within peaks without GWAS SNPs (peaks 1, 3, 4, and 11) did not attenuate the association signal. Peaks 2 and 7 had no available SNPs. Adjustment for SNPs within peaks 5 and 12 did not completely account for the GWAS signal, with modest residual associations (p<0.001) in the remaining regions. However, conditional analysis for SNPs in peaks 6, 8, 9, and 10 all demonstrated local attenuation of association (P>0.001) (FIG. 10B).

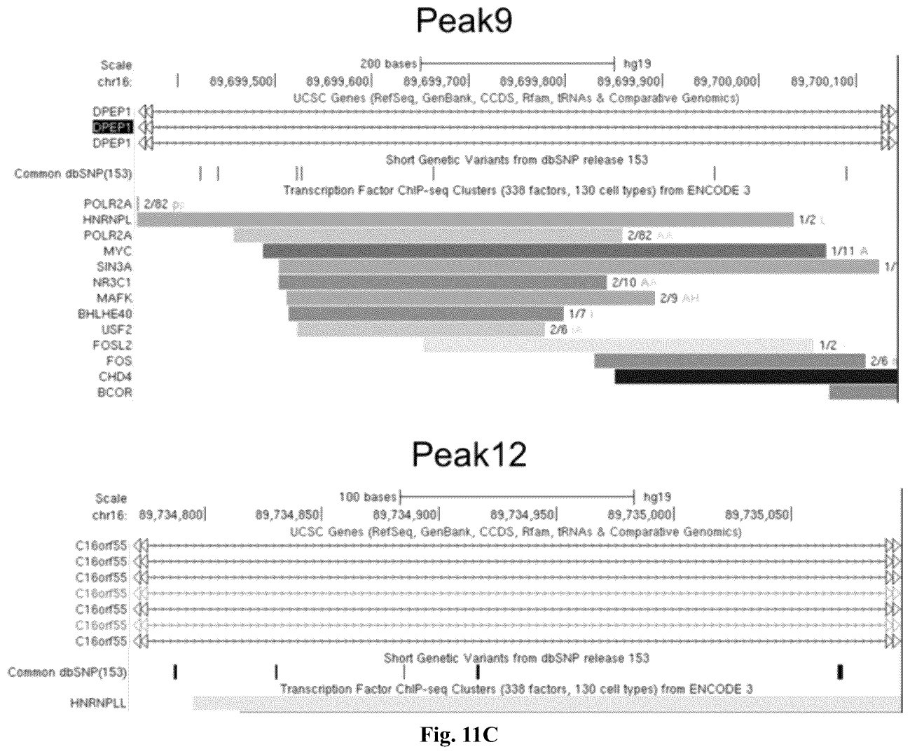

Next, CRISPR-based genome editing was performed in cultured kidney cells (HEK293) to define the role of these regions in regulating DPEP1 and CHMP1A expression (FIG. 2B). It was decided to delete the entire prioritized region that contained the likely causal variant rather than performing individual SNP editing, given most reports suggest that multiple SNPs play roles in gene regulation. Successful deletion of the region was confirmed by Sanger sequencing (FIG. 11A). DPEP1 expression was increased following the deletion of peaks 8 and 9, whereas the CHMP1A expression was decreased when the 8th and 12th peaks were deleted (FIG. 2C). Interestingly, the cicero-based co-accessible analysis of the snATAC-seq in mouse kidneys further confirmed the shared regulation of these peaks (FIG. 10A). TFs binding site profiling from the ENCODE database indicated potential binding of proximal tubule-specific TFs, such as HNF1A, HNF4A, and HNF4G (FIG. 1B) and YY1, which mediates long-distance DNA interactions (FIG. 11B).

Taken together, integration of kidney function GWAS and snATAC-seq followed by CRISPR/Cas9 genome editing prioritized causal regulatory regions influencing the expression of both DPEP1 and CHMP1A in human kidney proximal tubule cells.

### Example 1-4: Dpep1 Deficiency Ameliorates Toxic Renal Injury Induced by Cisplatin or Folic Acid

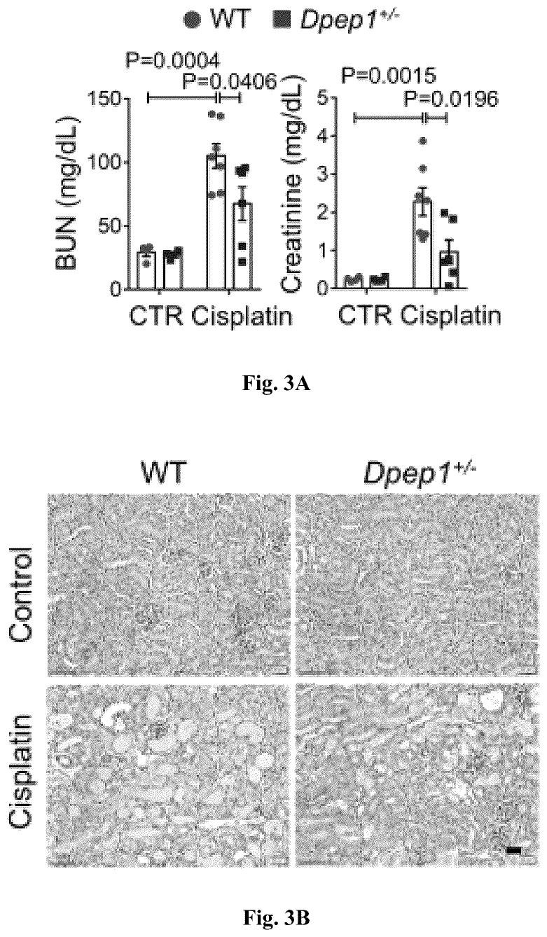

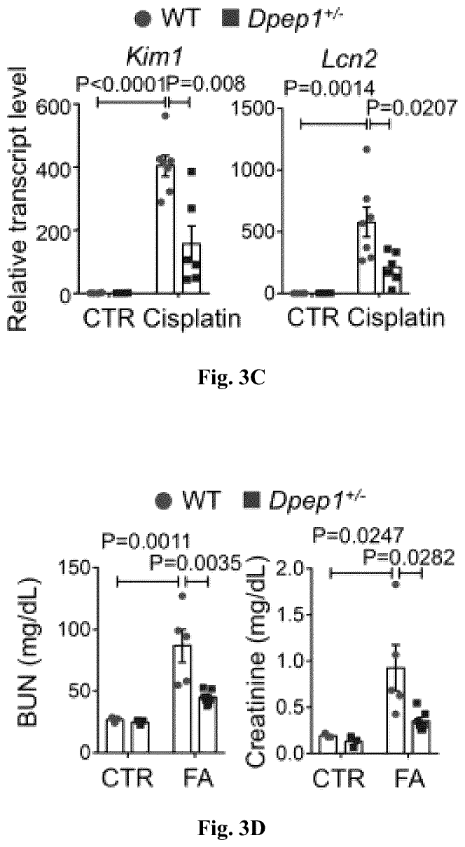

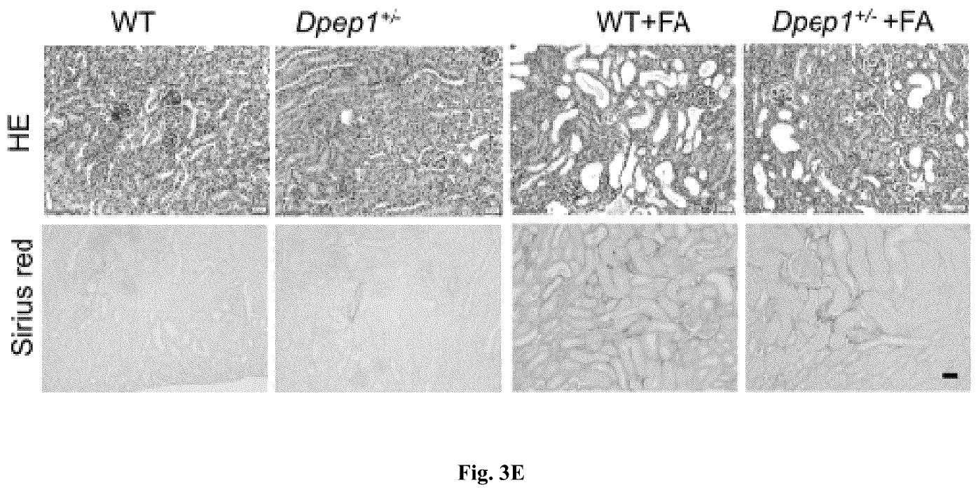

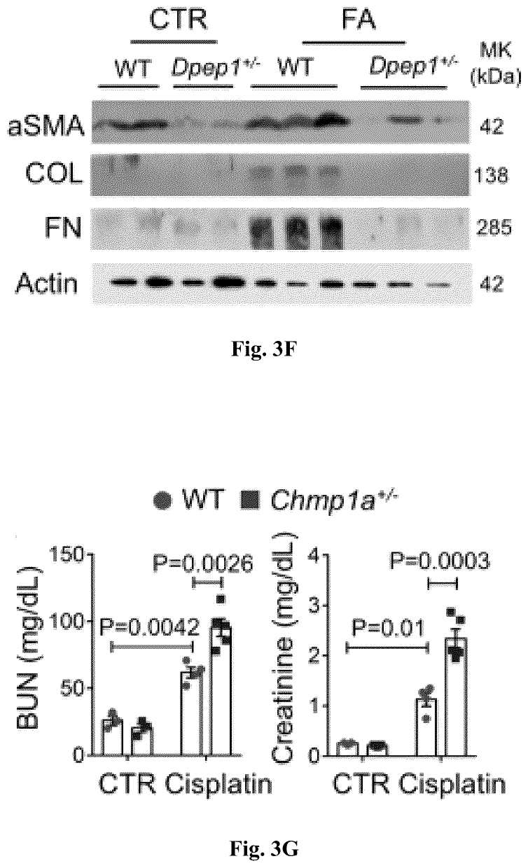

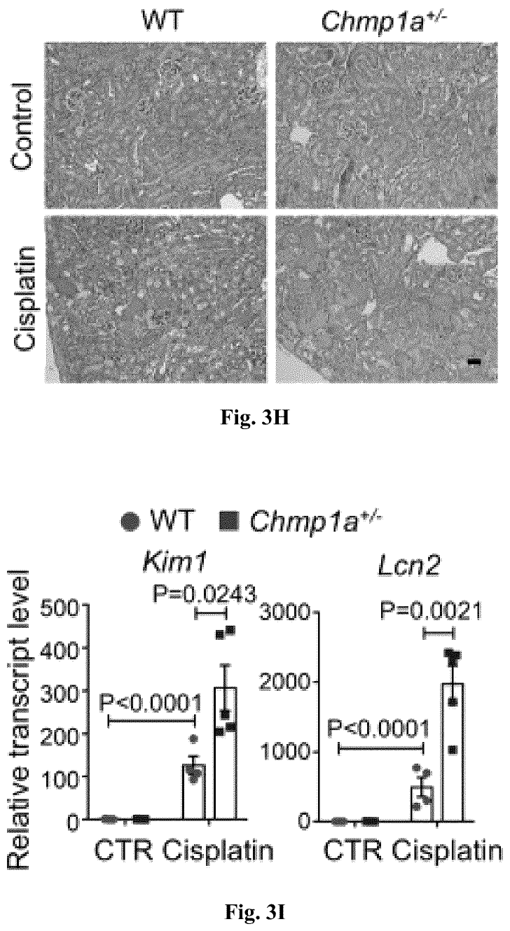

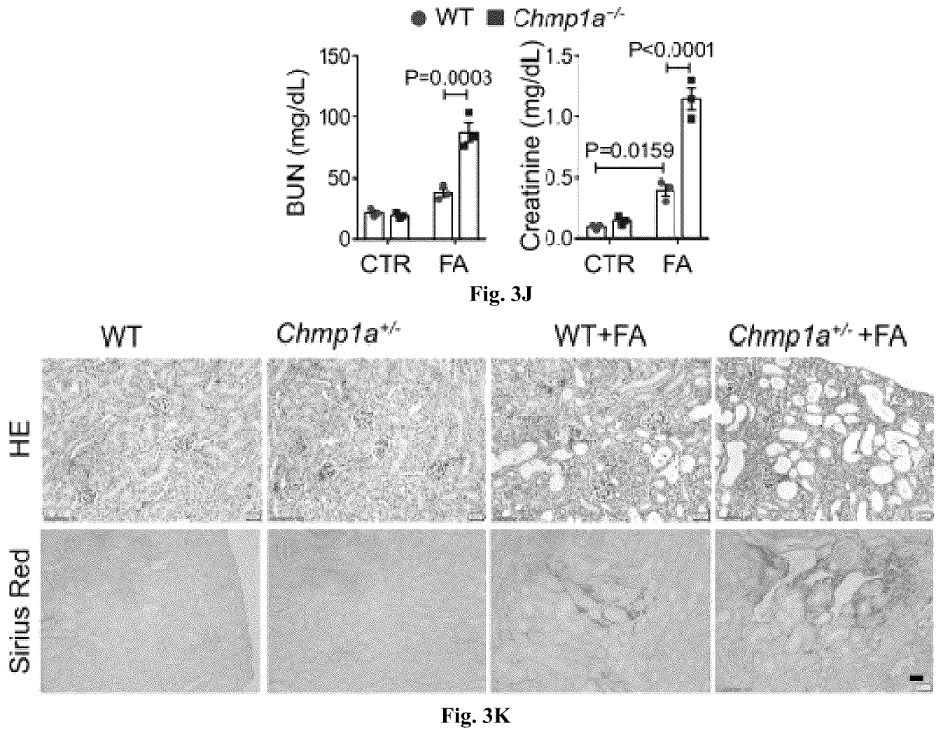

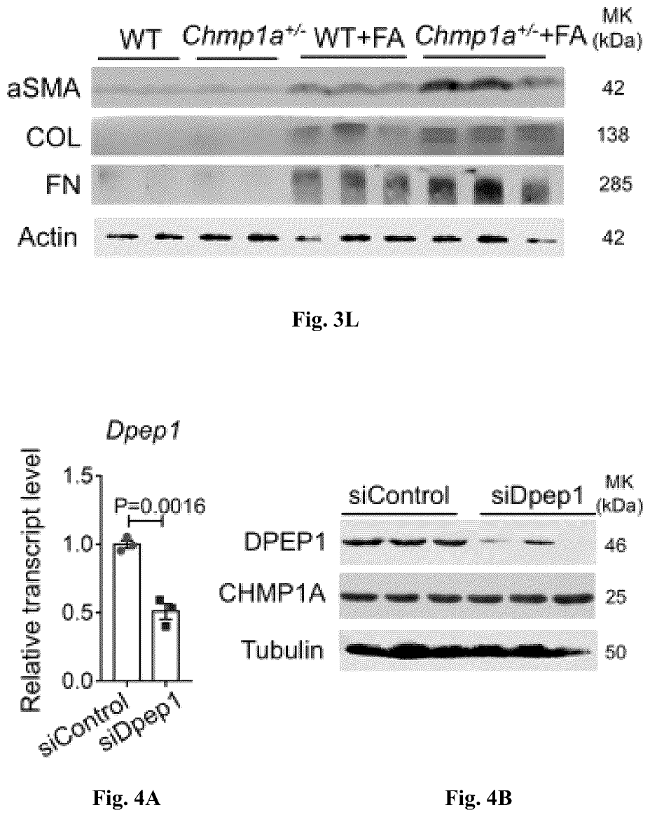

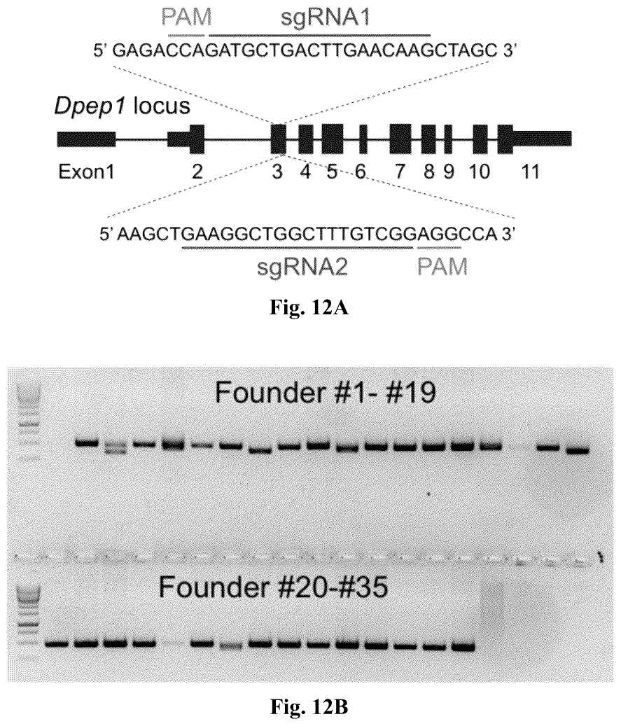

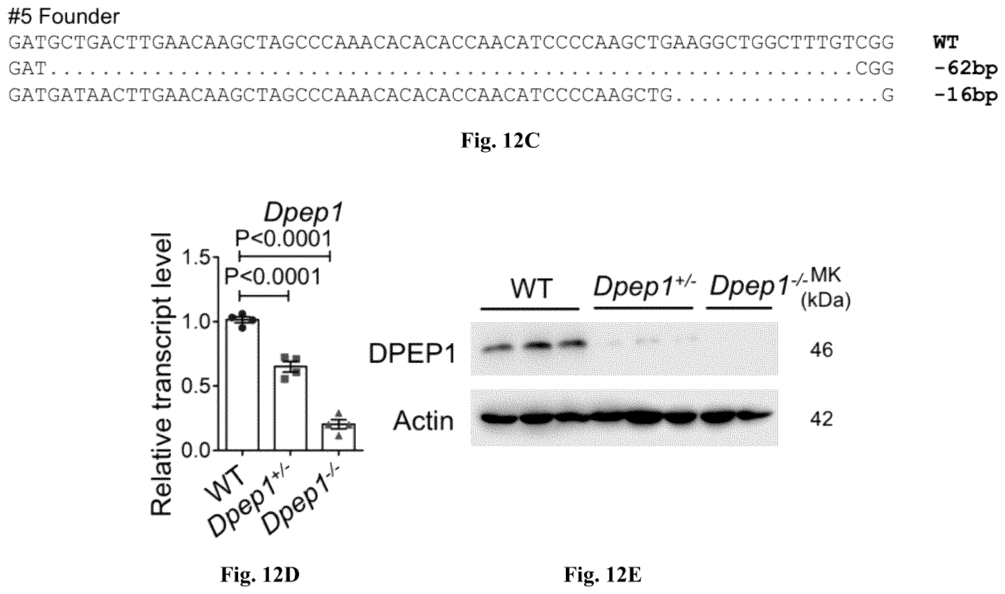

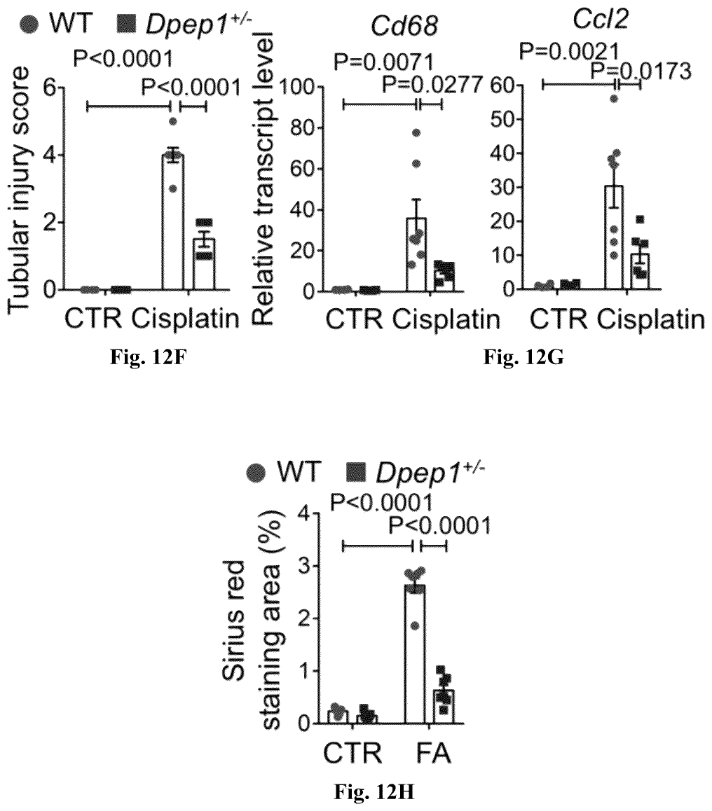

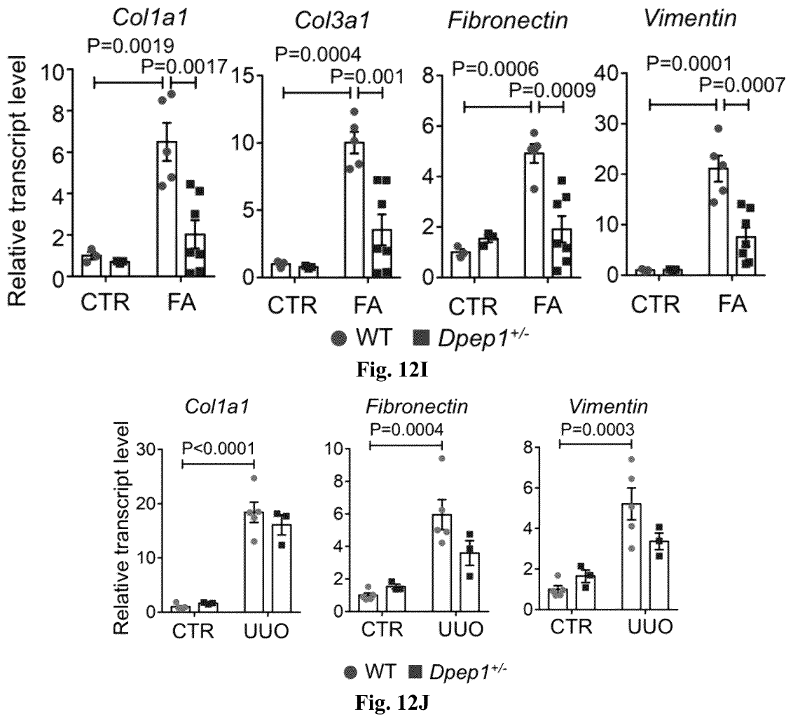

To understand the role of Dpep1 in kidney disease development, mice with genetic deletion of Dpep1 were generated using CRISPR/Cas9 technology. Exon 3 of Dpep1 was targeted by two sgRNAs (FIG. 12A). Six out of thirty-five founders showed successful deletion by Sanger sequencing (FIG. 12B). Founder #5 containing a 62 base pair deletion at exon 3 was used in experiments (FIG. 12C). Transcript and protein expression were decreased in Dpep1+/− and Dpep1−/− kidney tissue samples compared with control littermates (FIG. 12E), indicating the successful generation of knockout mice. Dpep+/− and Dpep1−/− mice were born at the expected Mendelian ratio and appeared normal. Gross phenotypic analysis of the Dpep1 mutant mice showed no obvious changes in blood urea nitrogen (BUN) level, serum creatinine level, and kidney histology (FIGS. 3A-3B).

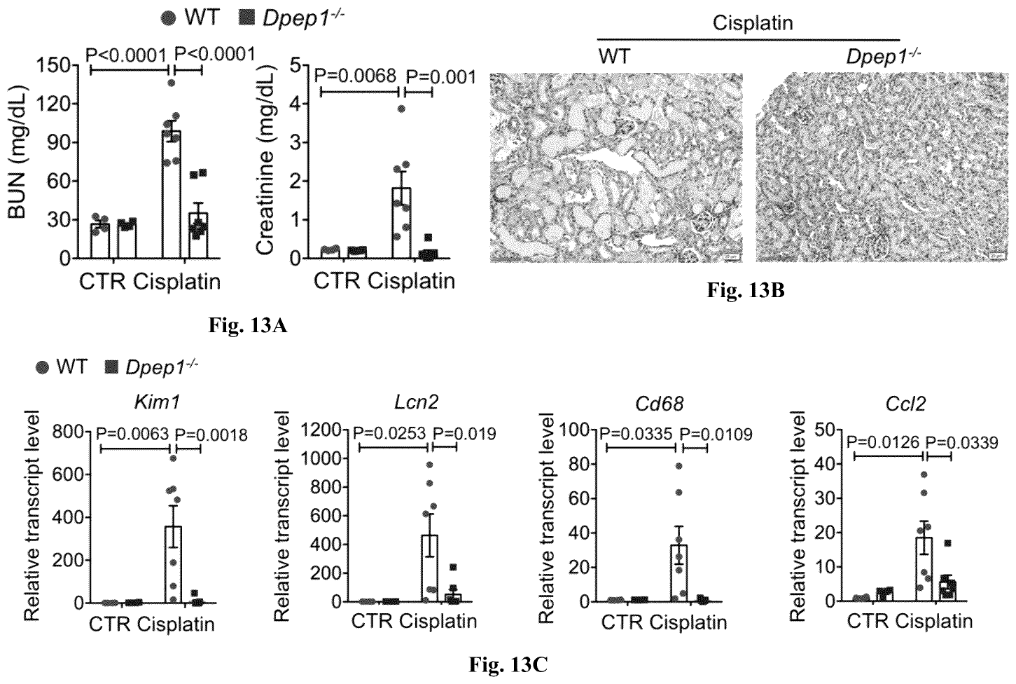

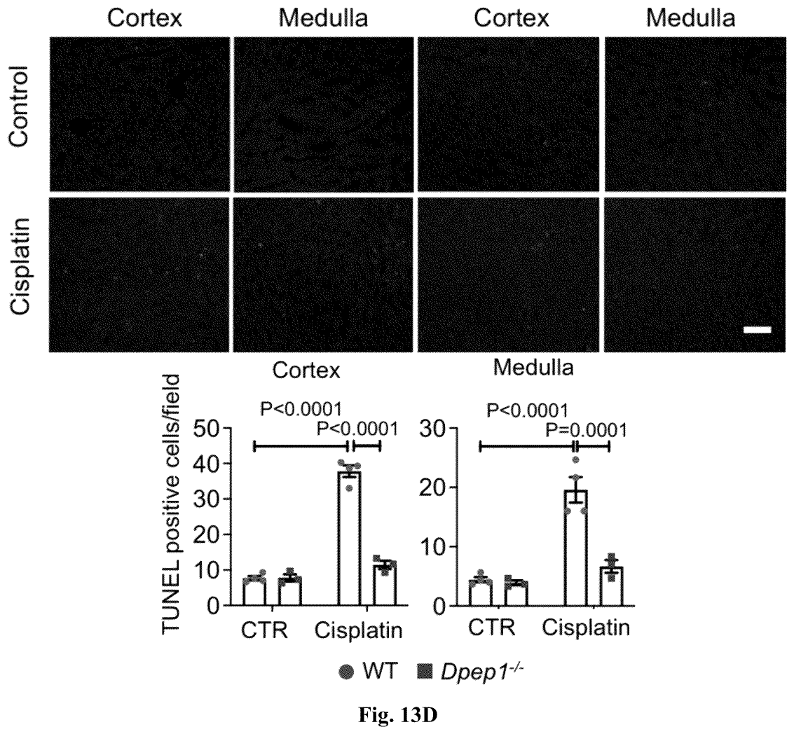

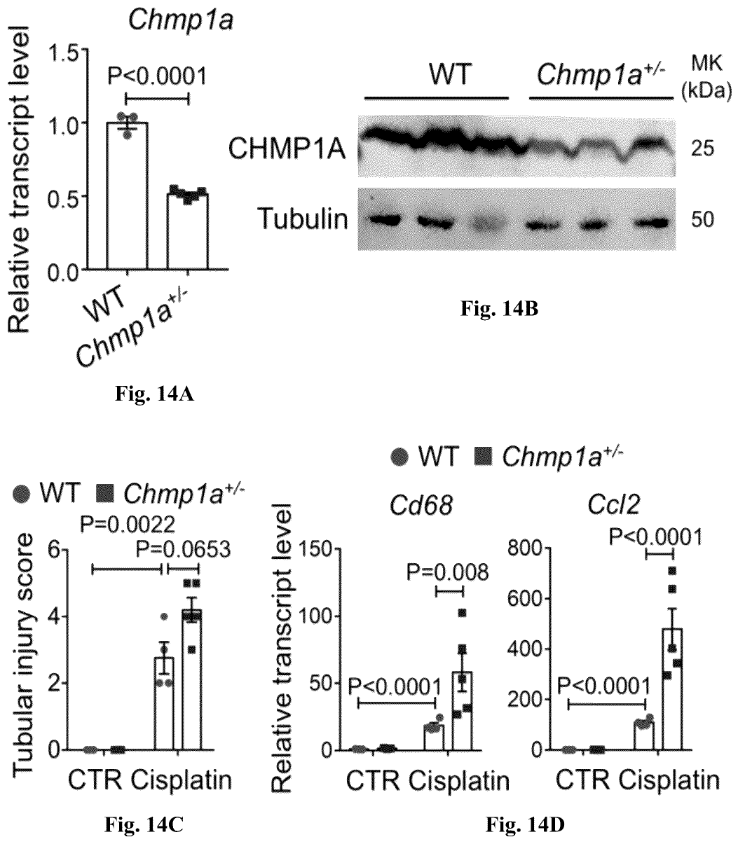

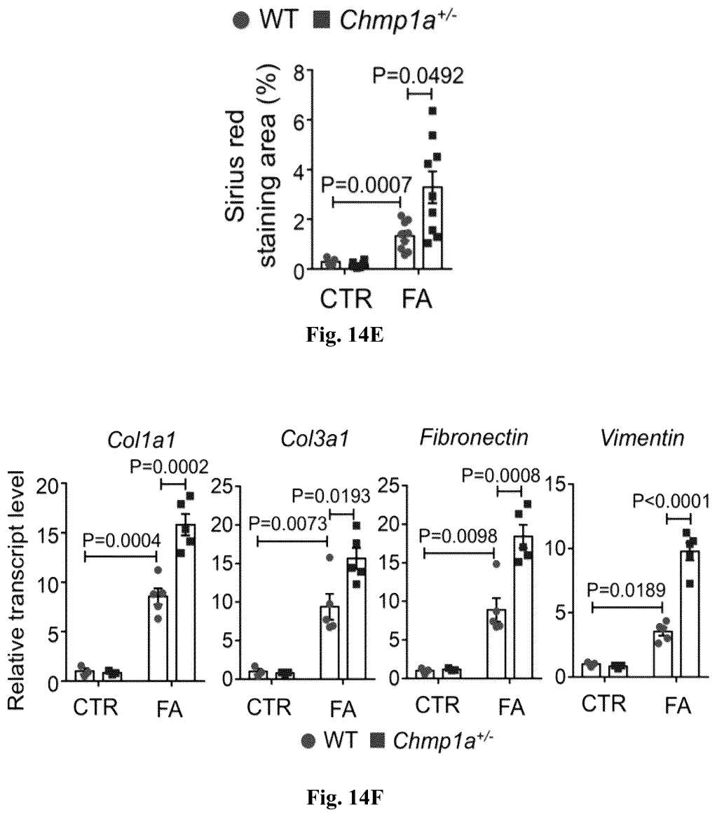

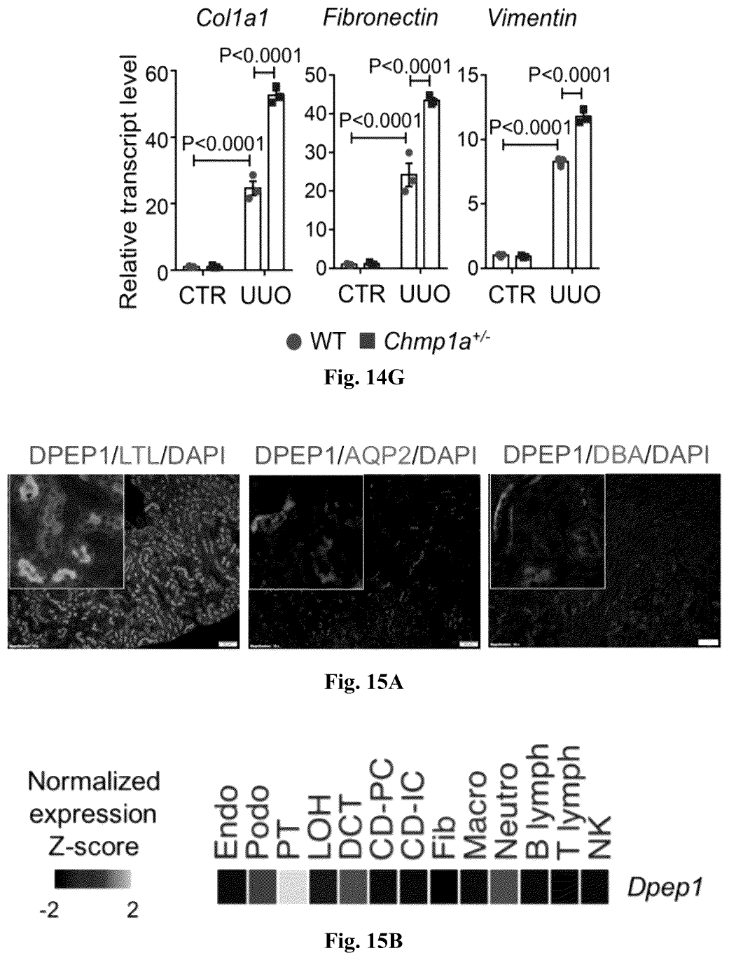

To understand the role of Dpep1 in kidney disease, wild-type, Dpep1+/−, and Dpep1−/− littermates were challenged with cisplatin, a chemotherapeutic with known proximal tubule toxicity. Following cisplatin administration, Dpep+/− and Dpep1−/− mice showed lower serum BUN and creatinine levels when compared to wild-type animals (FIG. 3A and FIG. 13A). Kidney histology, such as tubular dilation and immune cell infiltration was markedly lower in cisplatin-treated Dpep1^{+/-} and Dpep1−/− mice when compared to wild-type mice (FIG. 3B, FIGS. 12F and 13B). Expression levels of acute kidney injury markers Kim1 and Lcn2, and cytokines Ccl2 and Cd68, were lower in the cisplatin-injected Dpep1^{+/-} mice when compared to wild-type littermates (FIGS. 3C, 12G, and 13C).

The role of Dpep1 was further investigated in the folic acid (FA) model, which is a mixed model of acute kidney injury leading to fibrosis development. Serum BUN and creatinine levels in Dpep1+/− mice with cisplatin treatment were lower than control mice (FIG. 3D) Structural changes analyzed on HE-stained kidney sections indicated attenuated tubule injury in Dpep+/− kidneys following FA injection (FIG. 3E) and less collagen on Sirius red-stained sections (FIGS. 3E, 12H). Consistent with the histological changes, transcript and protein levels of profibrotic genes, including Col1a1, Col3a1, Fibronectin, and Vimentin, were markedly decreased in Dpep1+/− mice when compared to wild-type littermates after FA injection (FIGS. 3F, 12I). Dpep1 mutant mice also showed lesser kidney damage when compared to controls in the unilateral ureteral obstruction model (FIG. 12J). In summary, in vivo studies indicated that deletion of Dpep1 ameliorated kidney injury in mice.

### Example 1-5: Dpep1 Knockdown Protects from Cisplatin-Induced Injury without Affecting Necroptosis and Pyroptosis

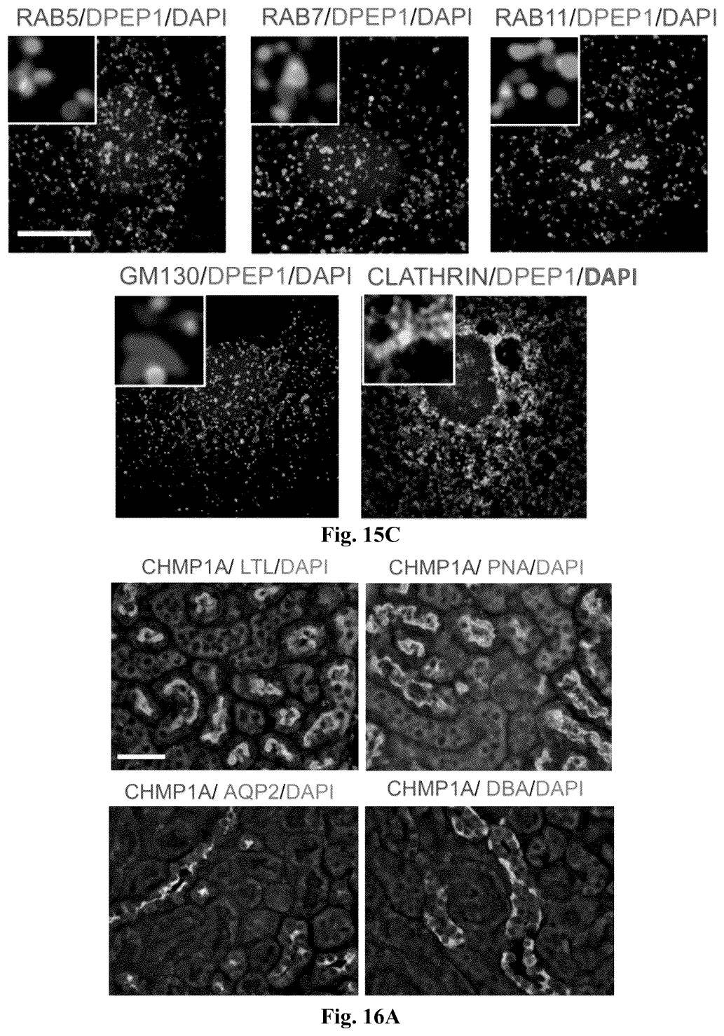

Next analyzed was Dpep1 expression in mouse kidneys and cultured kidney cells. Immunofluorescence staining indicated colocalization of DPEP1 with the proximal tubule marker Lotus Tetragonolobus Lectin (LTL), but not with collecting duct marker AQP2 or distal convoluted tubule marker Dolichos biflorus Agglutinin (DBA) (FIG. 15A). The immunostaining results were consistent with the single-cell RNA and ATAC sequencing results (FIG. 15A). Next, the subcellular expression of DPEP1 was analyzed in cultured kidney tubule cells by double staining with organelle markers. No colocalization was observed with endosome markers RAB5, RAB7 and RAB11, and Golgi marker GM130. DPEP1 expression overlapped with Clathrin, a protein that plays a major role in the formation of coated vesicles near the plasma membrane in kidney proximal tubules (FIG. 15C).

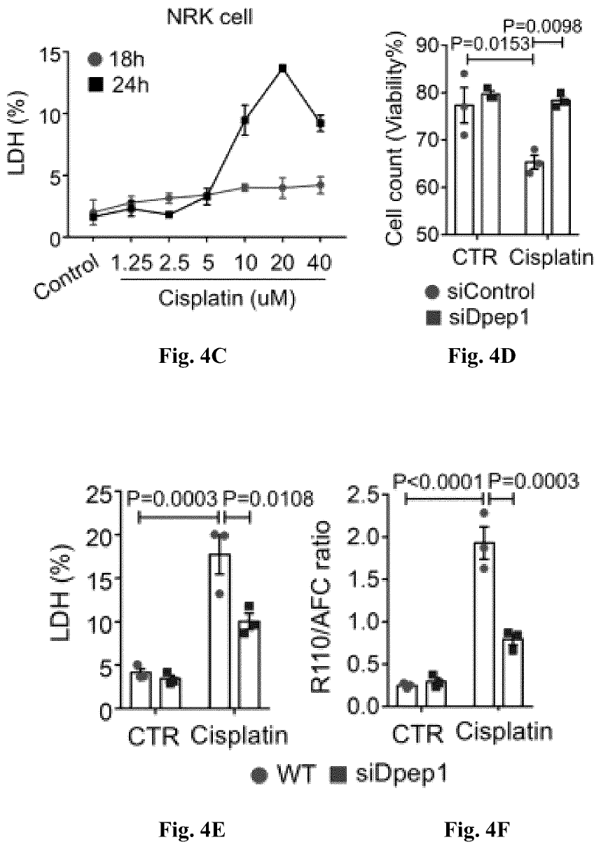

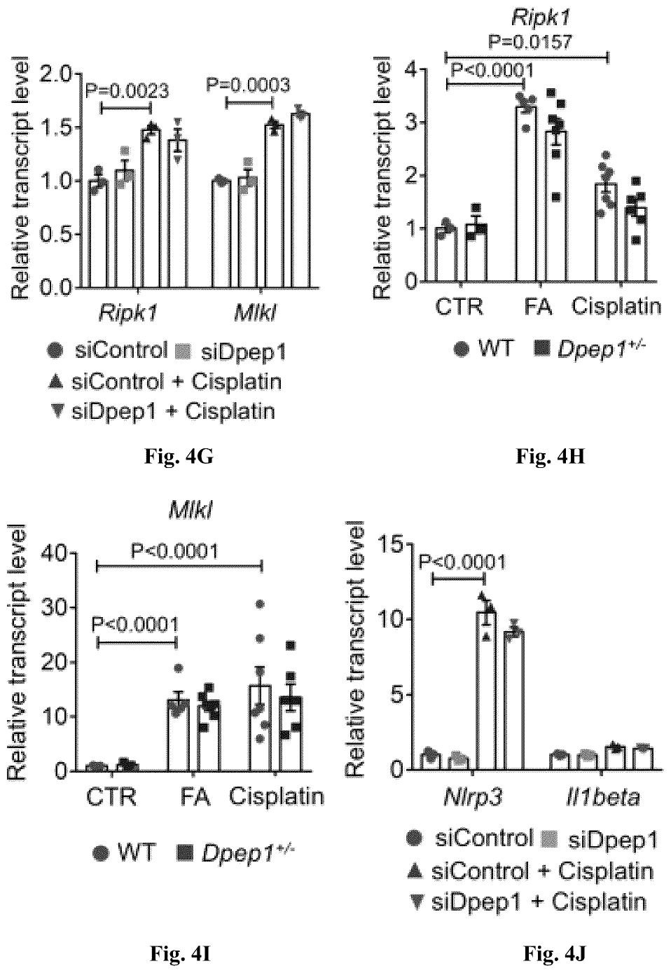

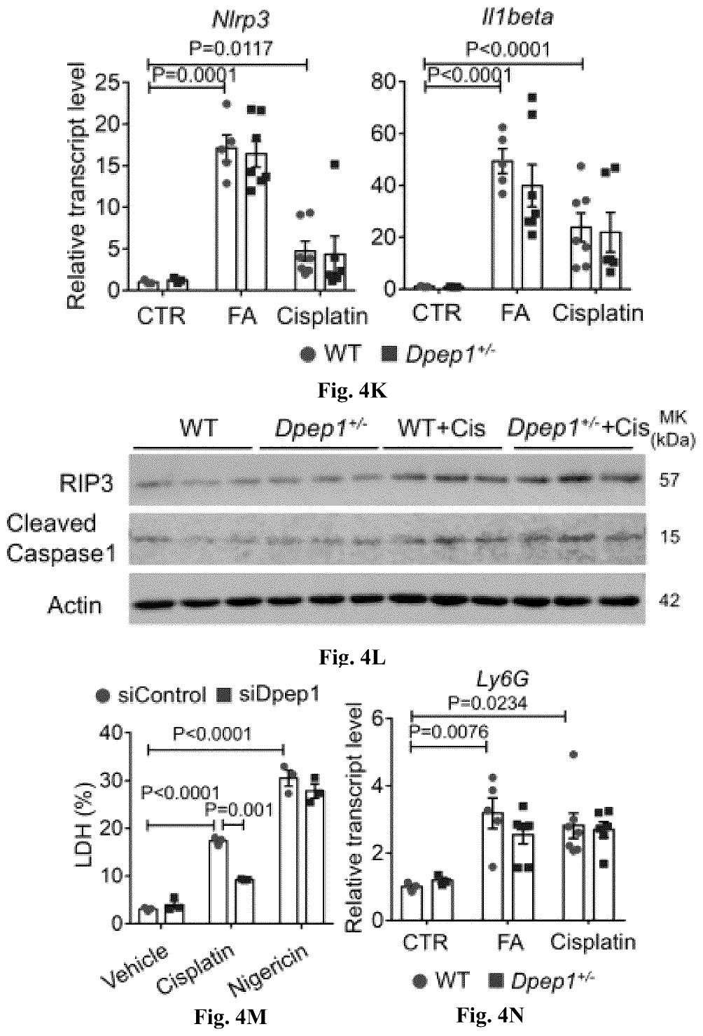

To define the mechanism of Dpep1-mediated kidney injury, rat renal epithelial cells were transfected with siRNA targeted to Dpep1 (siDpep1) or scramble siRNA (siControl). Dpep1 expression was decreased, while Chmp1a level was not affected, in siDpep1 transfected cells (FIG. 4A, 4B). To mimic tubule epithelial cell (TEC) injury observed in mice, TECs were treated with cisplatin. The highest cytotoxicity was observed with 20 μM cisplatin dose after 24 h of treatment (FIG. 4C). Cell count by trypan blue stain showed improved cell viability in siDpep1 transfected cells compared to siControl cells after cisplatin treatment (FIG. 4D). Cytotoxicity measured by LDH release confirmed the improved cell viability of siDpep1 transfected cells (FIG. 4E). Cytotoxicity measured as a ratio of dead cell substrate bis-AAF-R110 and live cell substrate GF-AFC showed a lower R110/AFC ratio in siDpep1 transfected cells after cisplatin treatment (FIG. 4F), again confirming the protective role of Dpep1 knockdown from cisplatin-induced cytotoxicity.

Next, it was sought to understand pathways that mediate the Dpep1 knockdown-afforded protection from TEC cytotoxicity. Transcript levels of Ripk1 and Mlkl, genes involved in the necroptosis, were increased following cisplatin treatment; however, they were not affected in siDpep1 cells (FIG. 4G). Similarly, expression of Ripk1, Ripk3, and Mlkl was increased in injured mouse kidney samples, but there was no difference between diseased wild-type and Dpep1+/− kidneys (FIG. 4H,4I,4L). Pyroptosis markers, such as Nlrp3, Il1beta, and cleaved caspase 1 were elevated in cisplatin-treated cells and kidneys of cisplatin- or FA-treated mice. No differences were observed when cisplatin-treated control cells were compared to Dpep1 knockdown cells or Dpep1 knockout kidneys (FIG. 4J-4L). No difference was observed in LDH release following nigericin-induced Nlrp3 activation when control and Dpep1 knockdown cells were compared (FIG. 4M). Recently, Dpep1 was shown to play a role in cell adhesion for neutrophil recruitment in the lung and liver; however, no difference was found in expression of the neutrophil marker Ly6G in kidneys of control and diseased Dpep1+/− mice (FIG. 4N). In summary, this data failed to support the role of pyroptosis and necroptosis in Dpep1 knockdown-mediated protection from cisplatin-induced cytotoxicity.

### Example 1-6: Chmp1a Knockdown Sensitizes Cisplatin-Induced Cell Death without Altering Necroptosis, Pyroptosis, and Apoptosis

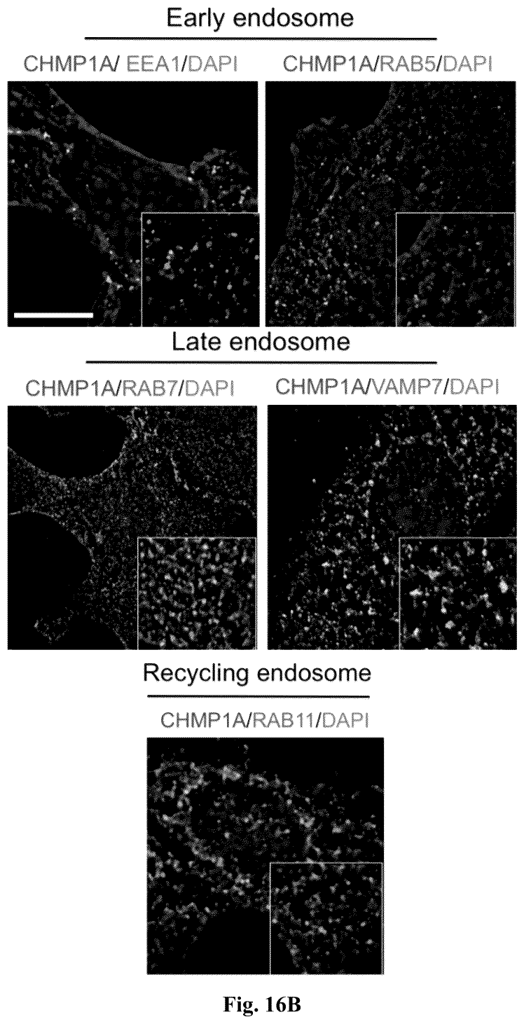

The expression of Chmp1a in mouse kidney tissue and cell lines was first examined. Immunofluorescence staining showed that CHMP1A colocalized with the proximal tubule marker LTL, loop of Henle marker Peanut Agglutinin (PNA), collecting duct marker AQP2, and distal convoluted tubule marker DBA, indicating that CHMP1A was expressed in all tubular segments (FIG. 16A). CHMP1A is a component of endosomal sorting complexes required for transport (ESCRT). It was found that CHMP1A was not colocalized with early endosome markers EEA1 and RAB5, and recycling endosome marker RAB11, but colocalized with late endosome markers RAB7 and VAMP7 (FIG. 16B).

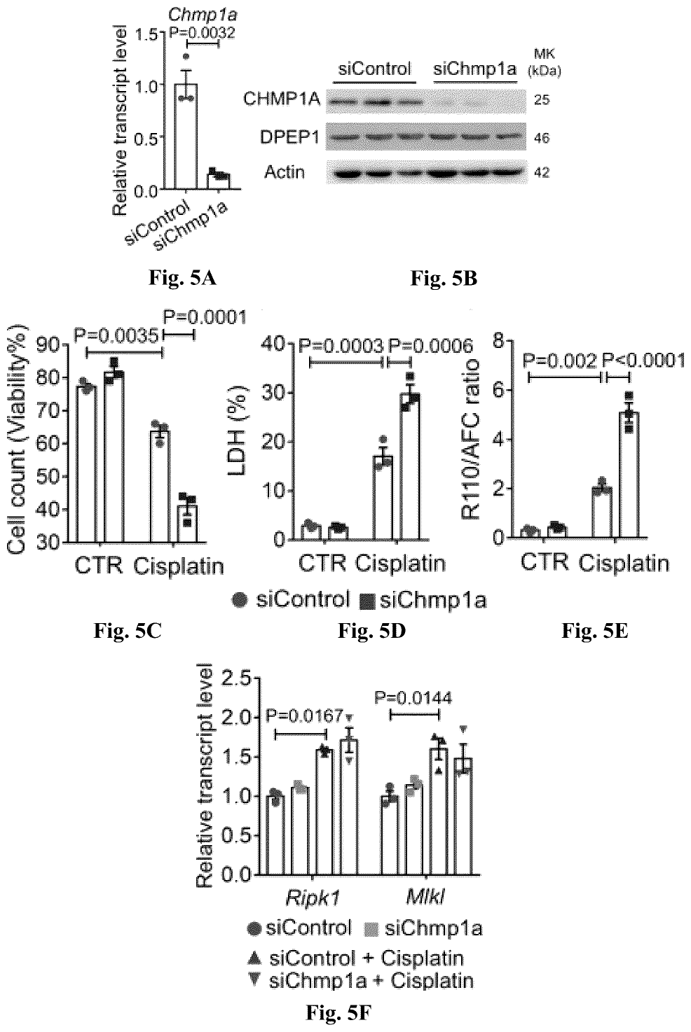

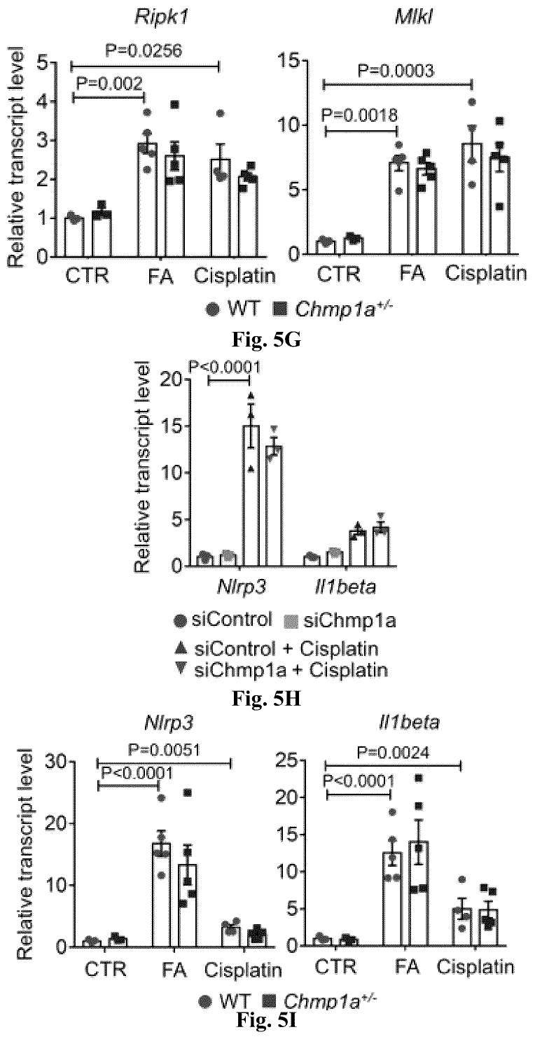

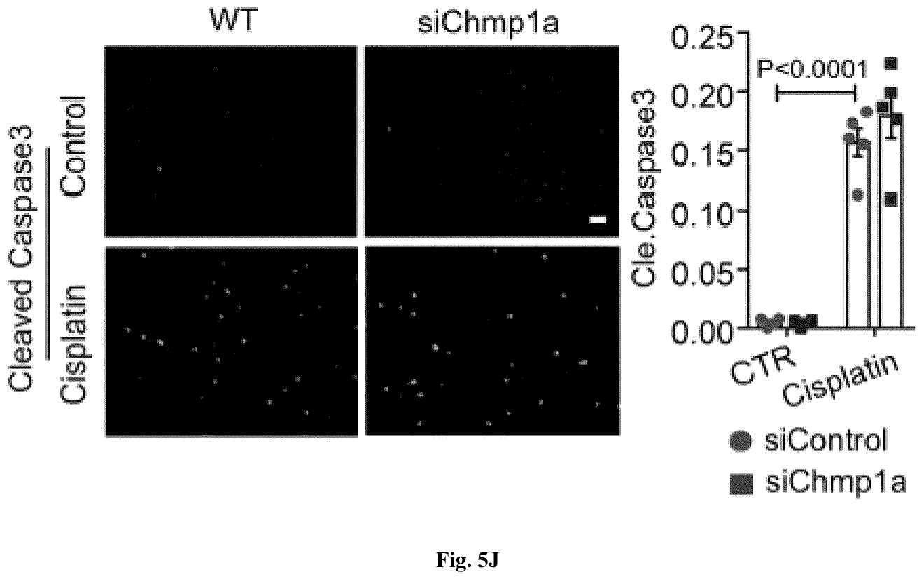

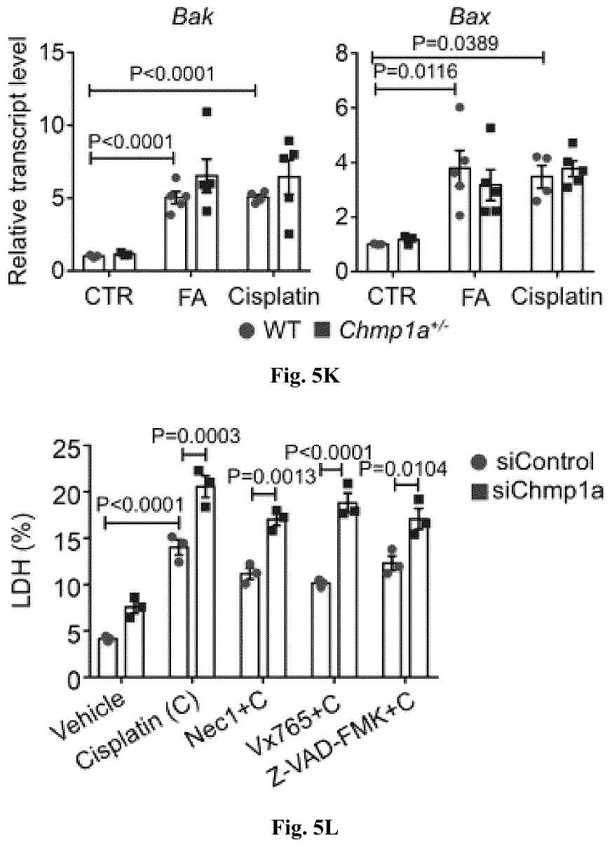

Consistent with the in vivo data, it was found that rat TEC cells NRK52E expressed high levels of Chmp1a. Chmp1a siRNA, which reduced Chmp1a but not Dpep1 levels (FIGS. 5A-5B) was used. It was that cell viability was lower in siChmp1a treated cells when compared to controls, following cisplatin treatment (FIG. 5C). Both LDH release and cell death indicator, R110/AFC, were significantly increased in the siChmp1a cells after cisplatin treatment (FIGS. 5D,5E). These results indicated increased cytotoxicity in Chmp1a deficient kidney tubule cells.

It was found that markers of necroptosis and pyroptosis, such as Ripk1, Mlkl, Nlrp3, and Il1beta were increased in cisplatin-treated cells and mice; however, comparable change in their expression levels in siChmp1a treated cells and Chmp1a^{+/-} kidneys (FIGS. 5F-5I) was observed. Looking at genes associated with apoptosis, no obvious differences of cleaved caspase 3-positive cells were found when siControl and siChmp1a cells were compared after cisplatin treatment (FIG. 5J). Transcript levels of Bak and Bax were comparable in injured control and Chmp1a^{+/-} kidneys (FIG. 5K). To confirm the role of Chmp1a in necroptosis, pyroptosis and apoptosis, siControl and siChmp1a cells were pretreated with the necroptosis inhibitor necrostatin-1 (Nec-1), the pyroptosis inhibitor Vx765 and a pan-caspase inhibitor Z-VAD-FMK followed by cisplatin treatment. No change was observed in LDH release following co-incubation with any of these inhibitors (FIG. 5I). In summary, Chmp1a deficiency increased cisplatin-induced cell death with no observable change in classical genes involved in necroptosis, pyroptosis, and apoptosis.

### Example 1-7: Dpep1 Knockdown Ameliorates Cisplatin-Induced Apoptosis and Ferroptosis

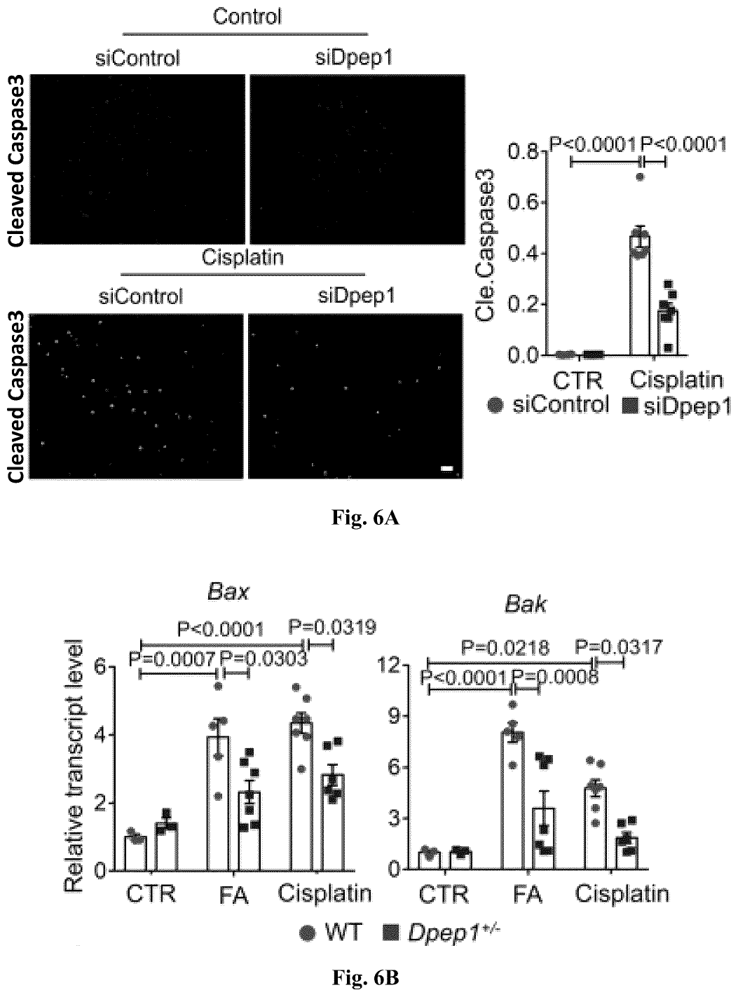

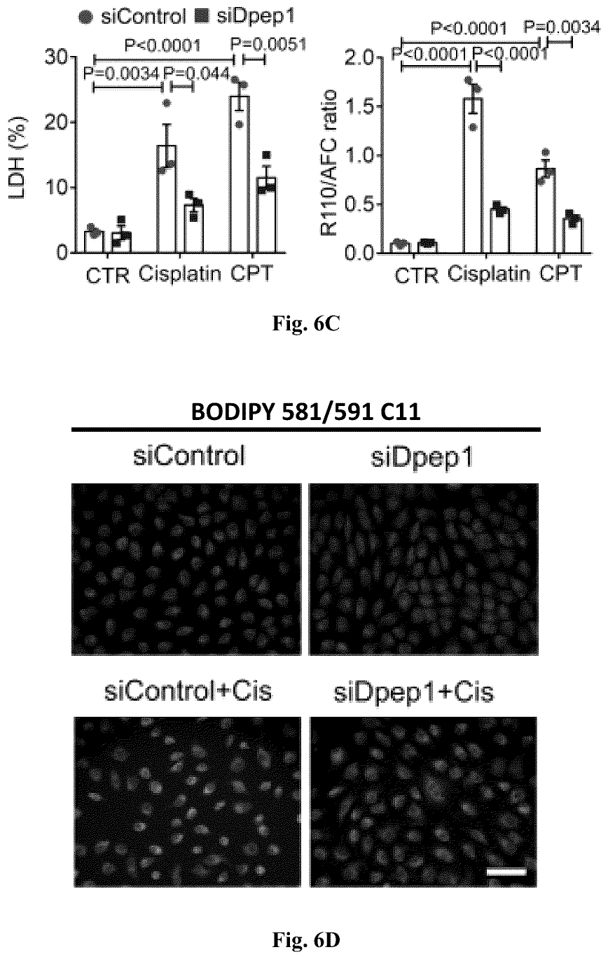

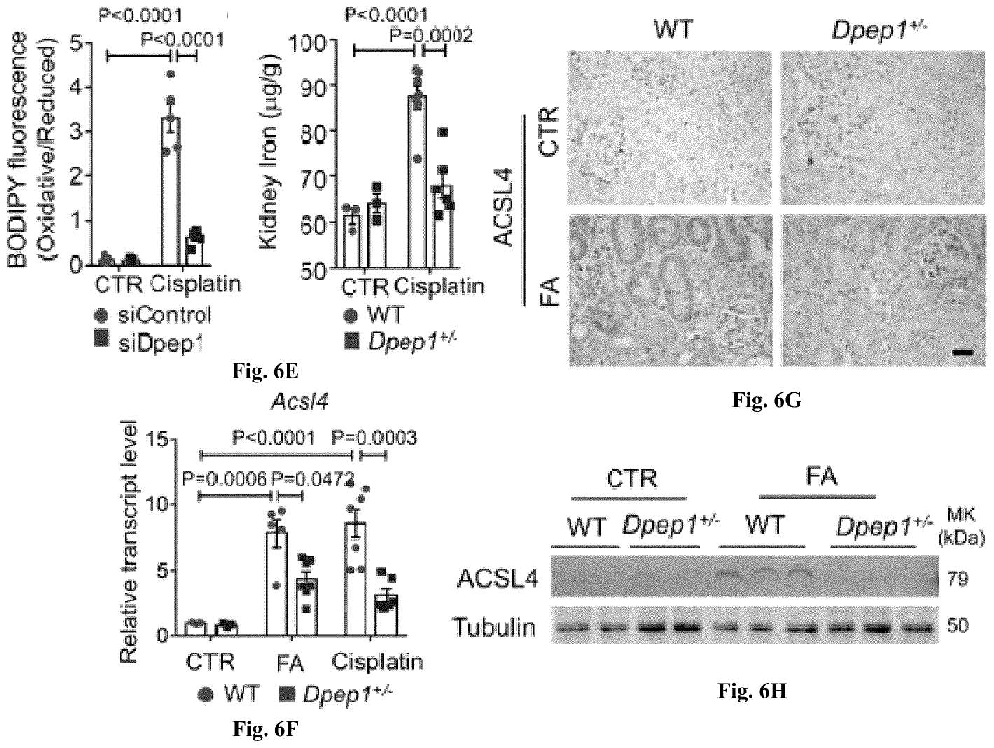

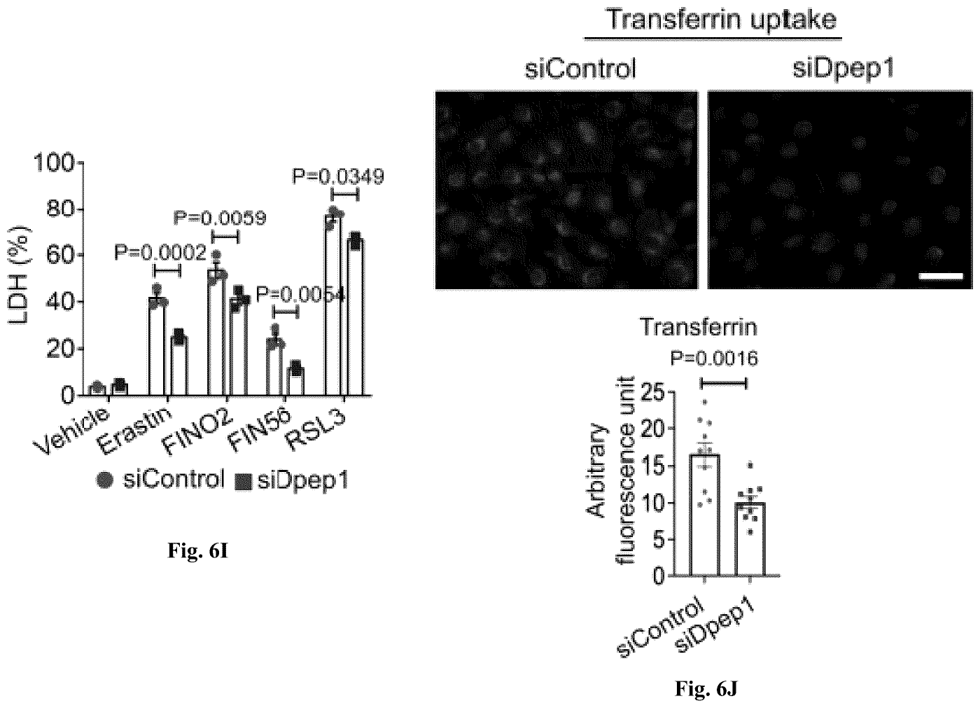

Next, the role of Dpep1 in cisplatin-induced programmed cell death was examined. The number of cleaved caspase 3-positive cells was lower in siDpep1 transfected cells compared to siControl cells following cisplatin administration (FIG. 6A). Transcript expression of Bax and Bak was lower in Dpep1^{+/-} kidneys both in the FA and cisplatin models compared to controls (FIG. 6B). LDH release and the ratio of R110/AFC indicated increased cytotoxicity following treatment with the apoptosis-inducer camptothecin (CPT), Improved cell viability was observed in Dpep1 knockdown TEC when compared to control cells (FIG. 6C). TUNEL assay further confirmed the lower cell death rate in kidneys of cisplatin-treated Dpep1−/− mice when compared with wild-type littermates (FIG. 13D). Taken together, these data indicated that Dpep1-mediated cell death was associated with caspase 3 cleavage.

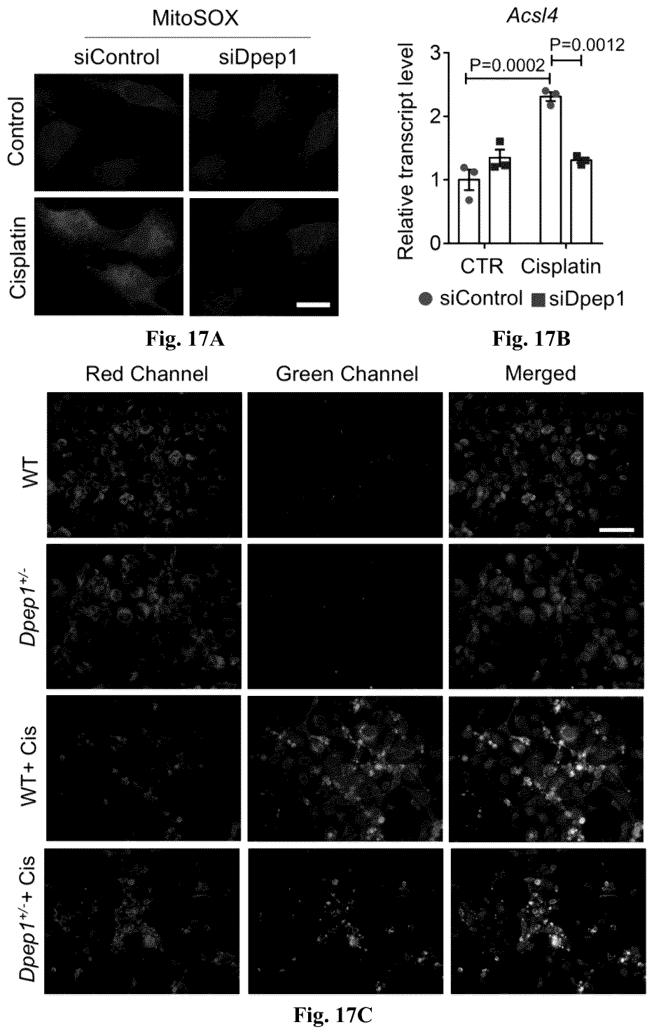

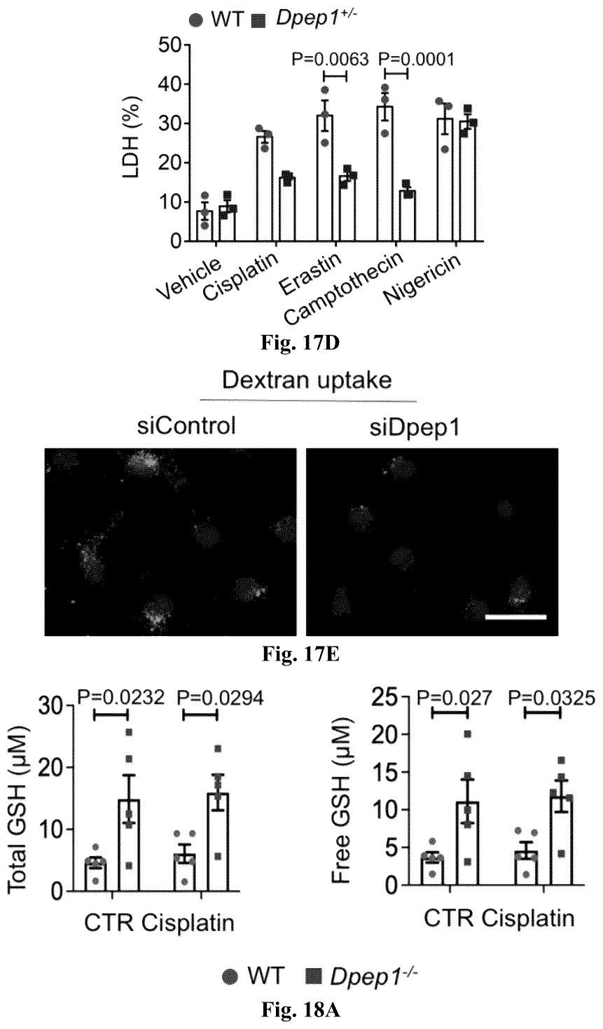

The potential role of ferroptosis in Dpep1-loss offered protection from cell death was next examined. Toxic lipid ROS accumulation is a feature of ferroptosis, which can be monitored by C11 BODIPY 581/591. siControl-transfected cells treated with cisplatin exhibited a robust green signal indicating lipid oxidation, whereas siDpep1 transfection had lower lipid oxidation upon cisplatin treatment (FIG. 6D). Iron catalyzes ferroptosis, and lower iron content was observed in Dpep1^{+/-} kidney lysates after cisplatin administration than in control kidneys (FIG. 6E). Ferroptosis is associated with mitochondrial damage. MitoSOX staining was increased upon cisplatin treatment; however, siDpep1 transfection attenuated the increase (FIG. 17A). In addition, a marked increase of long-chain-fatty-acid-CoA ligase 4 (Acsl4) expression, an important marker of ferroptosis, in diseased wild-type kidneys but not in Dpep1^{+/-} kidneys (FIG. 6G-6H) was also observed. Similarly, the expression of Acsl4 was increased in the cisplatin-treated siControl but not in siDpep1 cells (FIG. 17B). To further explore the role of Dpep1 in ferroptosis, the effect of the ferroptosis activators erastin, FINO2, FIN56, and RSL3 was analyzed. Dpep1 knockdown cells had lower LDH release following erastin, FINO2, FIN56, and RSL3 when compared to siControl-transfected cells (FIG. 6I). BODIPY 581/591 probe labeling showed lower lipid peroxidation in cisplatin-treated primary tubular epithelial cells cultured from Dpep1^{+/-} mice (FIG. 17C). The primary tubular epithelial cells obtained from Dpep1^{+/-} mice also showed less LDH release following cisplatin, CPT, and erastin treatment (FIG. 17D). As Dpep1 was reported to regulate radiographic contrast uptake in TEC, it was explored whether Dpep1 regulated dextran and transferrin uptake. Lower dextran (FIG. 17E) and transferrin uptake was observed in siDpep1 TEC compared to scramble siRNA cell (FIG. 6J), suggesting that Dpep1 deficiency results in decreased iron import, leading to lower intracellular iron concentration higher lipid peroxidation threshold and lower ferroptosis.

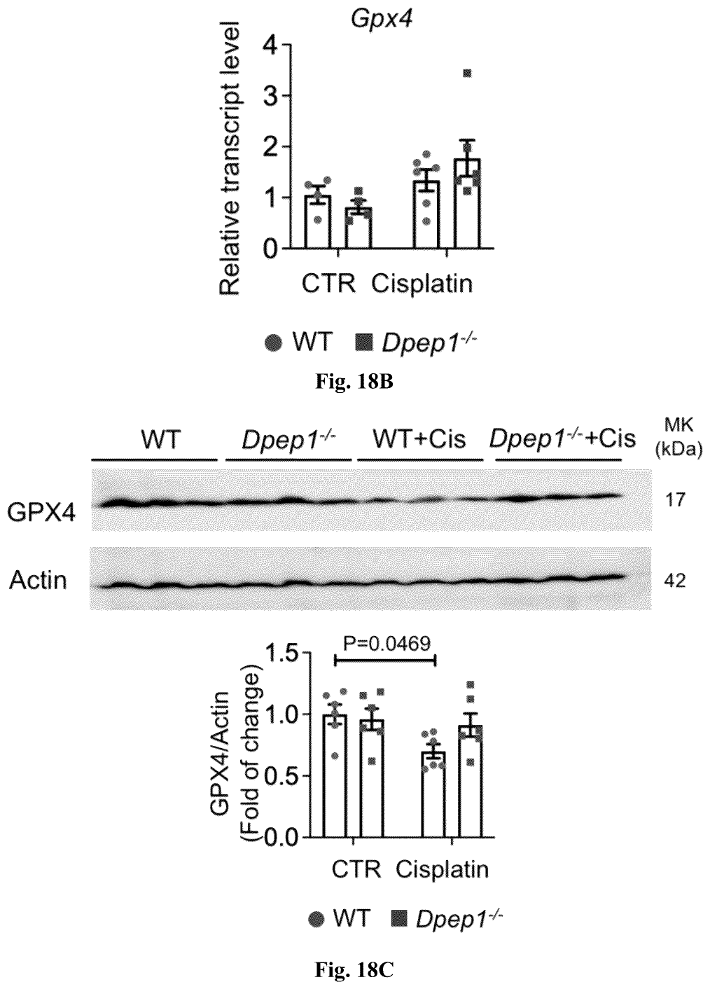

Dpep1 has been proposed to regulate glutathione (GSH) levels, which is the main substrate of the key ferroptosis regulator Gpx4. As shown in FIG. 18A, the total and free GSH level was higher in kidneys of Dpep1^{-/-} mice compared to the wild-type mice with or without cisplatin treatment. Interestingly, cisplatin had no effect on the total and free GSH level and Gpx4 mRNA level in the Dpep1^{-/-} mice (FIG. 18A-18B). In addition, no change was observed in mRNA and protein level of GPX4 level in Dpep1^{-/-} mice compared with the wild-type mice (FIG. 18B,18C). Protein level of GPX4 was slightly lower in the kidneys of the wild-type cisplatin-treated mice (FIG. 18C).

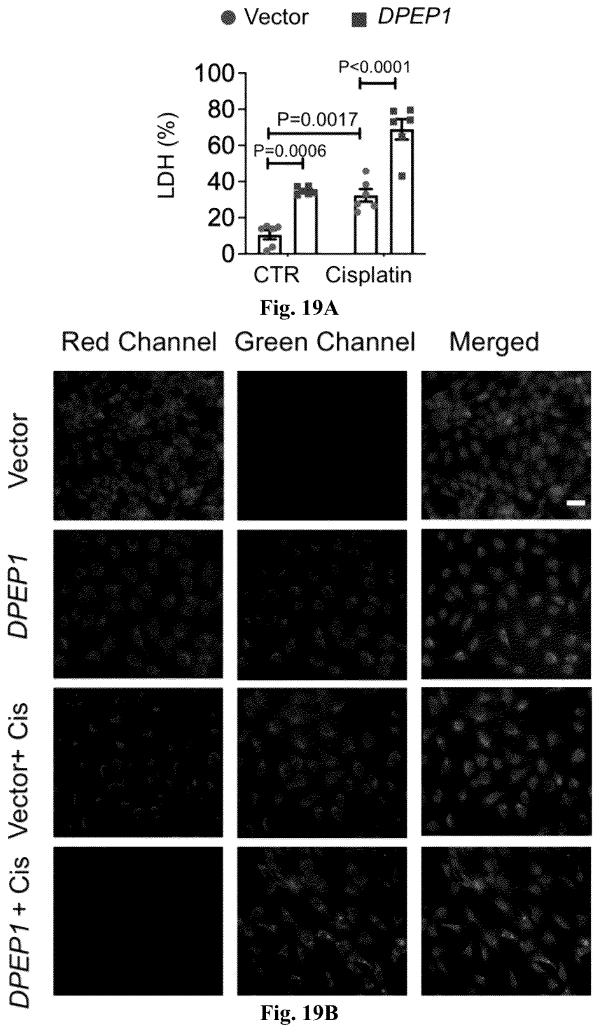

To further confirm the role of Dpep1, NRK-52E cells were transfected with DPEP1 plasmid. Markedly higher LDH release in DPEP1 transfected cells was observed, which was further increased following cisplatin treatment (FIG. 19A). Lipid peroxidation by BODIPY 581/591 C11 was also higher in DPEP1 transfected cells (FIG. 19B), supporting the deleterious role of DPEP1. An important limitation of this experiment remains the supraphysiological level of DPEP1 in these cells.

### Example 1-8: Chmp1a Knockdown Enhances Ferroptosis Through Increased Iron Accumulation

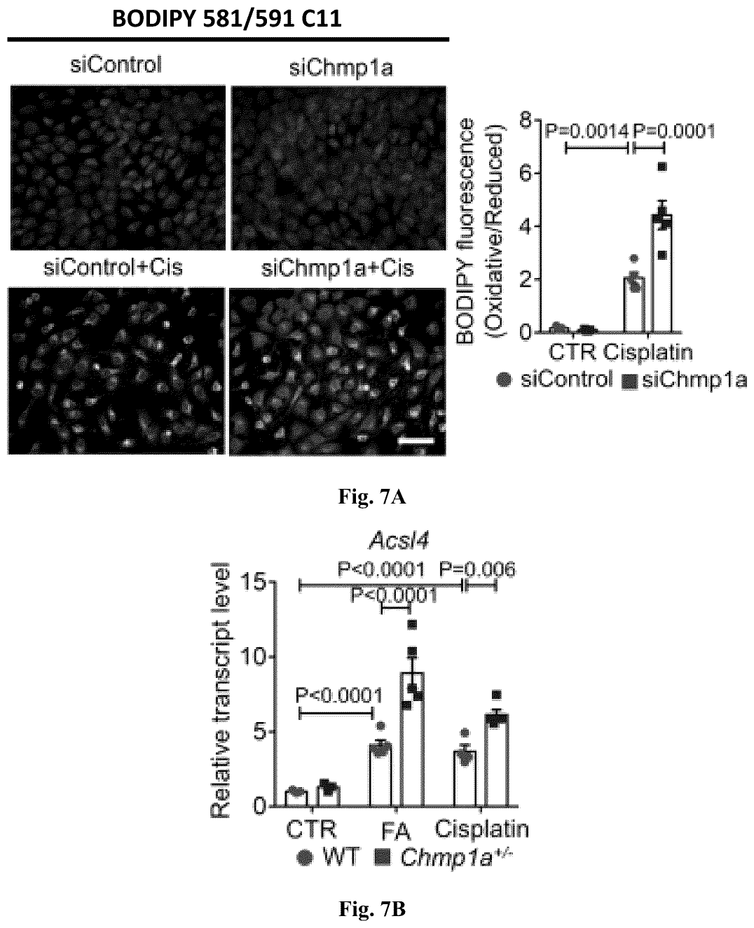

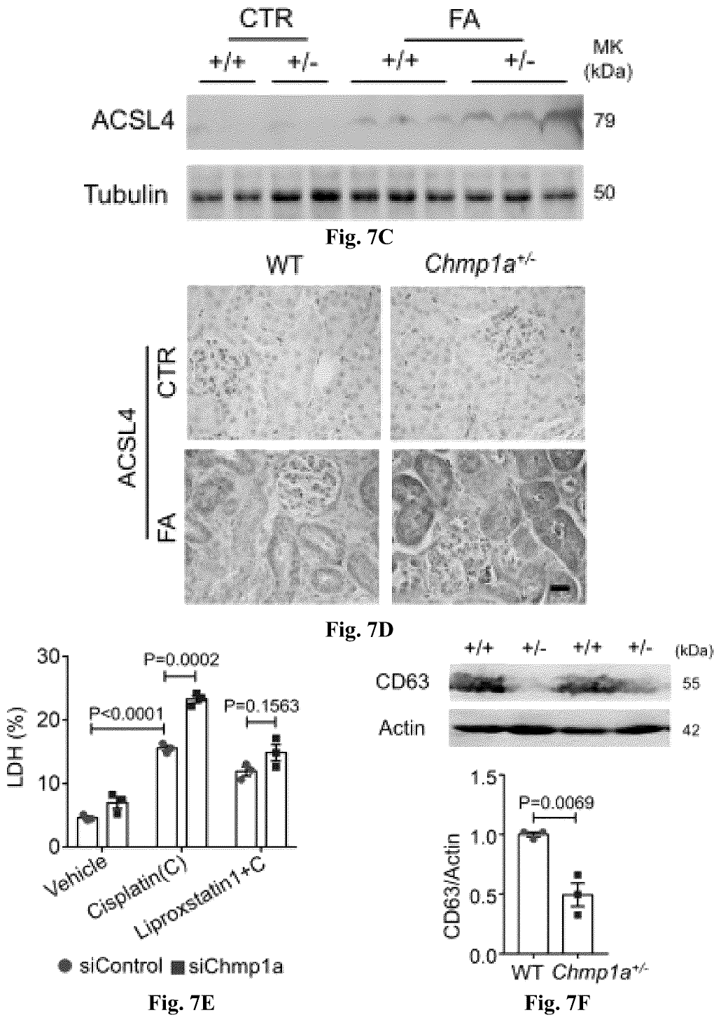

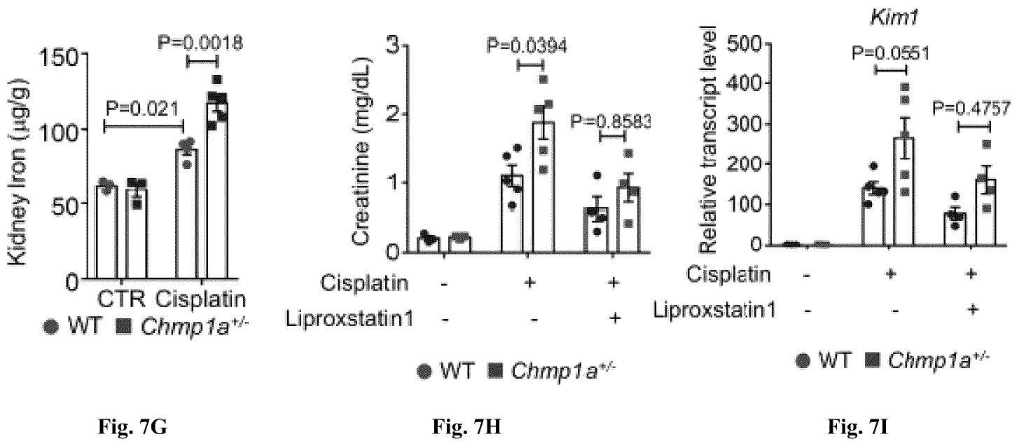

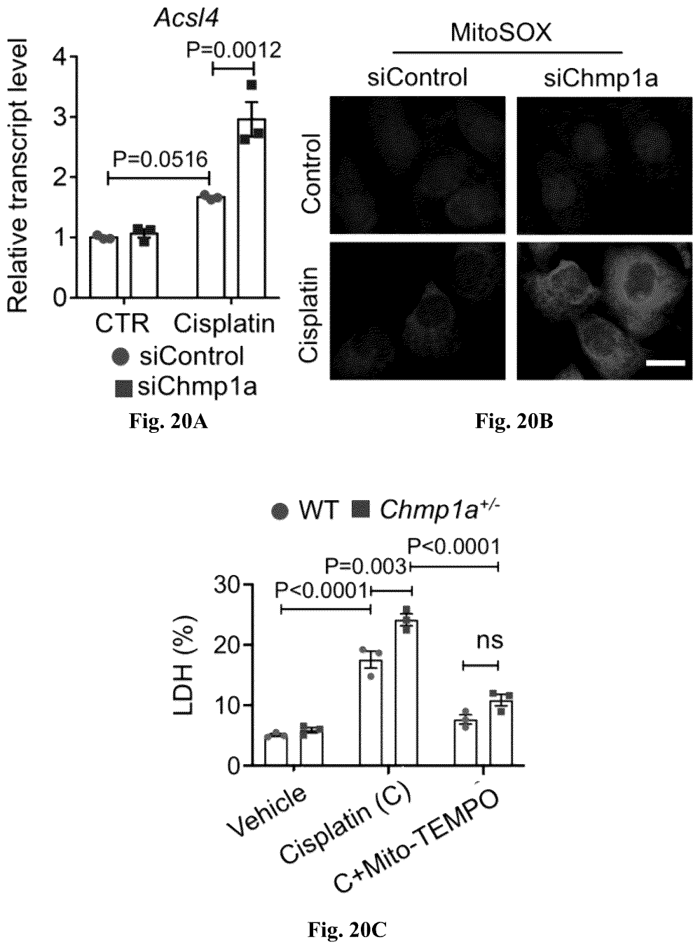

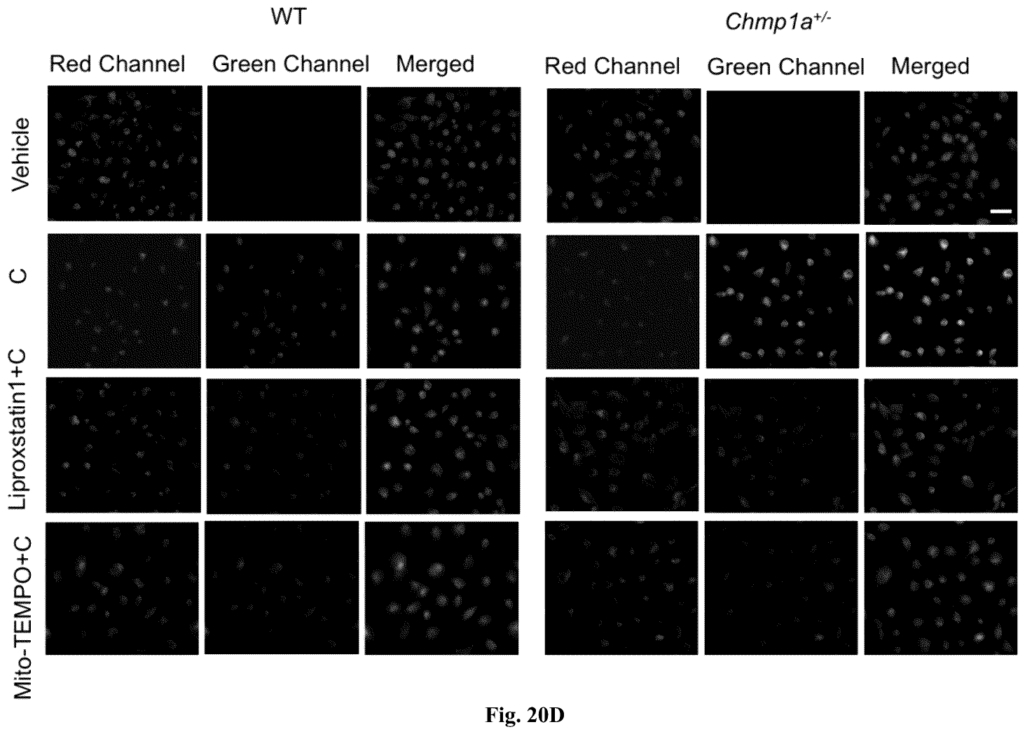

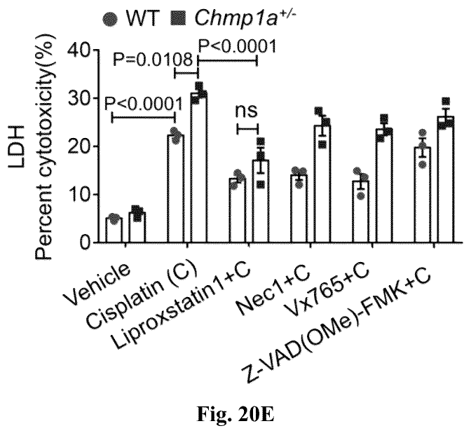

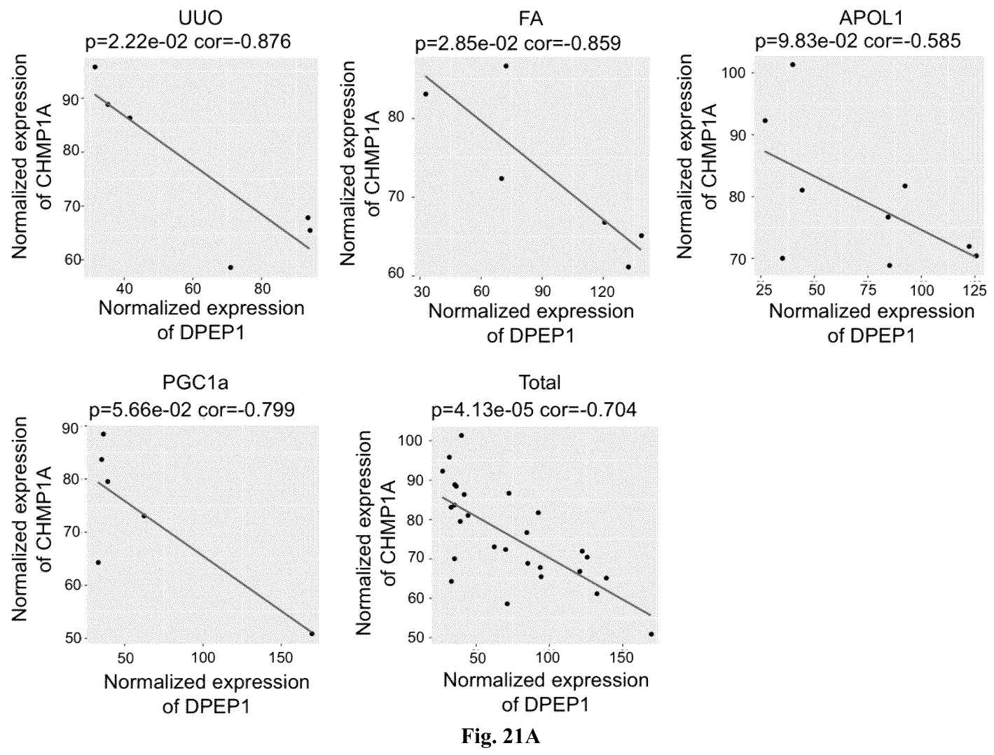

Next, the potential role of Chmp1a in ferroptosis was examined. Chmp1a knockdown (siChmp1a) cells, compared to siControl after cisplatin treatment, showed higher lipid peroxidation (oxidized BODIPY 581/591 C11 fluorescence) (FIG. 7A). The transcript level of Acsl4 was higher in Chmp1a^{+/-} kidneys and siChmp1a cell after cisplatin or FA administration (FIGS. 7B-7D, 20A). Moreover, Chmp1a knockdown cells showed increased mitochondrial damage following injury by mitoSOX fluorescence (FIG. 20B). Administration of Mito-TEMPO, an inhibitor of mitochondrial ROS production, markedly lowered LDH release (FIG. 20C) and lipid peroxidation (FIG. 20D) induced by cisplatin in Chmp1a^{+/-} primary TECs. To further assess the potential role of Chmp1a in ferroptosis, siControl, and siChmp1a cells were pretreated with the ferroptosis inhibitor liproxstatin1 prior to cisplatin treatment. While cisplatin-induced LDH release was increased upon Chmp1a knockdown, liproxstatin reduced this effect (FIG. 7E), indicating that the cisplatin cytotoxicity effect on tubule cell is mediated by Chmp1a. Primary TECs obtained from wild-type or Chmp1a^{+/-} mice with liproxstatin1 treatment also showed lower lipid peroxidation (C11 BODIPY 581/591) as siRNA treated cells (FIG. 20D). Treatment of primary TEC with inhibitors of ferroptosis, necroptosis, apoptosis, and pyroptosis indicated that only the ferroptosis inhibitor liproxstatin1 alleviated the cisplatin-induced cytotoxicity in Chmp1a^{+/-} primary TECs (FIG. FIG. 20E).

Next, it was sought to understand the mechanism of Chmp1a-mediated ferroptosis protection. Chmp1a has been reported to be involved in intraluminal vesicle formation in multivesicular bodies (MVBs). Chmp1a null cells have been shown to shed fewer CD63 positive exosomes. Formation of ferritin-containing MVBs/exosomes controls iron efflux and ferroptosis, therefore, impairment of exosome formation in the Chmp1a^{+/-} kidneys might explain iron accumulation and increased sensitivity to ferroptosis. The exosome formation was examined by CD63 in Chmp1a^{+/-} kidneys (FIG. 7F). Consistent with the potential role of Chmp1a in exosome formation, iron concentration in cisplatin-injected Chmp1a^{+/-} kidney lysates was higher than cisplatin-injected control kidneys (FIG. 7G).

To confirm the role of ferroptosis in vivo in cisplatin-induced Chmp1a-mediated cell death, wild-type and Chmp1a^{+/-} mice were injected with liproxstatin and sham. Liproxstatin1 protected Chmp1a^{+/-} mice from cisplatin-induced injury and reduced serum creatinine level (FIG. 7H). Kidney injury marker Kim1 was lower in liproxstatin injected Chmp1a^{+/-} mice (FIG. 7I). In summary, these data suggested that Chmp1a plays a role in exosome formation and iron export.

### Example 1-9: DPEP1 and CHMP1A Levels Strongly Correlated with Each Other and Other Ferroptosis Genes in Human Kidney Samples

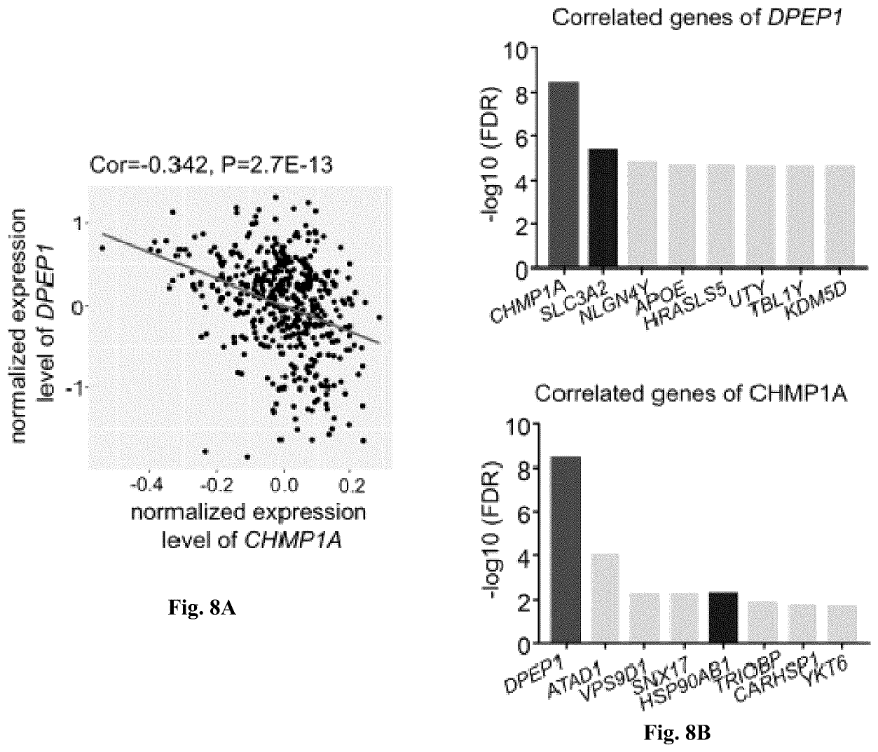

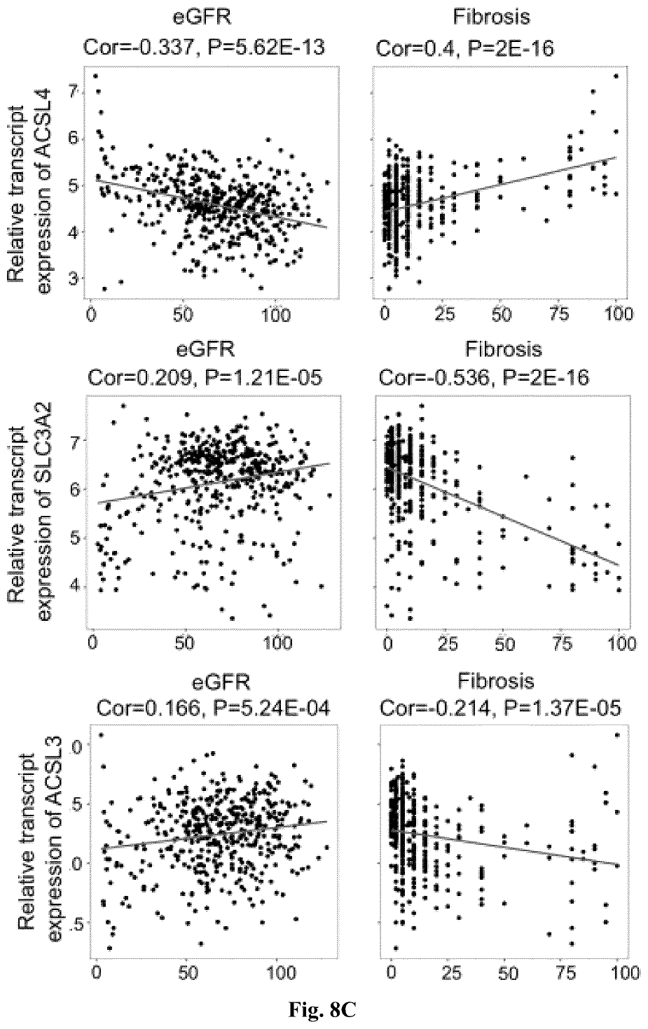

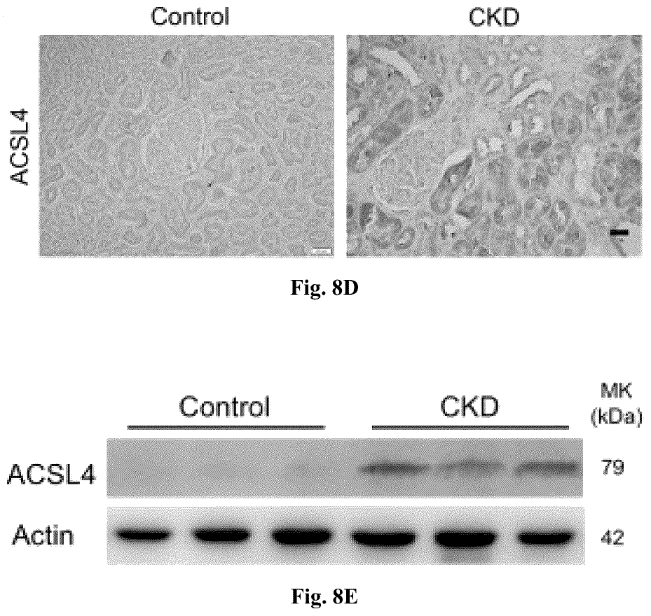

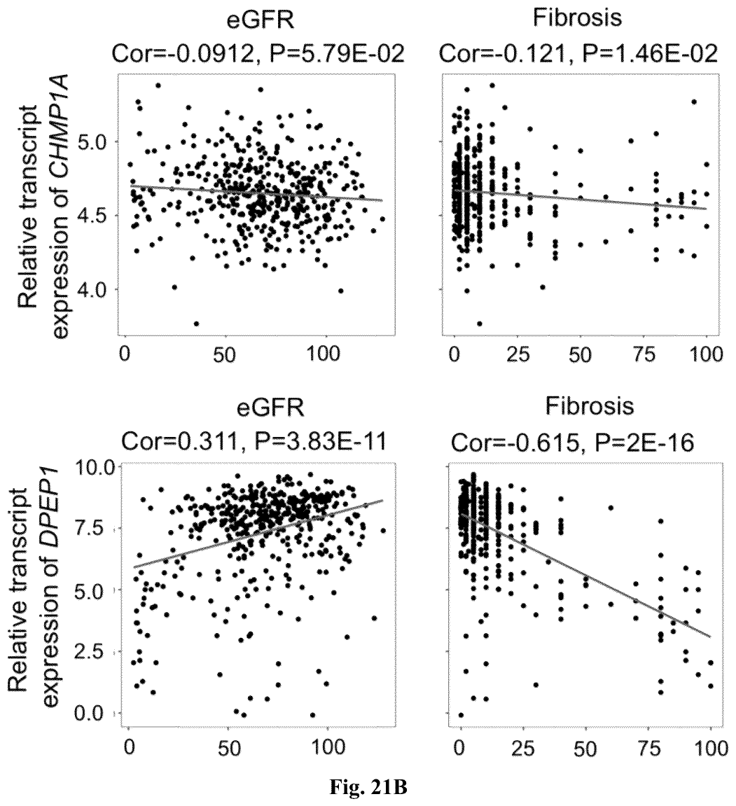

Finally, it was sought to understand if it was possible to recapitulate changes observed in Dpep1 and Chmp1a mice in human kidney samples. First, RNA sequencing data from 432 microdissected human kidney samples was examined. The collection included subjects with normal kidney function and absence of fibrosis and also samples with diabetic or hypertensive kidney disease. The clinical information is shown in supplementary data on www.ncbi.nlm.nih.gov/pmc/articles/PMC8382756. Those genes whose expression were strongly correlated with DPEP1 were first identified. In keeping with the results, indicating a common regulatory region for both genes, it was found that the top correlating gene for DPEP1 was CHMP1A (P=2.7E-13) (FIG. 8A). Dpep1 and Chmp1a levels also strongly and negatively correlated in mouse models of kidney disease induced by UUO, FA, APOL1, and PGC1a (FIG. 21A). The correlations of DPEP1/CHMP1A expression with eGFR and fibrosis of bulk human kidney tissue were less consistent, albeit cell fraction changes could have influenced this (FIG. 21B).

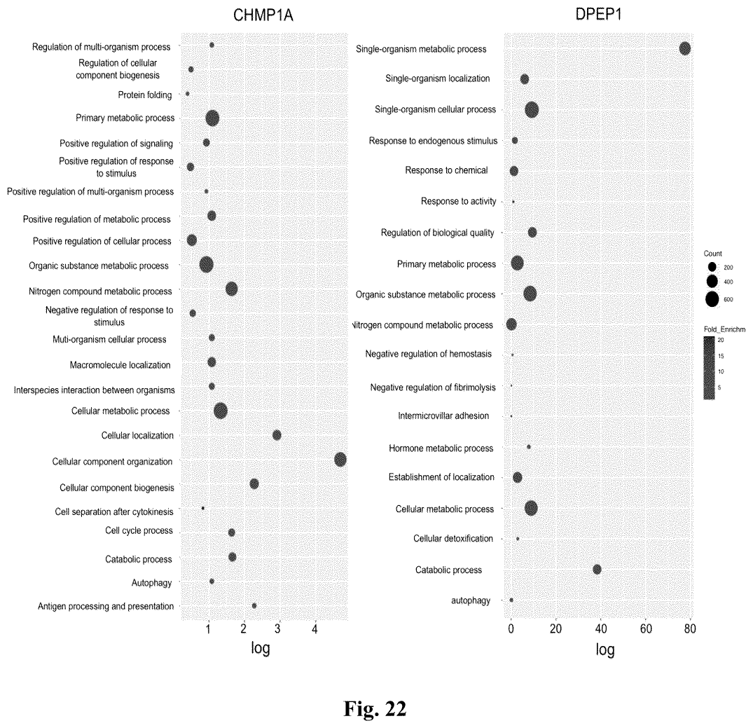

Next was analyzed the list of genes that showed strong correlation with DPEP1 or CHMP1A expression (FIG. 8B). One of the top genes that correlated with DPEP1 level was SLC3A2 (FIG. 8B), a neutral amino acid transporter important for cysteine transport with a key role in ferroptosis. Amongst the top genes that correlated with CHMP1A level was HSP90 (FIG. 8B), a chaperone that was shown to regulate ferroptosis. Gene ontology analysis for genes that correlated with either CHMP1A or DPEP1, respectively, showed enrichment for metabolic processes (FIG. 22).

It was found that the expression of ferroptosis activator ACSL4 showed a strong negative correlation with kidney function (eGFR) and positive correlation with fibrosis, and the expression of ACSL3 and SLC3A2 showed a positive correlation with eGFR and negative correlation with fibrosis (FIG. 8C), supporting an important role for ferroptosis in patients with CKD and fibrosis. An increase in protein expression of ACSL4 was also found in human kidney samples with CKD (FIG. 8D,8E). In summary, these results indicate a strong correlation between the expression of DPEP1 and CHMP1A in human kidney samples, supporting their shared regulation. In addition, their levels correlated with several key ferroptosis regulators.

### Example 1-10

Here DPEP1 and CHMP1A were identified as kidney disease genes via the triangulation of genome-wide association studies, human kidney mQTL and eQTL data. The locus was fine mapped via single-cell chromatin accessibility annotation, conditional analysis of the GWAS and CRISPR/Cas9 mediated genome deletion studies to identify common regulatory regions for DPEP1 and CHMP1A in proximal tubules. Analysis of Dpep1 and Chmp1a haploinsufficient mice demonstrated the functional role of both Dpep1 and Chmp1a in kidney injury. On the molecular level, it was that both genes are crucial regulators of ferroptosis. While Dpep1 altered iron import, Chmp1a interfered with iron export, indicating an important mechanistic convergence and likely explaining the linked regulation of these genes.

Defining the key genes and regulatory mechanism for kidney disease is a critically important next step for therapeutic development. Kidney function heritability is estimated to be around 50%. There are more than 250 loci showing genome-wide significant association with kidney function. As of now, functional annotation of target genes has only been performed for a handful of loci, such as UMOD, DAB2, SHROOM3, DACH1, and MANBA. These studies indicated the critical role of the endolysosomal system in proximal tubule cells in disease development.

While the GTEx compendium highlighted that a single variant might show association with the expression of multiple genes, this study is one of the first studies to demonstrate that multiple genes actually contribute to disease development at a single fine-mapped genetic locus. While the GWAS, methylation, and expression of DPEP1 and CHMP1A share common causal variants using Bayesian molocalization analysis, transcriptome-wide association analysis and Mendelian randomization further confirmed that DPEP1 and CHMP1A mediate the effect of genotype on kidney disease development. Single-cell epigenome analysis, GWAS conditional analysis and genome editing studies defined that the expression of DPEP1 and CHMP1A are controlled by a shared genomic region. Furthermore, DPEP1 and CHMP1A expressions strongly correlates in human kidney tissue samples, further supporting their shared regulation in the human kidney. This work indicates that a more comprehensive approach is needed for target variant and gene characterization.

As of now, almost all cell death pathways have been observed in mouse kidney injury models, including apoptosis, necroptosis, pyroptosis, and ferroptosis. These results indicate converging evidence on ferroptosis as a key pathway in kidney function heritability. While injured kidneys and TECs showed enhanced pyroptosis and necroptosis, minimal changes were observed in these pathways in mice with genetic deletion of Dpep1 or Chmp1a. Ferroptosis is a recently described form of regulated necrosis. It is characterized by iron-catalyzed intracellular accumulation of lipid hydroperoxides. Ferroptosis is proposed to play a role in the pathogenesis of Huntington's disease, Parkinson's disease, hemochromatosis, and acute kidney injury. The abundance of polyunsaturated fatty acids, within the kidney tubular compartment, likely contributes to the kidney's high susceptibility to ferroptosis. This study now indicates the critical causal role of ferroptosis in CKD and kidney function regulation in patients as defined by kidney genetic studies.

It was demonstrated that Dpep1 and Chmp1a are important regulators of ferroptosis with opposing directions. Quantification of lipid peroxidation is an important feature of ferroptosis. Knockdown of Dpep1 was associated with lower lipid peroxidation, whereas Chmp1a knockdown showed an increase in lipid peroxidation. ACSL4, which suppresses polyunsaturated fatty acid incorporation into phospholipid membranes, is another key player of ferroptosis. It was found that expression of Acsl4 was lower in Dpep1^{+/-} when compared to wild-type after injury, whereas it was higher in Chmp1a^{+/-} heterozygous mice. These results are consistent with previous publication showing that ACSL4 expression correlates with renal function in patients with acute kidney tubular injury. DPEP1 expression is also strongly correlated with the ferroptosis marker SLC3A2 in human microdissected kidney samples. In the kidney, both SLC3A2 SLC7A11 and SLC3A1 SLC7A9 are the transport system for cystine. SLC3A1 SLC7A9 heterodimeric complex is present in the apical surface of renal proximal tubules. This system plays a role in cysteine reabsorption.

CHMP1A is a member of the ESCRT-III family that functions in the sorting of receptor proteins via the formation of endosomal MVBs. Recently the role of the ESCRT-III in necroptosis and pyroptosis was proposed, however, no marked alterations were observed in these pathways in Chmp1a mutant mice. Treatment of Chmp1a heterozygous mice with liproxstatin offered marked protection from kidney disease, indicating the key role of ferroptosis in Chmp1a-mediated tubule injury in vivo. Dpep1 is a membrane-bound glycoprotein responsible for hydrolyzing dipeptides. Here it is shown that Dpep1 colocalized with endocytic vesicle marker clathrin and plays role in transferrin endocytosis. Loss of Dpep1 protected from cisplatin-induced ferroptosis. Dpep1 has also been reported to play roles in radiocontrast-induced kidney injury and cisplatin-induced tubule cell death. Future studies shall determine the relative contribution of apoptosis and ferroptosis to DPEP1-afforded disease protection.

In conclusion, it was shown that a single fine-mapped GWAS locus controls the expression of two target genes using computational integration of GWAS, kidney mQTL, eQTL, single nuclei ATAC sequencing, and CRISPR-based genome editing. DPEP1 and CHMP1A were identified as kidney disease genes and important regulators of ferroptosis. These studies indicate that pharmacological targeting of ferroptosis through Dpep1 or Chmp1a in kidney tubule cells could offer therapeutic benefits for patients with kidney disease.

### Example 1-11: Prioritization of CASP9 as a Kidney Disease Gene

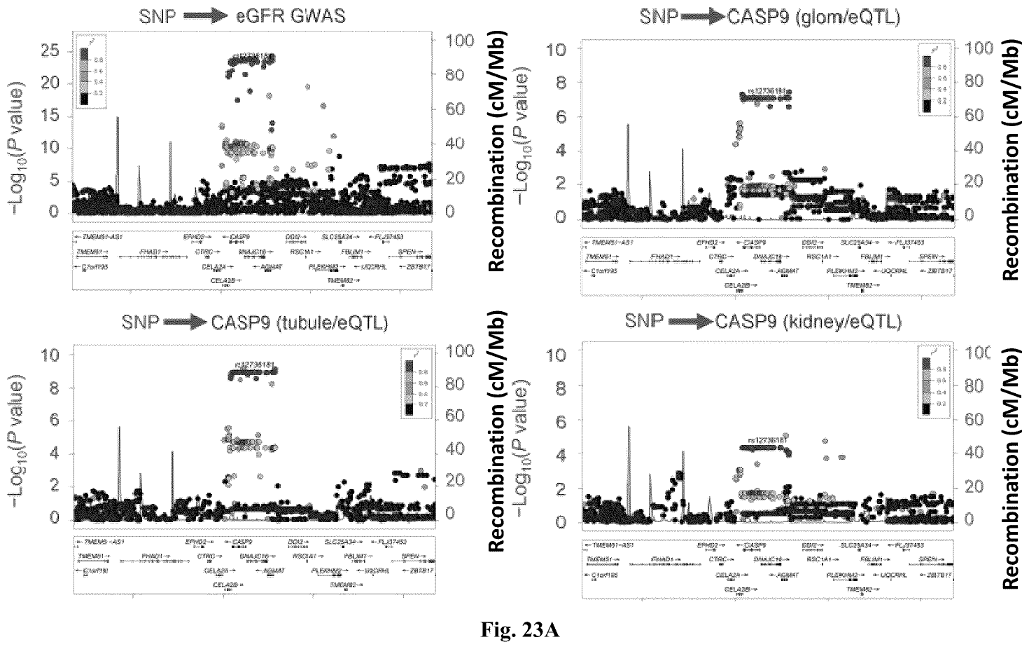

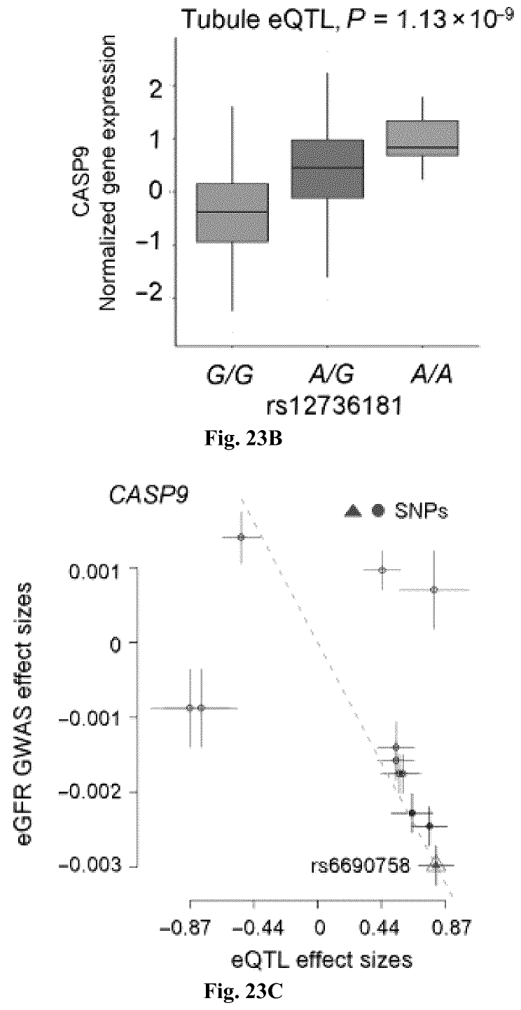

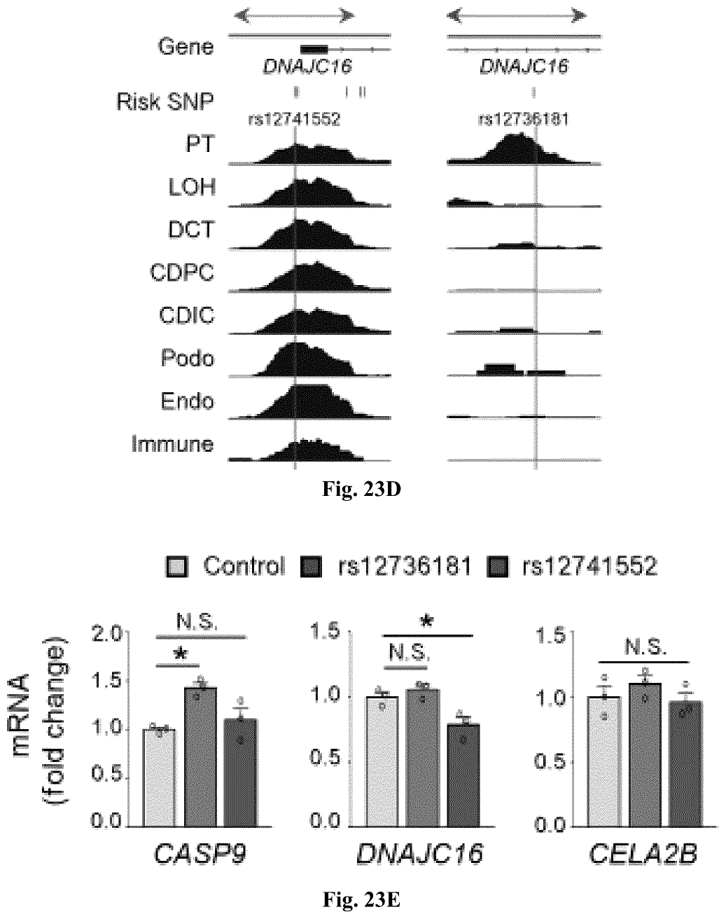

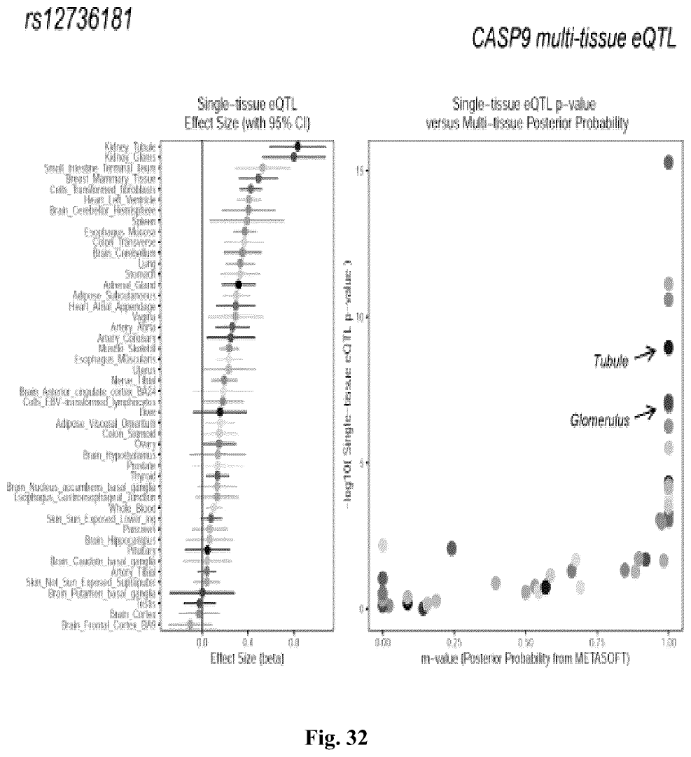

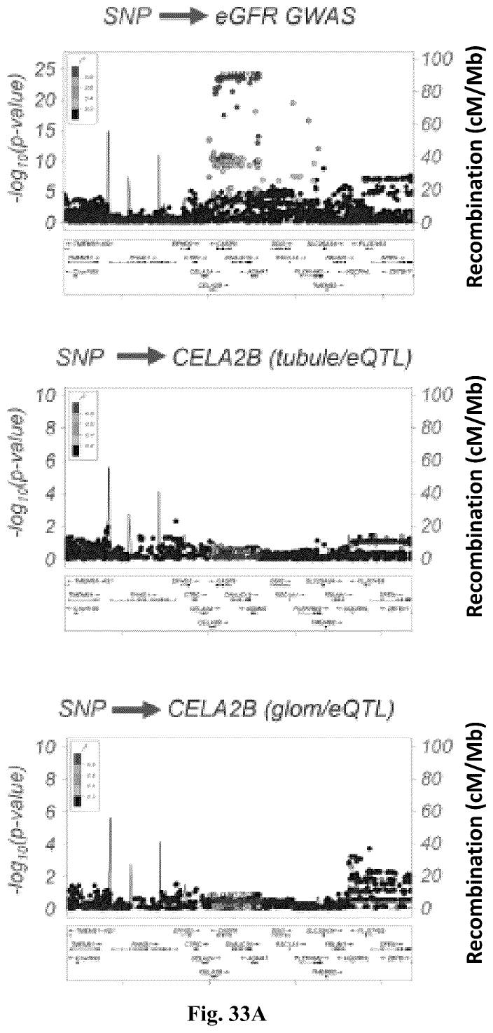

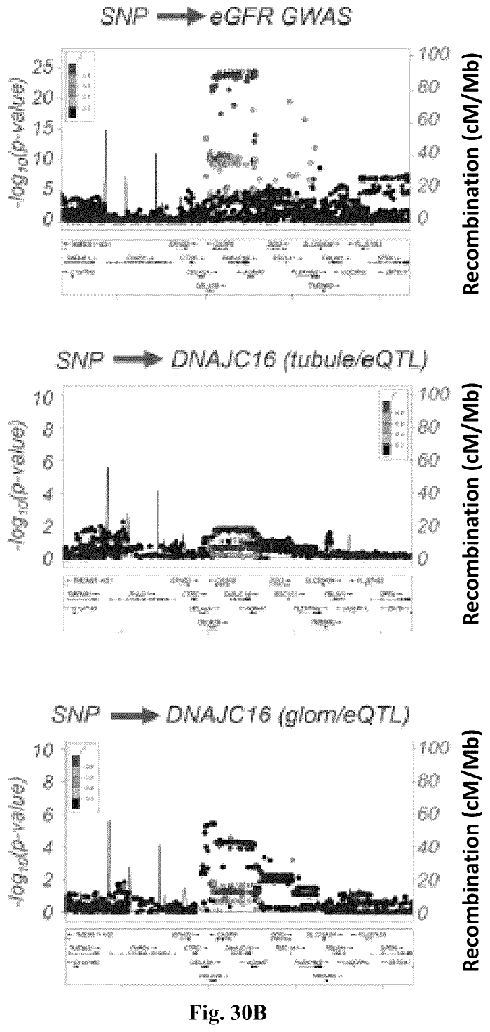

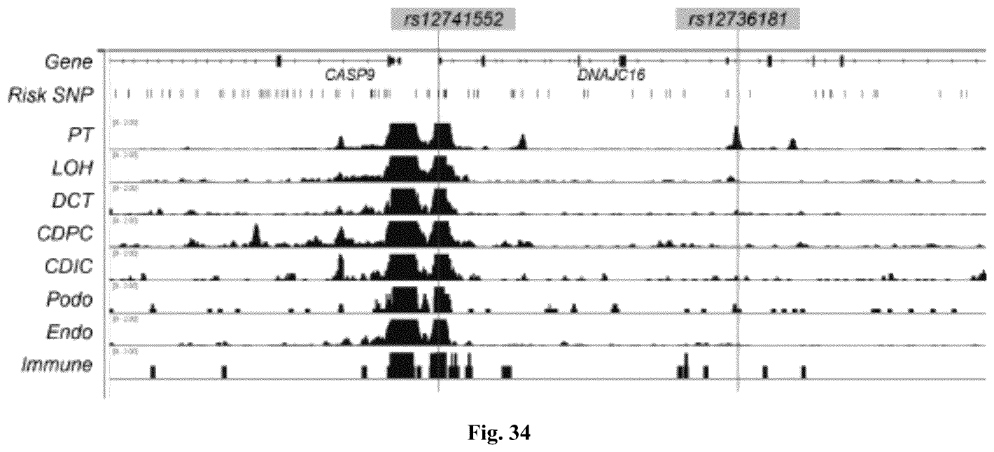

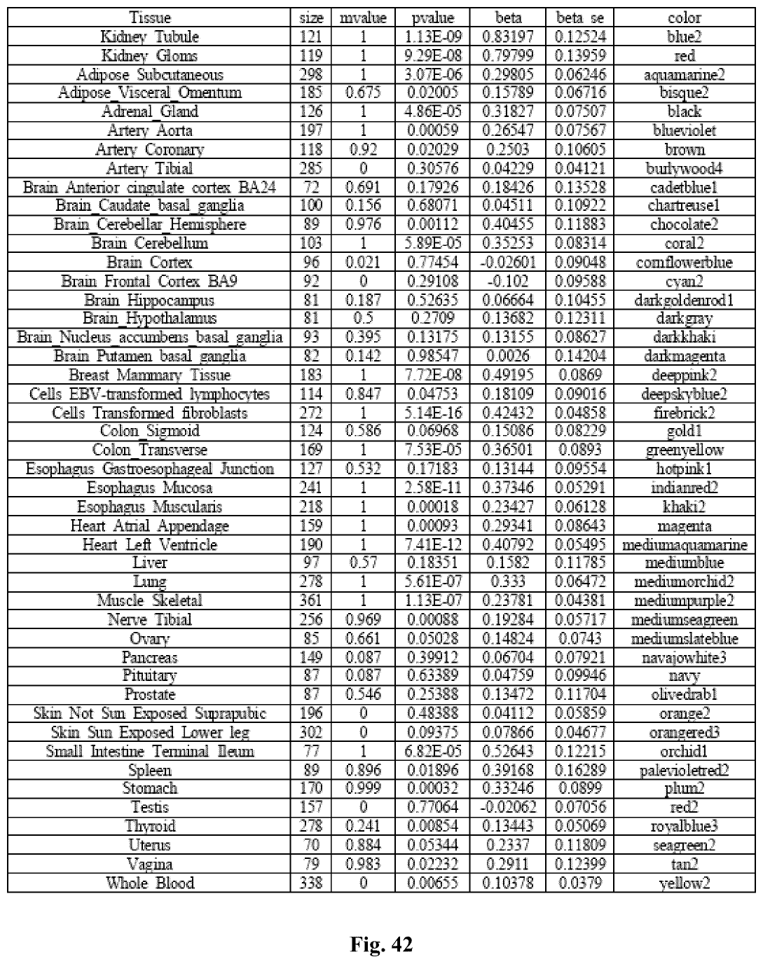

In this study, it was decided to focus on the chromosome 1 eGFR GWAS locus, which showed a strong and reproducible association with kidney function (FIGS. 23A-23C). Regional plot of eGFR GWAS indicated significant association of genetic variants on chromosome 1 with eGFR (rs12736181, P=7.0×10⁻²⁴) (FIG. 23A). The same genetic locus also showed significant association with CASP9 expression in microdissected renal tubules (P=1.1×10⁻⁹), glomeruli (P=9.3×10⁻⁸), and whole-kidney samples (P=9.0×10⁻⁶) (FIG. 23A). Multitissue eQTL analysis performed in GTEx (genotype-tissue expression) showed strong association between genetic variant (rs12736181) and CASP9 expression levels in multiple tissues including kidney tubules and glomeruli (m value, >0.90) (FIGS. 32, 42). On the contrary, no association was observed between the genetic variant (rs12736181) and expression of DNAJC16 and CLEA2B, genes located next to CASP9 (FIG. 33A-33B).

To test whether the two traits (eGFR and CASP9 expression) share causal variants at this locus, Bayesian colocalization analysis was conducted using coloc. A strong evidence that variants associated with kidney function and CASP9 expression in kidney tubules were shared (PP4=0.95 and PP3=0.05) was found. CASP9 expression in the kidney were strongly genotype dependent (rs12736181, eGFR GWAS risk allele A, P=1.13×10⁻⁹) (FIG. 23B). Transcriptome-wide association studies (TWAS) and summary-based Mendelian randomization (SMR) indicated that the effect of the genetic variants on eGFR was mediated via CASP9 expression. In the SMR analysis, eQTL effect sizes negatively correlated with eGFR GWAS effect sizes, suggesting that higher CASP9 expression was associated with lower eGFR levels (FIG. 23C).

While multiple variants showed association with eGFR and CASP9 expression, it has been shown that the SNP with the best eQTL effect size or P value is not always the causal SNP but, rather, SNPs located in the regulatory region such as enhancer are likely to be causal. To address this, human kidney single-nuclear assay for transposase-accessible chromatin (snATAC) data was used to fine-map the region and further prioritize risk variants. Variants that overlapped with open chromatin regions were prioritized. With this prioritization strategy, the likely causal SNPs (rs12736181 and rs12741552) were narrowed. Rs12736181 overlapped with open chromatin regions in proximal tubule (PT) cell, while rs12741552 was located on an open chromatin region in all analyzed cell types (FIG. 23D, 34). Both SNPs showed significant association with eGFR and CASP9 expression, and they were in high linkage disequilibrium (LD) (r²=1).

Next, experimental validation was performed. To examine whether the two prioritized variants regulate the expression of CASP9, open chromatin regions containing these SNPs were deleted and CASP9 expression (FIG. 23D) were examined. CASP9 expression was higher upon deleting open chromatin region harboring rs12736181 but unaffected when open chromatin region harboring rs12741552 was deleted. The deletion of the open chromatin region harboring rs12736181 did not affect the expression level of DNAJC16 and CELA2B. The deletion of the open chromatin region harboring rs12741552 did not affect CELA2B, while it reduced DNAJC16 level as it was located on exon1 of DNAJC16 (FIG. 23D,23E). In summary, integrative genetic analysis, single-nuclear epigenome mapping, and CRISPR-Cas9 gene editing prioritized CASP9 as a kidney disease risk gene in kidney tubule cells where increased CASP9 expression was associated with renal disease risk.

### Example 1-12: Expression of Casp9 Correlates with Fibrosis in a Variety of Mouse Kidney Disease Models

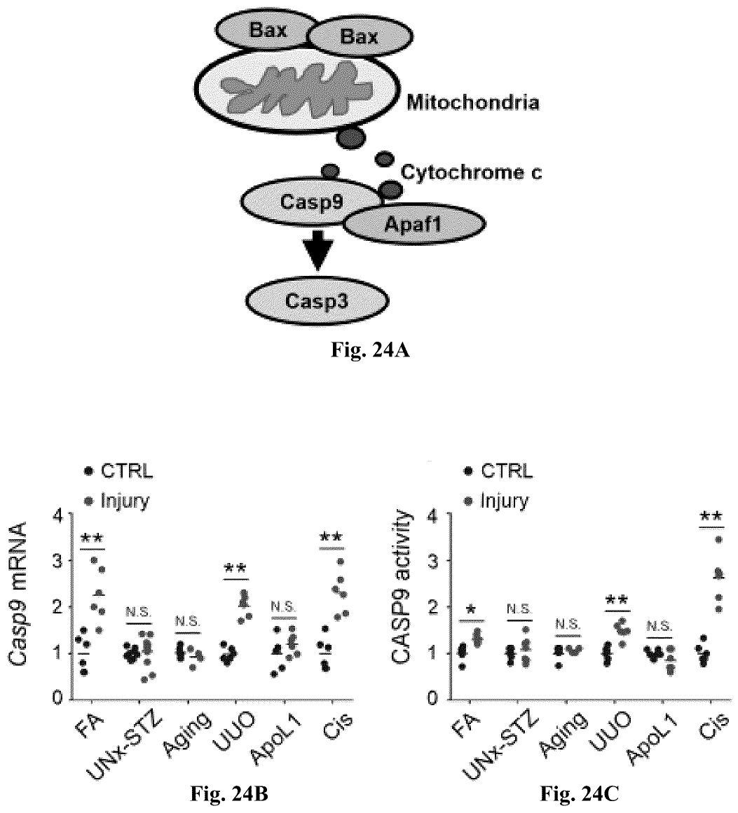

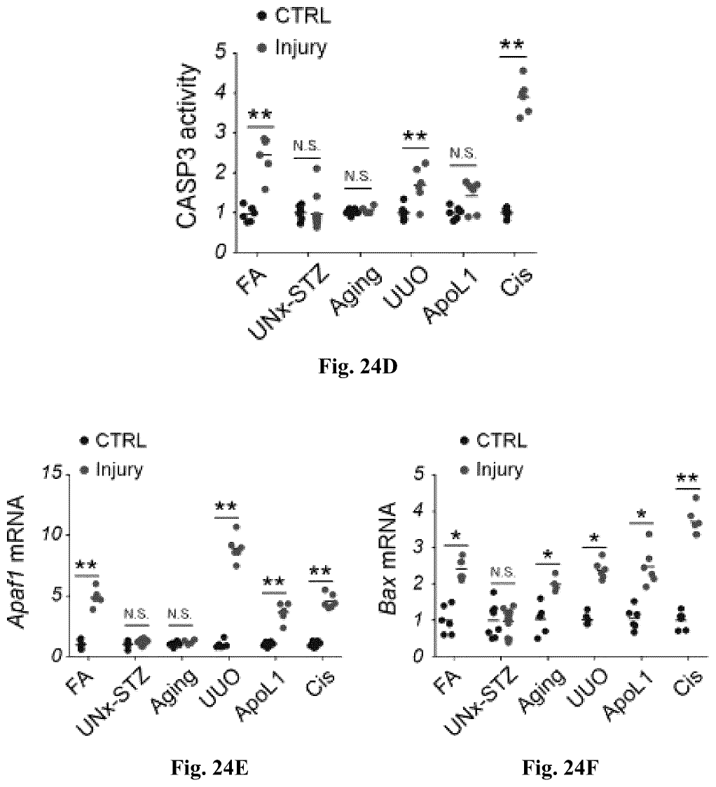

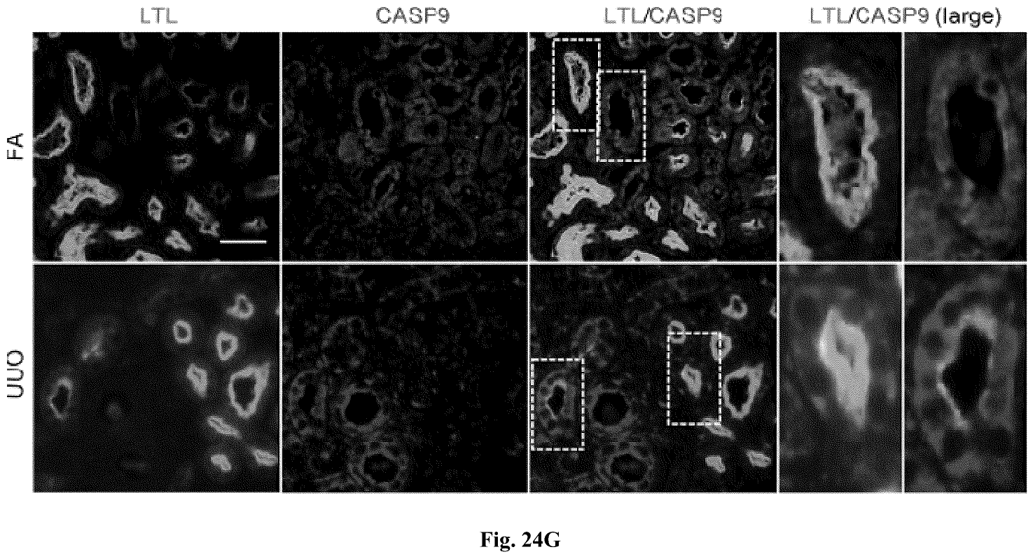

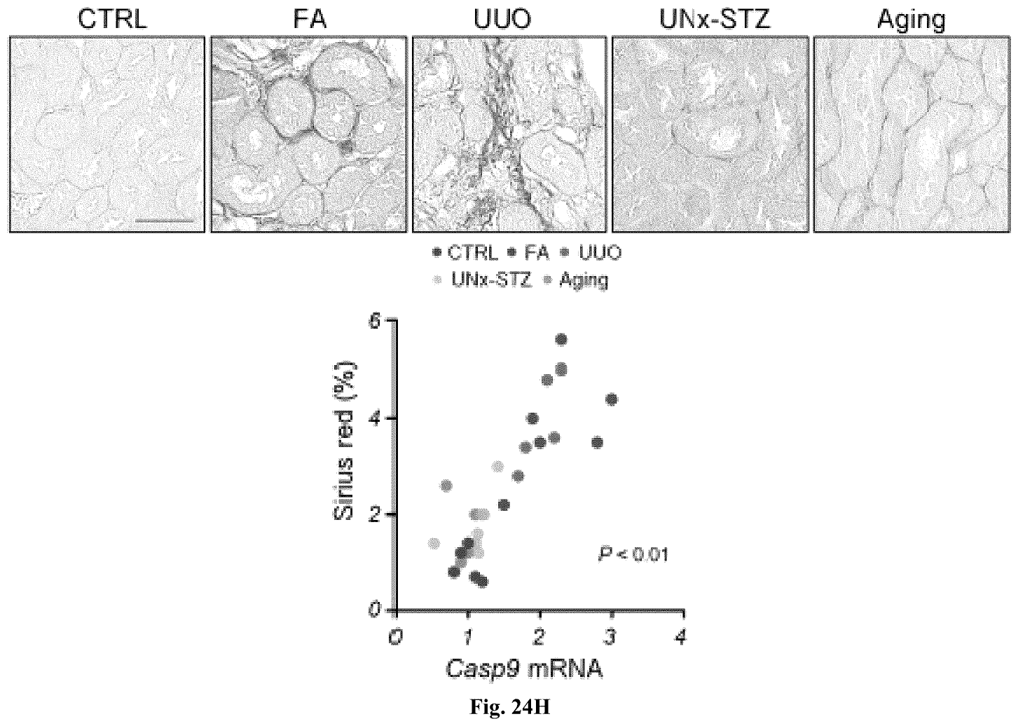

While computational integration of multiple genetic studies such as GWAS, eQTL, snATAC, and CRISPR-Cas9 gene editing is an important first step for gene prioritization, experimental validation of target genes is equally critical. CASP9 is the prototypical initiator caspase of intrinsic apoptosis triggered by mitochondrial injury (FIG. 24A). The expression of Casp9 and other apoptosis-associated genes in a variety of kidney disease models, including the folic acid (FA)-induced kidney fibrosis, the uninephrectomy and streptozotocin (STZ)-induced diabetes (UNx-STZ), renal aging, UUO, podocyte-specific expression of APOL1 risk variant-induced glomerulosclerosis (APOL1) and the cisplatin-induced toxic AKI (Cis) models were quantified. It was found that Casp9 transcript expression was higher in the FA, UUO, and Cis models (FIG. 24B). CASP9 activity showed consistent changes with transcript levels (FIG. 24C). Furthermore, CASP3 activity and expression of Apaf1 and Bax were also higher in kidneys of FA, UUO, and Cis models, indicating the activation of the entire intrinsic apoptosis cascade in these models (FIG. 24D-24F).

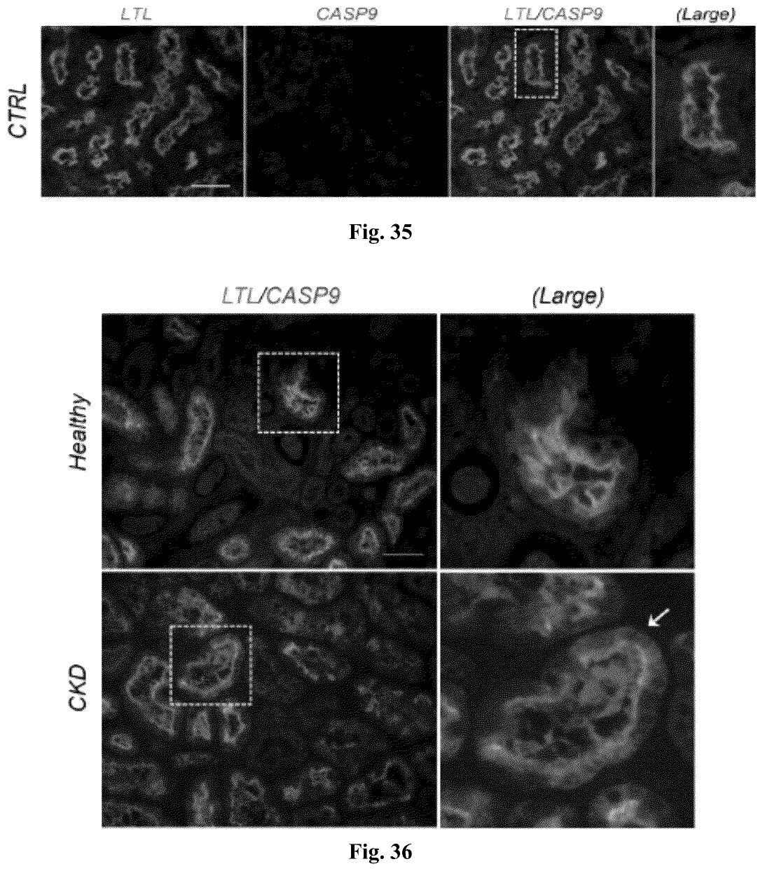

Next, the cellular localization of CASP9 in healthy and disease kidney samples was examined. Immunofluorescence staining showed higher CASP9 expression in FA and UUO fibrosis models compared to healthy control kidneys (FIGS. 24G, 35). In addition, double immunofluorescence staining using the proximal tubule marker LTL (Lotus tetragonolobus lectin) demonstrated CASP9 expression in renal proximal tubules (FIGS. 24G, 35). Tubules with higher CASP9 expression showed weaker LTL signal, indicating that they were likely injured (FIG. 24G). Consistently, CASP9 expression was observed in the proximal tubules in human chronic kidney disease (CKD) kidney samples as assessed by double immunostaining of CASP9 and proximal tubule marker LTL (FIG. 36).

To examine the correlation between Casp9 expression and the degree fibrosis, next evaluated was collagen accumulation in the FA, UUO, UNx-STZ, and aging models of kidney disease. Severe fibrosis was observed in the FA and UUO models, but it was milder in kidneys of the UNx-STZ, and aging models (FIG. 24H). Sirius red-positive area showed a strong correlation with kidney Casp9 expression (FIG. 24H).

### Example 1-13: Casp9 Heterozygous Mice were Protected from AKI

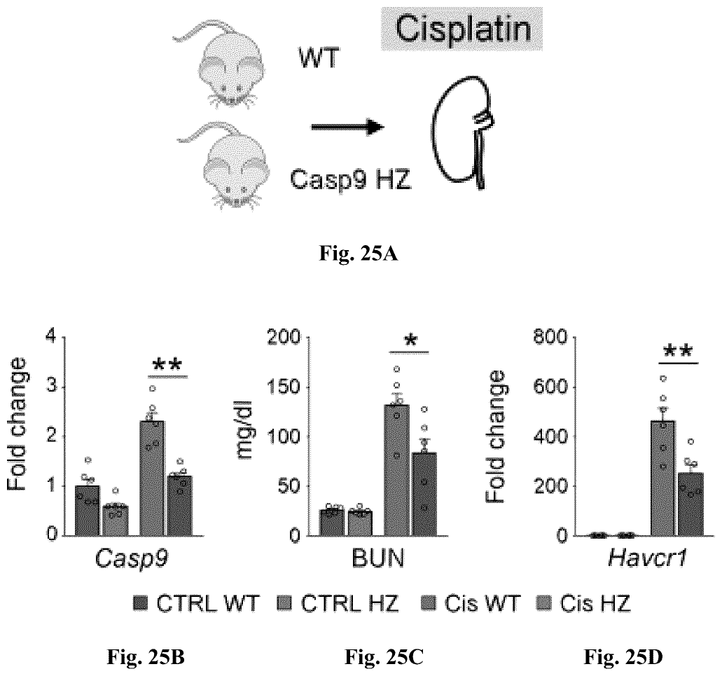

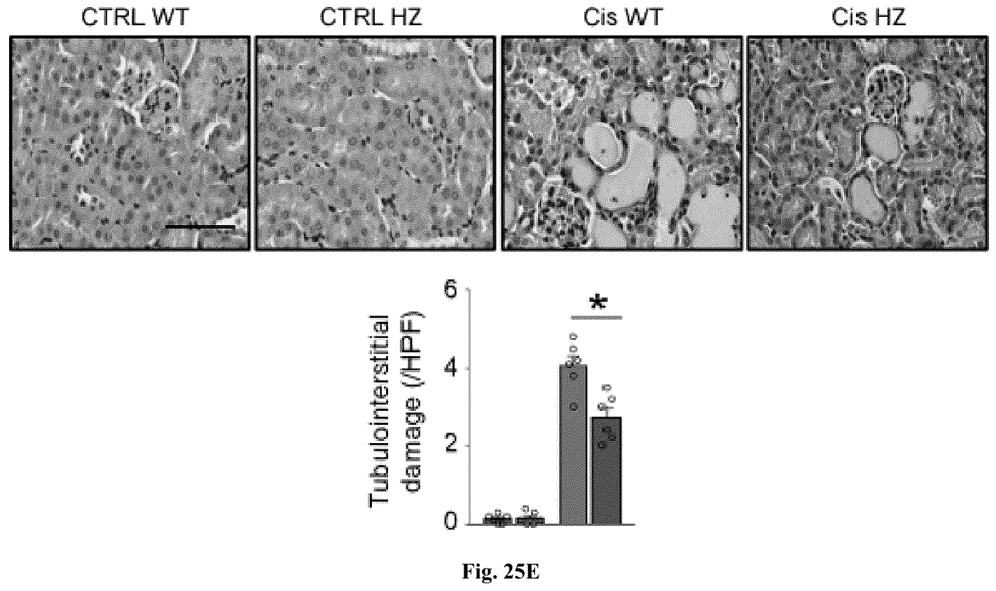

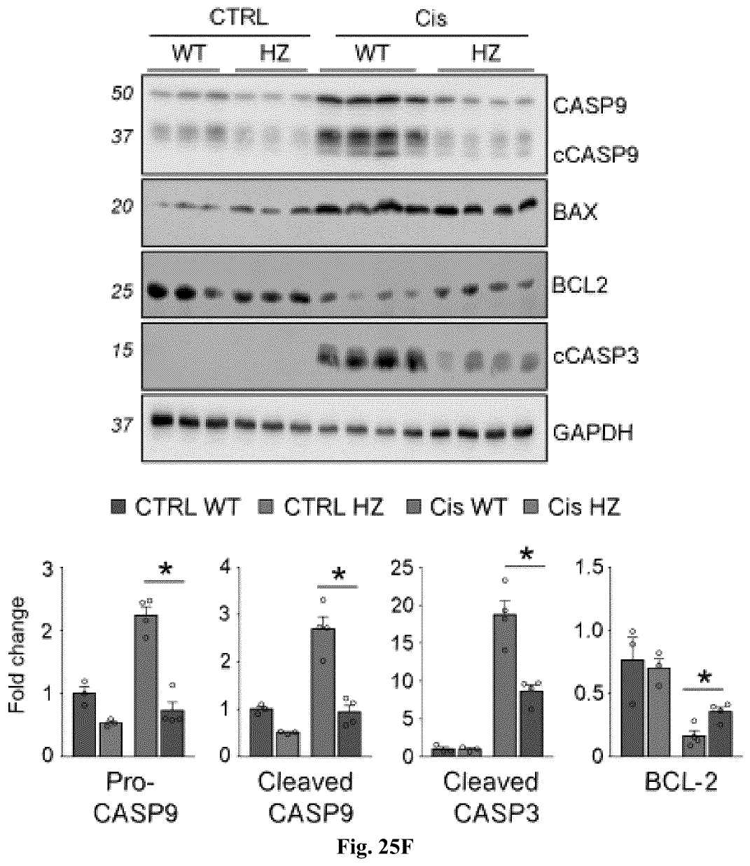

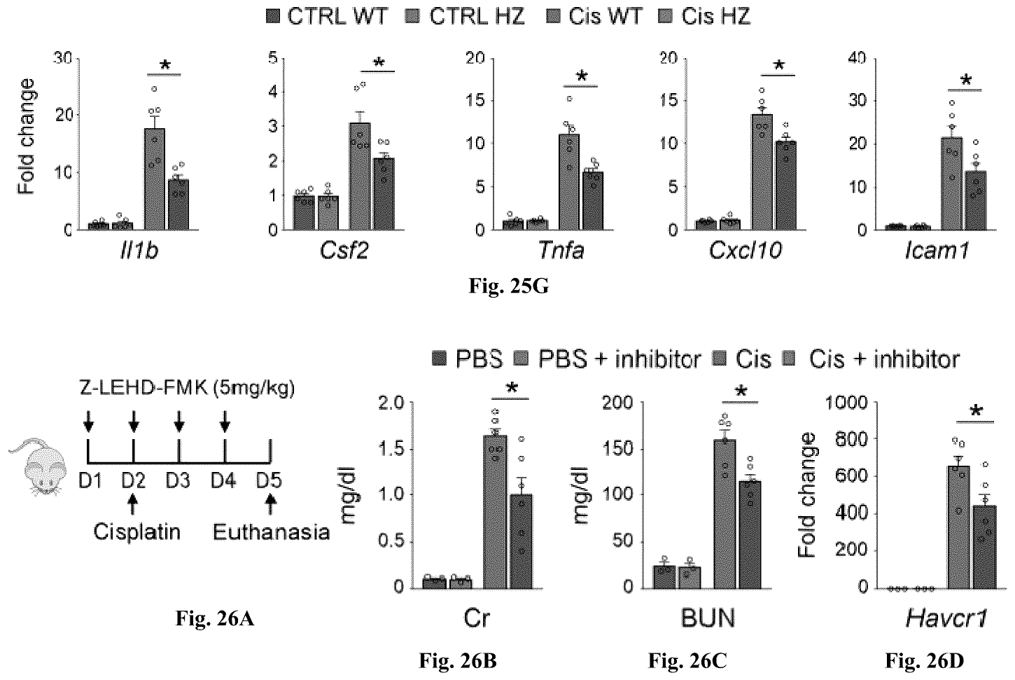

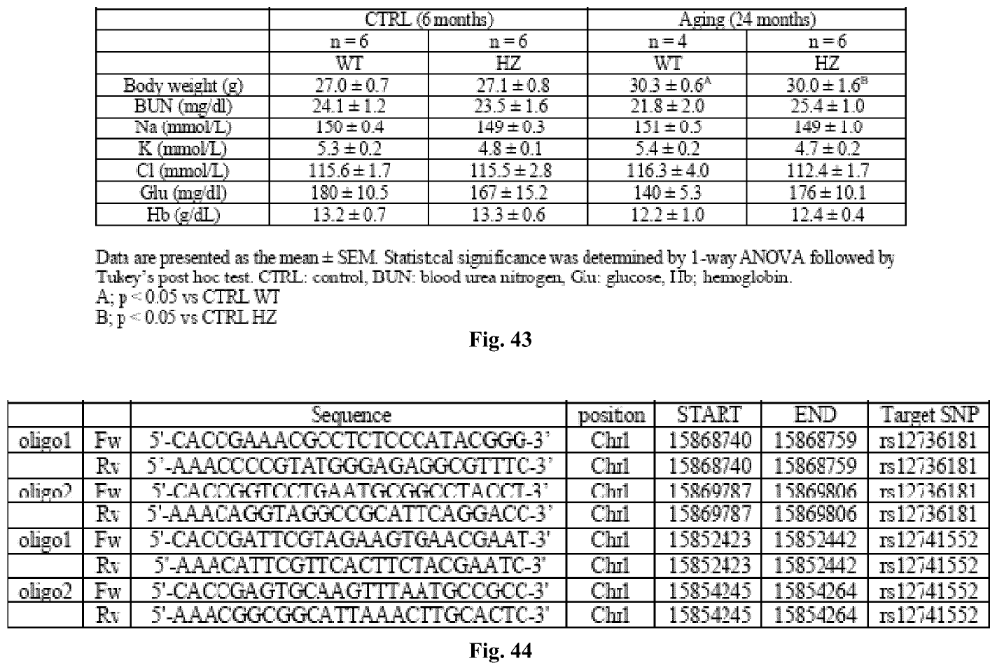

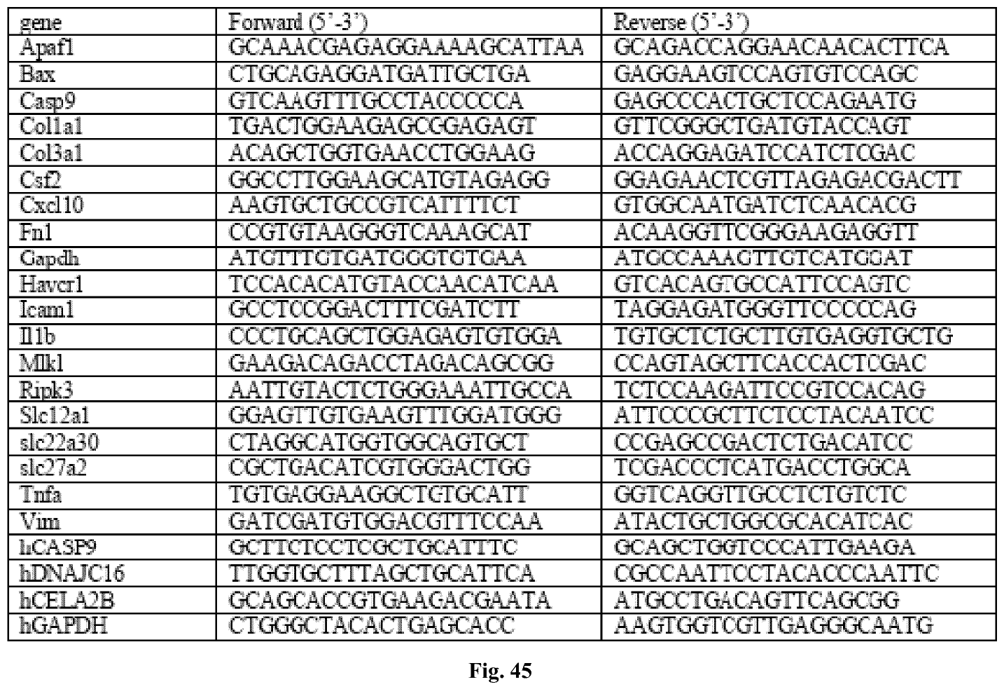

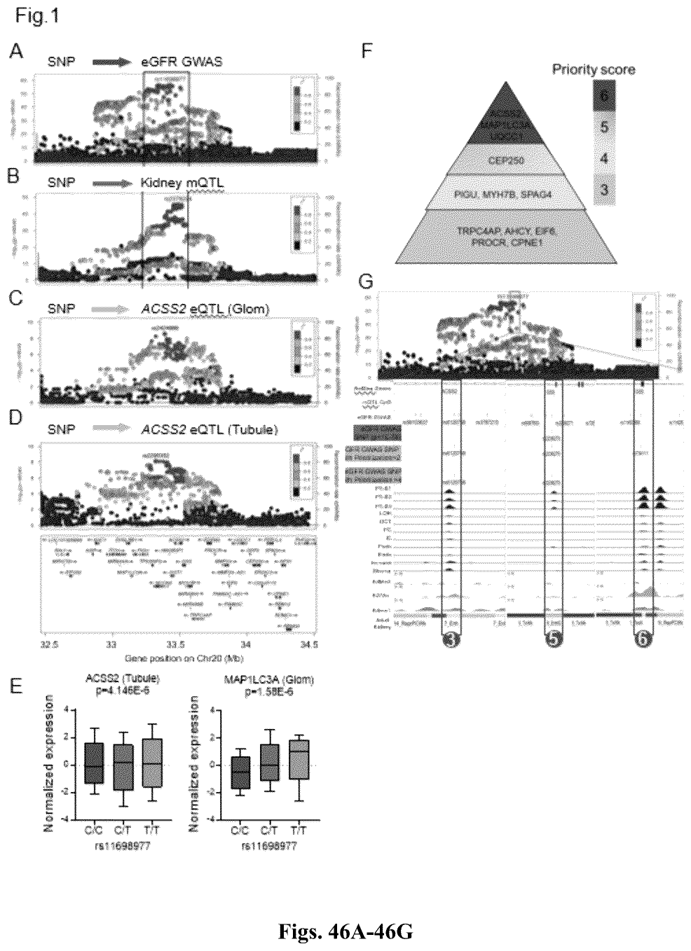

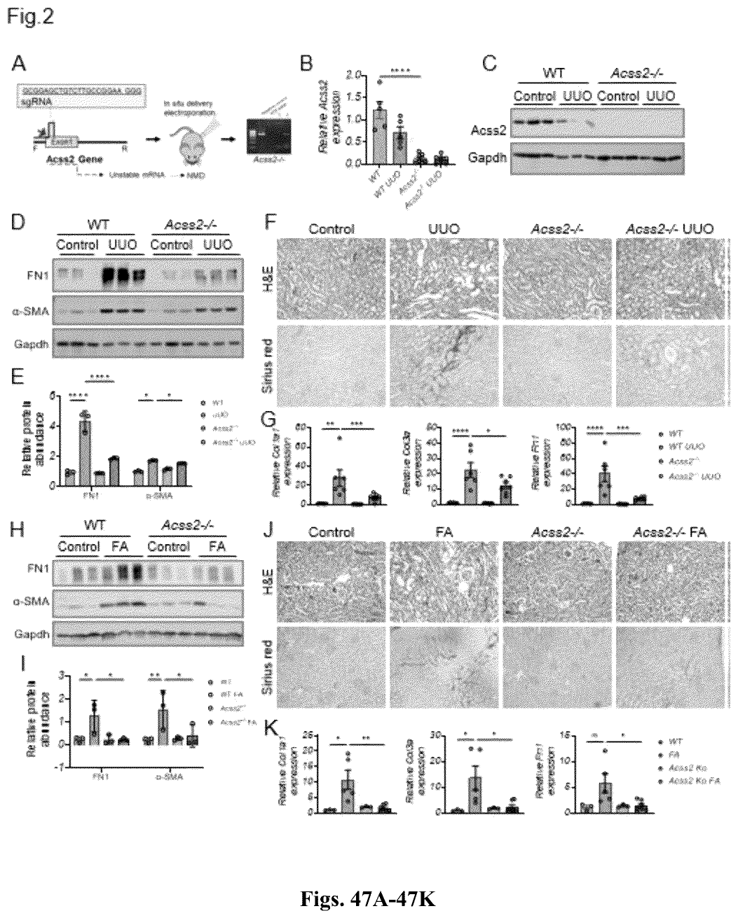

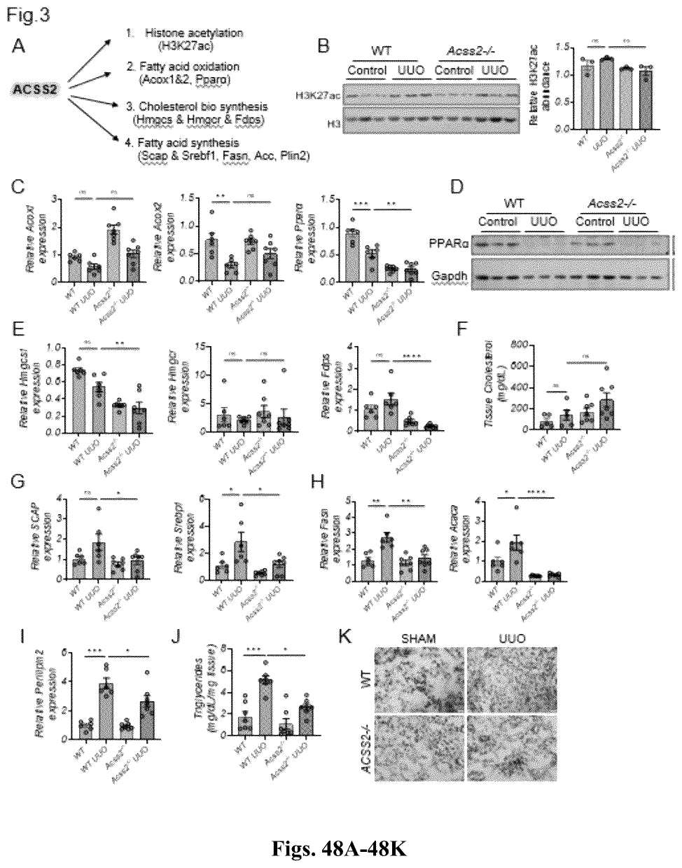

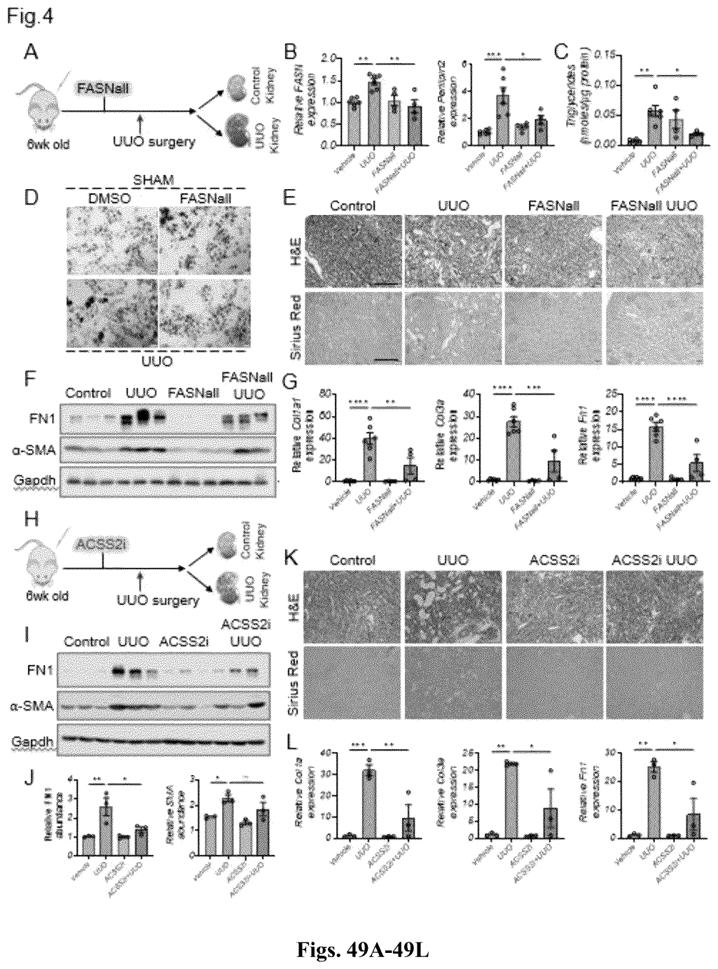

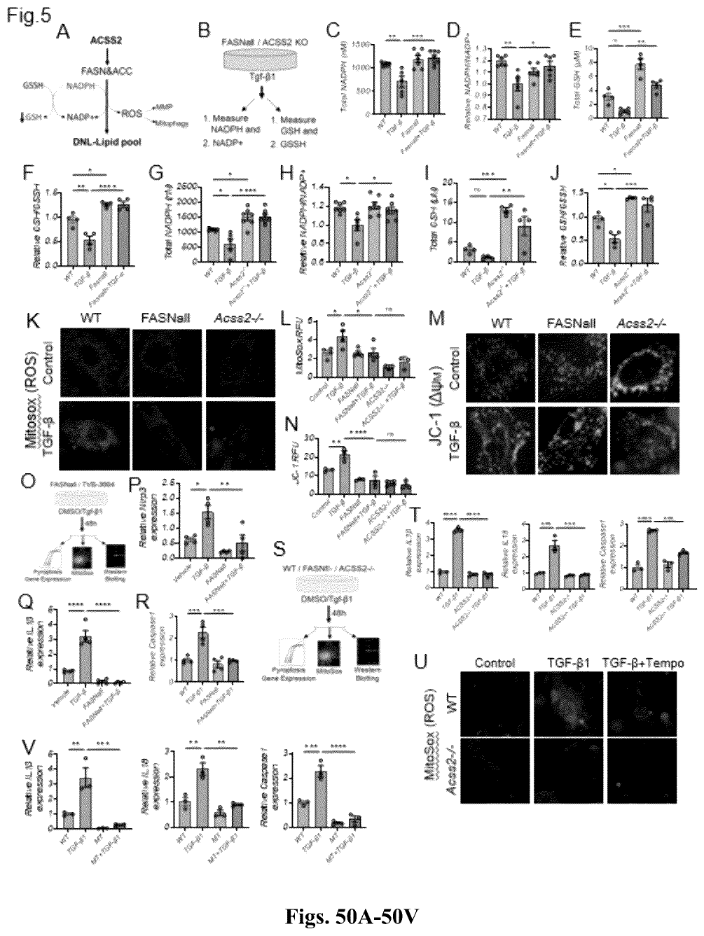

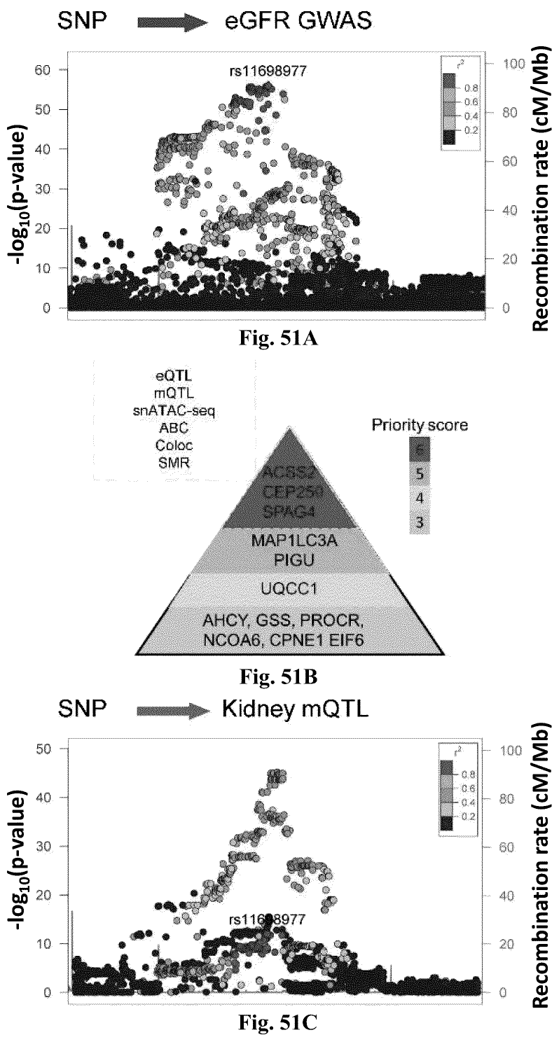

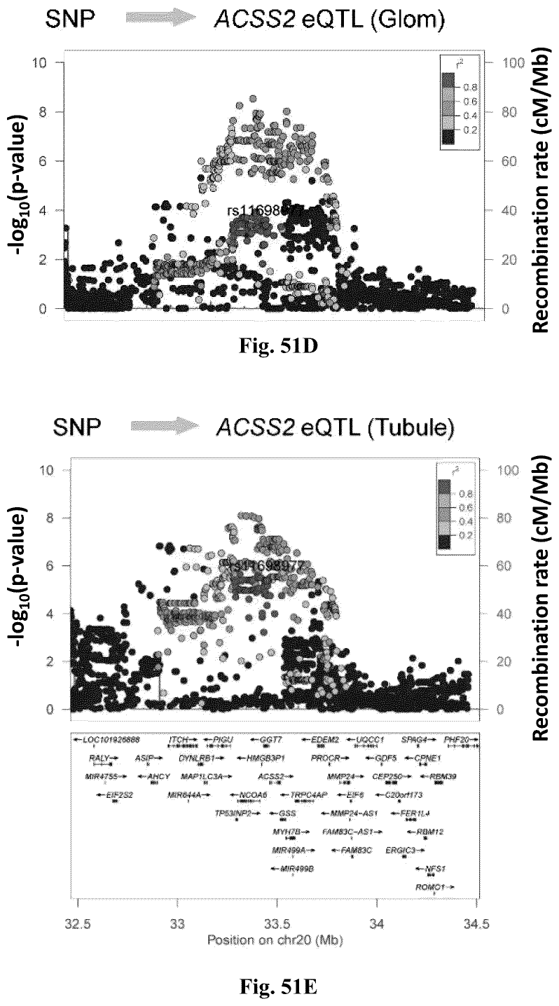

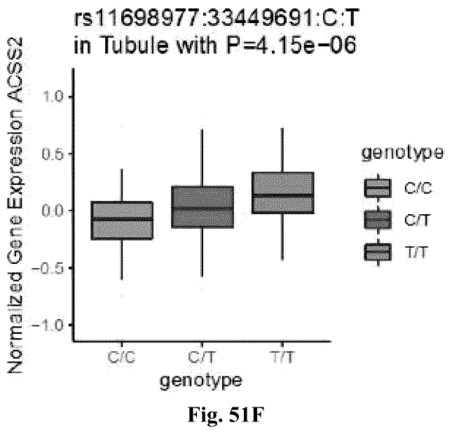

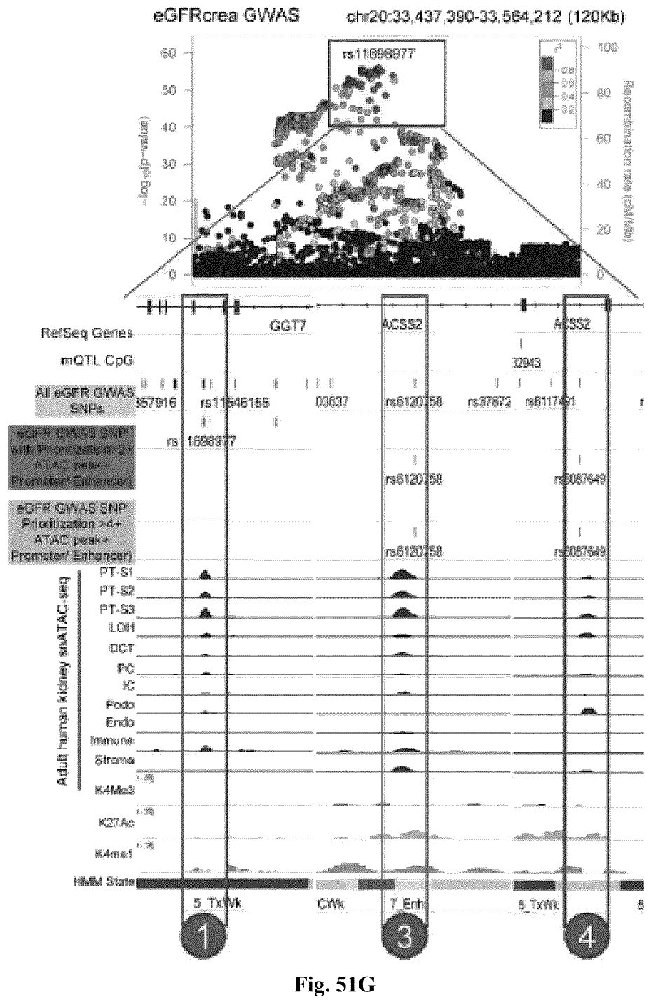

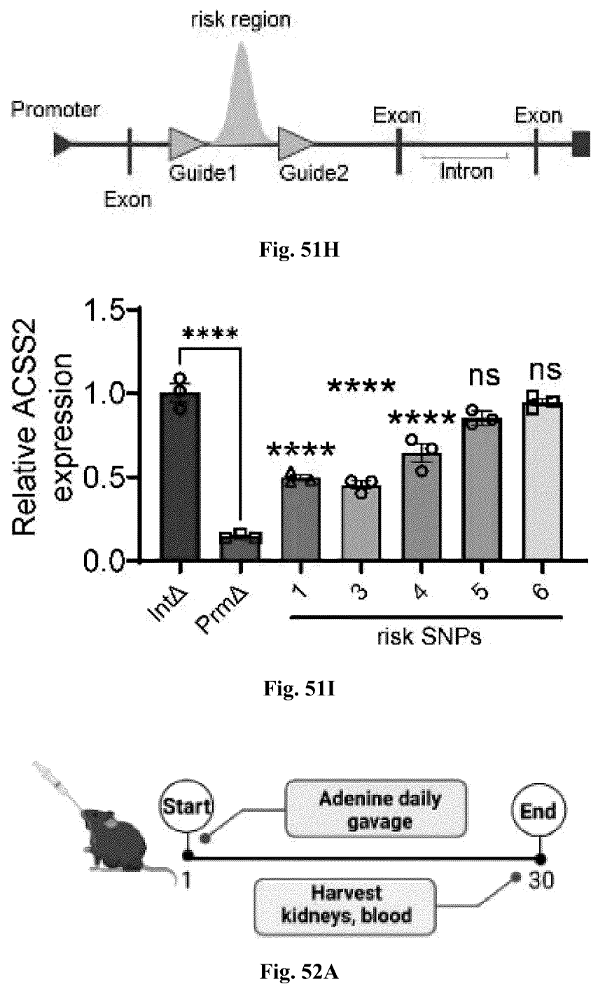

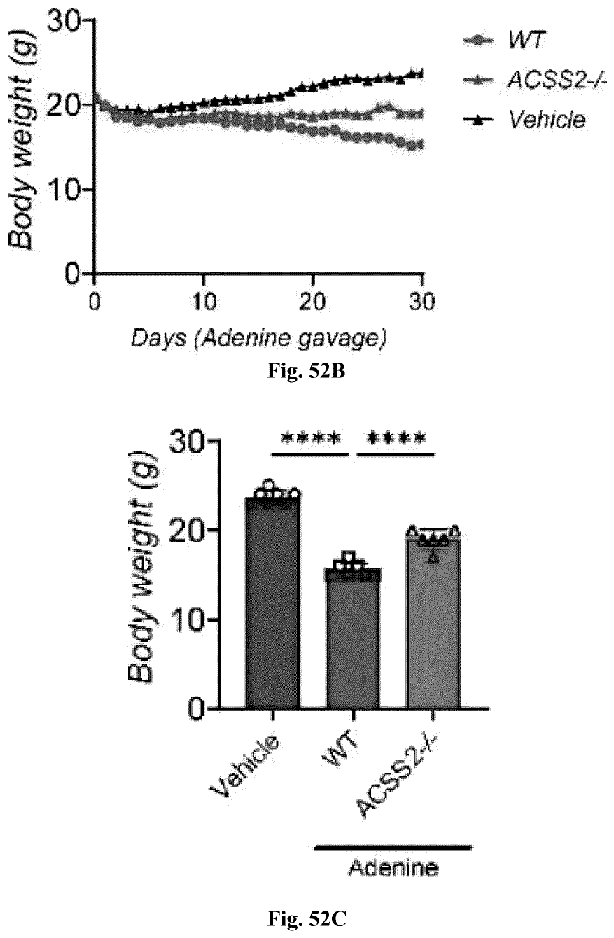

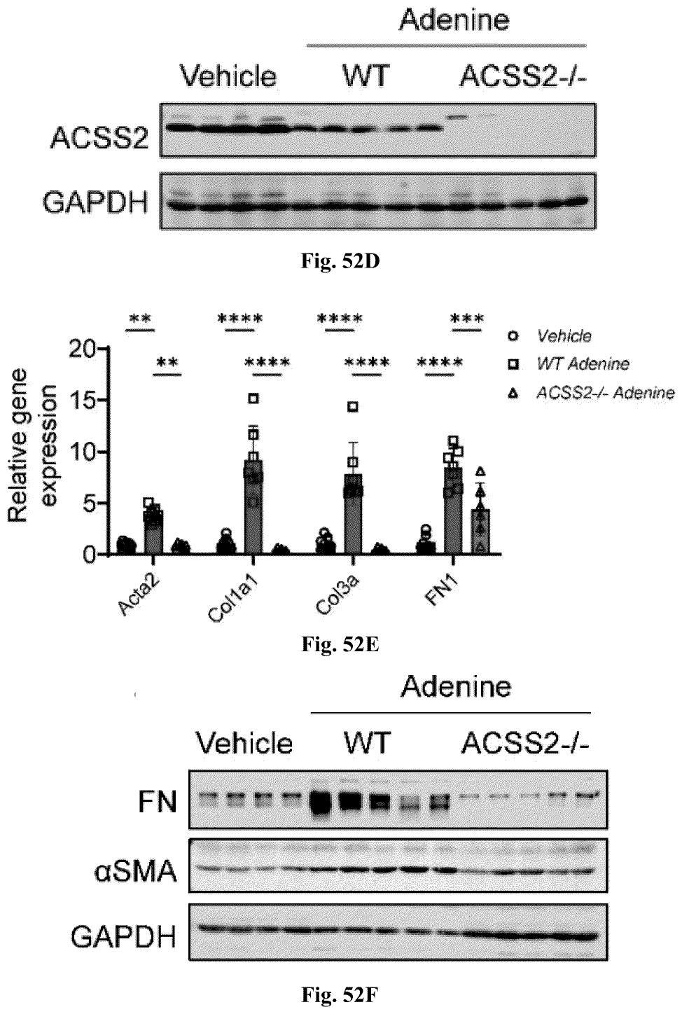

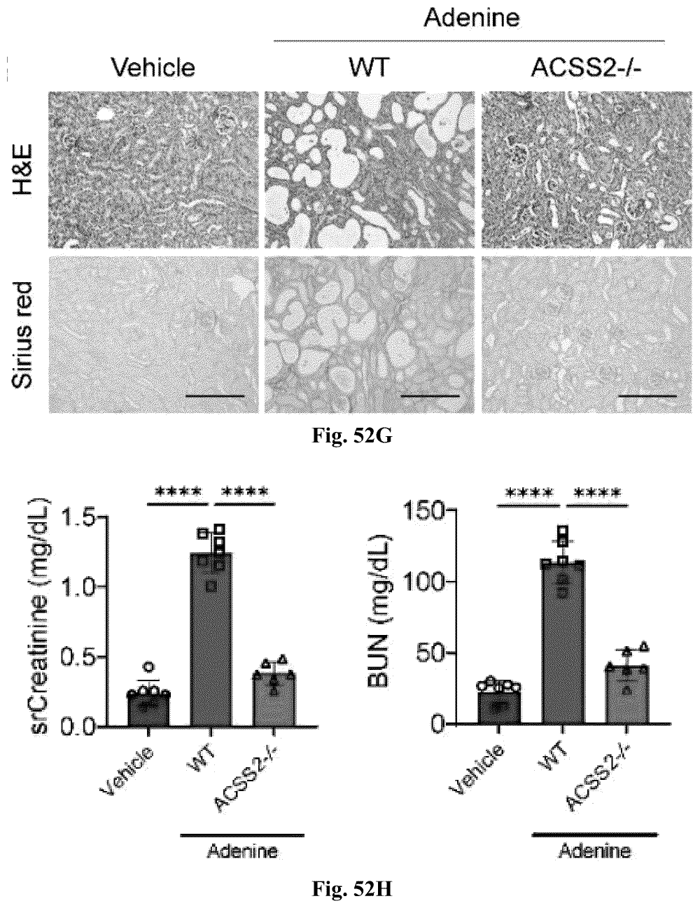

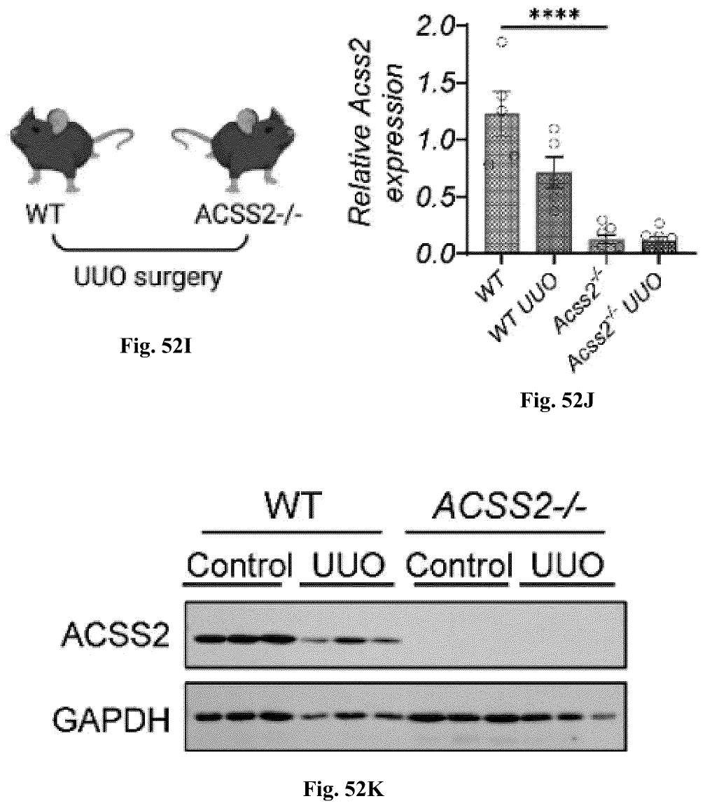

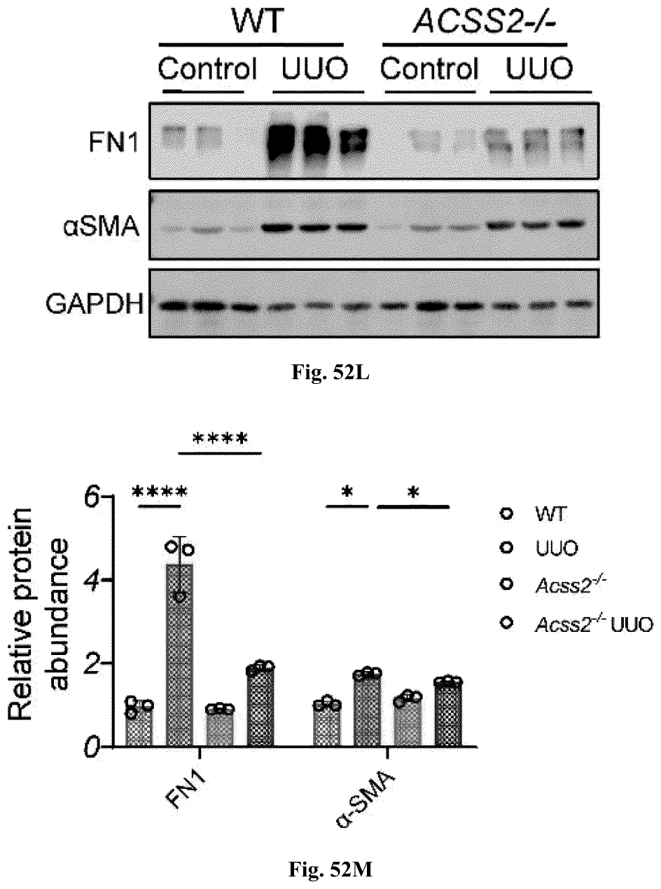

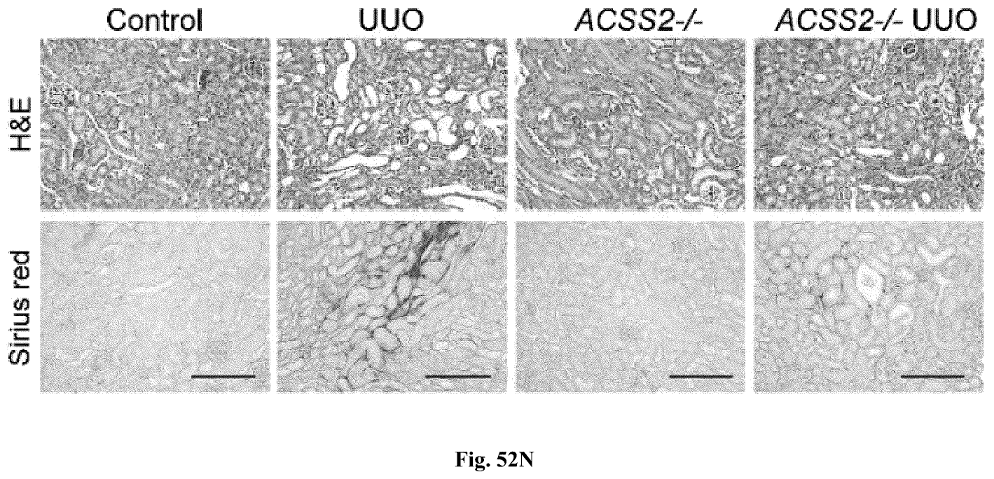

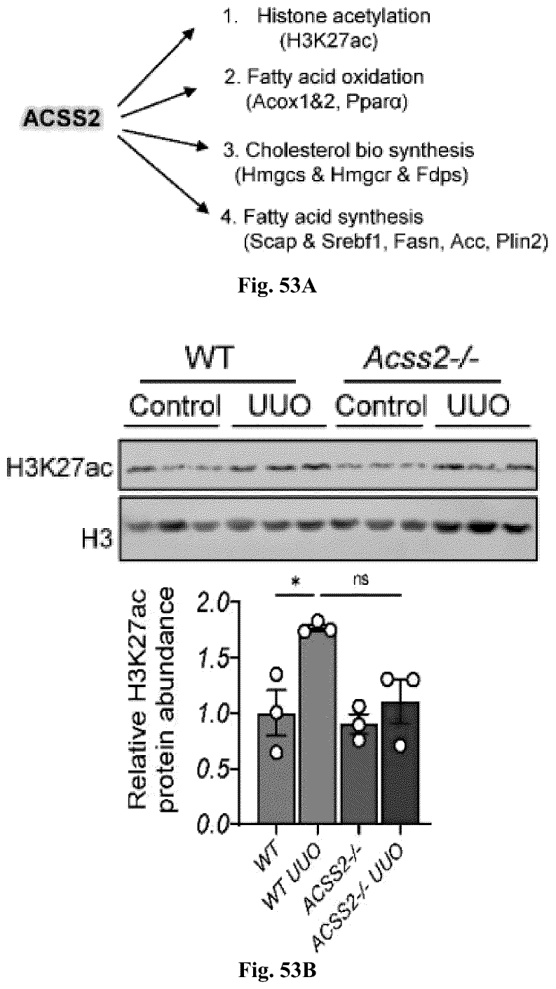

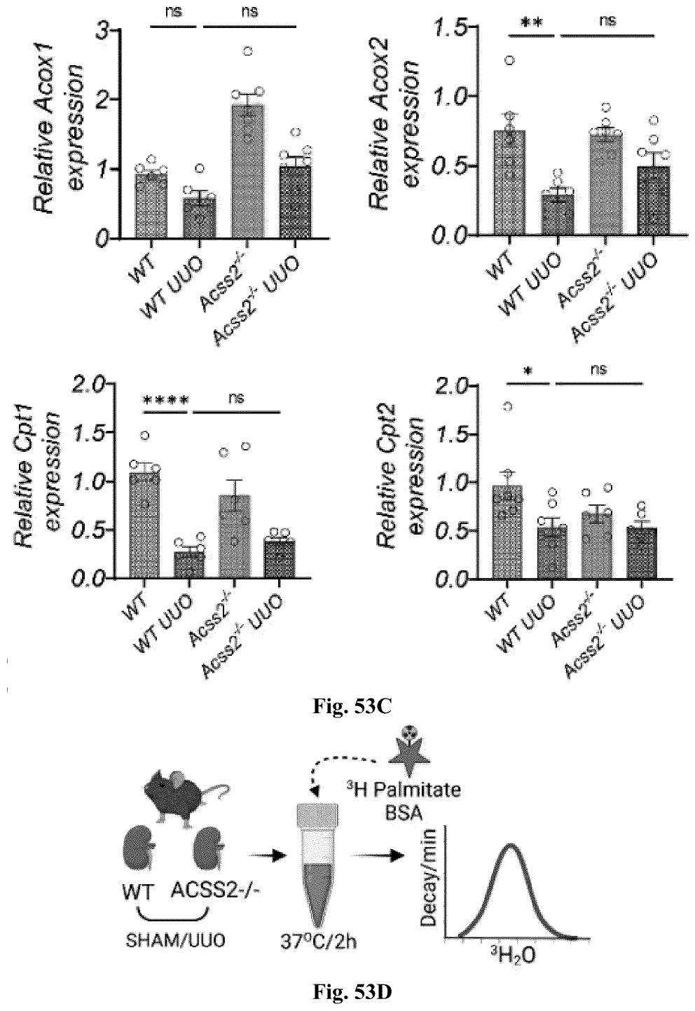

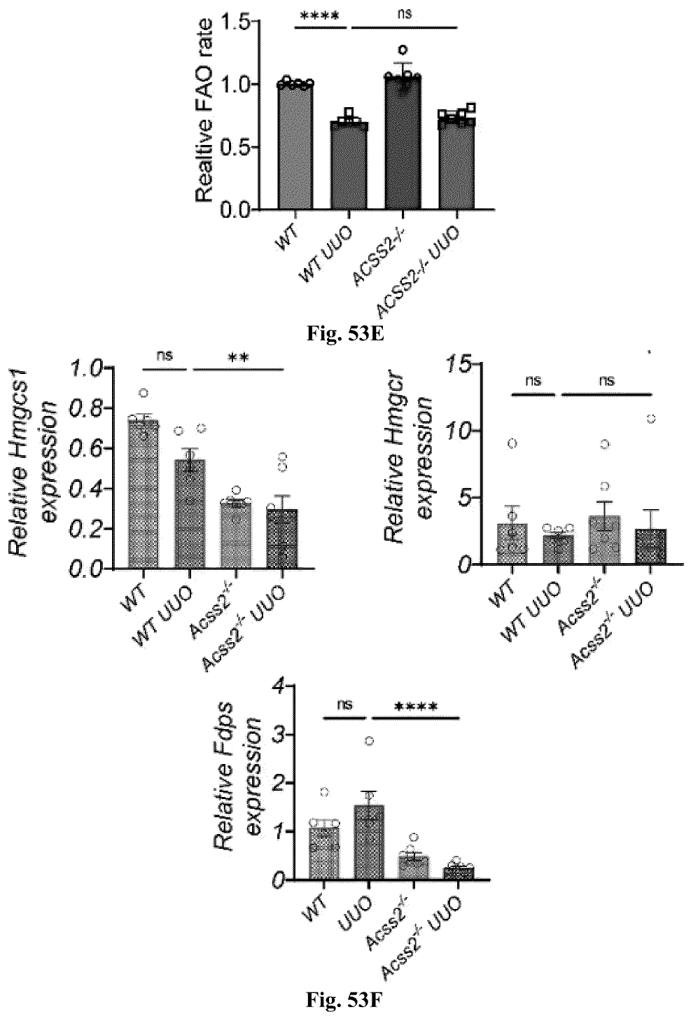

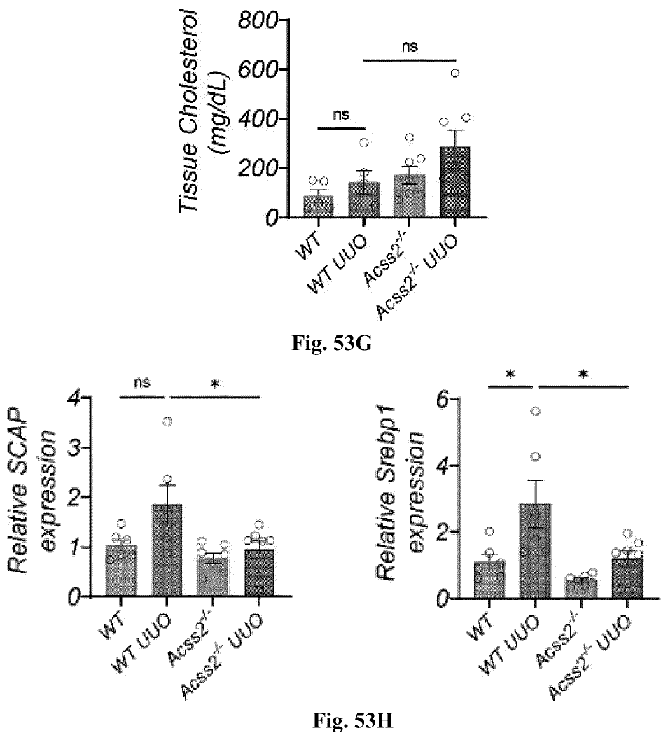

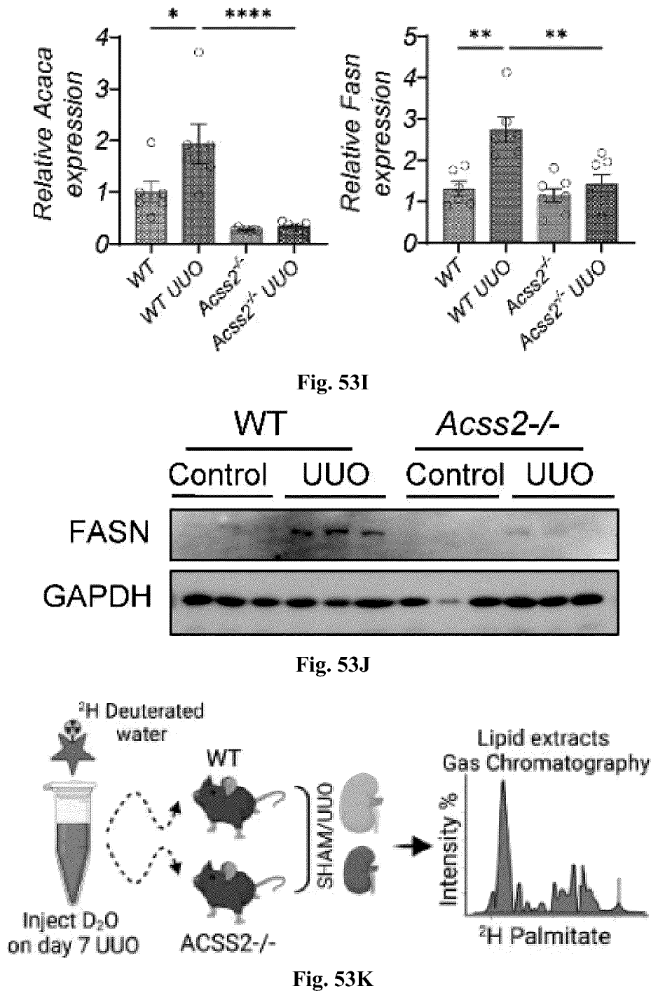

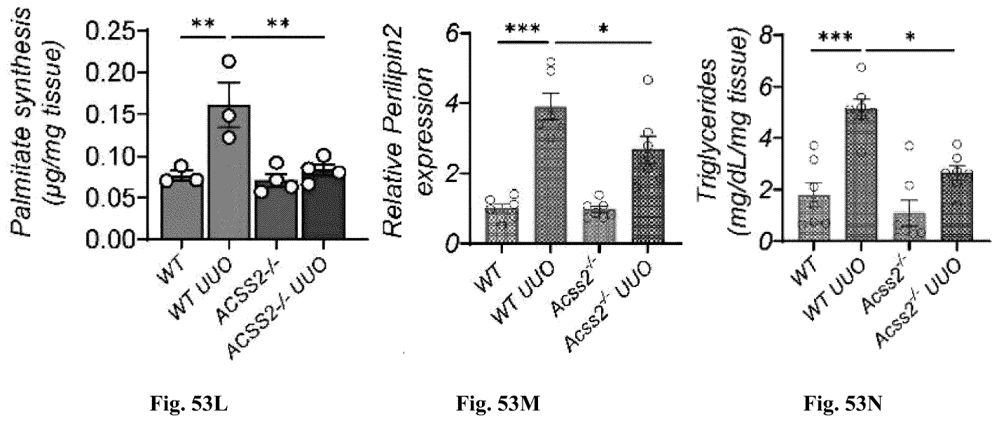

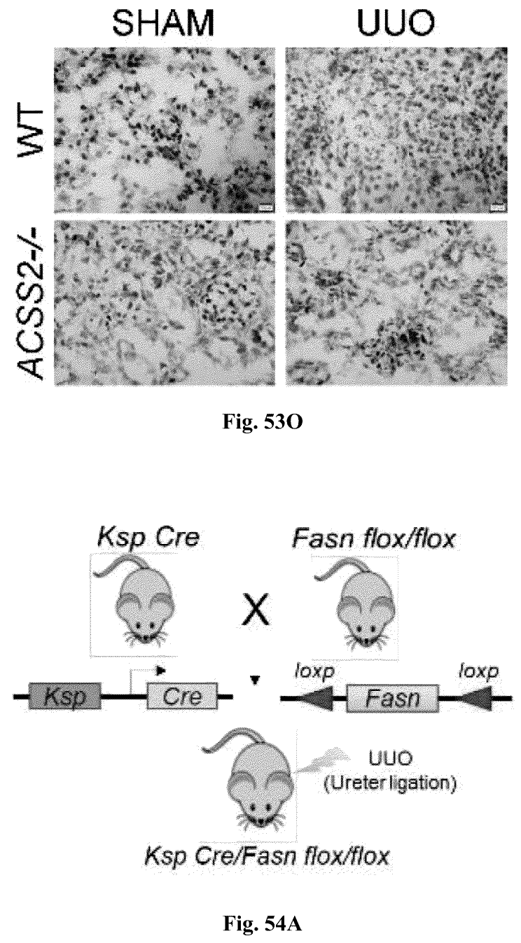

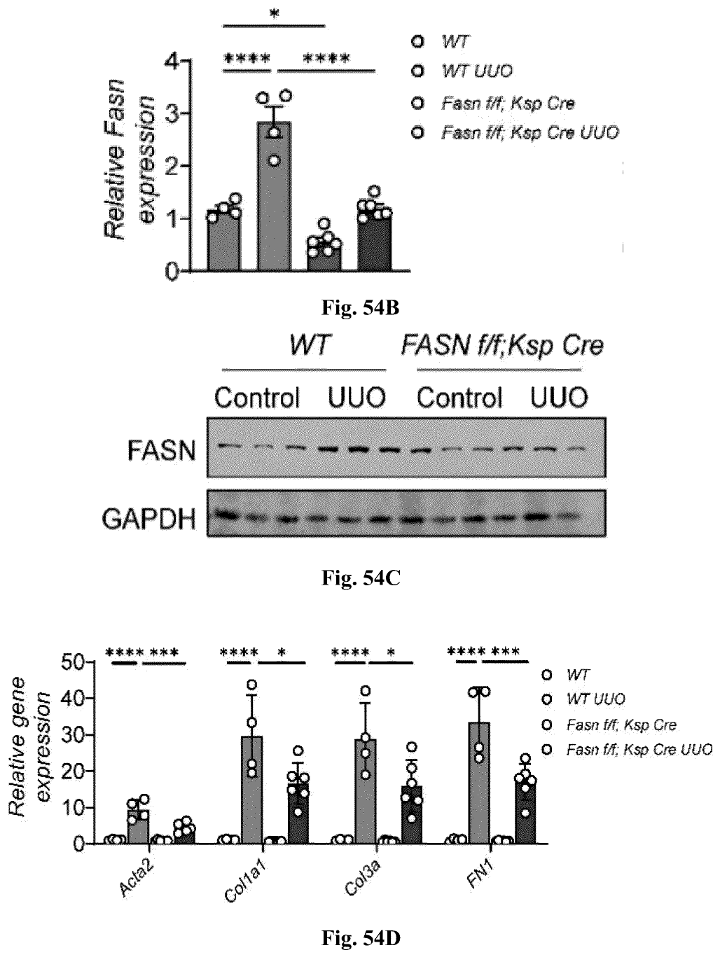

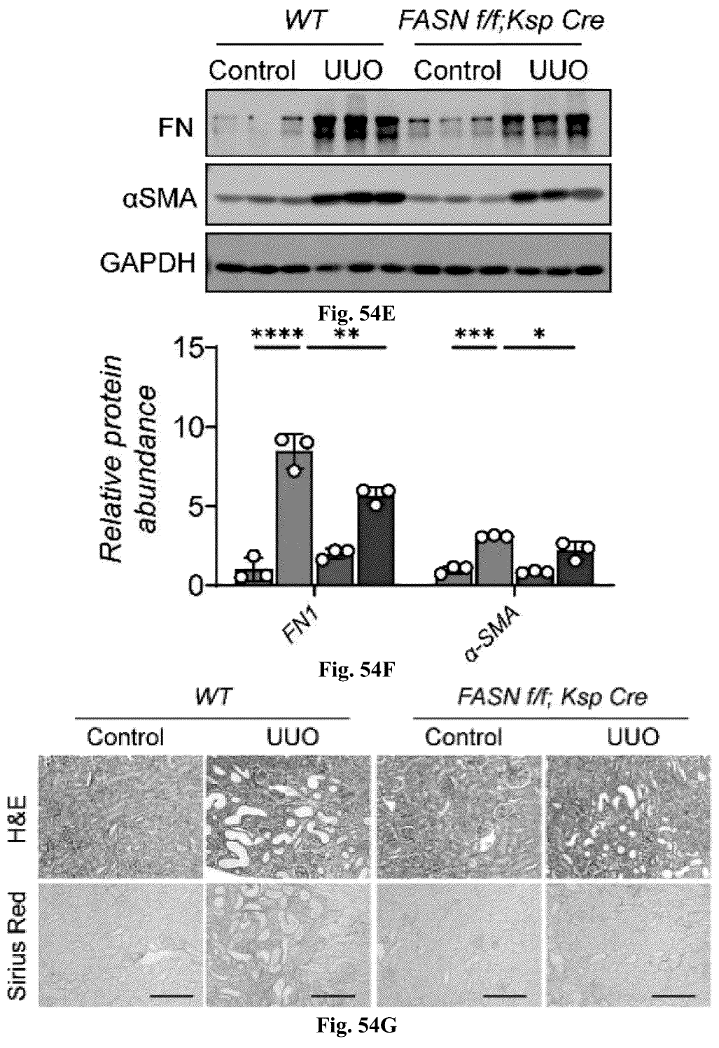

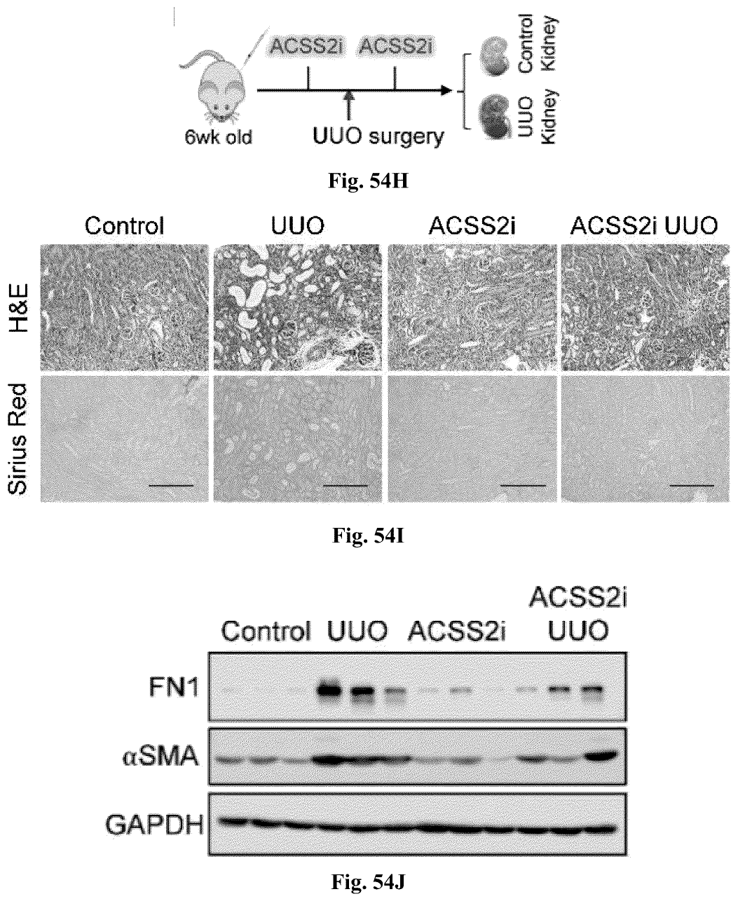

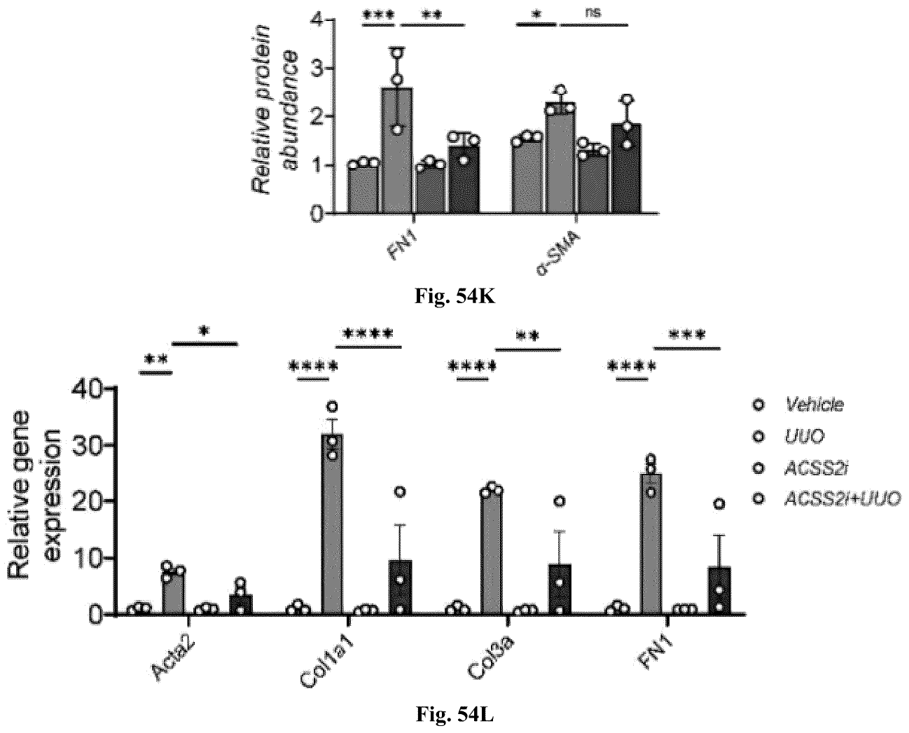

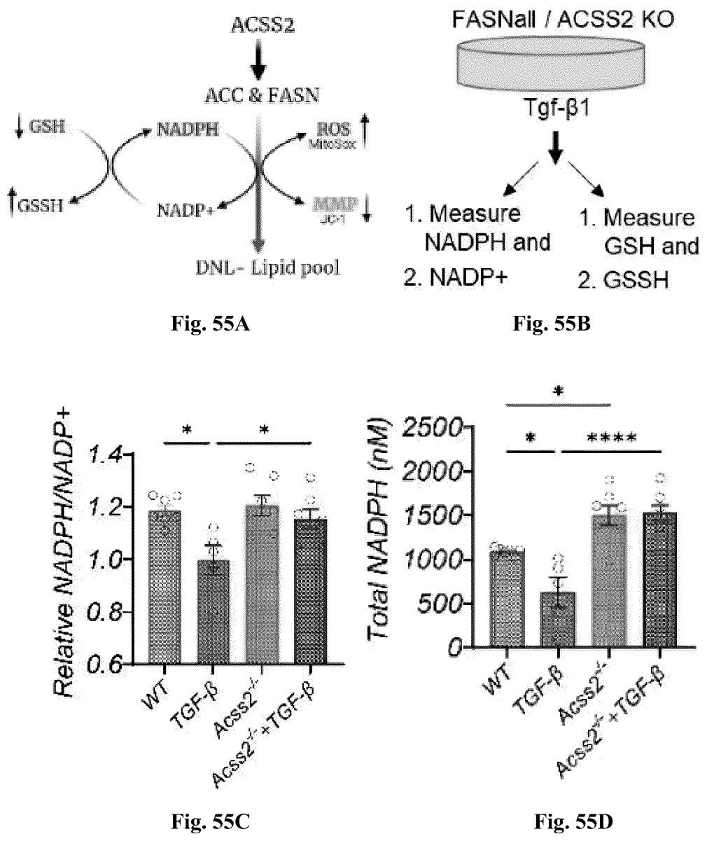

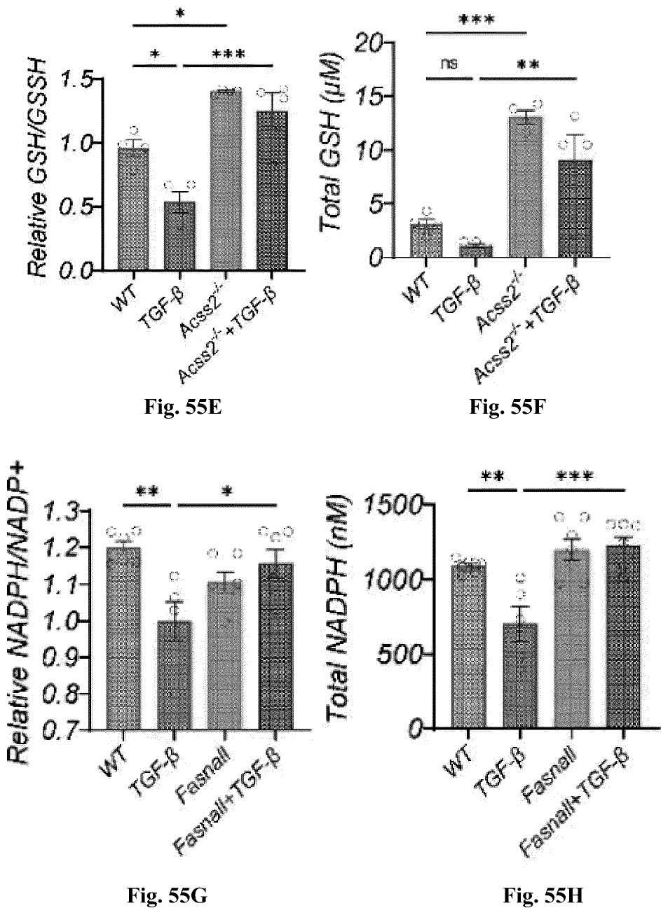

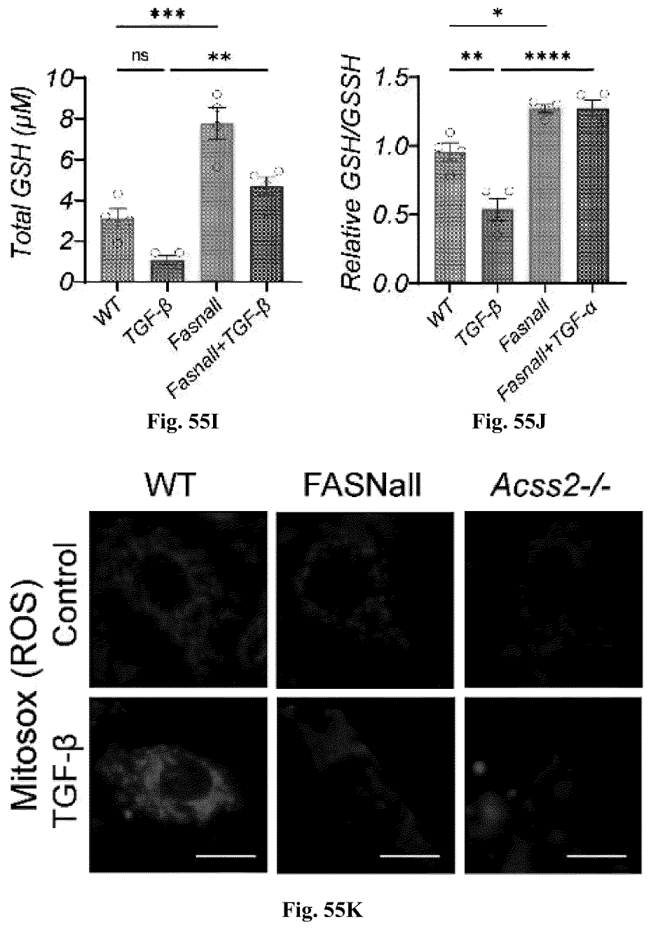

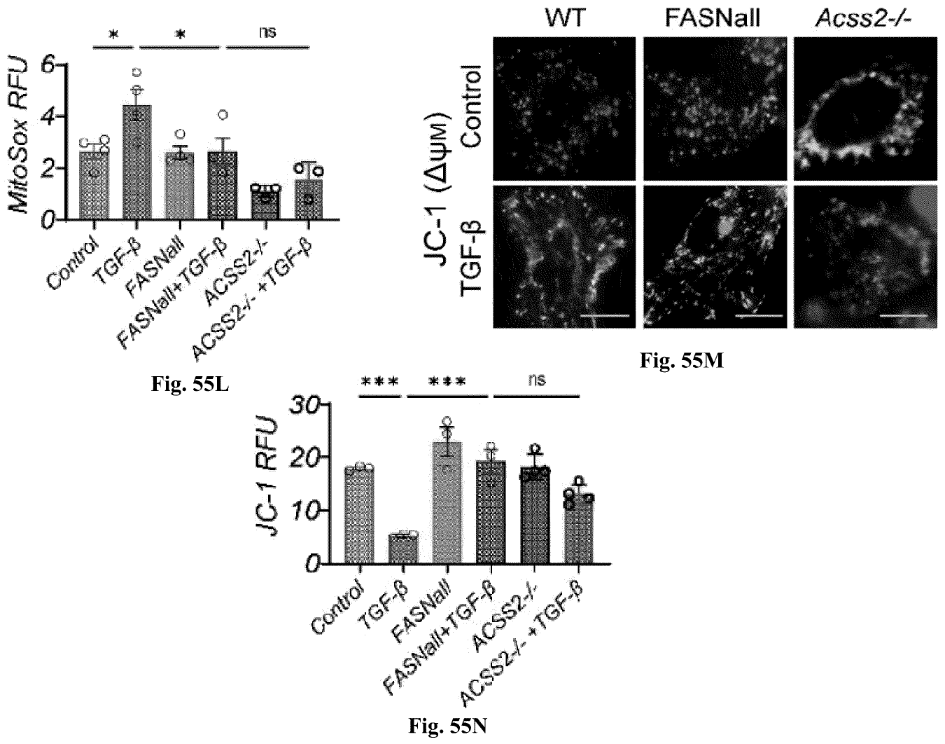

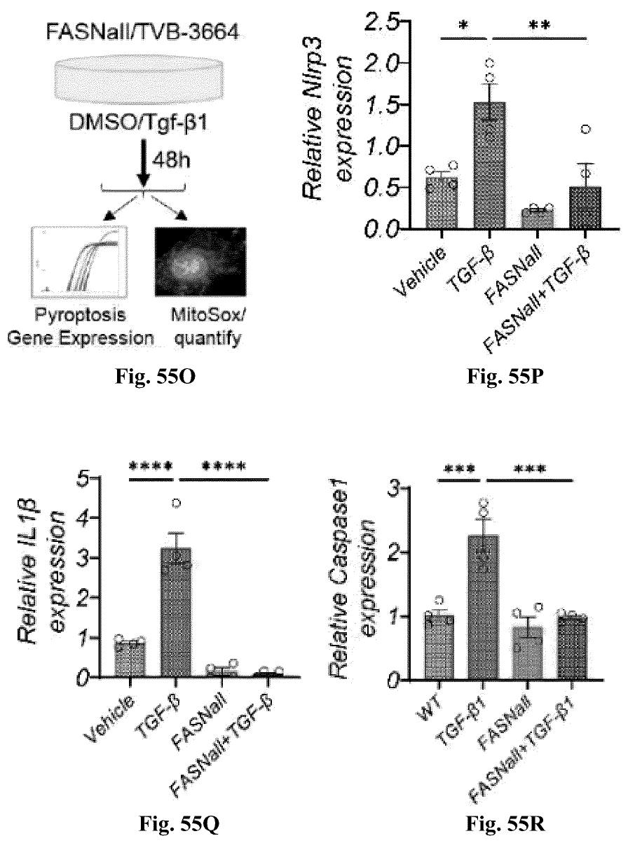

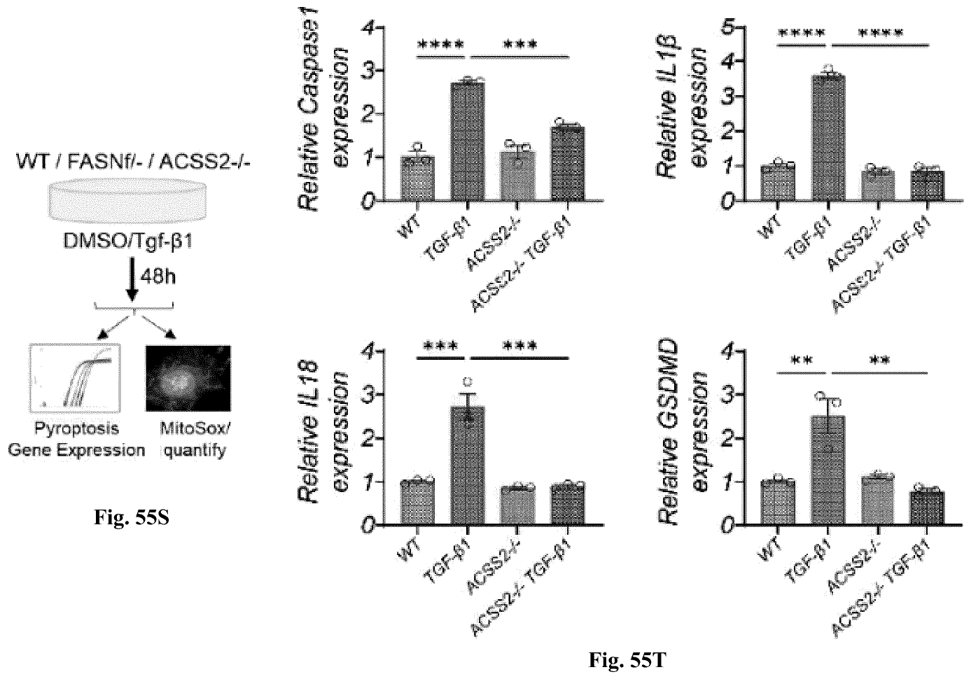

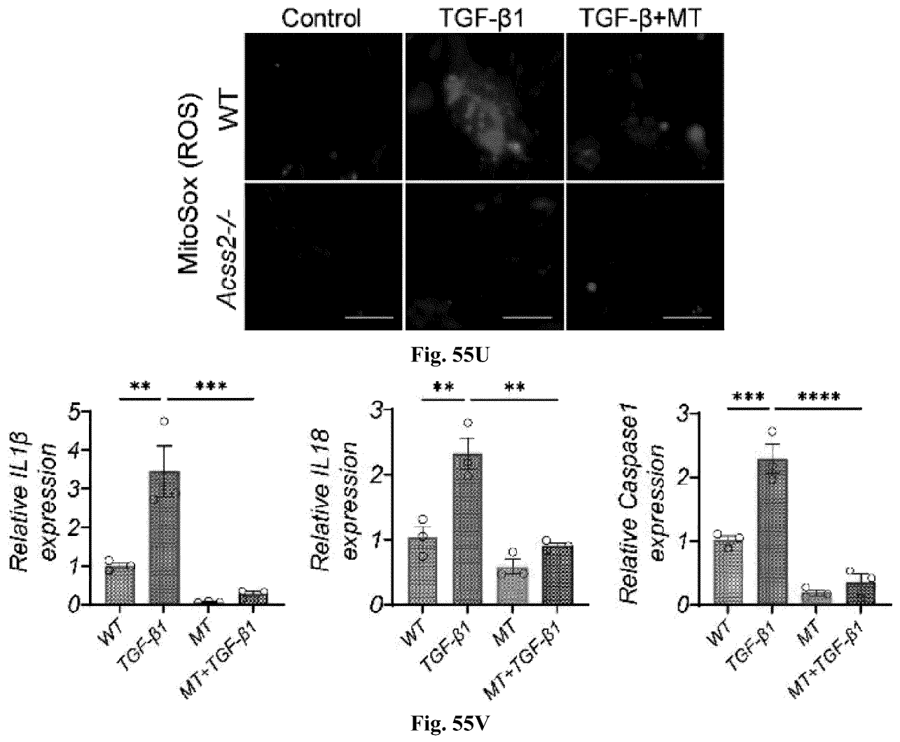

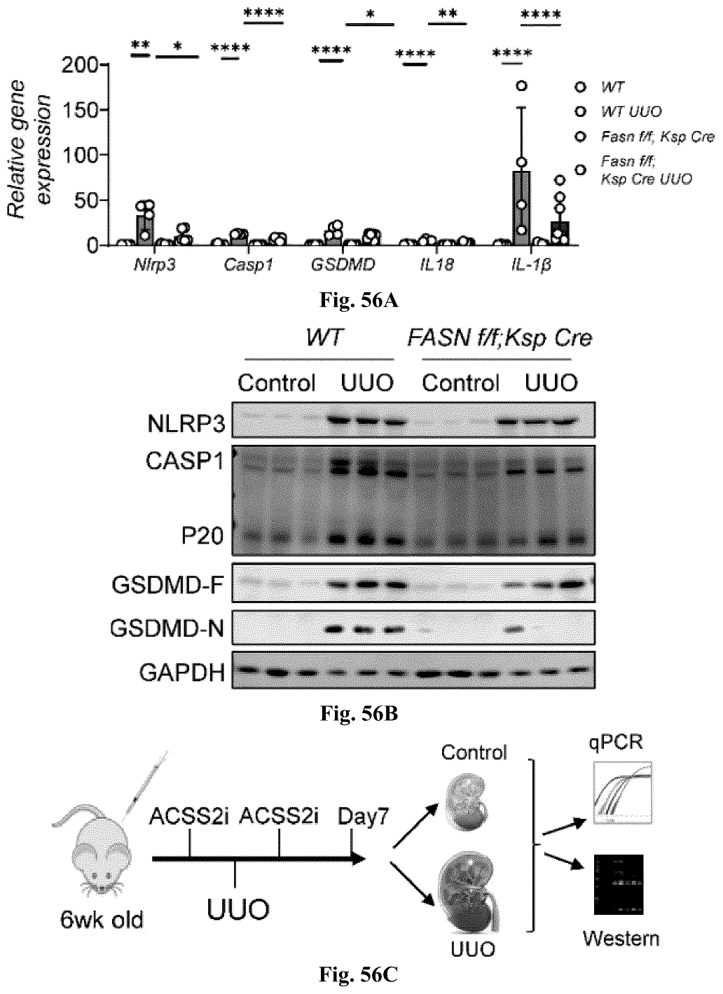

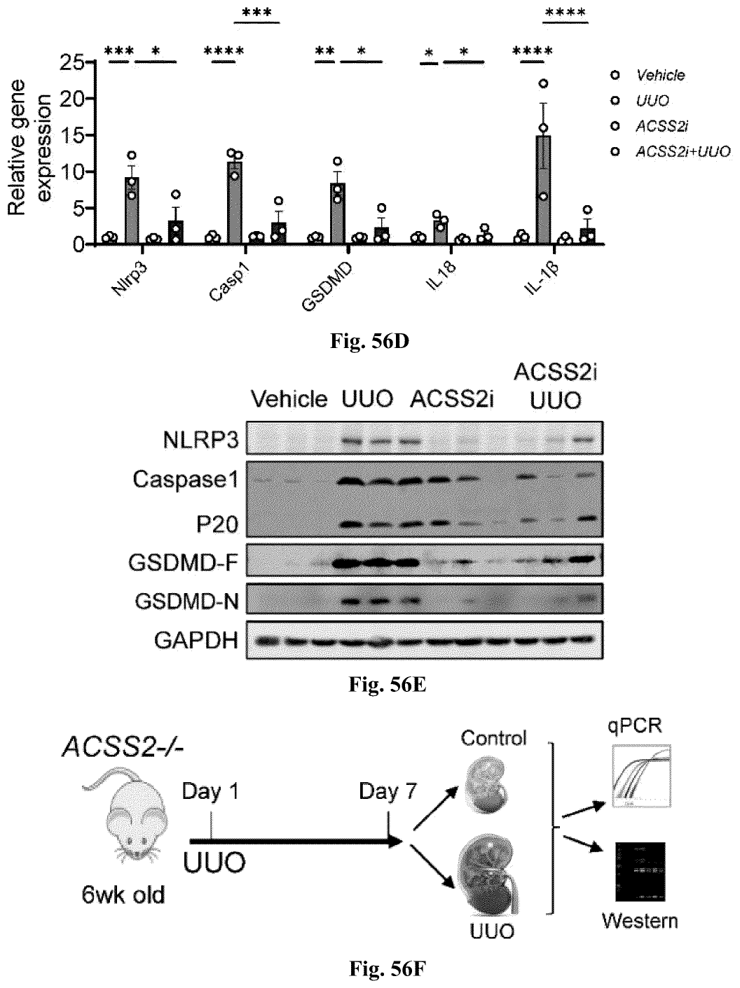

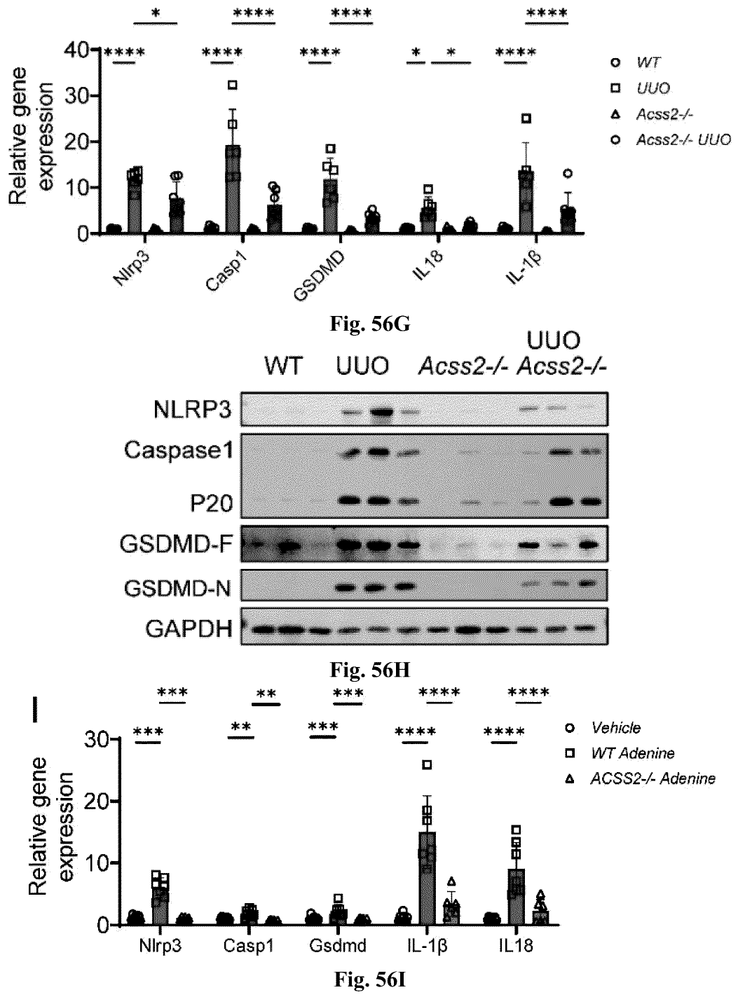

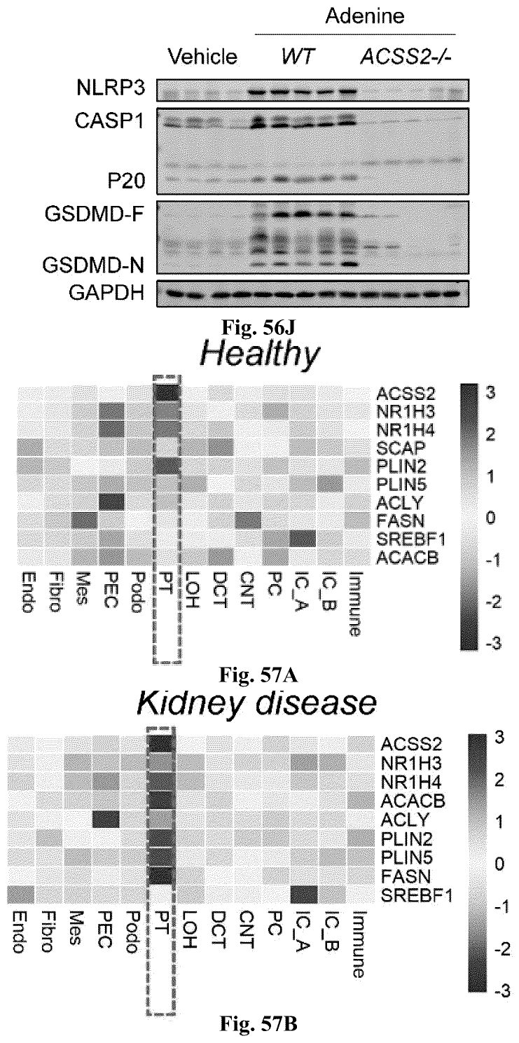

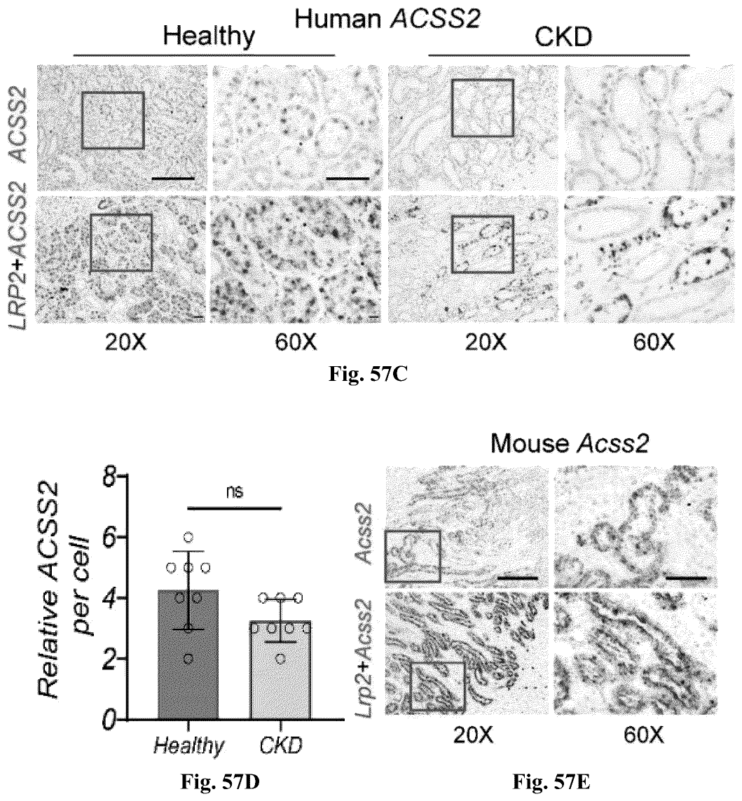

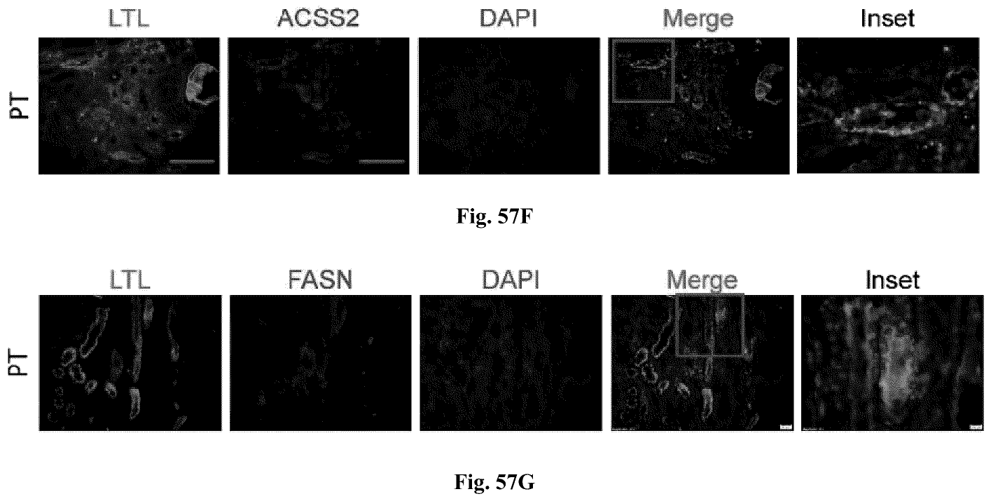

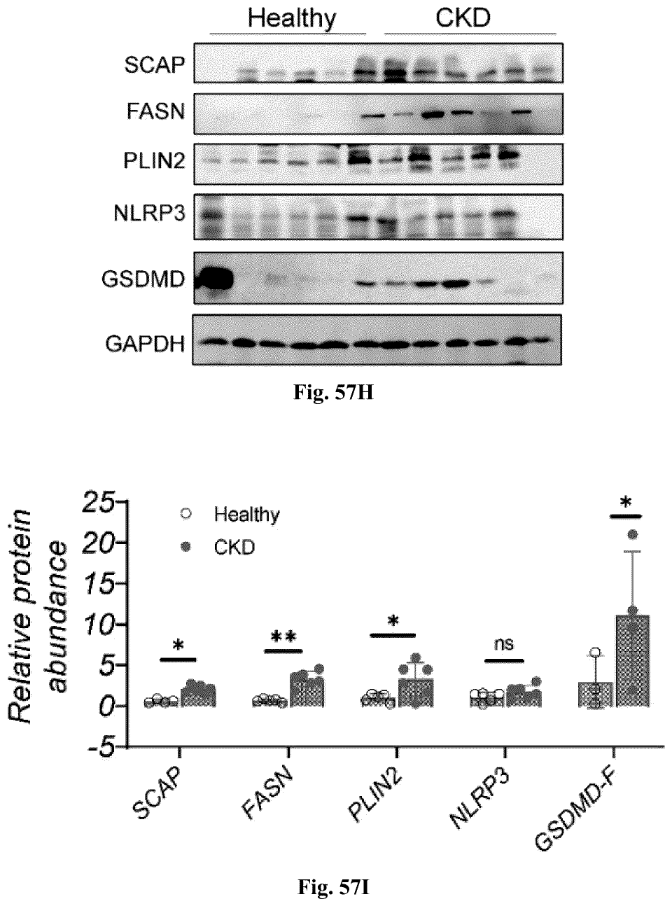

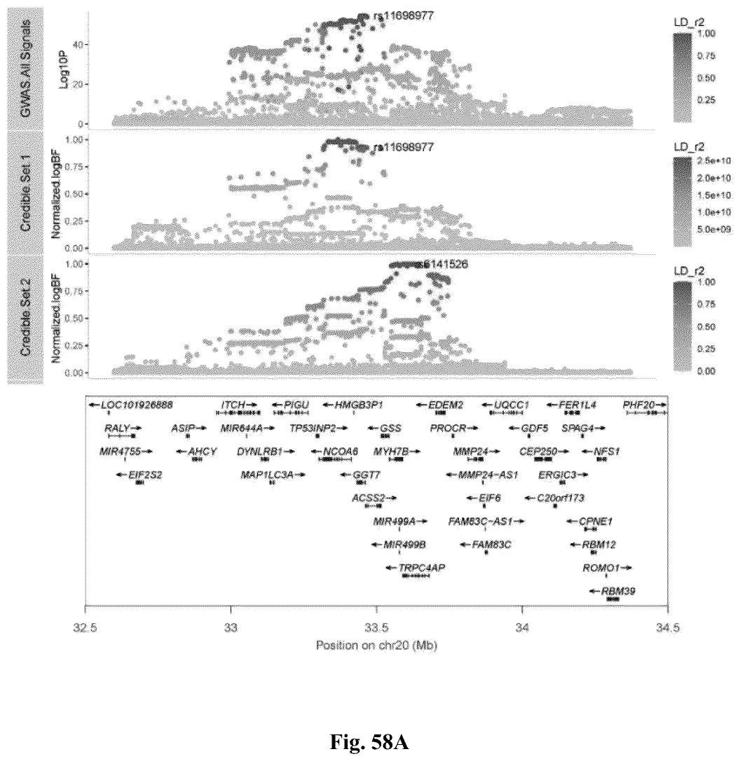

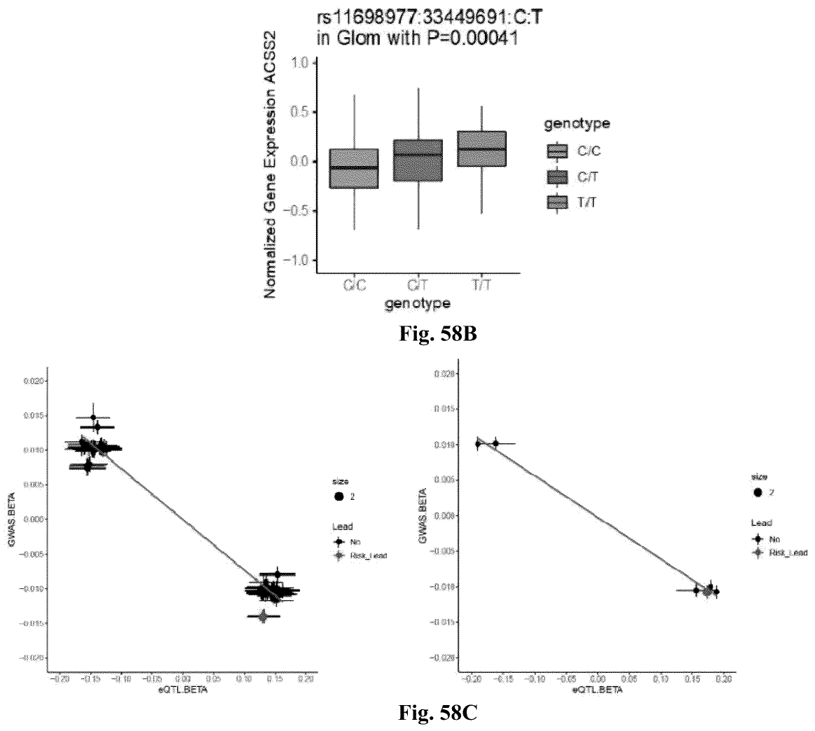

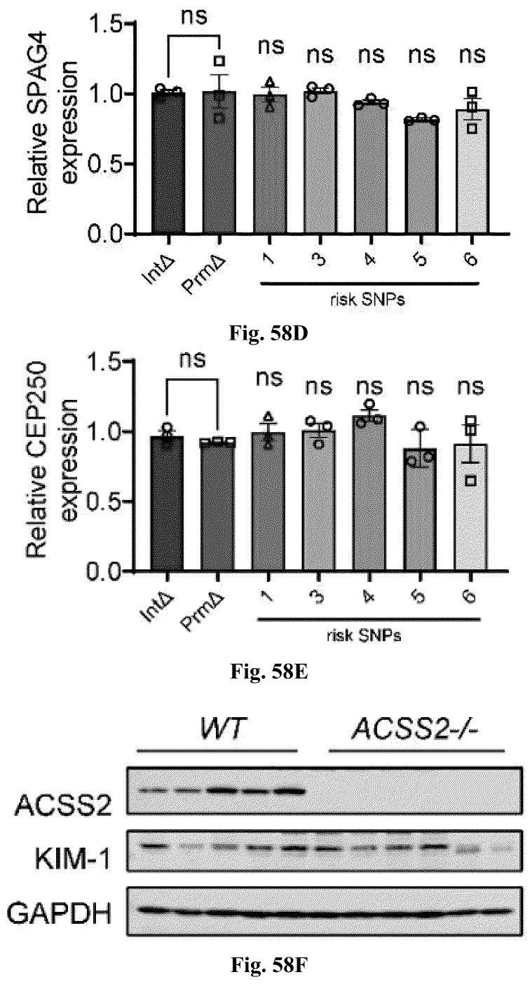

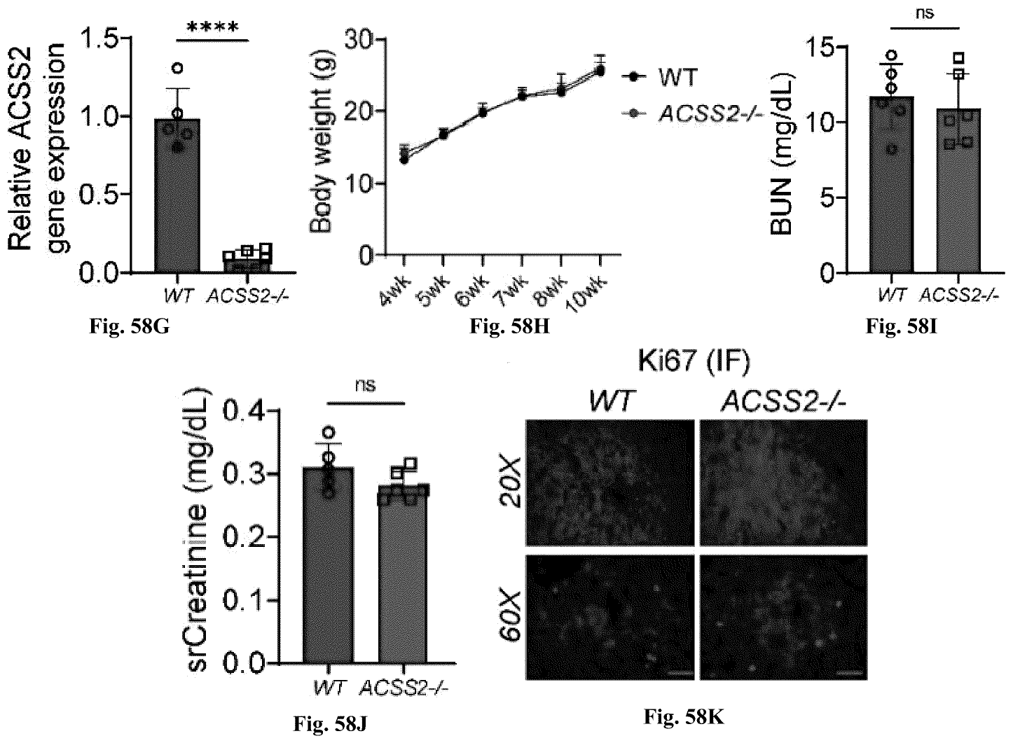

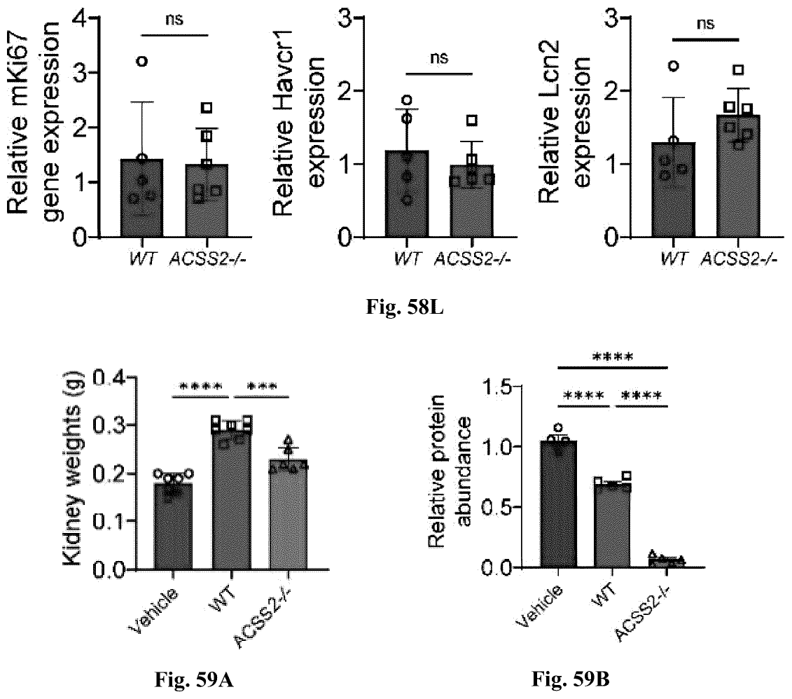

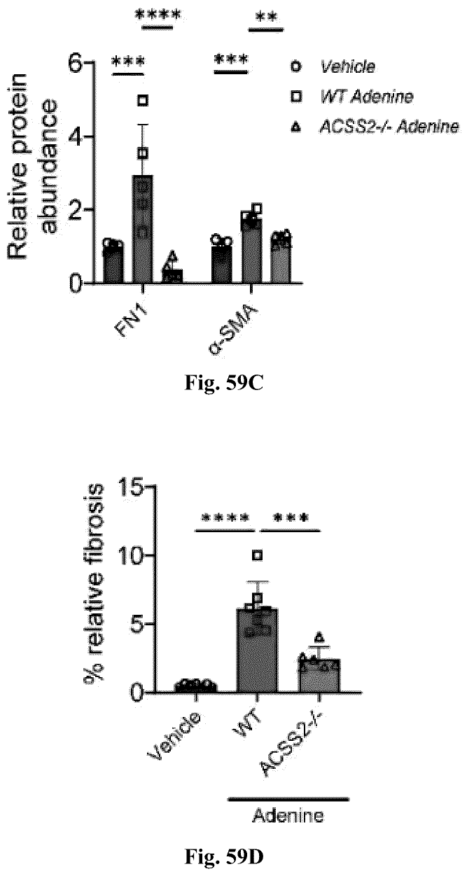

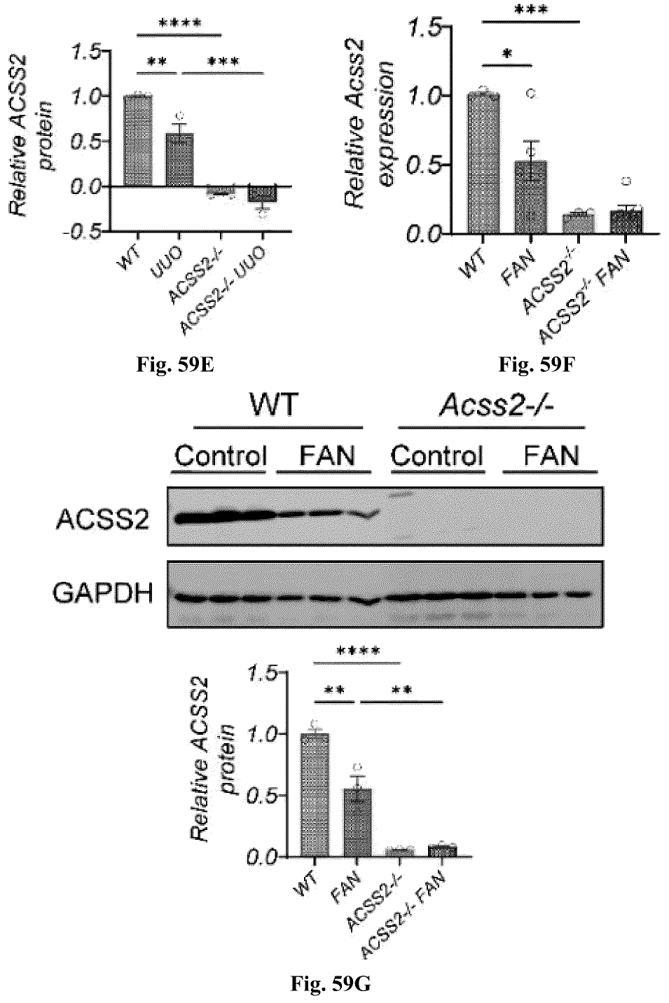

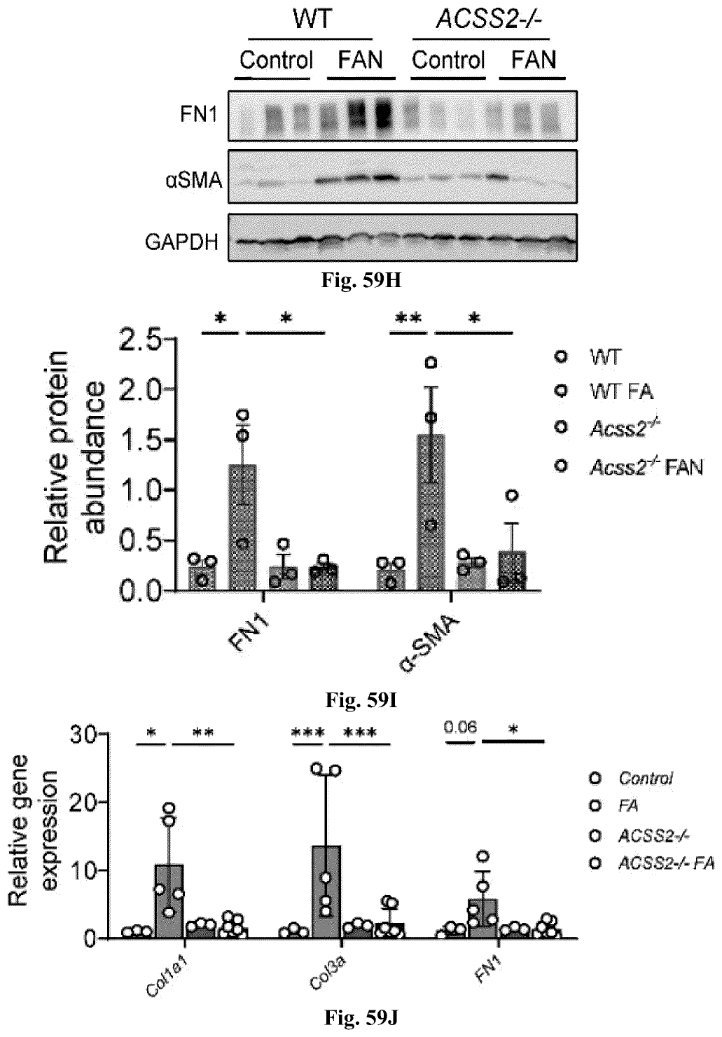

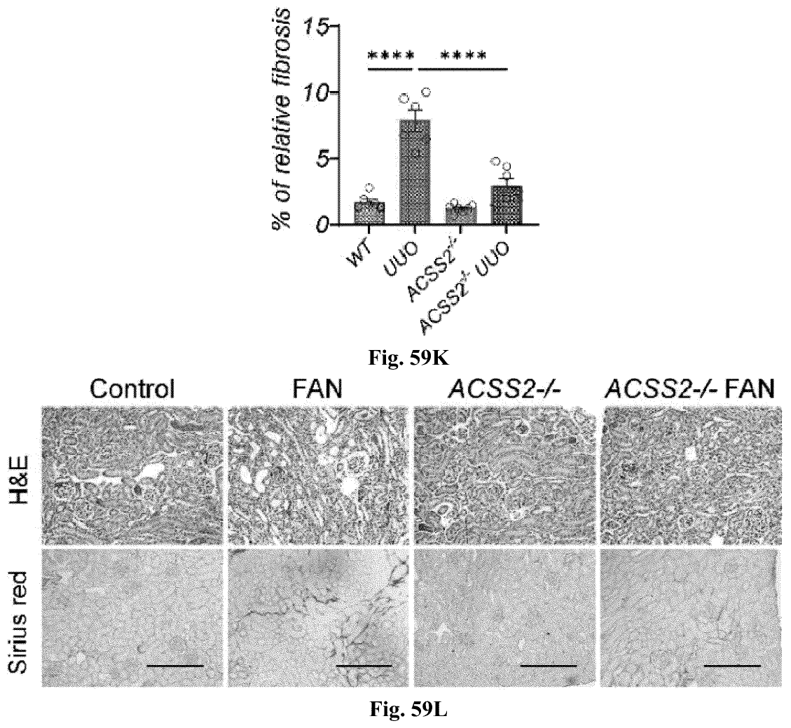

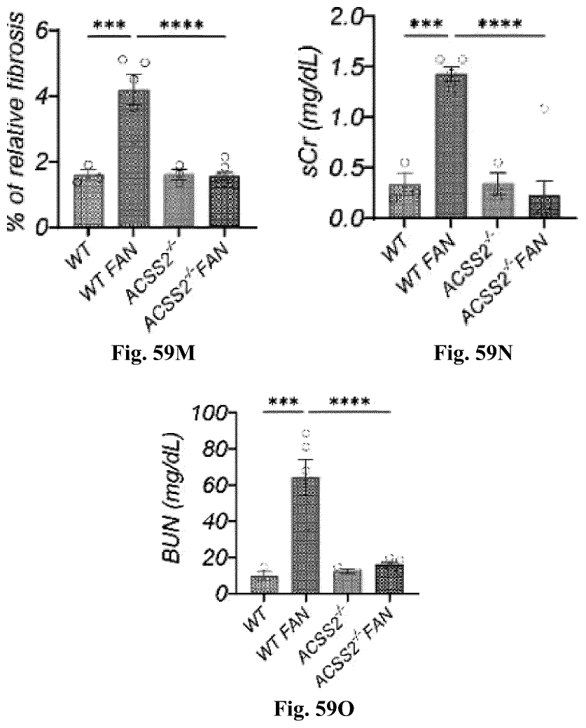

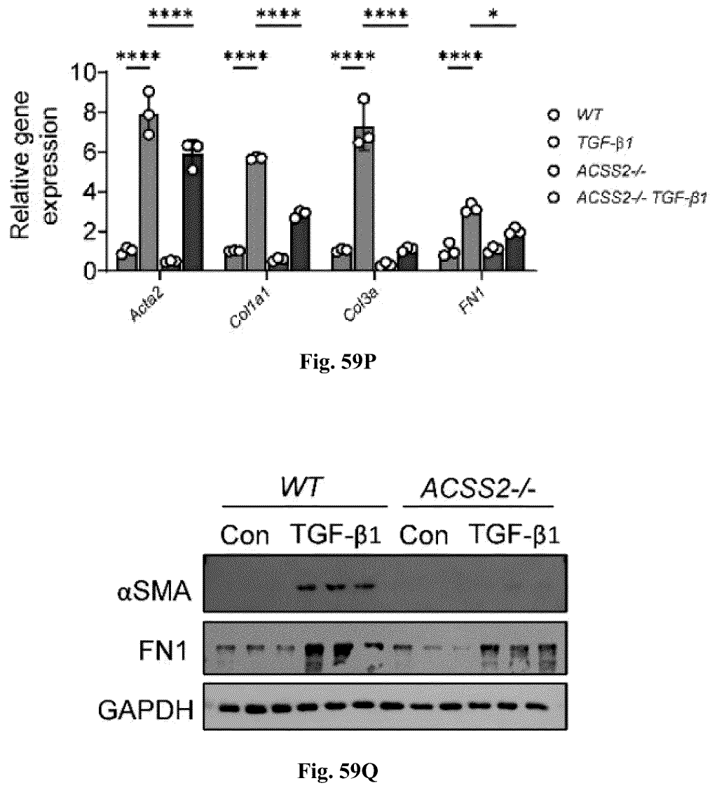

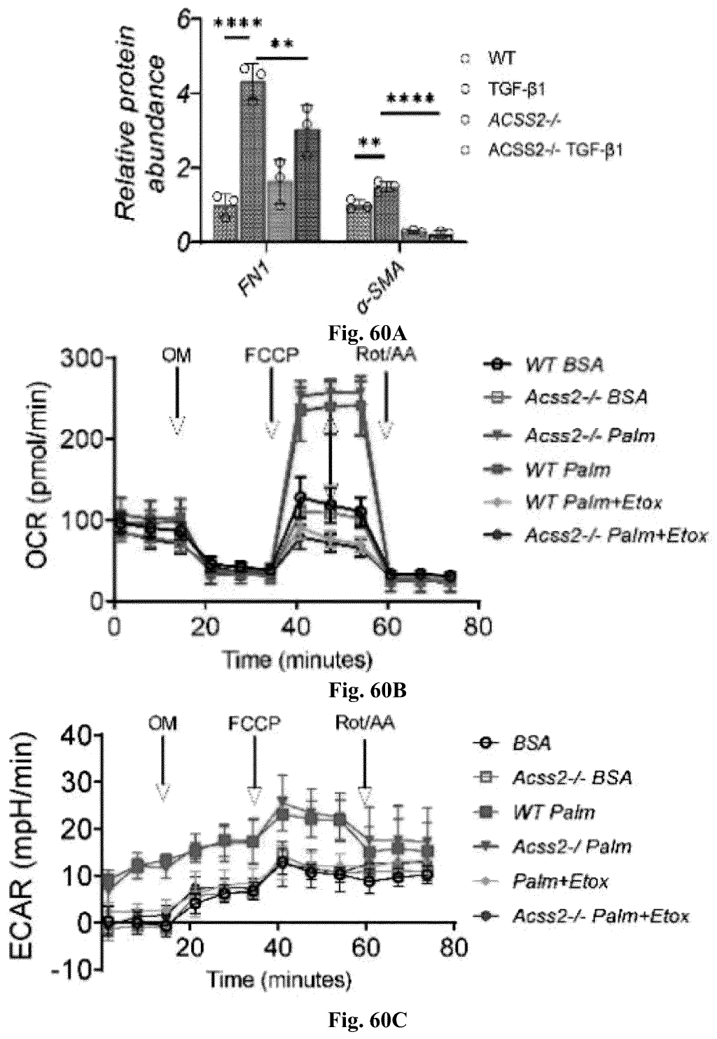

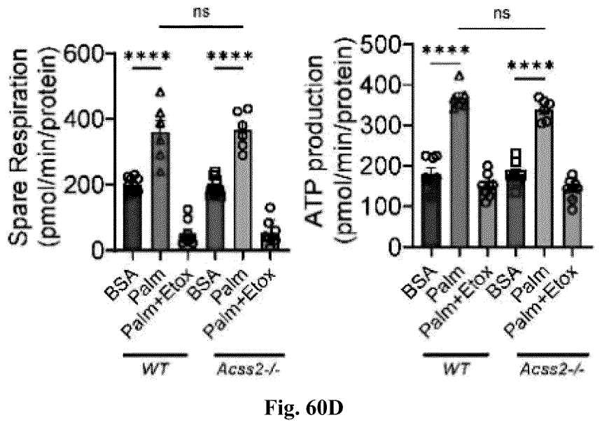

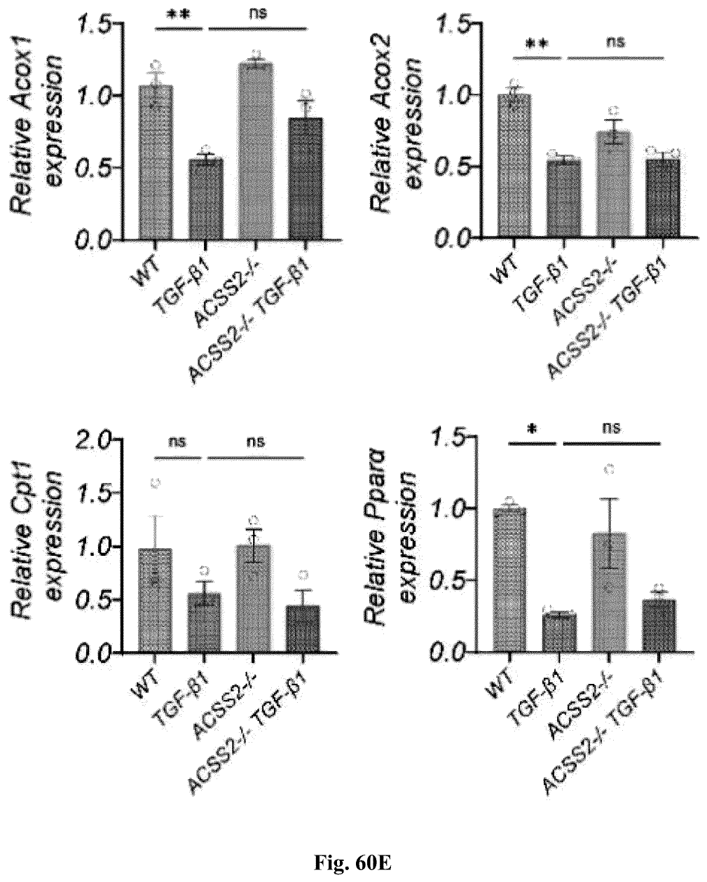

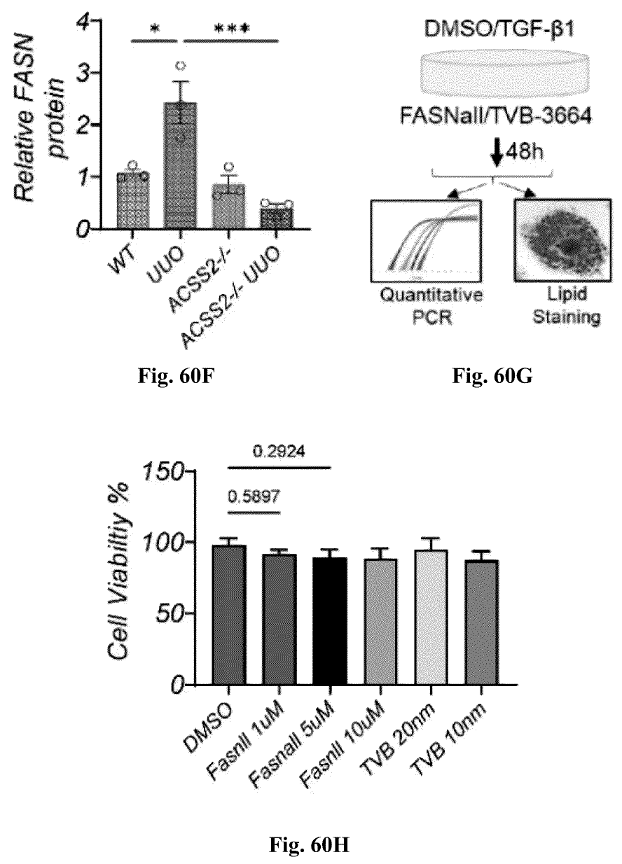

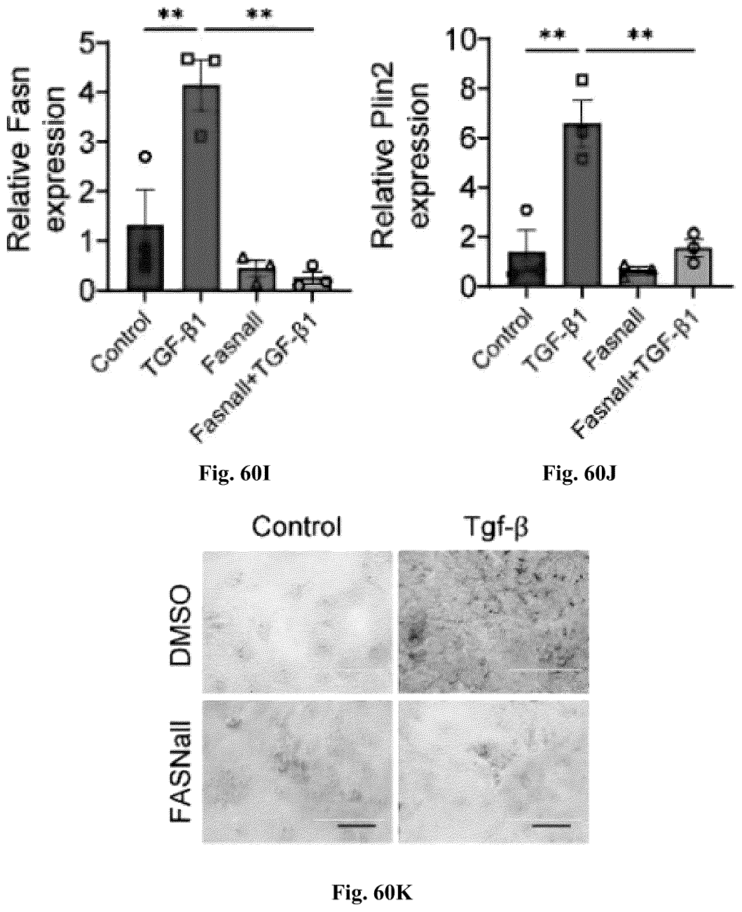

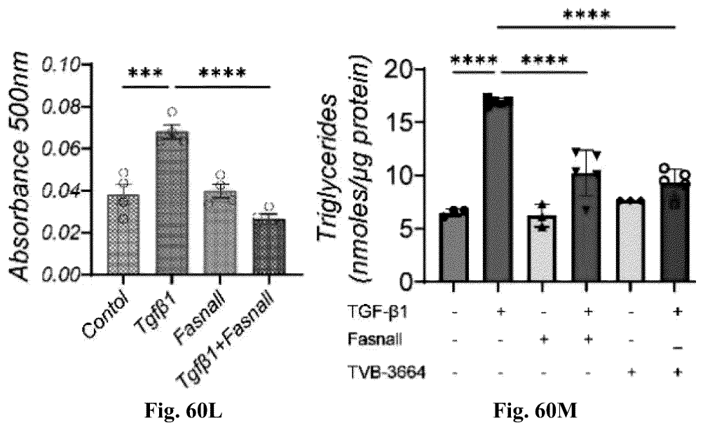

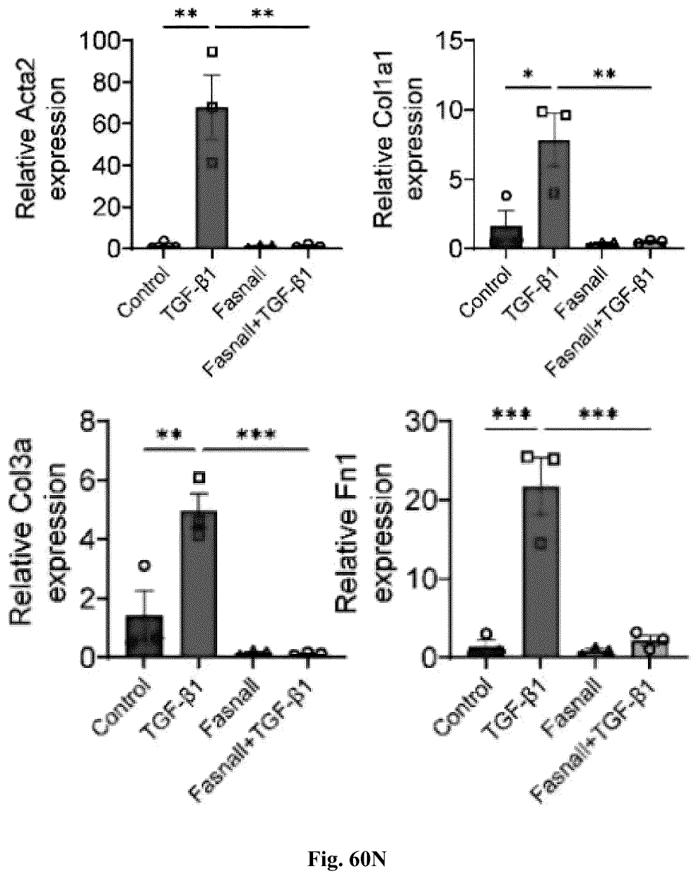

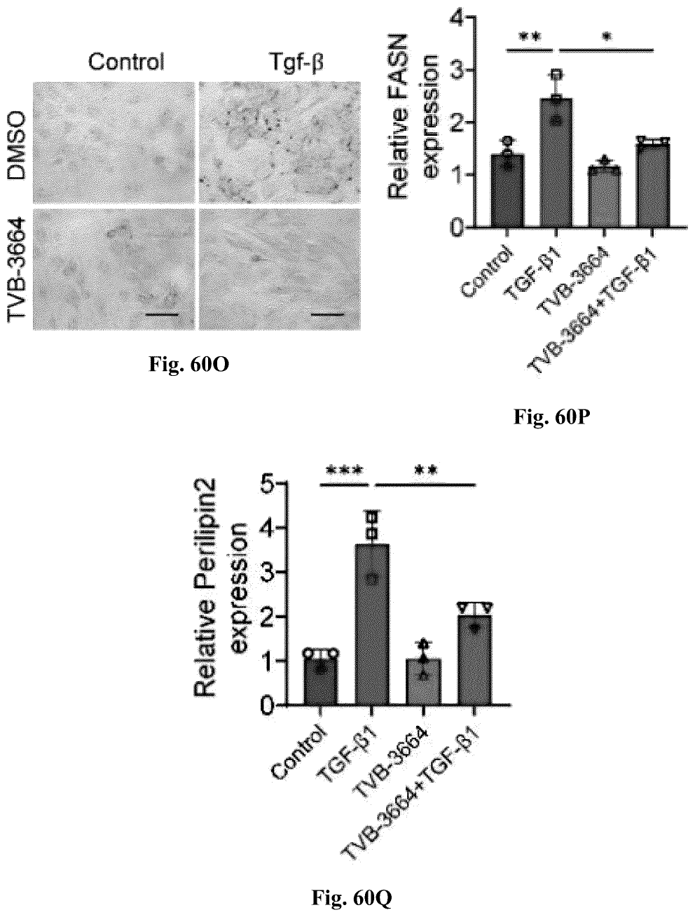

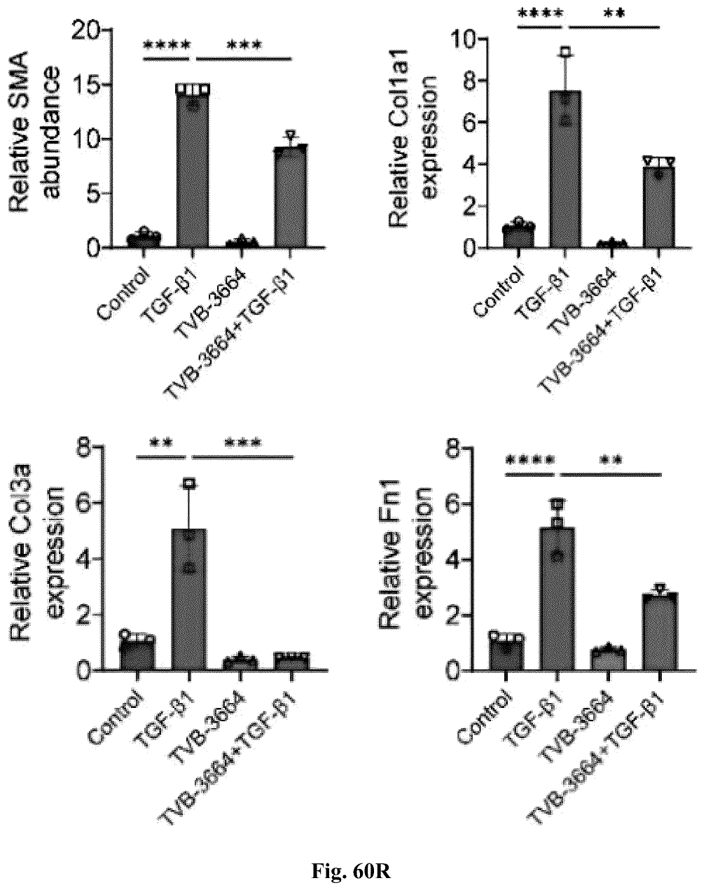

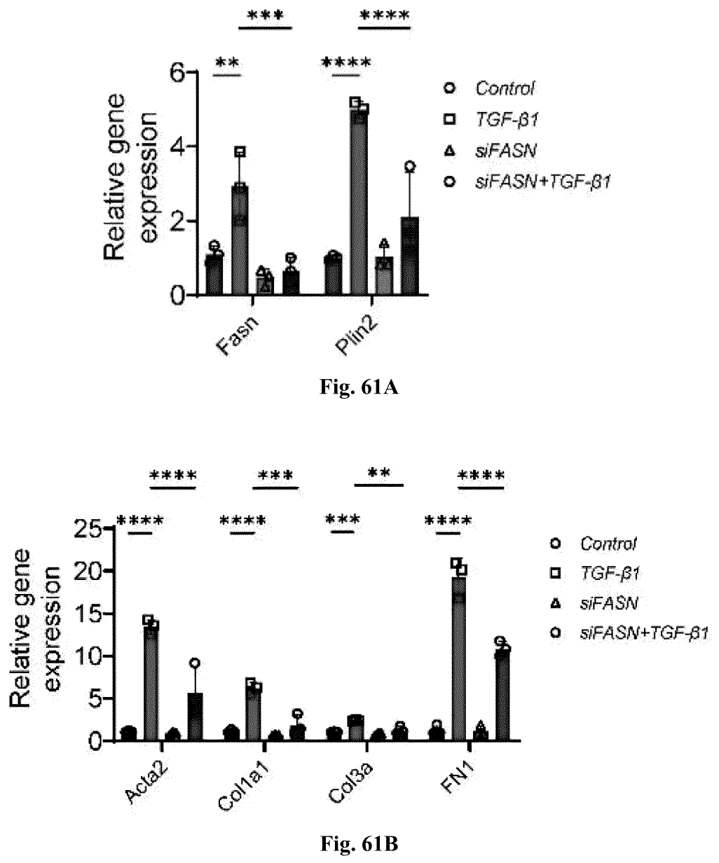

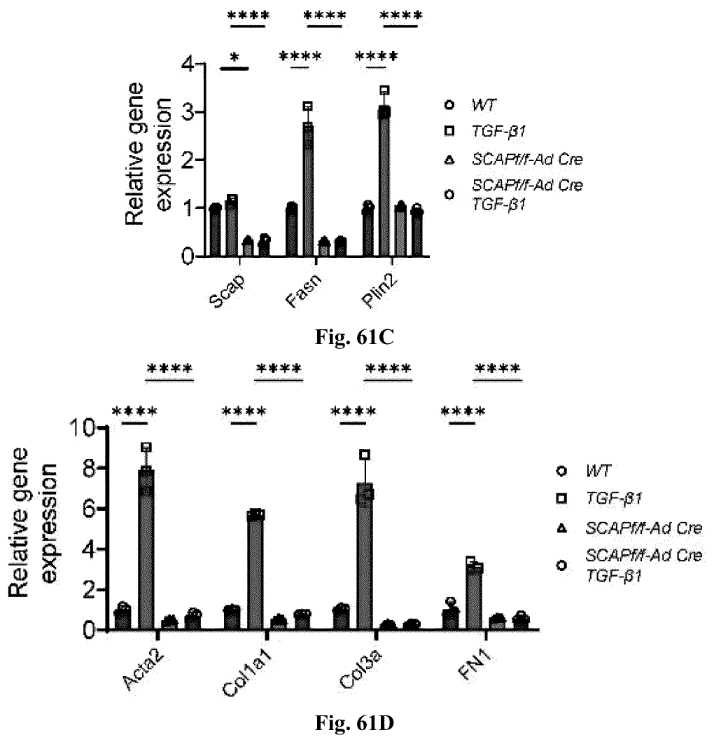

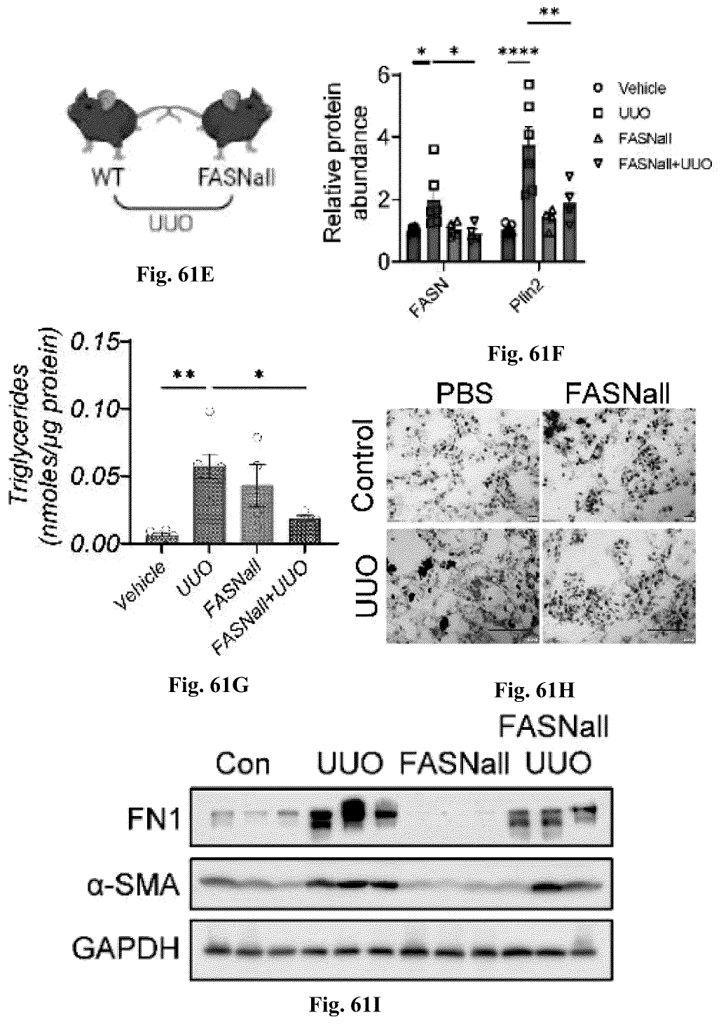

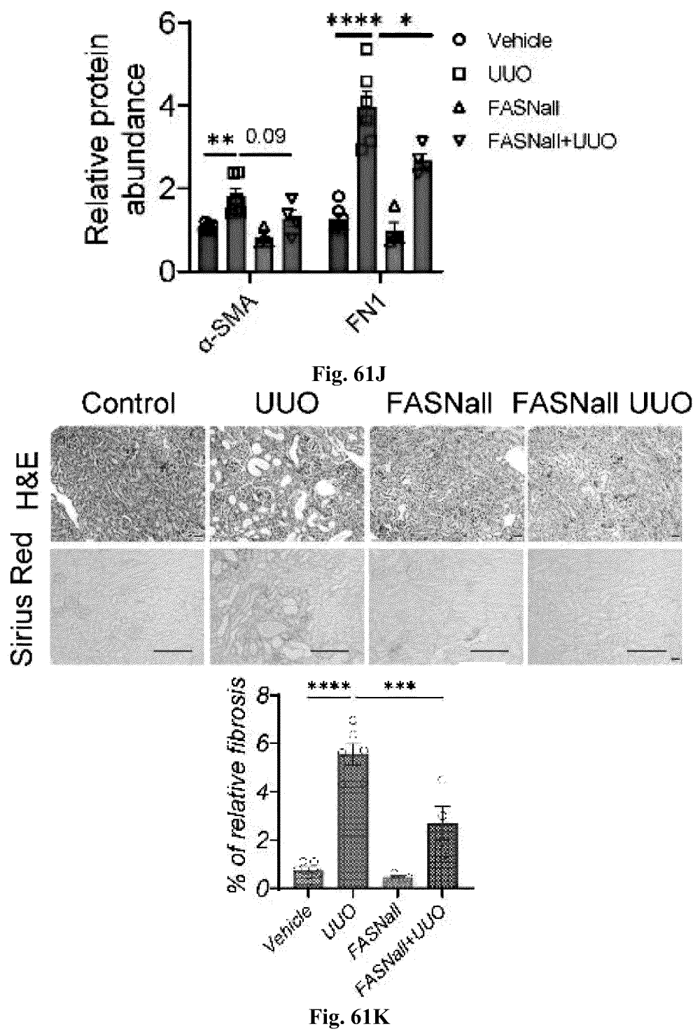

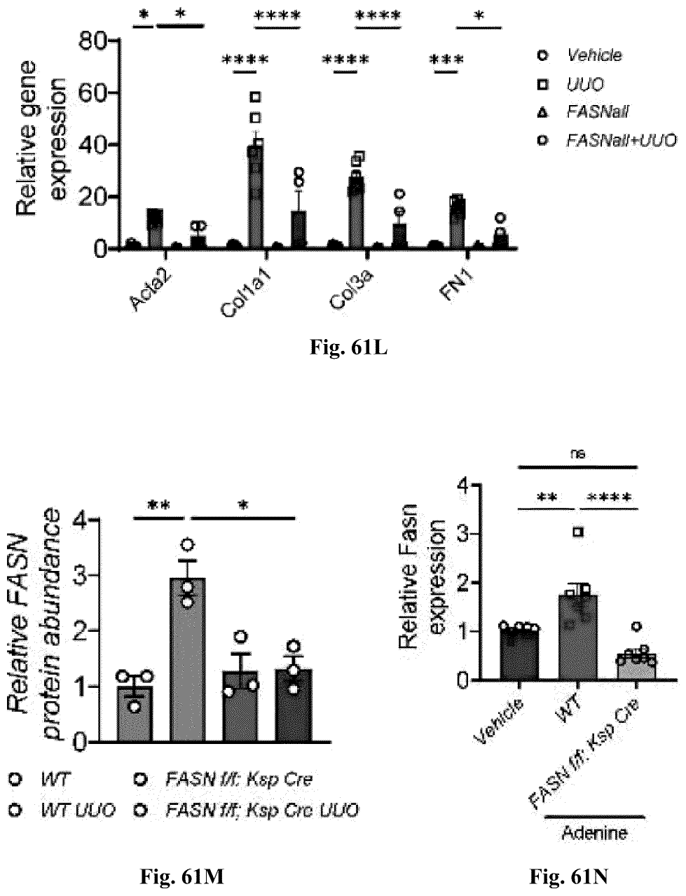

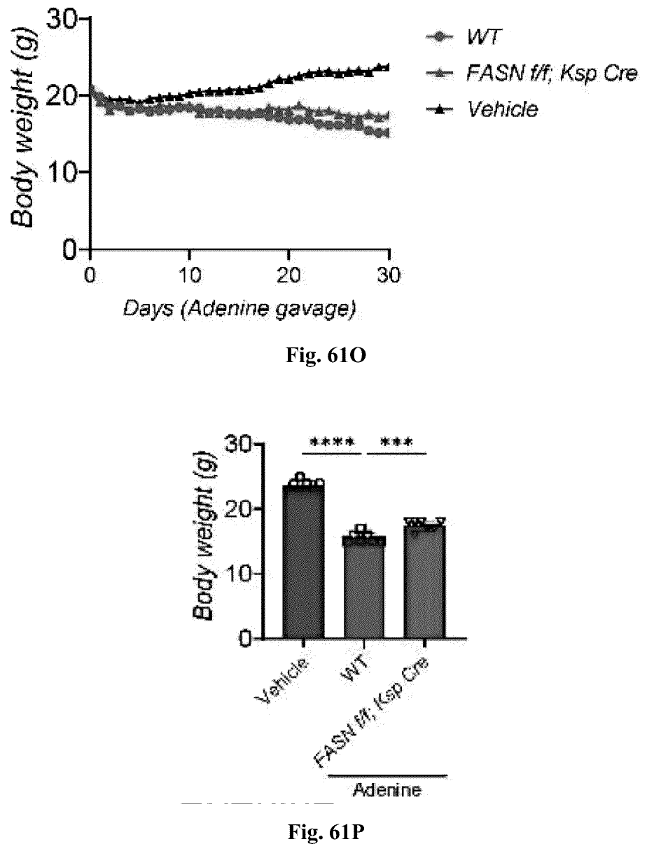

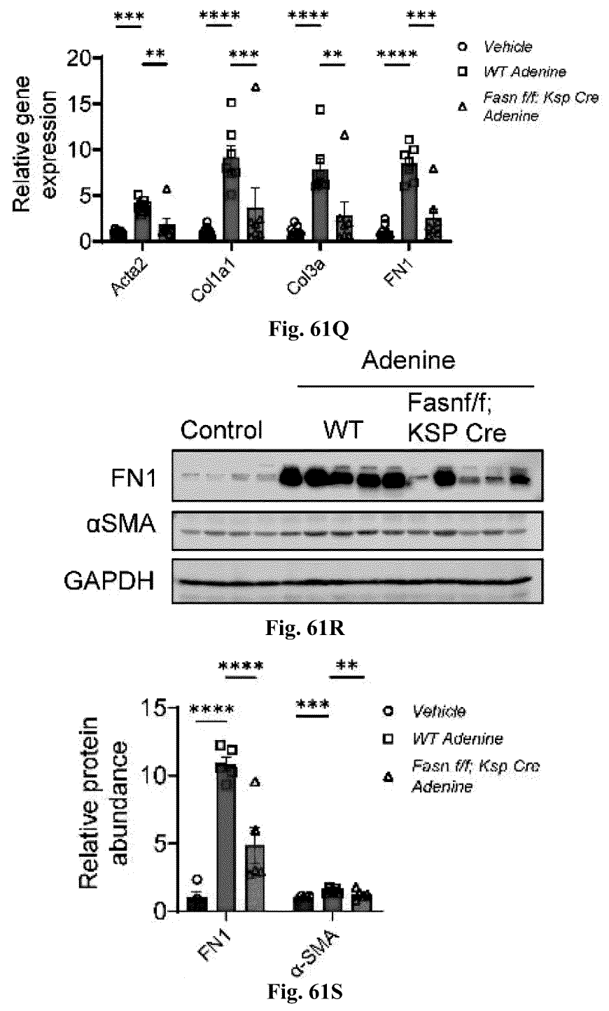

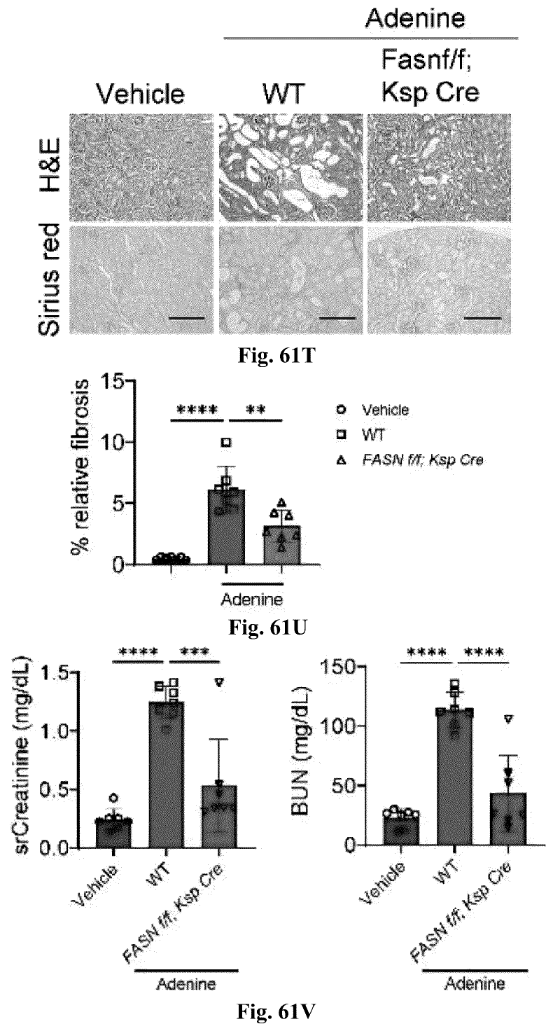

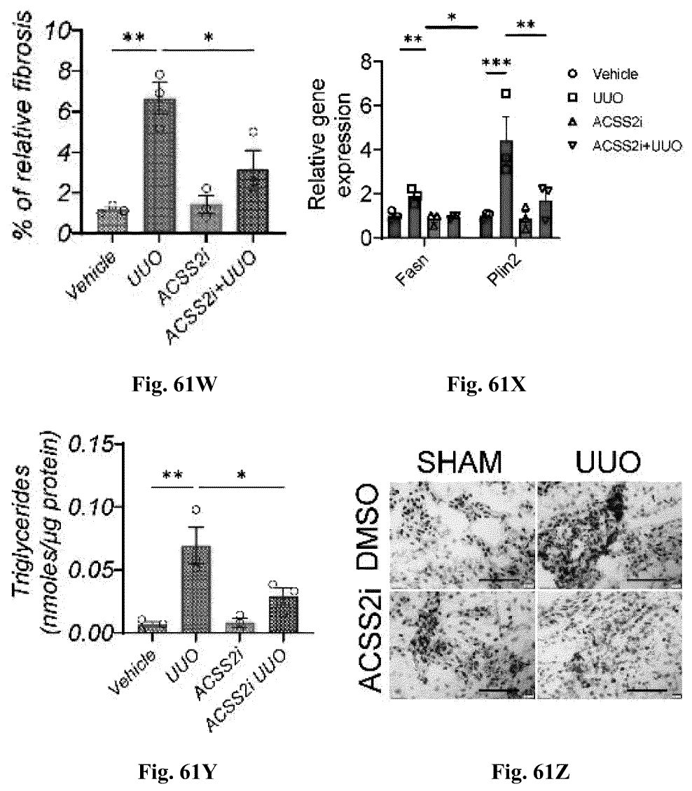

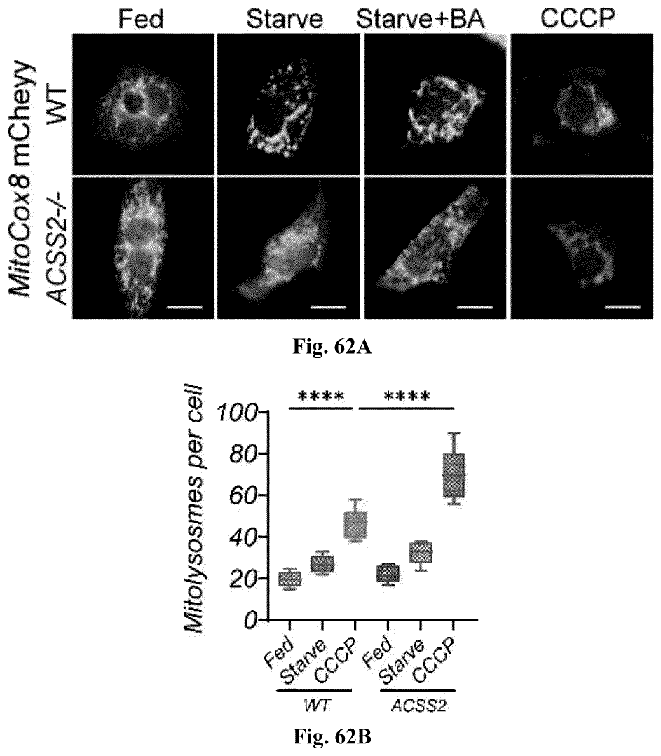

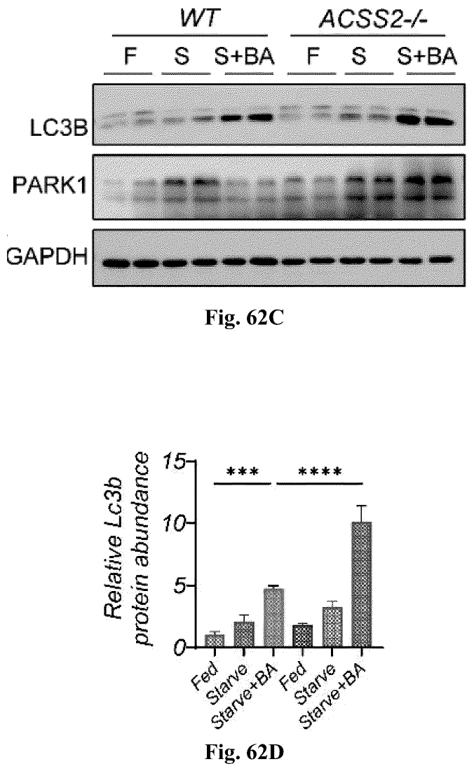

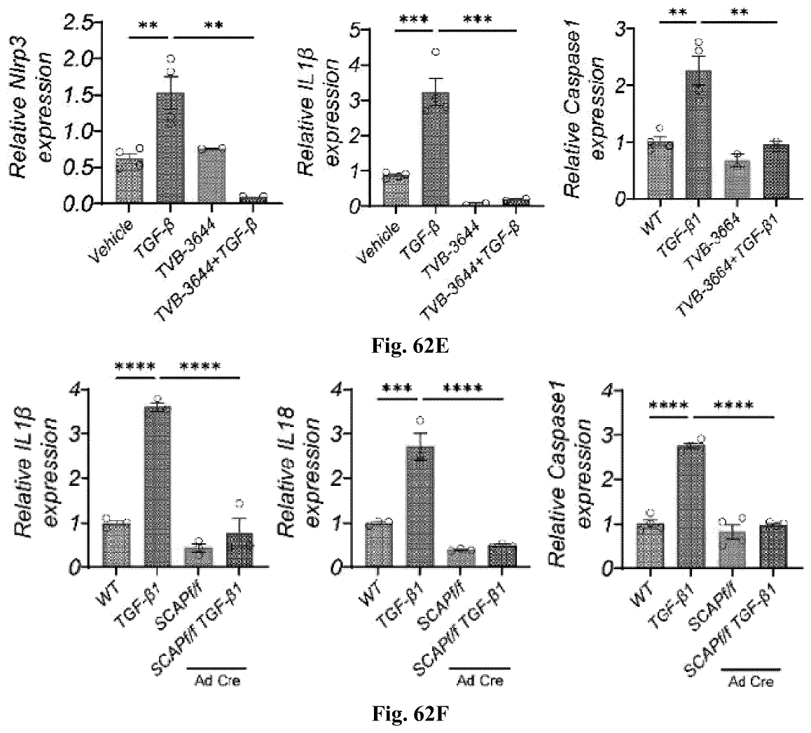

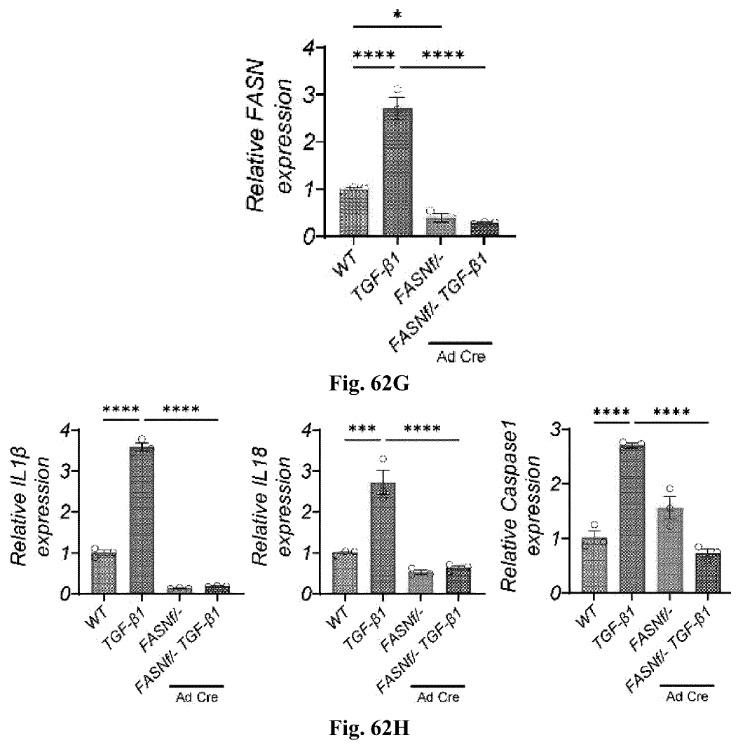

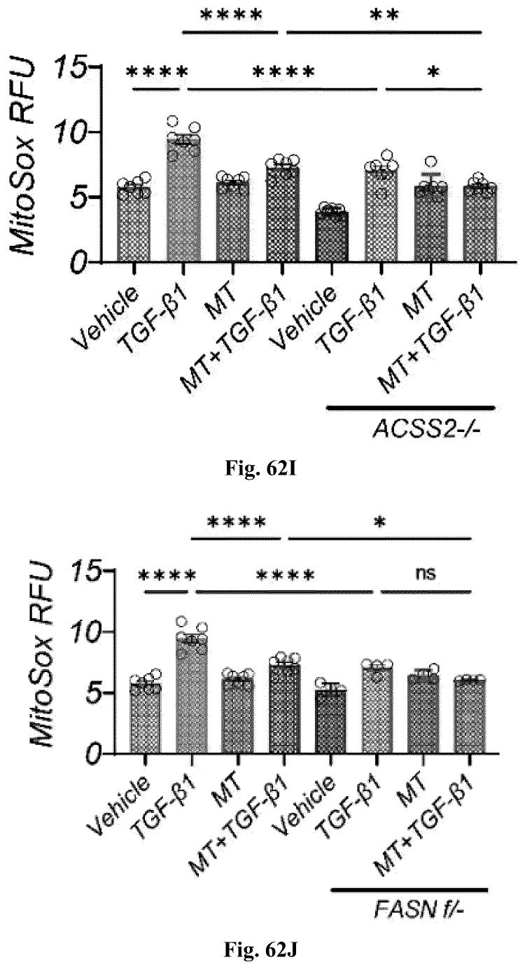

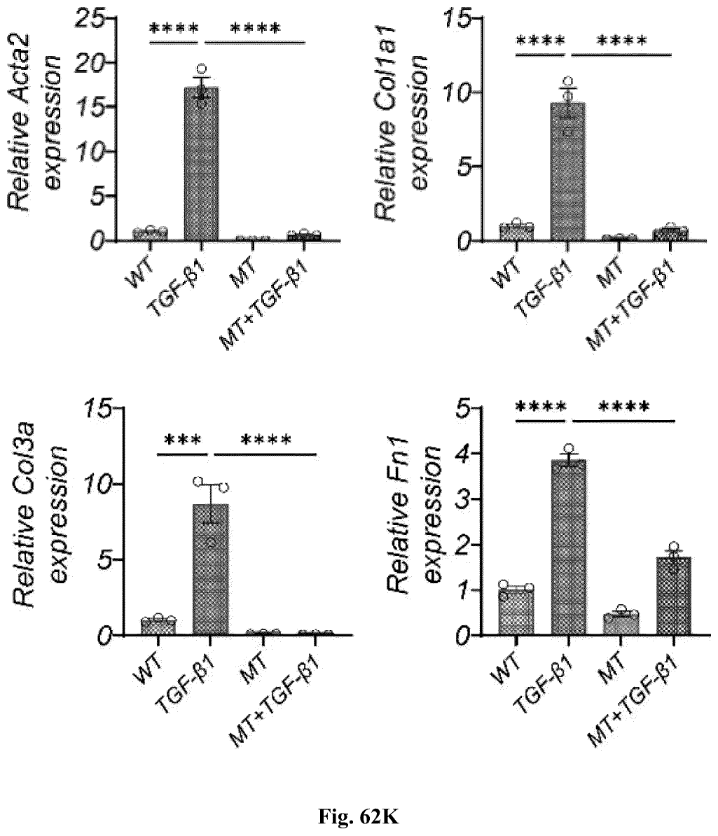

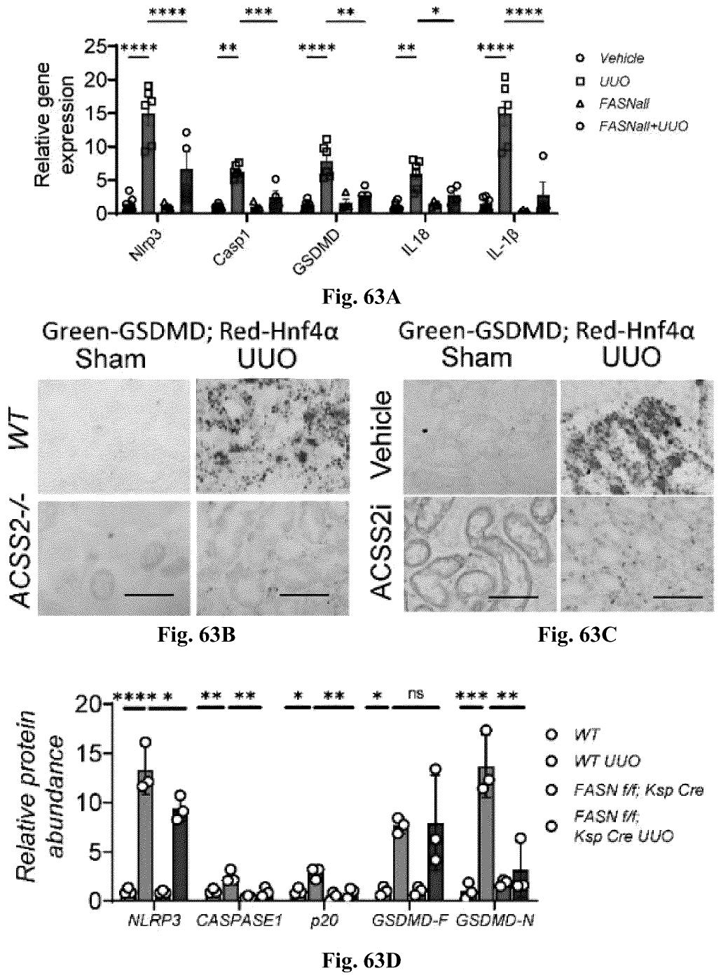

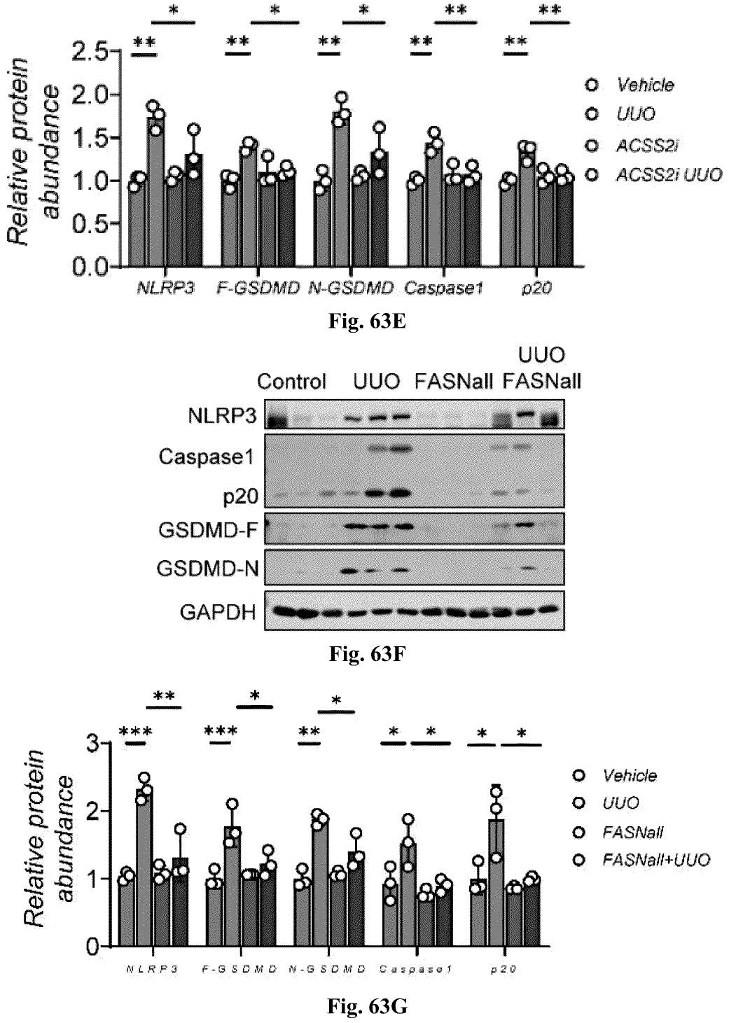

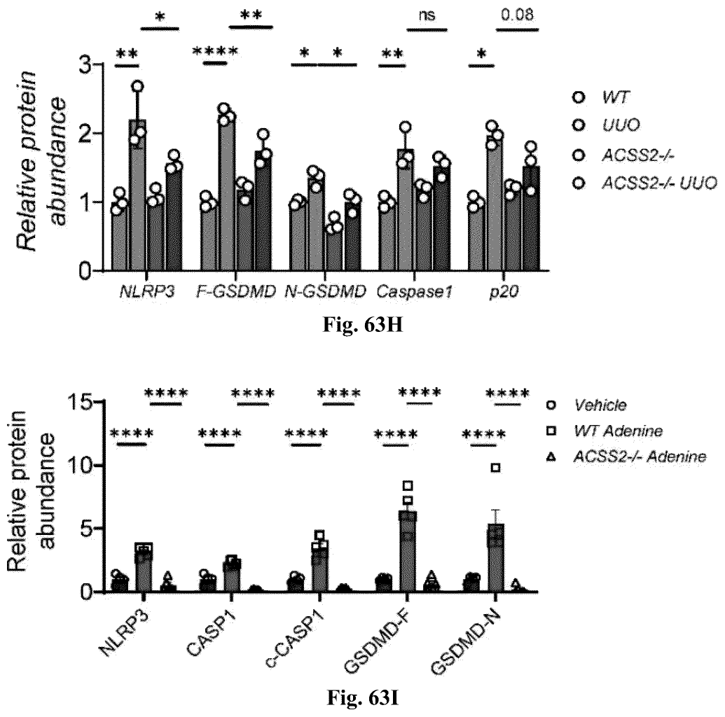

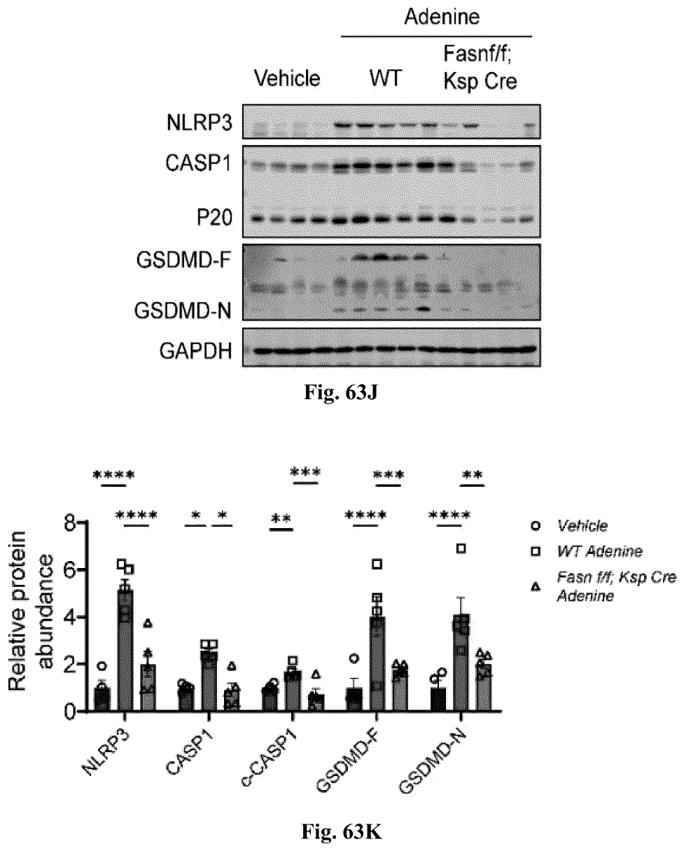

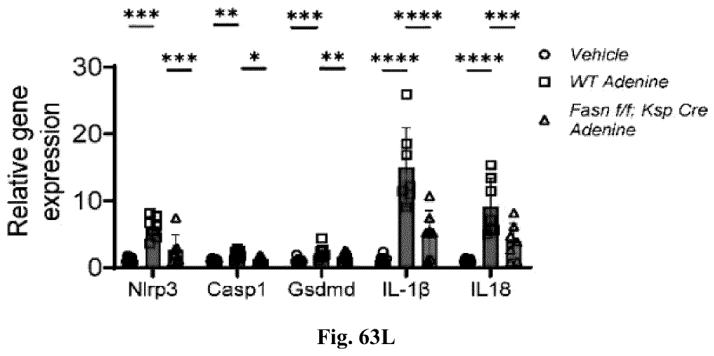

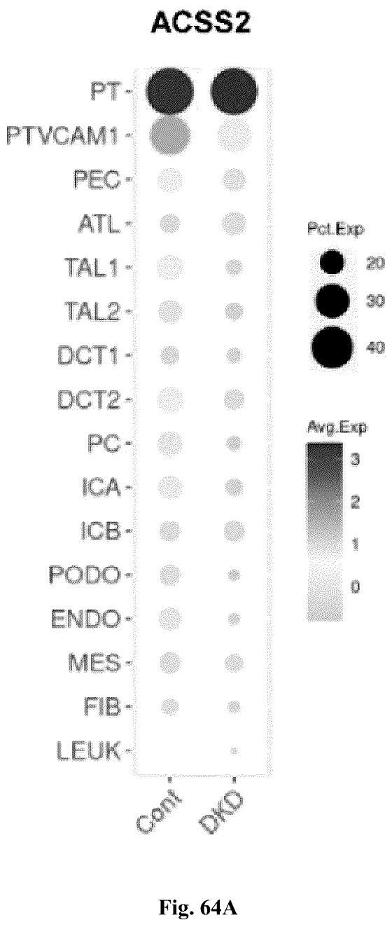

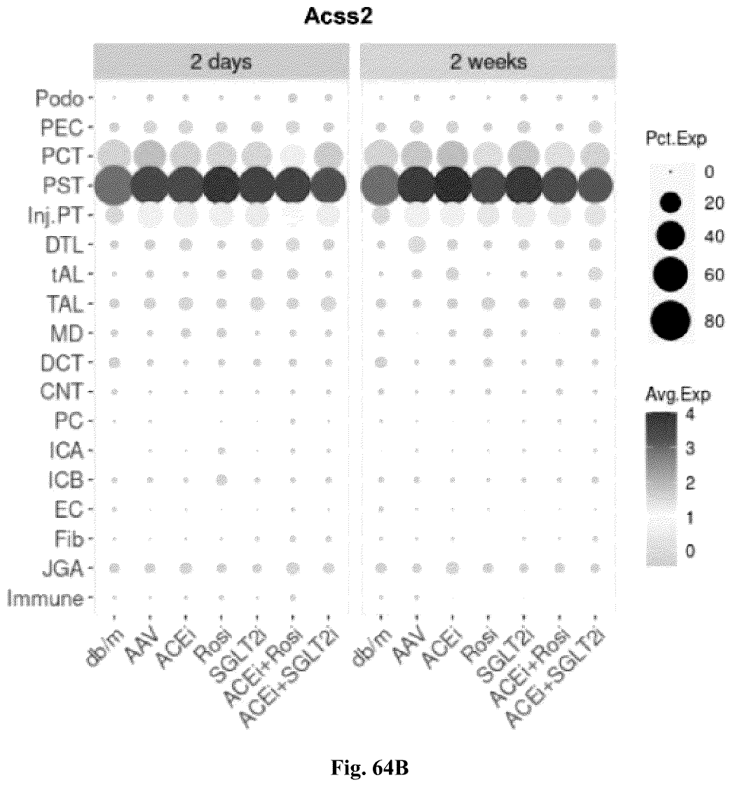

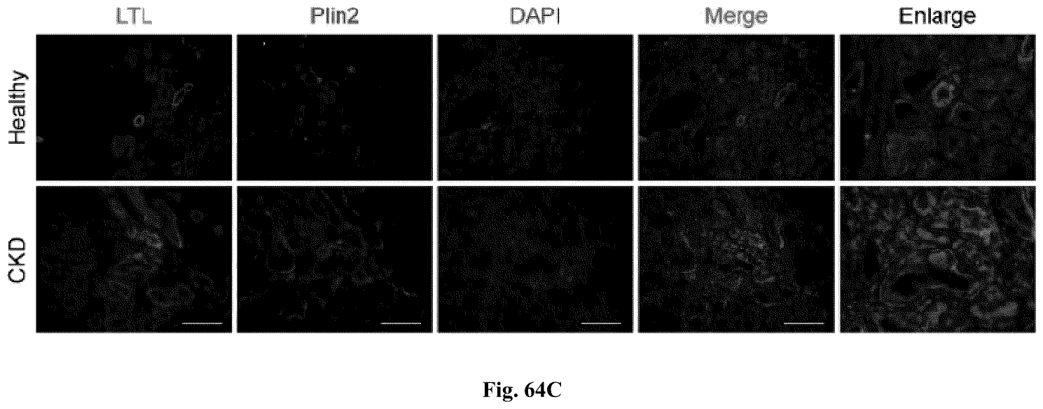

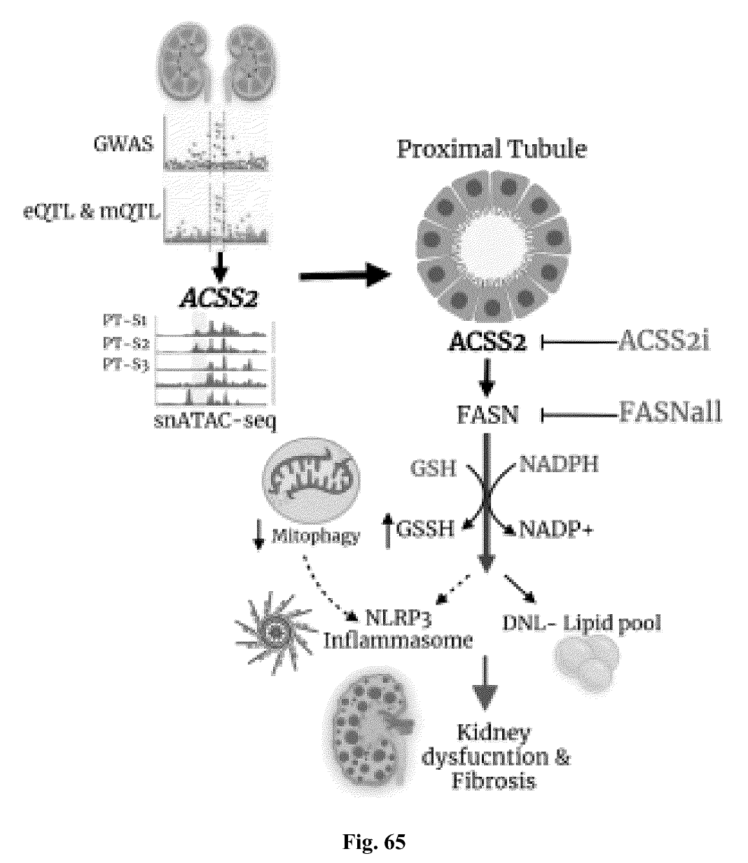

Kidney disease risk allele was associated with a roughly 30% higher CASP9 expression in human kidney tubule samples. Casp9 heterozygous (Casp9 HZ) mice was analyzed with about 50% change in Casp9 expression (FIG. 25A). At baseline, no significant differences were observed in survival, body weight, serum electrolyte, and kidney function parameters such as blood urea nitrogen (BUN) between wild-type (WT) and Casp9 HZ mice at 6 and 24 months of age (FIG. 43).

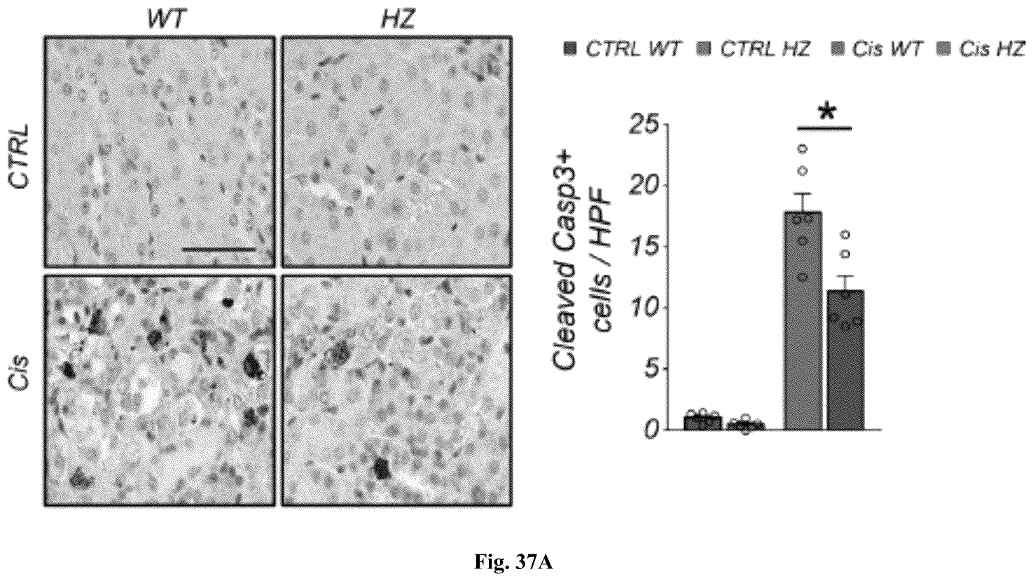

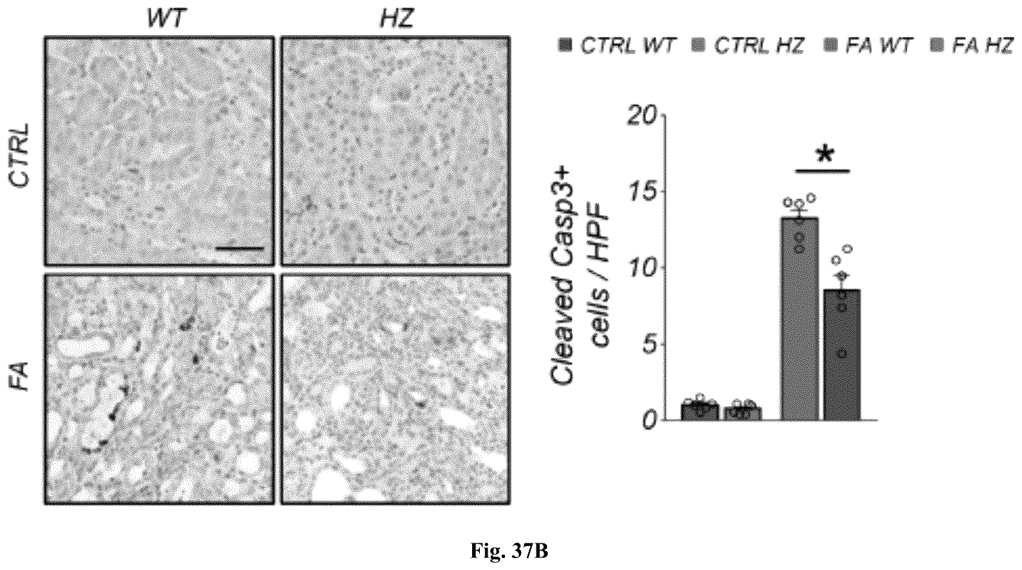

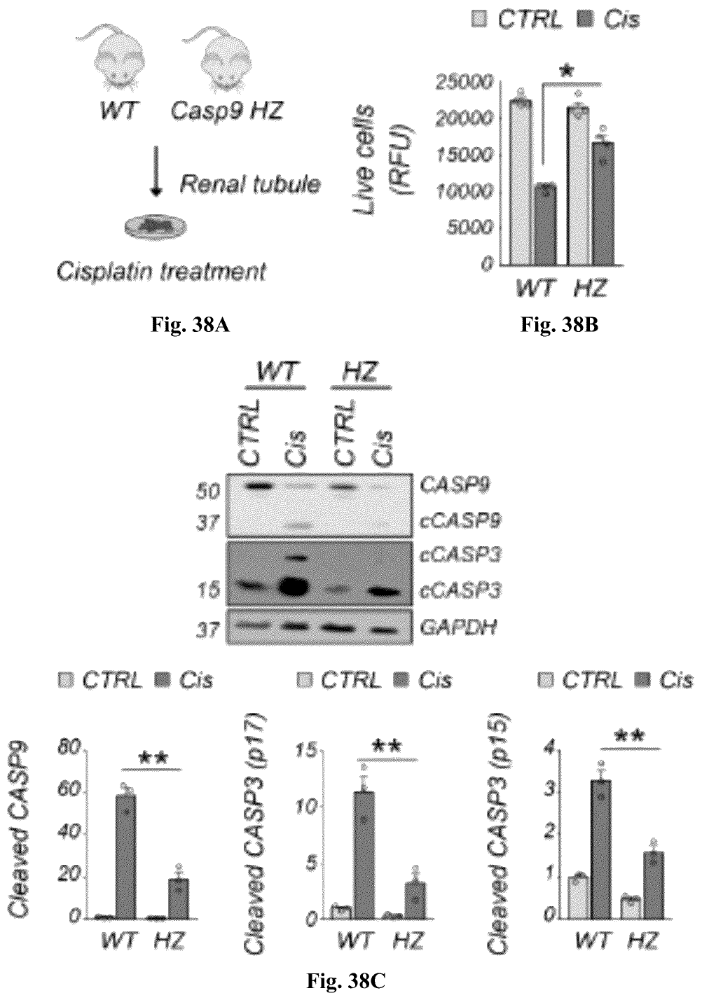

Next analyzed were changes in injury setting, such as induced by cisplatin injection. The kidney function marker BUN was lower in Casp9 HZ when compared to WT mice after cisplatin injection (FIG. 25A-25C). The hepatitis A virus cellular receptor 1 (Havcr1 or Kim1) expression, a typical renal tubular injury marker, was markedly lower in kidneys of cisplatin treated Casp9 HZ mice (FIG. 25D). Kidneys of cisplatin-treated WT mice showed marked tubule epithelial damage, such as loss of brush border, epithelial cell death, tubular dilation, tubular cast, and infiltration of inflammatory cells (FIG. 25E). Renal injury score was lower in cisplatin-treated Casp9 HZ compared to cisplatin-treated WT mice (FIG. 25E). The expression levels of apoptosis markers such as cleaved CASP9 and cleaved CASP3 were lower, and BCL2 was higher in cisplatin-treated kidneys of Casp9 HZ compared to WT mice (FIGS. 25F and 37A). Similarly, cultured Casp9 HZ renal tubule cells showed lower expression of apoptotic caspases and improved viability following cisplatin treatment (FIG. 38A-38C). Although apoptosis per se is a noninflammatory cell death mechanism, expression of cytokines (Il1b, Csf2, Tnfa, and Cxcl10) and Icam1 was also markedly lower in kidneys of cisplatin-injected Casp9 HZ mice compared to cisplatin-injected WT mice (FIG. 25G).

### Example 1-14: Pharmacological Inhibition of CASP9 Ameliorated Cisplatin-Induced AKI

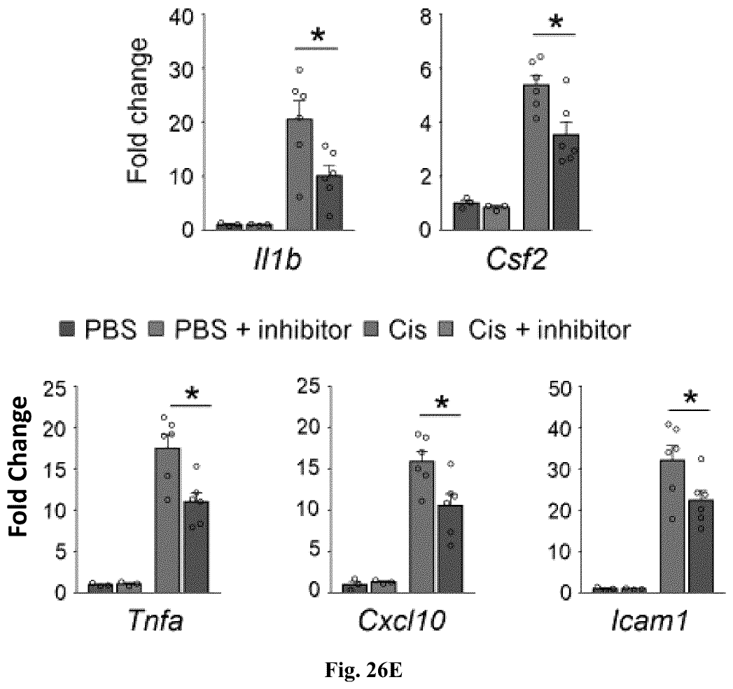

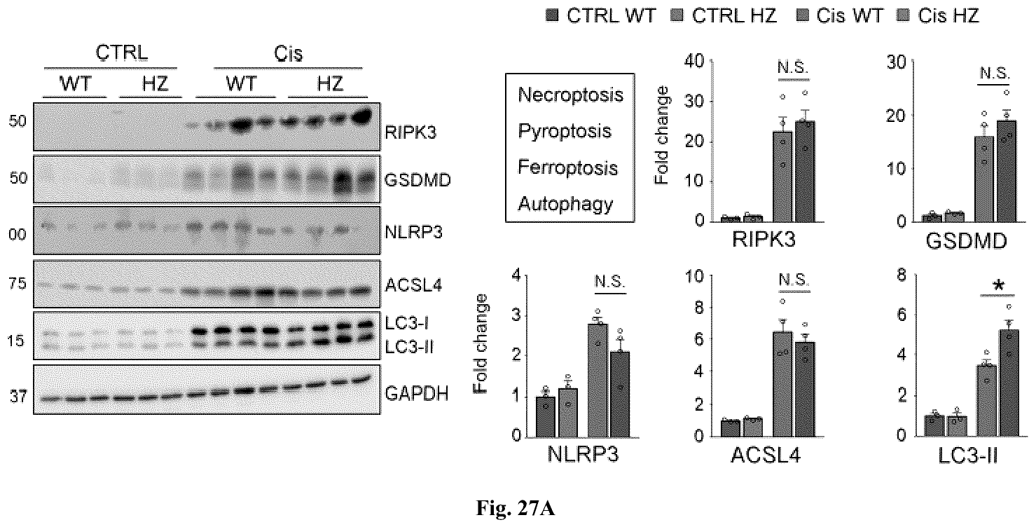

Several small molecular inhibitors that target CASP9 have been developed with good specificity. Effects of Z-LEHD-FMK (a CASP9 inhibitor) in the cisplatin model of kidney injury were investigated. Mice were injected with CASP9 inhibitor or vehicle daily starting 1 day before cisplatin administration. Serum creatinine and BUN levels were lower, and renal expression of Havcr1 was dampened in Z-LEHD-FMK-treated mice. Furthermore, it was also observed that inflammatory cytokines (Il1b, Csf2, Tnfa, and Cxcl10) and Icam1 expression were lower in inhibitor-treated mice after cisplatin injury (FIG. 26A-26E). Collectively, it was found improved kidney function and lessened kidney damage in the cisplatin-injected mice treated with Z-LEHD-FMK.

### Example 1-15: Enhanced Autophagy in Cisplatin Treated Casp9 HZ Mice

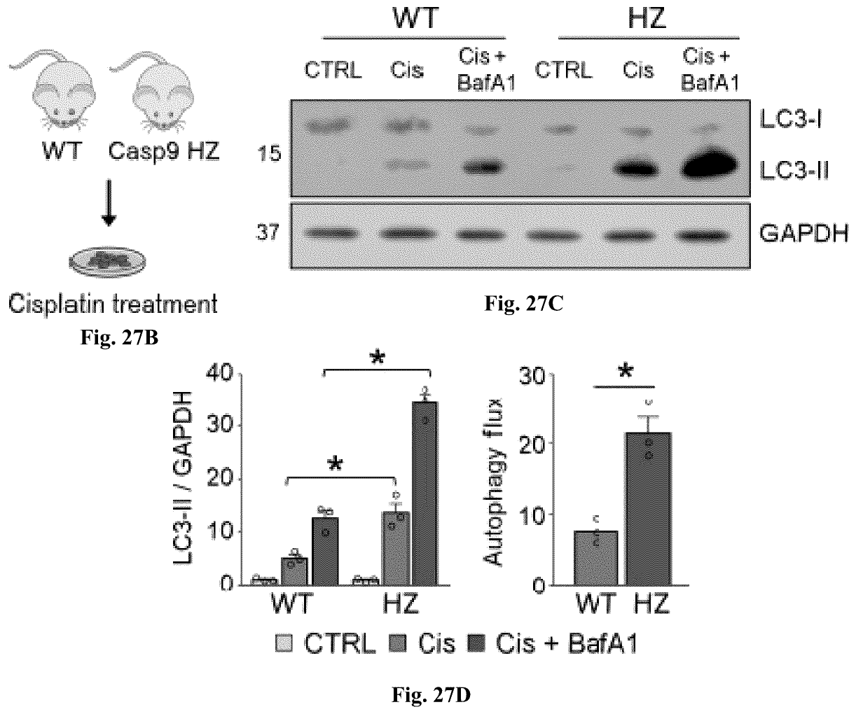

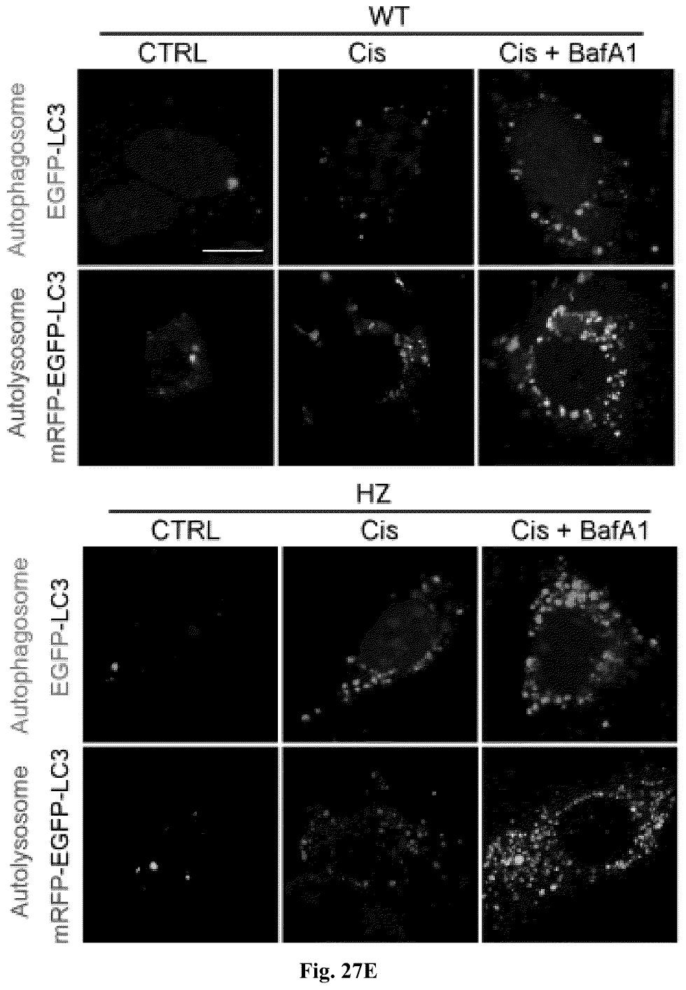

The role of apoptosis in kidney disease development is controversial. While lower apoptosis is associated with cellular preservation, absence of apoptosis can divert to more inflammatory cell death pathways. It was surprising to find that both genetic and pharmacological inhibition of CASP9 was associated with dampened inflammation; therefore, it was decided to investigate changes in inflammatory cell death pathways such as necroptosis, pyroptosis, and ferroptosis. The expression levels of critical regulators of necroptosis (RIPK3), pyroptosis [NLRP3 and gasdermin D (GSDMD)], and ferroptosis [acyl-coenzyme A synthetase long-chain family member 4 (ACSL4)] were higher in kidneys of cisplatin-injected mice. On the other hand, no differences were observed in inflammatory cell death pathways between cisplatin-treated WT and Casp9 HZ mice (FIG. 27A).

At the same time, the expression levels of LC3-II, a key molecule in autophagy, were higher in cisplatin-treated kidneys of WT mice, and it was further increased in cisplatin-treated kidneys of Casp9 HZ mice (FIG. 27A). Therefore, analyzed were autophagy markers in renal tubule cells isolated from WT and Casp9 HZ mice at baseline and following cisplatin treatment. Autophagy flux was examined by treating cells with bafilomycin A1 (BafA1), which interferes with lysosomal function halting autophagosome-lysosomal fusion, enabling the quantification of the rate of autophagy. The LC3-II expression was higher in Casp9 HZ renal tubule cells exposed to cisplatin, and its level further increased following BafA1 treatment (FIGS. 27B,C) indicating an improved autophagy flux in cisplatin-treated Casp9 HZ renal tubule cells (FIG. 27D). To better understand changes in autophagy, renal tubule cells were transfected with enhanced green fluorescent protein (EGFP)-LC3 plasmid. Autophagosome number, represented by EGFP-positive dots, was markedly higher in Casp9 HZ renal tubule cells exposed to cisplatin, which again further increased following BafA1 treatment (FIGS. 27E and 27F). The mRFP-EGFP-LC3 plasmid is another tool to evaluate autophagy flux by using different pH stability of GFP and red fluorescent protein (RFP). Under acidic conditions such as autolysosome, the GFP signal is quenched and only the RFP signal is visible. Autolysosome numbers indicated by RFP puncta were observably higher in Casp9 HZ renal tubule cells exposed to cisplatin (FIGS. 27E and 27F). BafA1 treatment restored the GFP signal.

### Example 1-16: Suppression of the Cytosolic Nucleotide Sensing Pathways (cGAS and STING) by Mitophagy

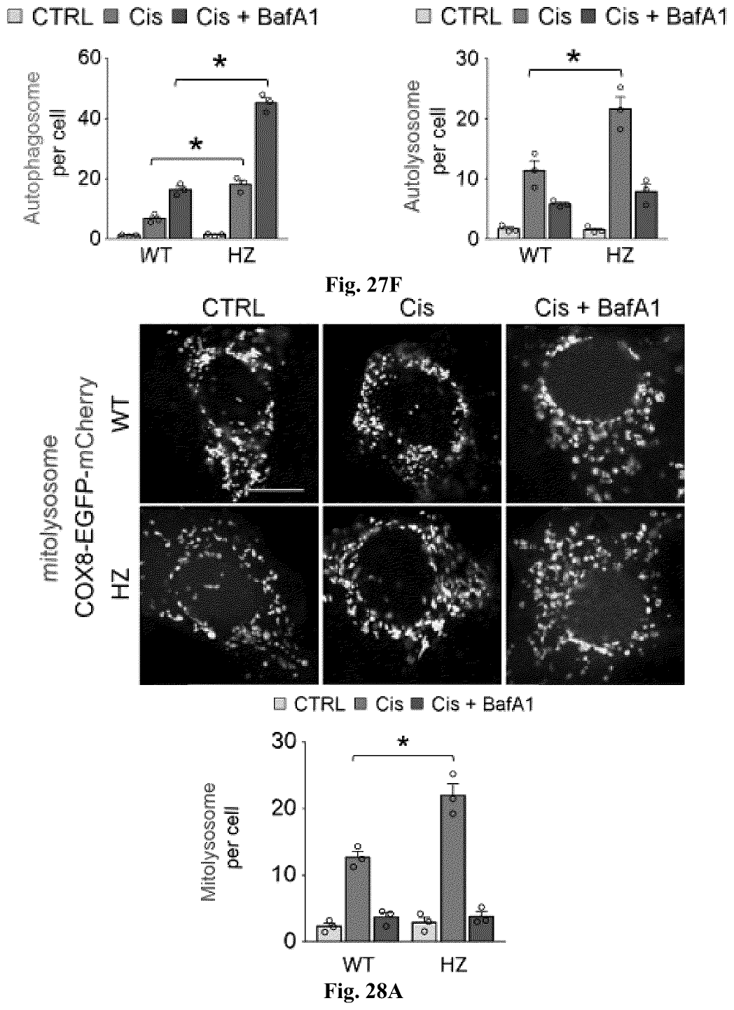

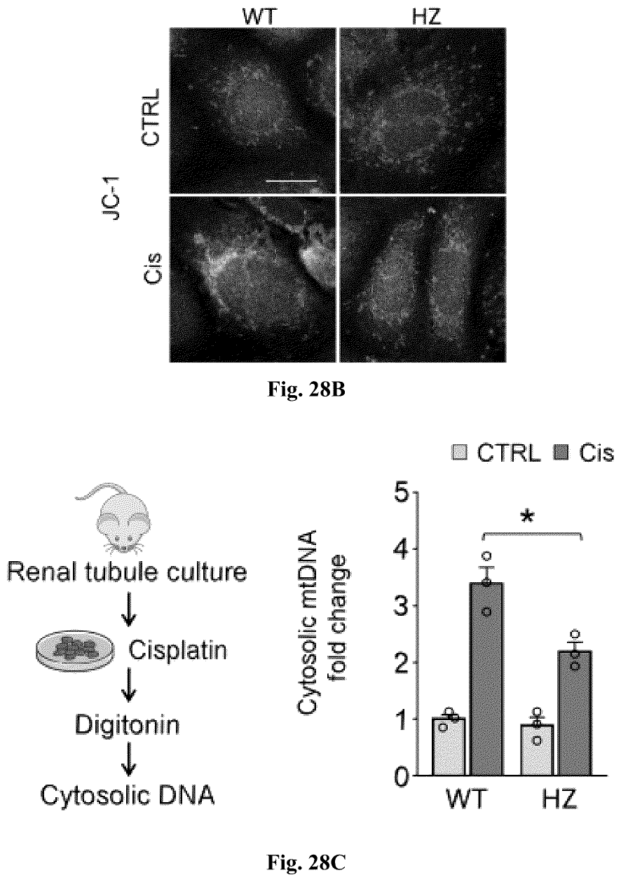

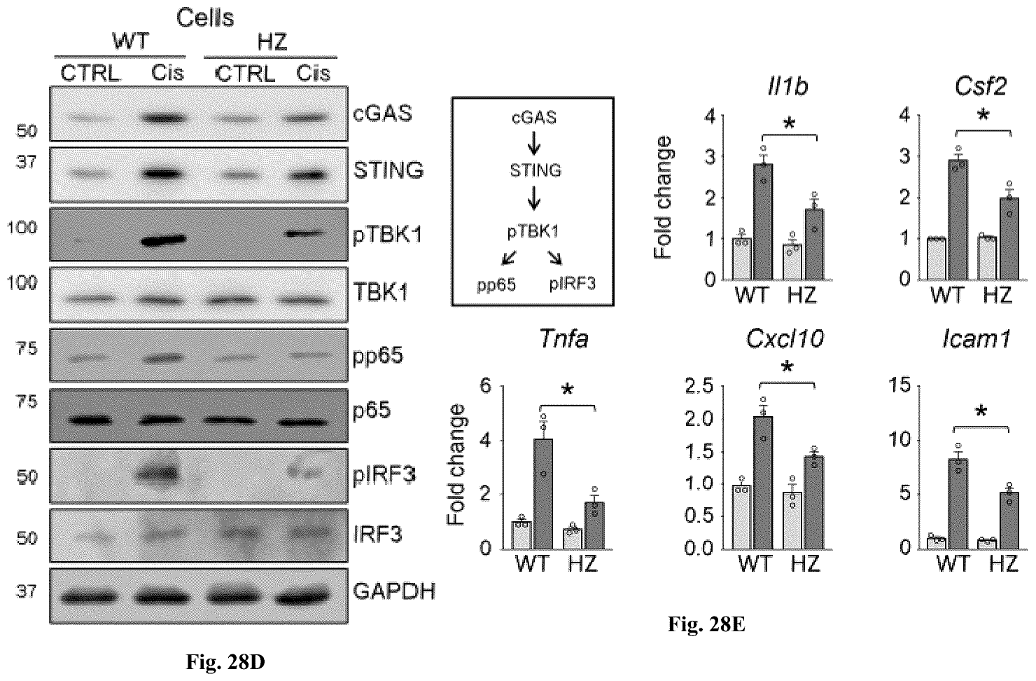

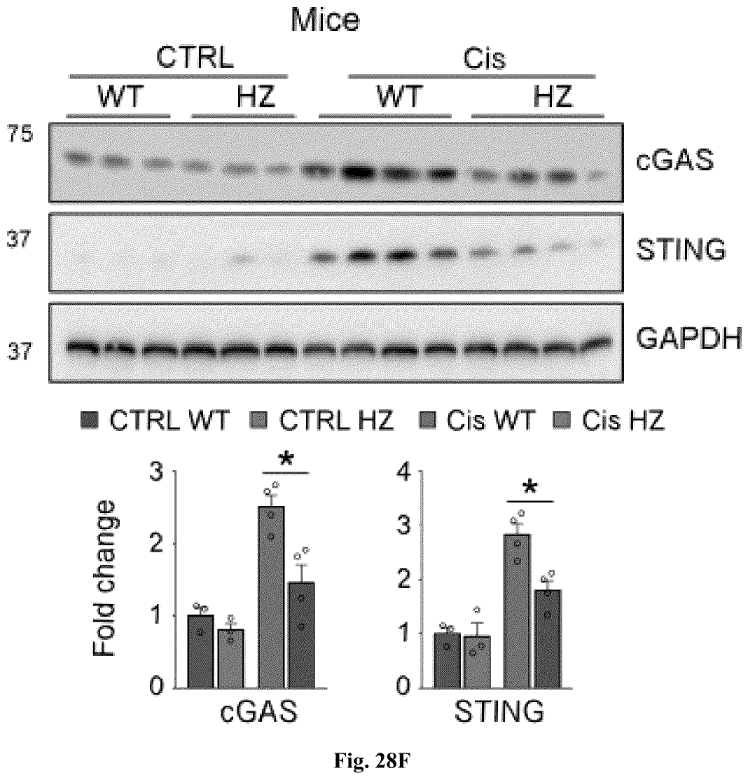

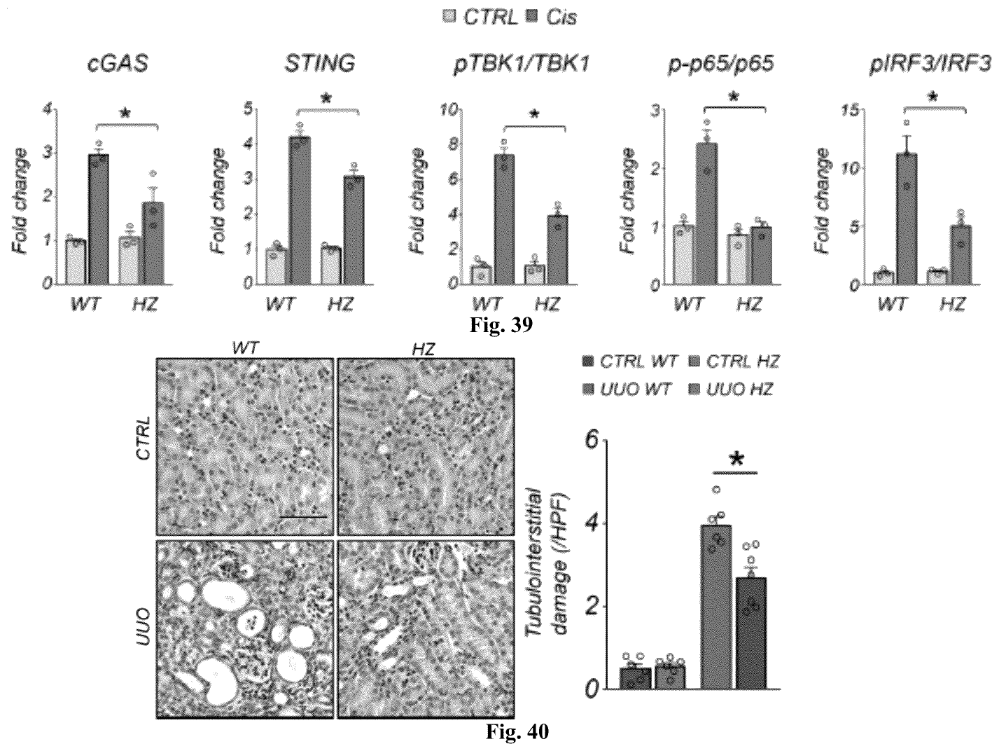

As changes were observed in autophagy, next analyzed was mitophagy using COX8-EGFP-mCherry plasmid. While cisplatin induced a severe mitophagy defect, mitolysosomes represented by the mCherry signal were markedly higher in Casp9 HZ renal tubule cells exposed to cisplatin (FIG. 28A). Cisplatin induced marked mitochondrial depolarization in kidney tubule cells, which was ameliorated in Casp9 HZ renal tubule cells (FIG. 28B). The cisplatin-induced mitochondrial damage has been shown to release mitochondrial DNA, which is sensed by the cytosolic nucleotide sensing pathways such as cGAS (cyclic GMP-AMP synthase) leading to STING (stimulator of interferon genes) activation. It was found that the cytosolic mitochondrial DNA release was lower in Casp9 HZ renal tubule cells when compared to WT following cisplatin treatment (FIG. 28c). Subsequently, protein levels of cGAS and STING were lower in cisplatin treated Casp9 HZ renal tubule cells. Downstream signaling molecules, such as phosphorylated TANK-binding kinase 1 (pTBK1), phosphorylated p65 (pp65), and phosphorylated interferon regulatory factor 3 (pIRF3) were lower in cisplatin-treated Casp9 HZ renal tubule cells (FIGS. 28D, 28E, 39A). Furthermore, expression of downstream proinflammatory cytokines such as Il1b, Csf2, Tnfa, Cxcl10, and Icam1 was also lower in cisplatin treated Casp9 HZ renal tubule cells (FIG. 28F).

Last, validated were the changes observed in cytosolic nucleotide sensing pathways in kidneys of cisplatin-treated WT and Casp9 HZ mice. Protein levels of cGAS and STING were also lower in cisplatin-treated Casp9 HZ mice when compared to cisplatin-treated WT mice (FIG. 28F).

### Example 1-17: Casp9 HZ Mice are Protected from Kidney Fibrosis

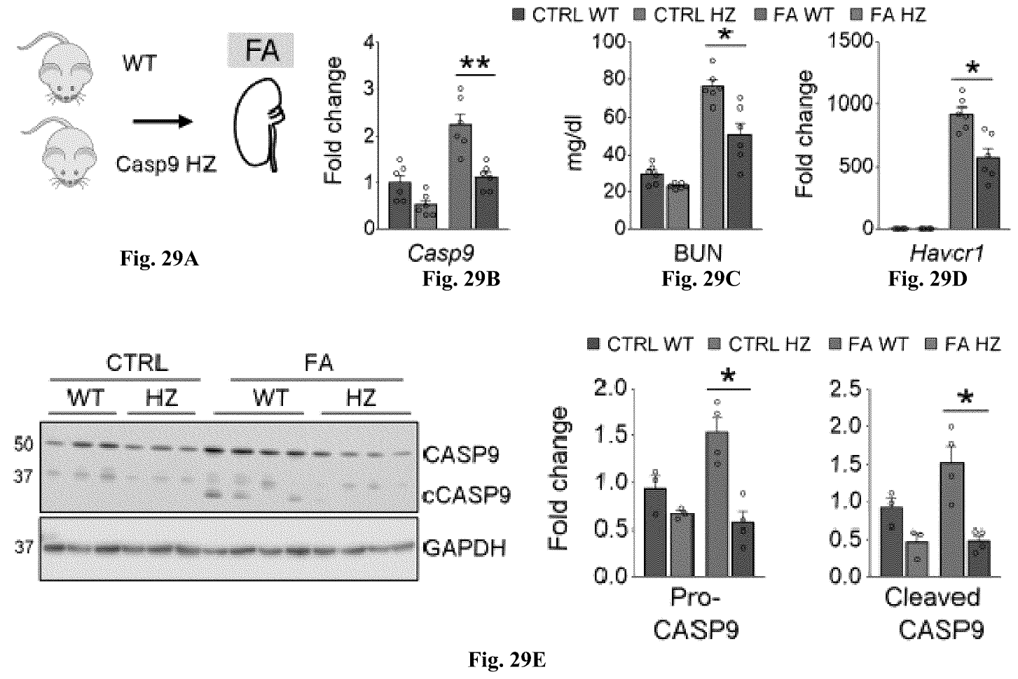

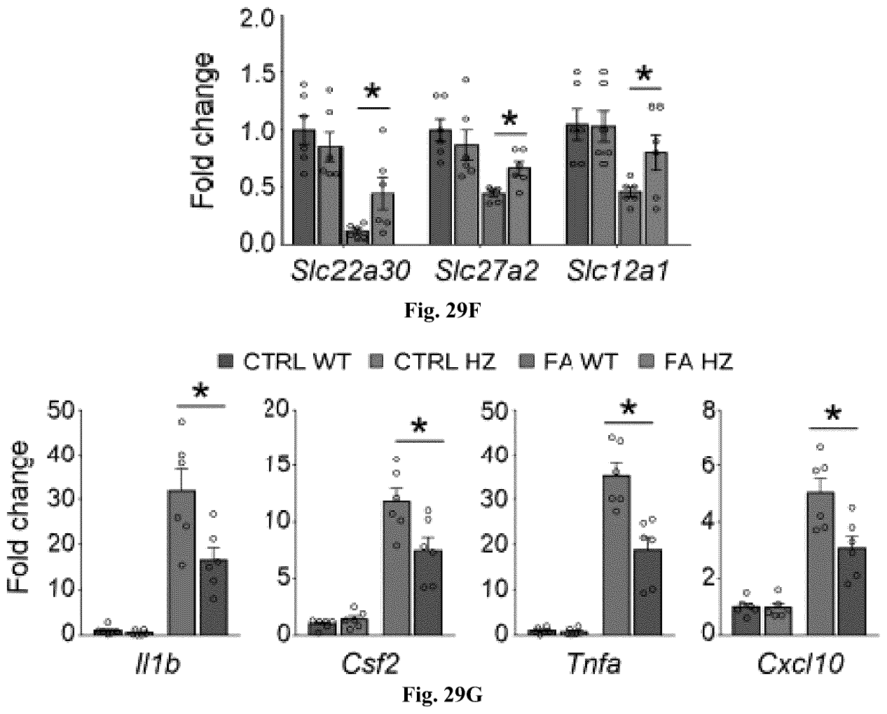

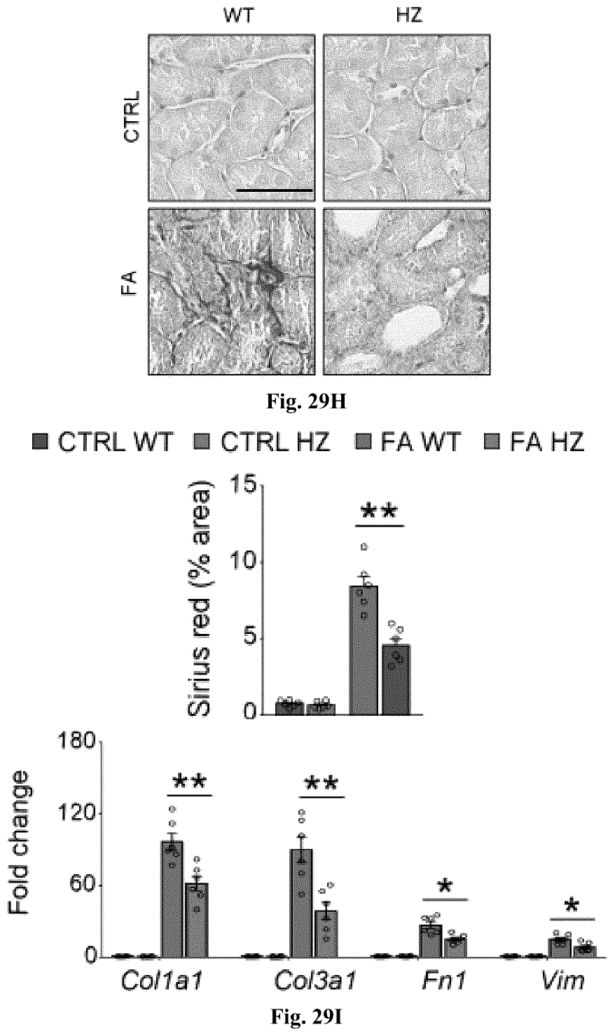

While these studies using the cisplatin, model demonstrated the role of CASP9 in AKI, next examined was whether CASP9 plays a role in kidney fibrosis, a feature of CKD. The FA injection crystal precipitation mode was evaluated as an increase in kidney Casp9 levels in was earlier found in this model (FIGS. 24B and 29A, 29B). It was found that markers of kidney function (BUN) and expression of tubule injury markers (Havcr1) were improved in FA-treated CASP9 HZ mice (FIGS. 29C and 29D). An increase was observed in expression of proapoptotic proteins such as CASP9 and cleaved CASP3 in kidneys of FA-treated mice, which were attenuated in Casp9 HZ mice (FIGS. 29B, 37B). Consistent with the lower apoptosis rate, expression of proximal and distal tubule marker genes (Slc22a30, Slc27a2, and Slc12a1) were preserved in the FA-treated Casp9 HZ mice (FIG. 29F). At the same time, expression of inflammatory cytokines (Il1b, Csf2, Tnfa, and Cxcl10), renal fibrosis, and profibrotic genes (Col1a1, Col3a1, Fn1, and Vim) was lower in the FA-treated Casp9 HZ mice (FIG. 29G-29I).

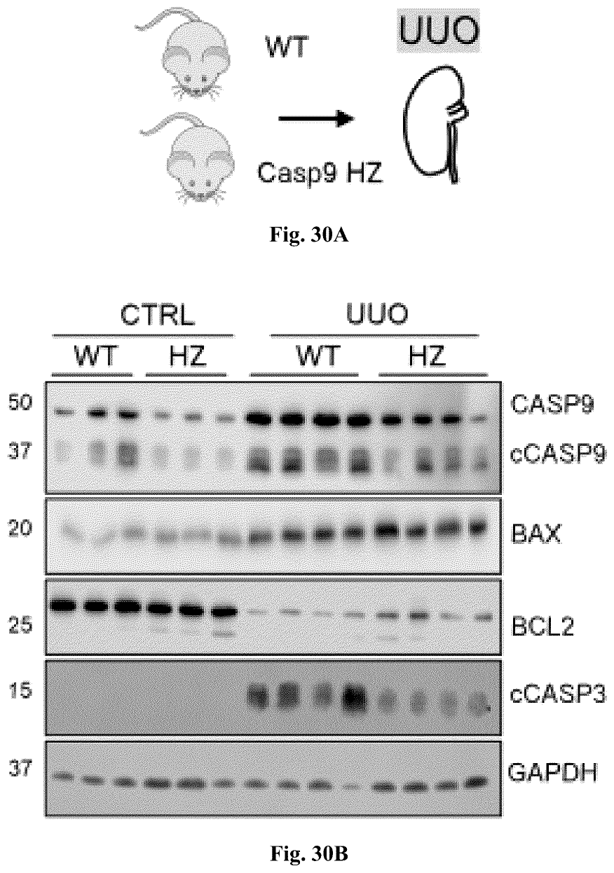

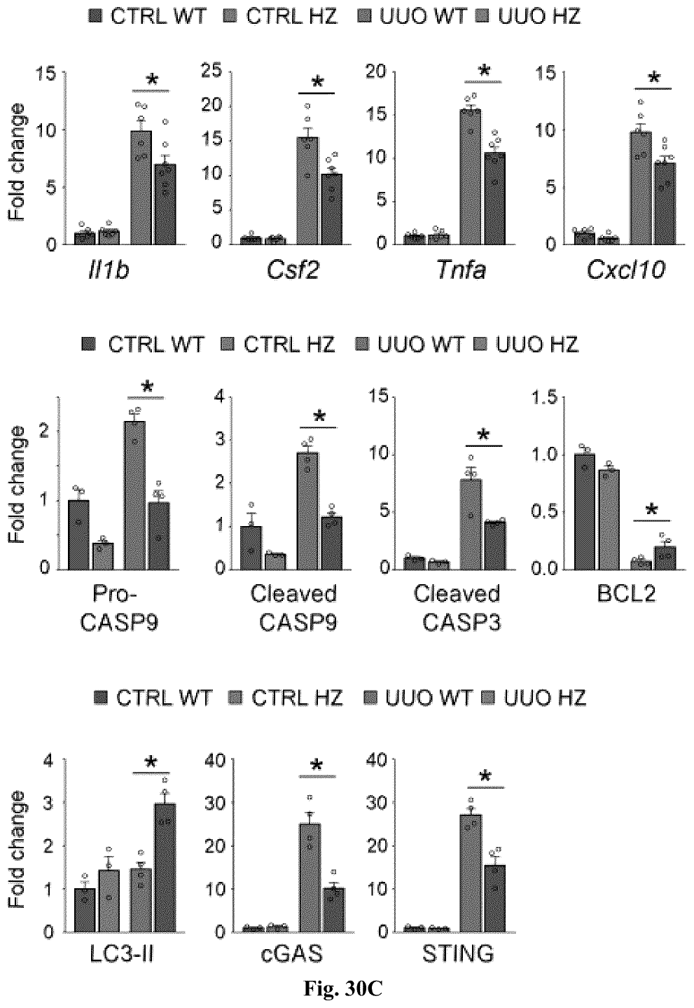

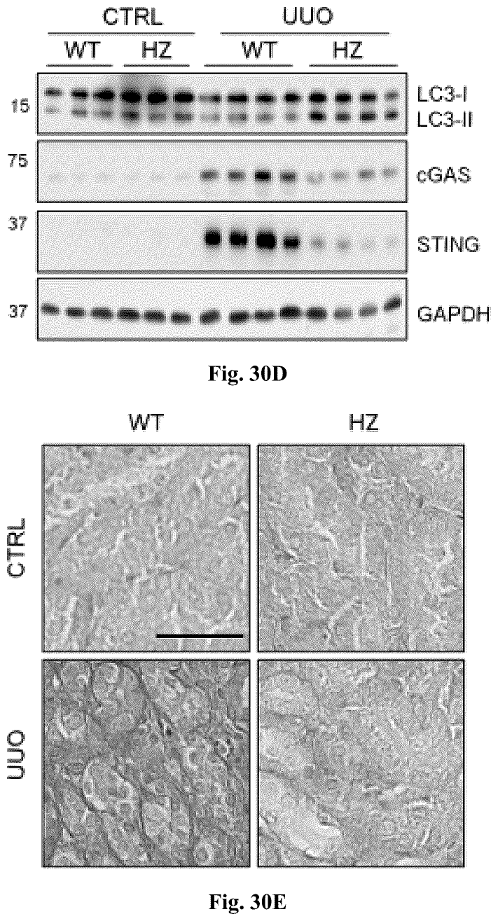

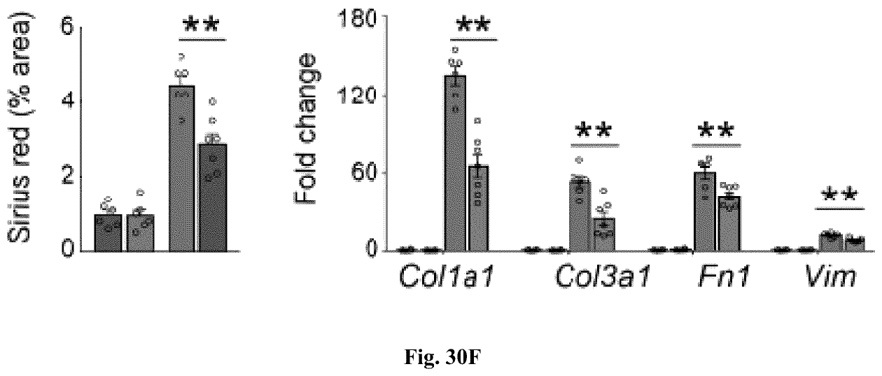

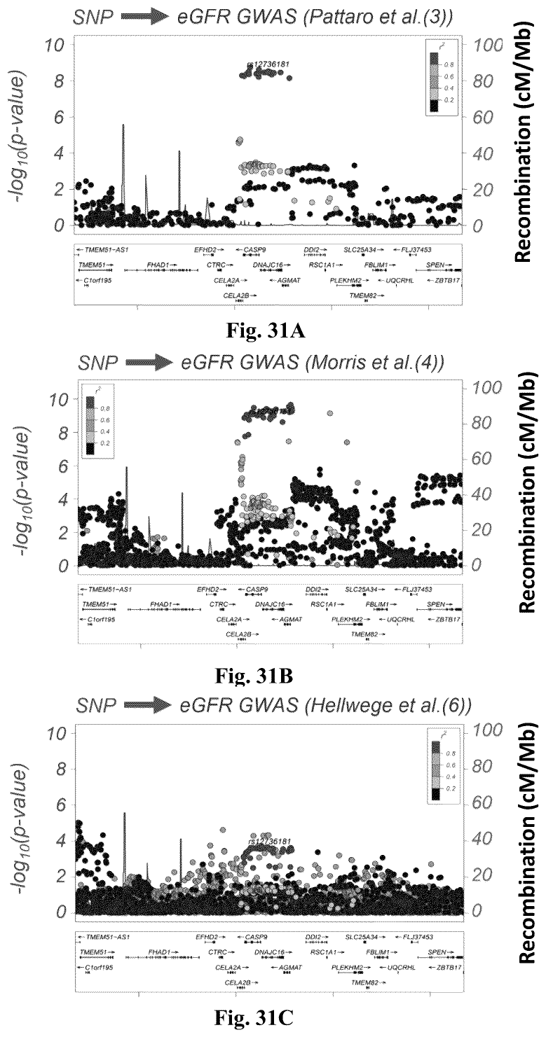

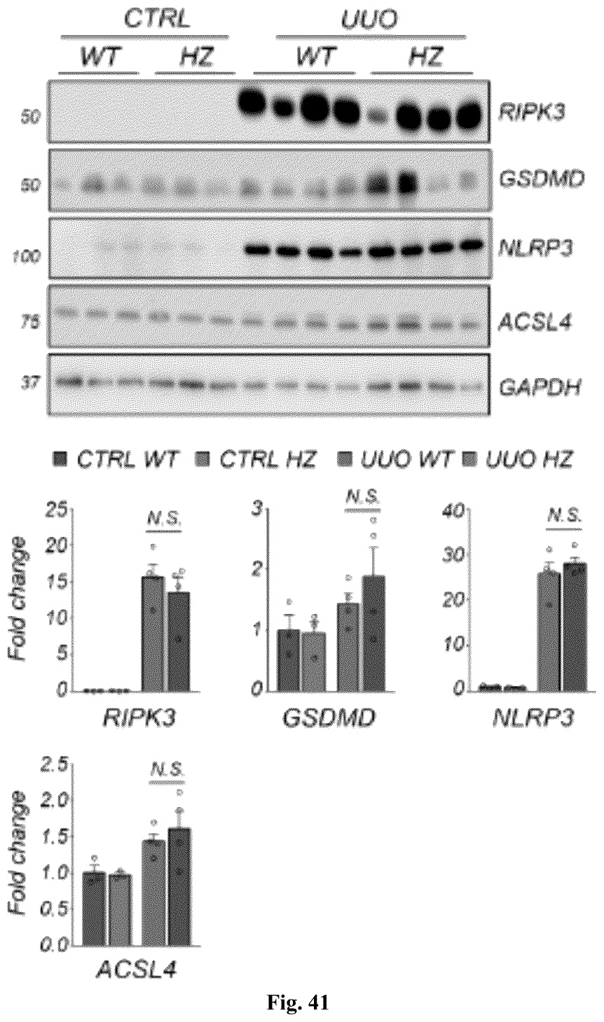

Similarly, less tubule injury and marked differences in apoptosis were observed in Casp9 HZ mice following UUO (FIGS. 30A, 30B and 40A). The expression of inflammatory cytokine levels (Il1b, Csf2, Tnfa, and Cxcl10) was lower in UUO kidneys from Casp9 HZ mice (FIG. 30C). There were no observed differences in necroptosis, pyroptosis, and ferroptosis regulators (examined by RIPK3, GSDMD, NLRP3, and ACSL4) in UUO kidneys when Casp9 HZ and WT mice were compared (FIG. 41A). The higher LC3-II levels in UUO kidney of Casp9 HZ mice were consistent with improved autophagy (FIG. 40D). Kidney expression of cGAS and STING was lower in UUO Casp9 HZ mice (FIG. 40D). Last, the lesser inflammation was associated with lower renal fibrosis and expression of profibrotic genes (Col1a1, Col3a1, Fn1, and Vim) in UUO kidney of Casp9 HZ mice (FIGS. 30E,30F).

### Example 1-18

Here, it is demonstrated that CASP9 is a kidney disease risk gene prioritized by a variety of multiomics approaches. Follow-up in vitro and in vivo studies indicate that CASP9 not only lowered renal tubule cell apoptosis but also improved mitophagy, resulting in the reduction of cytosolic mitochondrial DNA, cytosolic nucleotide sensing pathways (cGAS and STING), downstream inflammation, and fibrosis development.

While GWAS identifies risk regions in the genome, orthogonal datasets are needed for the functional interpretation of GWAS risk variants, such as kidney eQTL and single-nuclear epigenetic information. Bayesian colocalization analysis revealed that the GWAS risk variants had a shared effect on both eGFR and kidney tubule CASP9 expression levels, where higher CASP9 expression levels were associated with lower eGFR levels. SMR and TWAS analysis indicated that CASP9 expression mediated the effect of the genotype on kidney function. Integration of Bayesian colocalization signal with single-nuclei open chromatin information prioritized likely causal variant and indicated tissue-specific (kidney), and cell type-specific (tubules) regulation of CASP9 expression. CRISPR-Cas9 gene editing confirmed the functional role of the open chromatin region containing likely causal variants.

Here, it is shown that kidney Casp9 mRNA expression levels correlated with fibrosis severity. Higher Casp9 expression and activity and more severe fibrosis were observed in the FA and UUO models compared to UNx-STZ and aging mice. The FA and UUO models are widely recognized as kidney fibrosis models, while the diabetic and aging models only show mild histological changes and lack of GFR decline. CASP9 expression in proximal tubules of FA and UUO mice and patients with CKD supports the key role of proximal tubule in kidney disease development.

Via the use of mouse genetic models, it is demonstrated that the causal role of CASP9 in AKI, fibrosis, and inflammation, rendering CASP9 a kidney disease risk gene. Genetic deletion or pharmacological inhibitor of CASP9 lowered apoptosis and protected from acute injury induced by cisplatin and kidney fibrosis induced by FA and UUO. The protective role of CASP9 in kidney disease development can be explained by two mechanisms. First, apoptosis seems to contribute to tubule epithelial cell loss, such as proximal tubule and the loop of Henle cells. Preserved epithelial cell number likely results in functional preservation; however, as epithelial cells can regenerate, this might not fully explain the protection from disease development.

Second, the observations indicate that mechanisms outside of apoptosis play an important role in the CASP9-afforded protection. The Casp9 expression and CASP9 activity did not fully correlate with classic apoptosis genes such as Bax and Apaf1 levels. The present study observed improved mitophagy in Casp9 HZ renal tubule cells treated with cisplatin and UUO model of kidney fibrosis. Defect in mitophagy has been associated with cytosolic leakage of mitochondrial DNA and activation of the cytosolic nucleotide sensing pathways following subsequent inflammation. Marked differences were observed in cGAS and STING activation and lower inflammation in Casp9 HZ renal tubule cells and mice. These observations are consistent with previous reports showing the pathogenic proinflammatory role of the cGAS-STING pathway in kidney fibrosis.

In summary, CASP9 as an eGFR GWAS target gene were prioritized using computational and experimental tools, as well as cellular and animal models. It is demonstrated that the causal role of CASP9 in kidney disease development is via improving mitophagy and lowering inflammation and apoptosis. These results open new avenues for kidney disease therapeutics via the use of small molecular CASP9 inhibitors.

### Example 2: Acetyl coA Synthetase 2 (ACSS2) as a New Therapeutic Target for Kidney Disease

### Example 2-1: Material and Methods

### Prioritization of Kidney Disease Genes

The method for prioritizing and identifying target genes for kidney function GWAS loci comprises a priority scoring strategy by integrating evidence from any and/or all eight different datasets or analytical tools including significant SNP-gene associations by kidney eQTLs (FDR<0.05); (2) significant SNP-CpG-gene associations by kidney meQTLs (FDR<0.05) and eQTM (CpG-level FDR<0.05); (3) SNP-gene pairs by coloc analysis between eGFRcrea GWAS and eQTLs (H4>0.8); (4) SNP-gene pairs by moloc analysis among eGFRcrea GWAS, eQTLs and meQTLs (PPA.abc>0.8); (5) significant SNP-gene pairs by Mendelian randomization analysis between eGFRcrea GWASs and eQTLs (PSMR<1.38×10-4); (6) SNP-gene pairs passing the HEIDI test between eGFRcrea GWAS and eQTLs (PHEIDI>0.01); (7) co-accessibility (Cicero connections) identified using 57,229 snATAC-seq cells (co-accessibility score>0.2); and (8) enhancer-promoter contacts identified by an ABC model that predicts enhancers regulating genes based on estimating enhancer activity and enhancer-promoter contact frequency from epigenomic datasets (ABC scores>0.015). Promoters were defined as ±2,000 bp from the TSS of protein-coding transcripts from GENCODE v.351ift37 to annotate Cicero connections or ABC connections between gene promoters and eGFRcrea GWAS variants.

For each significant eGFRcrea GWAS variant, protein-coding genes were extracted within 1 Mb of the SNP as potential targets. For each SNP-gene pair, a priority score was defined by counting the number of datasets supporting the association. For each variant, the gene with the highest priority score was assigned as its target gene. If multiple genes shared the highest priority score, the closest gene with the most significant eQTL was assigned as the target gene. For each independent locus, the top target gene was determined according to the highest priority score from all variant gene pairs in the same locus. If multiple genes shared the highest priority score, the gene targeted by the variant with the most significant GWAS association was assigned as the top target gene for the locus. Newly prioritized loci were defined if they did not overlap with 309 independent signals (using gene prioritization score≥1) prioritized in eGFRcrea GWAS by Stanzick et al. (Stanzick, K. J. et al.; Nat. Commun. 12, 4350 (2021)) or 53 creatinine-associated exome rare variants identified in exome association studies by Backman et al. (Backman, J. D. et al. Nature 599, 628-634 (2021). or Barton et al. (Barton, A. R., Nat. Genet. 53, 1260-1269 (2021).

Furthermore, 328 GWAS loci with 559 target genes with a priority score of at least 3. First, 110 loci were inspected with 2 or more target genes by counting the number of independent signals (fine-mapped in 1 million European ancestry individuals) and co-expression gene pairs (FDR<0.05 accounting for all correlation tests) for each locus. To explore the function of prioritized genes, gene-set enrichment was performed for tissue specificity and GWAS catalog genes using GENE2FUNC of FUMA (Watanabe, K. et al., Nat. Commun. 8, 1826 (2017)). with protein-coding genes as a background gene set. Functional enrichment analysis for these genes was performed using DAVID Bioinformatics Resources (v.6.8) (Huang da, W., Nat. Protoc. 4, 44-57 (2009)). For enrichment to the cell type-specific genes, mouse orthologues were obtained and overlapped with cell type-specific expressed genes identified using mouse single-cell RNA-seq (Park, J. et al. Science 360, 758-763 (2018)). The cell-type enrichment significance was determined using a hypergeometric test.

Further details of epigenomic and transcriptomic analyses that define core cell types, genes and targetable mechanisms for kidney disease are presented in Liu et al. Nature Genetics 54.7 (2022): 950-962 and incorporated herein in their entireties. A list of genes that were identified as the genes that cause kidney disease is as shown in Table 1 above.

### Generation of Mouse Models to Study Effects of Alerted ACSS2 Levels.

The genetic locus containing ACSS2 was identified as being important for further study by expression quantitative trait loci (eQTL and mQTL) analysis. eQTL and mQTL analysis uses computational integration (coloc and moloc) to combine genomic data with transcriptomic data. Then, to see which kidney cell types express the genetic variants associated with ACSS2 expression, kidney single cell RNA and ATAC-seq data were integrated. To study the effects of altering ACSS2 levels, mice with a targeted deletion of exon 1 of ACSS2 were generated, using CRISPR technology. These mice were studied at baseline (i.e. without inducing injury) and after inducing injury. The injury models chosen included folic acid (a model of chronic nephropathy) and Unilateral ureter obstruction (UUO). The findings were also validated in vitro, through analysis of primary tubule cells isolated from control (WT) and knockout (Acss2−/−) mice.

### Example 2-2: Prioritization of ACSS2 as a Kidney Disease Gene

In the study, the focus was on chromosome 20 eGFR GWAS locus, as it showed a strong association with kidney function FIGS. 46A-46C). Regional plot of eGFR GWAS indicated significant association of genetic variants on chromosome 20 with eGFR (rs11698977, P=3.566×10-58) FIG. 46A. The same genetic locus also showed significant association with ACSS2 expression in microdissected renal tubules (P=3.01×10-9) (rs2295352), glomeruli (P=3.09×10-8). A SNP (rs2424999) in linkage disequilibrium with the causal SNP (rs11698977). On the contrary, an association between the genetic variant (rs11698977) and expression of neighboring genes to ACSS2 was observed. To determine if two traits (eGFR and ACSS2 expression) share causal variants at this locus, Bayesian colocalization analysis was conducted using coloc and moloc analysis. It was discovered that variants associated with kidney function and ACSS2 expression in kidney tubules were shared. While multiple variants showed association with eGFR and ACSS2 expression, SNPs located in the regulatory regions such as enhancers are more likely to be causal. Therefore, human kidney single-nuclear assay was used for transposase-accessible chromatin (snATAC) data to fine-map the region and further prioritize risk variants. Variants that overlapped with open chromatin regions were prioritized. With this prioritization strategy the likely causal SNPs to further co-localize within the kidney tubule were narrowed, more specifically within the Proximal Tubule).

### Example 2-3: Targeted Deletion of ACSS2 Protected from Kidney Fibrosis

To understand the role of ACSS2 in the kidney, CRISPR mediated genetic knockout mice were generated. These mice were born at normal Mendelian ratio, no birth defects and no signs of proteinuria or kidney function defects at adultery. No growth defects were seen during life span of 50 wk of aged mice. To understand whether ACSS2 has any role during kidney disease particularly, tubule interstitial fibrosis, UUO surgery and folic acid induced nephropathy were performed. Both models are useful to study the fibrotic kidney disease. Gene and protein expression analysis confirmed the reduction in ACSS2 expression compared to littermate controls. Moreover, mRNA and protein levels of ACSS2 were further reduced with kidney injury in both UUO and FAN models in WT mice. Fibronectin (FN1) and smooth muscle actin (α-SMA), the most widely studied fibrosis markers, protein levels were comparatively similar with no significance in both WT and Acss2−/− mice without kidney injury. However, FN1, α-SMA levels were significantly elevated in UUO and FAN model and deletion of ACSS2 reduced their expression. Gene expression analysis of fibrosis markers including collage type1a1 (Col1a1), collagen type 3a (Col3a) and fibronectin (Fn1) were comparative between the WT and Acss2−/− mice in SHAM kidneys. However, their expression was elevated in UUO and FAN, both models and this elevation was diminished in mice with ACSS2 deletion suggesting that despite ACSS2 levels were decreased itself when kidney injury but the remnant ACSS2 still could be causal for the aggravating kidney injury.

### Example 2-4: ACSS2 Reinforce DNL and Lipid Storage to Develop Kidney Fibrosis

To understand the role of ACSS2 in kidney fibrosis, the biological functions of ACSS2 in cell physiology were studied. ACSS2 mainly involved in the generation of acetyl CoA from intracellular or intranuclear acetate pools. Intranuclear acetyl CoA generated by ACSS2 are associated with the acetylation of histone post translational modification (PTM). Intracellular acetyl CoA pools supports for the fatty acid oxidation or energy generation by oxidative phosphorylation, cholesterol biosynthesis and fatty acid synthesis. Therefore, H3K27ac levels were checked, fatty acid oxidation and cholesterol biosynthesis gene expression and found no significant differences in Acss2−/− mice compared to WT. Fatty acid synthesis is one of the rudimentary pathways operated in the kidney at lesser extent than liver DNL. Acetyl CoA carboxylase (Acaca) and fatty acid synthase (Fasn) are the major enzymes in the fatty acid synthesis. However, sterol regulatory binding protein1 (Srebp1) and Srebp cleavage protein (Scap) determines DNL by regulating the gene expression of Acaca, Fasn and other genes in involved in the fatty acid synthesis. Gene expression analysis of Scap, Srebp1, Acaca and Fasn were all trending towards low by Acss2−/− gene deletion however, these genes expression was significantly attenuated in the Acss2−/− UUO mice compared WT UUO. Perilipin2 (Plin2) is the triglyceride (TAG) coat protein that encloses TAG and facilitate their export or uptake. In contrast, Acss2−/− mice UUO kidney significantly attenuated Plin2 gene expression thus TAGs levels suggesting that fatty acid synthesis is the mechanism that ACSS2 is regulating during kidney injury.

### Example 2-5: Inhibition of Fatty Acid Synthase Suppress Kidney Fibrosis

To test whether FASNall could attenuate fibrosis gene expression Tubular epithelial cells (TECs) were treated with TGF-β1, a pro-fibrotic molecule that induces collagen and fibronectin gene expression at transcriptional level. TGF-β1 treatment increased Plin2 gene expression and TAG levels in TECs confirming that TGF-β1 can also induces fatty acid synthesis. TGF-β1 could not induce Plin2 gene expression and TAGs accumulation in the presence of FASNall. However, TGF-β1 treatment elevated the Col1a1, Col3a, Fn1 and α-SMA gene expression and FASNall significantly prevented these genes expression suggesting that inhibition of FASN prevents fibrosis.

Next, it was tested whether pharmacological inhibition of FASN inhibits kidney fibrosis in mouse model of kidney injury. Gene expression analysis of FASN and Plin2 increased in UUO kidneys and single dose of FASNall significantly prevented FASN and Plin2 expression. TAGs levels measured in the kidneys of UUO and FASNall UUO revealed that FASNall prevented that TAGs accumulation in the injured kidneys. Oil red ‘O’ staining showed that lipid accumulation in the UUO kidneys and injection of FASNall attenuated lipid accumulation. Protein levels of FN1 and α-SMA in these kidneys showed that FASNall suppressed UUO induced fibrosis. Sirius Red staining indicate that tissue scarring is less in Acss2−/− mice kidneys compared to WT UUO kidneys, likewise the quantification of the relative fibrosis area percentage. Finally, the gene expression analysis of fibrosis markers. Col1a1, Col3a, and Fn1 are lower in FASNall UUO mice kidneys suggesting that inhibition of FASN by FASNall prevents kidney injury and fibrosis development.

### Example 2-6: Pharmacological Inhibition of ACSS2 Prevents Kidney Fibrosis

Pharmacological inhibition of ACSS2 prevents kidney fibrosis. Next, checked was whether inhibition of ACSS2 by pharmacologically reduce kidney fibrosis. ACSS2i is a specific inhibitor of ACSS2 shown to reduce the obesity-induced multiple myeloma growth in cell and animal models. Therefore, this inhibitor was injected into mice before performing UUO surgery and continued to inject weekly twice for five days and mice were euthanized in day 6th. H&E analysis indicate that mice injected with ACSS2i prevented UUO induced kidney injury compared to vehicle injected mice. Sirius Red staining indicate that ACSS2i injected mice kidneys showed reduced collagen fibers staining than mice injected with vehicle alone. Gene expression and protein expression analysis of fibrosis markers showed reduced fibrosis in ACSS2i injected mice upon UUO kidney injury confirming thatACSS2 deletion or inhibition prevents kidney injury or fibrosis.

### Example 2-7: Mitochondrial Injury Induces Pyroptosis and Inflammation

It has been demonstrated that mtROS or mtDNA activates NLRP3 inflammasome pathway leading to inflammation. NLRP3 inflammasome assembly triggers proteolytic cleavage of dormant procaspase-1 into active caspase-1, which then cleaves the precursor cytokine into mature and biologically active cytokine including IL-1β and IL18. To demonstrate whether NLPR3 pathway has been activated in cells with severe mitochondrial defects upon treatment with TGF-β1, the present study employed primary TECs from WT and Acss2−/− mice. These cells were treated with TGF-β1 and studied the gene expression changes of NLRP3, IL-1β, IL18, Caspase1 and Gasdermin D (GSDMD). It was observed that NLRP3, IL-1β and Caspase1 expression levels were elevated in TGF-β1 treated cells and that were reduced in cells treated with FASN inhibitors, FASNall or TVB-3664.

To further substantiate this observation, primary TECs were generated from SCAPf/f mice, cells were treated with Cre recombinase to delete SCAP and then treated with TGF-β1. Gene expression analysis of SCAP and DNL genes confirm that the SCAP was deleted. While pyroptosis genes such as IL-1β, IL18 and caspase1 levels were markedly increased with TGF-β1 treatment in WT cells and this was attenuated in cells lacking SCAP or DNL. To bring specific evidence that FASN is playing a role in this pathway, FASNf/− TECs were generated and treated with TGF-β1. It was observed that cells lacking a single copy of FASN was sufficient to suppress pyroptosis induced by TGF-β1, suggesting that pyroptosis can be manipulated by ROS or mitochondrial damage via FASN or DNL.

### Example 2-8

Integration of GWAS and human kidney eQTL dataset prioritized ACSS2 as potential kidney disease risk gene, with lower ACSS2 levels being protective. Single cell and immunofluorescence studies highlighted strong ACSS2 expression in kidney tubules. At baseline Acss2−/− showed no phenotypic differences, however exhibited less fibrosis both in the folic acid and UUO models compared to WT animals. In vitro, Acss2−/− cells showed less fibrosis gene expression upon TGF-β1 treatment, a cell model of fibrosis compared to WT cells. Decreased fatty acid synthesis genes and lipid droplets were observed inAcss2−/− cells compared to Acss2++ cells. Increased fatty acid synthesis increased mitochondrial oxidative stress that activates NLRP3-mediated pyroptosis. Ultimately, it is shown that inhibiting fatty acid synthase (Fasn) or deleting de novo lipogenesis (DNL) or depleting mitoROS prevented the fibrosis and lipid toxicity in WT cells.

Via the integration of kidney function GWAS and eQTL, mouse model and cell culture studies ACSS2 was identified as a new kidney disease risk gene. Reducing ACSS2 levels is protective against fibrosis, by offering protection against the fibrosis by offering string antioxidant system in proximal tubule cells of the kidney.

### Example 3: ACSS2 is a Renal Disease Risk Gene by Controlling De Novo Lipogenesis in Kidney Tubules

Worldwide, over 800 million people are affected by kidney disease, yet its pathogenesis remains elusive, hindering the development of novel therapeutics. In this study, the present study employed kidney specific expression of quantitative traits and single-nuclear open chromatin analysis to show that genetic variants linked to kidney dysfunction on chromosome 20 target the acyl-CoA synthetase short-chain family 2 (ACSS2). By creating ACSS2 knock-out mice, the present study demonstrated their protection from kidney fibrosis. The analysis of primary tubular cells herein revealed that ACSS2 regulates de novo lipogenesis (DNL), causing NADPH depletion and increasing ROS levels, ultimately leading to NLRP3-dependent pyroptosis. Additionally, the present study discovered that pharmacological inhibition or genetic ablation of fatty acid synthase safeguarded kidney cells against profibrotic gene expression and prevented kidney disease in mice. Lipid accumulation and the expression of genes related to DNL were elevated in the kidneys of patients with fibrosis. The findings herein pinpoint ACSS2 as a critical kidney disease gene and reveal the role of DNL in kidney disease.

Over 800 million people in the world suffer from chronic kidney disease (CKD). CKD is a major cause of cardiovascular death and if left untreated leads to end-stage kidney disease necessitating dialysis or kidney transplantation. CKD is one of the most rapidly growing common causes of death, accounting for over one million fatalities each year. Despite its considerable impact on public health, the mechanisms driving CKD pathogenesis remains largely unknown, impending the development of effective treatments.

The kidney is a highly metabolically active organ responsible for filtering and reabsorbing a vast amount of electrolytes and fluids, including 180 liters of water and nearly one kilogram of sodium chloride daily. In particular, the proximal tubule (PT) segment of the kidney relies predominantly on fat as an energy source and mitochondrial oxidative phosphorylation for efficient energy production. However, kidney lipid content is markedly elevated in disease states such as kidney disease (DKD). Indeed, Kimmelstein and Wilson identified lipid deposition as a characteristic of DKD. The mechanism underlying lipid accumulation and alterations in lipid metabolism in kidney disease, however, remains poorly understood.

Kidney tubule cells take up fatty acids through CD36 and FATP2 transporters and increased lipid uptake believed to contribute to disease development. A defect in fatty acid oxidation has been identified as an important contributor to tubule atrophy. Improving fatty acid oxidation by expression of PPARA, PPARGC1A, CPT1A or pharmacologically stimulating these pathways improves kidney function.

Lipid accumulation is a notable characteristic of various fibrotic diseases, including non alcoholic steatohepatitis (NASH). The mechanisms underlying lipid accumulation in NASH are not fully understood, but several factors have been implicated, such as increased uptake of circulating lipids, enhanced de novo fatty acid synthesis, decreased beta oxidation and reduced lipid export However, the specific contribution of each lipid metabolic alteration in the development of liver fibrosis remains to be elucidated.

Large scale genome-wide association studies (GWAS) have identified more than 800 genetic loci where single nucleotide variants (SNPs) are associated with kidney function as estimated by glomerular filtration rate (eGFR). However, GWAS has several limitations: most regions contain large number of significant variants those level show close correlation; the identified GWAS variants are in the non-coding region, making it challenging to pinpoint to causal variants, target genes and cell types. Functional annotation of GWAS requires multiple orthogonal datasets. Recently expression quantitative trait locus (eQTL) analysis has emerged as a valuable tool for identifying target genes by identifying disease-associated variants that also regulate gene expression. Epigenetic datasets including open chromatin annotation have been used to narrow down likely causal variants, and single cell epigenome and expression data can even indicate the disease causal cell types. While computational target gene prioritization has significantly improved, cellular and animal models remain critical for validation studies.

The present study conducted a computational analysis of the chromosome 20 eGFR GWAS locus, using expression, methylation QTL, and single-cell open chromatin data to prioritize ACSS2 as a kidney disease risk gene. The gene knock-out and cellular model experiments herein revealed the critical role of ACSS2 in de novo lipogenesis (DNL), which consumes NADPH and induces changes in cellular redox homeostasis leading to NLRP3-dependent pyroptosis and fibrosis. Moreover, the mouse model studies demonstrated that pharmacological inhibition of ACSS2 or fatty acid synthase (FASN) can mitigate kidney fibrosis.

### Example 3-1: Gene Prioritization Analysis Indicates that ACSS2 is a Kidney Disease Gene

A recent GWAS of kidney function (eGFRcrea) revealed a strong association between genetic variants in gene dense region of the chromosome 20 (FIG. 51A). Genetic fine mapping of the region indicated 2 independent regions at this locus (the top SNPs being rs11698977 and rs6141526) (FIG. 58A). To understand potential causal variants, genes, and cell types at this locus, the present study employed a comprehensive integrated annotation approach. For this, the present study examined the closest genes, human kidney gene expression and methylation quantitative trait (eQTL, mQTL), human kidney snATAC-seq, open chromatin peak co-accessibility activity by contact (ABC) method, Bayesian colocalization and summary mendelian randomization. These analyses highlighted ACSS2, CEP250 and SPAG4 with six supporting pieces of information from 8 different methods (FIG. 51B).

The present study chose to focus on ACSS2 (acetyl coA synthase2) as the top fine mapped SNP (rs11698977) was strongly associated with local cytosine methylation levels and ACSS2 gene expression in human kidney tubule compartment (FIGS. 51C-51F and 58B). Bayesian colocalization analysis of the locus indicated that the eGFR GWAS identified variants and variants that influence local methylation and the expression of ACSS2 were shared (PPH4>0.88, and PPAabc>0.82). Summary Mendelian randomization analysis indicated that ACSS2 expression as an effect mediator of genetic variants influencing kidney function at this locus, suggesting that lower ACSS2 is associated with better kidney function (FIG. 58C).

To further pinpoint disease causal variants, the present study analyzed single-nuclear open chromatin (snATAC) data from adult human kidneys, and overlapped GWAS significant SNPs with human kidney single cell open chromatin information. The present study prioritized six SNPs as a candidate likely causal SNPs in the locus that could mediate the eGFR signal, ACSS2 expression, by based on their overlap with open chromatin area in proximal tubule cells (FIG. 51G). To demonstrate the causal role of these eGFR variants, the present study used the CRISPR/Cas9 system to delete genomic region containing the GWAS variants in open chromatin peaks using embryonic kidney cells (FIG. 51H). The present study found that genetic deletion of the risk variants including the index SNP (and two other variants), rs11698977 altered the ACSS2 gene expression (FIG. 51I), but not the level of CEP250 and SPAG4 indicating likely causal variants, and ACSS2 are the likely effect mediator of this GWAS signal (FIGS. 58D and 58E).

In summary, the present study identified a genetic signal for kidney function on chromosome 20, spanning over more than 20 genes. The complex gene prioritization strategy indicated ACSS2, CEP250 and SPAG4 as likely causal genes. CRISPR-based locus deletion studies highlighted genetic variants that directly regulated ACSS2 levels, prioritizing ACSS2, a likely kidney disease causing gene.

### Example 3-2: Genetic Deletion of ACSS2 Protects from Kidney Fibrosis

To investigate the role of ACSS2 in kidney disease, the present study generated mice with a genetic deletion of ACSS2 using the CRISPR/Cas9 knock-out system. The present study deleted the first exon of Acss2 gene. Gene and protein expression analysis confirmed the reduction in ACSS2 in kidneys of the Acss2 knock-out mice (Acss2−/−) compared to littermate controls (FIGS. 58F and 58G). Acss2−/− mice were born at the normal Mendelian ratio, without birth defects, growth abnormalities, or signs of kidney dysfunction (FIGS. 58F-58J). The present study did not observe differences in the expression of Ki67 (mKi67), Kim1 (Hacvr1) and N-gal (Lcn2) in the kidneys of and Acss2−/− mice (FIGS. 58K and 58L).

Genetic studies suggested a protective role for ACSS2, so the present study analyzed WT and Acss2−/− mice in adenine-induced kidney disease model (FIG. 52A). The body weight was lower in adenine treated WT mice compared to Acss2−/− or vehicle treated groups (FIGS. 52B and 53C). Kidney weights were increased in the WT adenine group compared to vehicle or Acss2−/− groups (FIG. 58A). Gene expression and protein levels of ACSS2 were lower in whole kidney lysates of mice with kidney disease (FIGS. 52D, 52J, 52K, 58B, 58E, 58F, and 58G). The change observed in whole kidney ACSS2 level was likely attributed to the loss of proximal tubule cells, as single cell data did not indicate a similar decline in ACSS2 expression in disease states (see FIGS. 57A, 57B and 64A). The gene expression of fibrosis markers such as collagen type1a1 (Col1a1), collagen type 3a (Col3a) and fibronectin (Fn1) were higher in the WT adenine group compared to adenine-treatedAcss2−/− mice (FIG. 52E). Consistent with the gene expression data, the protein levels of fibronectin (FN1) and smooth muscle actin (α-SMA), were higher in the WT adenine group compared to the adenine treated Acss2−/− mice (FIGS. 52F and 58C). Histological analysis indicated lower tubular injury and fibrosis in Acss2−/− mice than in WT adenine-treated mice (FIGS. 52G and 58D). Indicators of kidney function, such as serum creatinine (sCr) and blood urea nitrogen (BUN), were elevated in WT adenine-treated mice compared to adenine-treated Acss2−/− mice (FIG. 52H).

Next, the present study validated the findings in two different established kidney disease models, induced by unilateral ureteral obstruction (UUO) or folic acid injection (FAN) (FIG. 52I). The protein markers of fibrosis, including levels of FN1 and α-SMA, were higher in the UUO and FAN kidney fibrosis models, but their levels were observably lower in the kidneys of Acss2−/− mice with UUO and FAN injury (FIGS. 52L, 52M, 58H and 58I). Transcript levels of Col1a1, Col3a and Fn1 were higher in kidney disease models, but they were lower in Acss2−/− mice with kidney injury (FIGS. 58J and 58K). Histological changes such as tubule atrophy and interstitial fibrosis were also lower inAcss2−/− mice (FIGS. 52N and 58L-58N). Clinical markers of kidney injury, sCr and BUN both were increased in WT mice injected with folic acid but not in Acss2−/− mice (FIGS. 580 and 58P).

The present study next established an in vitro cell model of fibrosis by culturing primary kidney tubular epithelial cells (TECs) from WT and Acss2−/− mice in the presence of transforming growth factor beta1 (TGF-beta 1). TECs from WT mice showed higher expression of fibrosis markers, including Col1a1, Col3a, Fn1 and Acta2 when treated with TGF-beta 1, whereas cells lacking ACSS2 showed lower levels of the TGF-beta 1-induced fibrosis gene expression (FIG. 58Q). Consistently, immunoblotting performed with the lysates of primary TECs treated with TGF-beta 1 showed increased protein levels of alpha-SMA and FN1, which were lower in Acss2−/− cells (FIGS. 58R and 59A).

In summary, Acss2 knock-out mice and kidney tubule cells showed protection from kidney disease.

### Example 3-3: ACSS2 Expression Correlates with De Novo Lipogenesis and Kidney Fibrosis

ACSS2 is a multifunctional enzyme involved in the generation of acetyl CoA (ac-CoA) from intracellular or nuclear acetate pools and plays a role in variety of biochemical processes (FIG. 53A). Intranuclear ac-CoA is generated by ACSS2 can be used for histone post translational modification (PTM), specifically ACSS2 plays a role in histone 3 lysine 27 acetylation (H3K27ac). Therefore, the present study extracted total histones from kidneys of WT and Acss2−/− mice at baseline and following UUO injury and compared H3K27ac levels by western blotting. The present study found no observable changes in histone acetylation (H3K27ac) inAcss2−/− mice at baseline and following UUO injury (FIG. 53B). Fatty acid oxidation (FAO) genes such as Acox1, Acox2, Cpt1, and Cpt2 were measured in kidneys of WT and Acss2−/− mice following UUO injury. The present study observed lower expression of Acox1, Acox2, Cpt1, and Cpt2 in WT UUO kidneys (FIG. 53C). The reduction of Acox1, Acox2, Cpt1, and Cpt2 was not rescued in Acss2−/− kidneys (FIG. 53C). Finally, the present study tested FAO rates in the kidneys of WT and Acss2−/− mice using tritium labeled palmitic acid (³H-Palmitate) (FIG. 53D). The present study observed comparable FAO rates in kidneys of WT and Acss2−/− mice at baseline (FIG. 53E). The injured kidneys showed markedly lower FAO rates which were not rescued by Acss2−/− deletion (FIG. 53E). Furthermore, the present study tested FAO in primary tubular cells by a Seahorse based palmitic acid oxidation. The Seahorse based FAO analysis did not show a significant difference in OCR and ATP levels between WT and Acss2−/− primary tubule cells following palmitic acid supplementation (FIGS. 60B-60D). Consistently, in the cultured tubule cell system, the present study found that expression of Acox1, Acox2, Cpt1, and Ppar alpha was lower following TGF-beta 1 treatment, indicating a defect in fatty acid oxidation in kidney fibrosis, however, this defect was not rescued inAcss2−/− cells (FIG. 60E).

ACSS2 generated ac-CoA also fuels cholesterol biosynthesis. Hydroxy methyl glutaryl CoA synthase (HMGCS1) and HMGC reductase (HMGCR) are the major enzymes involved in synthesis of precursor mevalonate for cholesterol synthesis (FIG. 60A). The present study measured the expression of Hmgcs1, Hmgcr and farnesyl diphosphate synthase (Fdps) in the kidneys of WT and Acss2−/− mice with SHAM or UUO surgery. The present study did not observe a change in the expression of genes involved in cholesterol biosynthesis (FIG. 60F), which was consistent with the total kidney cholesterol levels measured in WT and Acss2−/− mice (FIG. 60G).

Finally, the present study examined de novo lipogenesis (DNL) since ac-CoA is utilized in the synthesis of fatty acids. Acetyl coA carboxylase (ACACA) and fatty acid synthase (FASN) are the key enzymes in fatty acid synthesis, with sterol regulatory binding protein1 (SREBP1) and SREBP cleavage protein (SCAP) being their key upstream regulators. The present study measured that the expression of Scap, Srebp1, Fasn, and Acaca was higher in the UUO model of kidney injury (FIGS. 53H and 53I). Loss of ACSS2 was associated with lower expression of Scap, Srebp1, Fasn, and Acaca compared to kidneys of WT UUO (FIGS. 53H and 53I). Protein levels of FASN were higher in WT UUO and were reduced inAcss2−/− mice with kidney injury (FIGS. 53J and 60F).

Next, the present study assessed DNL rate in vivo by quantifying the incorporation of deuterated water (D20) into palmitate in the kidney (FIG. 53K). The present study observed a higher DNL rate in WT UUO kidneys compared to Acss2−/− UUO kidneys (FIG. 53L). Consistently, tissue triglyceride (TG) the expression of perilipin2 (Plin2), a marker of TG accumulation was higher in UUO kidneys, but they were markedly lower in the UUO injury model of Acss2−/− mice (FIGS. 53M-53O). To test whether TGF-beta 1 induces DNL in primary TECs, the present study treated TECs with TGF-beta 1 and found higher Fasn and Plin2 gene expression, which was consistent with higher TG levels, confirming that TGF-beta 1 can also induces fatty acid synthesis (FIGS. 60G, 60I, 60J, 60K-60L, 60O, 60P and 60Q).

In summary, the present study observed marked changes in de novo lipogenesis in mouse kidney disease model, which were markedly improved in absence of ACSS2.

### Example 3-4: Pharmacological Inhibition of Fatty Acid Synthesis Protects from Kidney Fibrosis

Since the cell studies suggested the key role of ACSS2 in kidney DNL, the present study next examined whether direct inhibition of fatty acid synthase (FASN) could protect against kidney fibrosis. First, the present study tested two widely used drugs, FASNall and TVB-3664, in a cell culture model of fibrosis (FIG. 60G). Treatment of primary TECs with FASNall or TVB-3664 did not increase cell death (FIG. 60H). Treatment of tubule cells with FASNall prevented the TGF-beta 1-induced increase in FASN and Plin2 expression and TG accumulation (FIGS. 60I-60M). FASNall treatment protected against TGF-beta 1-induced profibrotic gene expression changes, including Col1a1, Col3a, Fn1 and Acta2 (FIG. 60N). The present study observed a similar effect with TVB-3664, a more potent FASN inhibitor on the profibrotic gene expression changes, Plin2, TG accumulation and total cell TG levels (FIGS. 60M and 60O-60R).

The present study verified the pharmacological studies by genetic knockdown of Fasn using FASN siRNA (siFasn) (FIG. 61A). Gene expression analysis of Fasn indicated successful knockdown (˜60%) (FIG. 61B). siFasn markedly protected not only against TGF-beta 1 treatment-induced Plin2, but also Acta2, Col1a1, and Fn1 increase (FIGS. 61A-61B). The present study observed similar results in Scap deficient tubule cells (FIGS. 61C-61D). Scap-deficient kidney tubule cells had lower expression of Fasn, and Plin2 confirming the dependency of DNL gene expression on SCAP/SREBP1 axis (FIG. 61C).

Next, the present study tested the effect of FASN inhibitor in the UUO mouse model of kidney fibrosis (FIG. 61E). The expression of Fasn and Plin2 was higher in UUO kidneys (FIG. 61F). FASNall ameliorated the rise in Fasn and Plin2 expression (FIG. 61F). Similarly, TGs levels were higher in UUO kidneys, but lower in animals treated with FASNall (FIG. 61G). Oil red O staining showed lipid accumulation in the UUO kidneys, which was lower in FASNall injected animals (FIG. 61H). Protein levels of FN1 and α-SMA were lower in FASNall-treated UUO mice compared to sham treated animals (FIGS. 61I and 61J). Histological analysis indicated severe tubule atrophy and fibrosis in vehicle treated UUO kidneys, which was lower in FASNall-treated UUO mice (FIG. 61K). Sirius red staining indicated less tissue scarring in FASNall treated UUO mice kidneys compared to vehicle treated UUO kidneys (FIG. 61K). Finally, fibrosis markers Acta2, Col1a1, Col3a, and Fn1 were lower in FASNall UUO mice kidneys (FIG. 61L) suggesting that inhibition of FASN by FASNall prevented kidney injury and fibrosis development.

### Example 3-5: Genetic Deletion of FASN Protects from Kidney Disease Development

Since FASN is the key enzyme in DNL, the present study next tested whether tubule specific genetic deletion of Fasn would protect mice from the kidney disease development.

The present study generated tubule specific Fasn knockout mice by crossing Ksp Cre with Fasn flox mice (FIG. 54A). While whole-body knockout of Fasn is embryonically lethal, tubule specific Fasn knockout (Fasn f/f Ksp Cre) mice were grossly indistinguishable from their WT littermates. Gene expression analysis revealed that Fasn expression was lower in Fasn f/f Ksp Cre compared to WT mice (FIG. 54B). The present study tested the role of FASN in adenine-induced kidney disease and the UUO models. Transcript and protein levels of FASN were increased in diseased kidneys of WT mice, but their levels were observably lower in mice with tubule specific Fasn deletion (FIGS. 54B, 54C, 61M and 61N). Body weight was lower in adenine-treated WT mice when compared to control, but it was less reduced in adenine treated Fasn knock-out mice (FIGS. 61O and 61P). Gene and protein expression analysis of Acta2, Col1a1, Col3a, and FN1 indicated that tubule specific deletion of Fasn lowered kidney fibrosis (FIGS. 54D, 54F and 61Q-61S). H&E and Sirius red staining analyses revealed tubular atrophy and collagen deposition in WT adenine, and UUO mice whereas tubule specific Fasn f/f Ksp Cre mice had less tubule damage and fibrosis (FIGS. 54G, 61T and 61U). Kidney function tests such as sCr and BUN were also improved in adenine-treated Fasn knock-out mice compared to WT adenine treated mice (FIG. 61V). Together these results indicate that deletion of FASN in tubule cells ameliorates kidney dysfunction, indicating the key role of tubule specific DNL in kidney dysfunction.

### Example 3-6: Pharmacological Inhibition of ACSS2 Prevents Kidney Fibrosis

Next, the present study checked whether pharmacological inhibition of ACSS2 would also reduce kidney fibrosis. The present study injected mice with ACSS2i prior to UUO surgery and on the 4th day following the UUO surgery (FIG. 54H). Histological analysis by H&E staining and fibrosis quantification by Sirius red staining indicated markedly lower fibrosis and tubule atrophy in ACSS2i treated mice (FIGS. 54I and 61W). Gene and protein expression analysis of fibrosis markers (Acta2, Col1a1, Col3a, and Fn1) further confirmed the protective effect of ACSS2i in kidney fibrosis (FIGS. 54K-54L). Importantly, ACSS2i injection also inhibited lipid accumulation, which was evidenced by lower expression levels of Fasn, Plin2, and TG accumulation in the UUO kidneys (FIGS. 61X-61Z).

### Example 3-7: Reduced NADPH Consumption and Lower Oxidative State in Absence of ACSS2

Next, the present study aimed to understand the mechanism of ACSS2 deletion and DNL inhibition afforded protection from kidney fibrosis. The experiments with tubule specific CD36 transgenic mice indicated that triglyceride accumulation alone in kidney tubules per se was not sufficient to cause full spectrum of fibrosis. Therefore, it was hypothesized that DNL might be associated with increased NADPH utilization leaving cells at higher risk of oxidative damage (FIG. 55A). The present study measured NADPH and NADP+ ratio in WT and Acss2−/− cells using a luminescent probe (FIG. 55B). TGF-beta1 treatment lowered NADPH/NADP+ ratio in WT cells but not in Acss2−/− cells (FIG. 55C). Total NADPH levels were higher in Acss2−/− cells than in WT cells (FIG. 55D). Oxidized (GSSH) to reduced glutathione (GSH) is an important measure of cellular redox state. Glutathione measurements revealed a lower GSH/GSSH ratio in TGF-beta 1 treated WT cells but not in Acss2−/− cells (FIG. 55E). Total GSH levels were higher in the Acss2−/− cells compared to WT cells (FIG. 55F).

The present study then tested the role of FASN and DNL in regulating NADPH+/NADP+ ratio. The present study treated cells with TGF-beta 1 in the presence or absence of FASNall and quantified NADPH and glutathione levels. The NADPH/NADP+ ratio was lower in TGF-beta 1 treated tubule cells, but it was preserved in FASNall treated tubule cells (FIGS. 55G and 55H). GSH levels and relative GSH/GSSH ratio were lower in TGF-beta 1 treated cells but preserved by FASN inhibitor (FIG. 55I), indicating that FASN uses significant amounts of NADPH for fatty acid synthesis, which scavenges NADPH, leaving more oxidized GSSH (FIG. 55J).

Next, the present study measured mitochondrial ROS using MitoSox. The present study observed increased ROS levels in TGF-beta 1-treated kidney tubule cells (FIGS. 55K and 65L). Tubule cells with genetic loss of Acss2 had lower ROS levels compared to WT cells following TGF-beta 1 treatment (FIGS. 55K and 55L). The present study observed similar results following FASNall treatment, indicating the role of fatty acid synthesis in modulating cellular ROS levels (FIGS. 55K and 55L). The present study then examined mitochondrial parameters. The present study observed a less negative mitochondrial membrane potential in TGF-beta 1 treated kidney tubule cells, as measured by monomeric JC-1 accumulation (FIGS. 55M and 55N). Acss2−/− cells or treatment of WT cells with FASNall attenuated this effect (FIGS. 55M and 55N).

Damaged mitochondria can stimulate mitophagy. The present study monitored mitophagy using Mito‘Q’, a plasmid that expresses mCherry-eGFP fusion construct under the COX4 promoter, which is selective to the mitochondria. This plasmid labels mitochondria in yellow when cells are in a fed state or treated with bafilomycin A (BA), whereas enhanced mitophagy induced by starvation or carbonyl cyanide m-chlorophenyl hydrazone (CCCP) turns the plasmid red due to the low lysosomal pH, which quenches GFP fluorescence. The present study found that nutrient deprived “starved” cells or CCCP treatment had increased mitophagy flux (a higher number of red mitochondria), and loss of ACSS2 further enhanced the number of mitolysosomes (FIGS. 55A and 55B). The present study confirmed the enhanced mitophagy flux in Acss2−/− cells compared to WT cells when analyzing mitophagy flux by LC3 and PARKIN1 immunoblotting in cells subjected to starvation or BA (FIGS. 55C and 55D).

In summary, the present invention observed an increased oxidative state in profibrotic tubule cells, inhibition of DNL, and ACSS2 protected against ROS accumulation and mitochondrial defects.

### Example 3-8: Mitochondrial Injury Induces Pyroptosis and Inflammation

To further understand the mechanism of ACSS2 and FASN mediated kidney fibrosis development, the present invention next examined whether the mtROS and mitochondrial defect mediated activation of the NLRP3 inflammasome pathway plays a role in the process (FIG. 55O). The present invention observed increased inflammasome activation in TGF-beta 1 treated cultured kidney tubule cells.

Expression of Nlrp3, IL-1B, and caspase1 was higher in TGF-beta1 treated cells (FIGS. 55P and 55Q). The inflammasome activation was dependent on DNL as levels of Nlrp3, IL-1B, and Caspase1 were lower in TGF-beta 1 treated tubule cells following FASN inhibitors, FASNall or TVB-3664 treatment (FIGS. 55P-55R and 62E). The present invention observed similar results in SCAP knock-out or in cells with heterozygous loss of Fasn (FIGS. 55S and 62F-62H). Furthermore, tubule cells obtained from Acss2−/− mice also showed protection from TGF-beta 1 induced NLRP3 inflammasome activation (FIG. 55T).

To understand whether changes in pyroptosis and fibrosis are dependent on TGF-beta 1 induced elevated mitochondrial ROS, the present invention tested the effect of MitoTempo (MT), a mitochondrial specific ROS scavenger. The present invention found that inflammasome activation, including IL-1B, IL18, and caspase1 increase following TGF-beta 1 treatment, was efficiently lowered by MT (FIGS. 55U and 55V). Consistently, MT treatment or Fasn genetic deletion lowered TGF-beta 1 induced expression of fibrosis markers, indicating the role of ROS in fibrosis (FIGS. 55I-55K). In summary, increased ACSS2 mediated DNL by FASN appears to lower NADPH and GSH, leading to elevated ROS levels and enhanced NLRP3 dependent pyroptosis in tubule cells.

### Example 3-9: Deletion of ACSS2 or DNL Attenuates Pyroptosis-Induced Inflammatory Fibrosis in Mice

To gain further insight into ACSS2- and DNL in development of kidney fibrosis, the present invention investigated whether the activation of NLRP3 inflammasome mediated mtROS and mitochondrial defects contributes to this process. The present invention analyzed Acss2−/− mice, Fasn f/f Ksp Cre mice and mice treated with ACSS2 or FASN inhibitors. Gene expression analysis of the inflammasome pathway revealed higher levels of NLRP3, GSDMD, IL-1B, IL18 and Caspase1 in the UUO model of kidney fibrosis (FIGS. 56A, 56D, 56F, 56G and 63A). The present invention found that the gene expression of NLRP3, IL-1B, IL18, caspase1 and Gsdmd was lower in the kidneys of Fasn f/f Ksp Cre mice (FIG. 56A), FASNall (FIG. 63A), ACSS2i treated mice (FIGS. 56C and 56D), and Acss2−/− mice with UUO (FIGS. 56F and 56G). GSDMD RNA was detected in proximal tubule cells (LDL receptor 2; Lrp2-positive) particularly, its expression was elevated in injured kidneys (FIGS. 63B and 63C). Immunoblotting of NLRP3, Caspase1, and GSDMD showed higher levels in the UUO kidneys, while they were lower in the kidneys of FASN f/f Ksp Cre mice with UUO injury (FIGS. 56B and 63D). The cleaved form of GSDMD (N-GSDMD) is the effector molecule of the pyroptosis. The GSDMD-N western blots showed a band at ˜25 kD, which was higher in the kidneys of WT UUO mice and that lower in the UUO kidneys of FASN f/f; Ksp Cre mice (FIGS. 56B and 63D). Protein levels of NLRP3, caspase1, cleaved caspase1 (p20), F-GSDMD, and N-GSDMD in the kidneys of mice treated with ACSS2i (FIGS. 56E and 63E), FASNall (FIGS. 63E and 63G), or Acss2−/− (FIGS. 56H and 63H) kidneys following UUO surgery were lower compared to the vehicle treated UUO kidneys. Similar results were observed in the adenine-induced kidney disease models with genetic deletion of ACSS2 or FASN suggesting that NLRP3 inflammasome activation is associated with kidney disease development in these models (FIGS. 56I, 56J and 63I-63K).

### Example 3-10: The DNL Gene Signature in Human Kidney Disease Samples are Highly Enriched in PT Cells

To understand the relevance of ACSS2 and DNL in patients with kidney disease, the present invention first analyzed the fatty acid synthesis gene signature in human kidney single cell expression data (http://www.susztaklab.com/hk_genemap/scRNA). Healthy controls and kidney disease samples showed strong expression of DNL genes, including ACSS2, NR1H3 (Liver X-receptor Alpha; LXRA), NR1H4 (Farnesoid X-receptor; FXR), SREBF1, SCAP, PLIN2, PLIN5, ACACAB, ACLY, and FASN in proximal tubule (PT) cells and some other tubule cells (FIGS. 57A-57B). ACSS2 was almost exclusively expressed by the PT cells compared to all other cells in the kidney and its levels were not different between healthy and diseased PT cells (FIG. 64A).

The present invention confirmed single cell gene expression results using in situ hybridization in healthy and disease human kidneys. The present invention found that ACSS2 was highly expressed in PT cells, as the present invention observed its co-expression with the proximal tubule marker megalin (LDL receptor 2; LRP2) (FIG. 57C). Consistent with single cell gene expression data, the quantification of RNA in situ hybridization showed a tendency towards decreased ACSS2 expression (FIG. 57D). The present invention observed a similar PT cell specific expression of Acss2 in mouse kidneys, specifically in Lrp2 positive PT cells (FIG. 57E). The present invention analyzed published mouse kidney single cell gene expression data and found that Acss2 expression was not different between PT cells of control, and diabetic mice (FIG. 64B). Immunofluorescence for the ACSS2 protein in human kidneys followed the pattern of the in-situ hybridization (FIG. 57F). In healthy human kidneys FASN was mostly expressed in LTL positive (proximal) tubule cells (FIG. 57G). PLIN2 expression was enriched at the basolateral aspect of LTL positive (proximal) tubule cells (FIG. 64B). Protein levels of SCAP, FASN, PLIN2, NLRP3, and GSDMD were higher in fibrotic human kidney tissue samples, likely indicating that DNL could be associated with kidney disease (FIGS. 57G and 57I).

### Example 3-11

Here, the present invention identified ACSS2 as human kidney disease risk gene via the integration of human genetic (GWAS), kidney QTL (eQTL and mQTL), and human kidney single cell expression (snATAC-seq) data. Using CRISPR knock-out mice and cultured kidney cell models, the present invention show that ACSS2 controls de novo lipogenesis and contributes to fibrosis development by regulating cellular redox state and inflammasome activation. Mice with tubule specific deletion of FASN, the key rate limiting enzyme in DNL, demonstrate the critical role of DNL in kidney tubule cells. Furthermore, the work identifies ACSS2 and FASN as potential novel pharmacological targets for kidney disease.

Here, the present invention used genetic mapping, kidney eQTL, mQTL, single cell open chromatin information, and advanced statistical methods such as Bayesian colocalization, mendelian randomization, and chromatin co-accessibility methods to prioritize ACSS2 as a kidney disease risk gene on chromosome 20. This is a large, highly gene-dense region with close to 20 genes. The present invention observed that the expression of genes at this locus shows strong correlation with each other. Even after applying multiple gene prioritization methods, the present invention were left with 3 genes prioritized by all available datasets and methods. Here, the present invention focused on ACSS2. The analysis of the other genes at this locus should be the focus of future studies as multiple genes could be responsible for disease development at a single locus, such as DPEP1 and CHMP1A.

Abnormal lipid metabolism is an important feature of CKD. Kidney tubular cells almost exclusively consume fatty acids to liberate energy for the active reabsorption of filtered electrolytes and metabolites. Proper fatty acid oxidation and mitochondrial respiration events are crucial for normal kidney function. Both pathways were defective in patients with CKD. The present invention also observed a decreased expression of fatty acid oxidation genes.

The role of fatty acid synthesis has gained an interest in fibrotic diseases such as the liver; however, it has not been studied in the kidney. ACACA and FASN are the major rate limiting enzymes controlling fatty acid synthesis. Increased FASN expression and activity has been consistently associated with steatosis induced liver fibrosis and bleomycin induced pulmonary fibrosis. Inhibition of FASN activity by TVB-2640 in NASH patients shows protection and its effects are being studied in phase-2b clinical trials. The present invention noted an increase in levels of genes in the fatty acid synthesis pathway, including FASN, and ACACA. Concomitantly, SREBP1 and SCAP are upstream regulators of fatty acid synthesis that were also higher in injured kidneys. Deletion of SCAP or FASN in tubule cells or pharmacological inhibition of FASN protected cultured tubular cell from profibrotic gene expression and mice from tissue fibrosis, highlighting the importance of DNL in kidney disease development.

The mechanistic link between DNL and fibrosis is less understood. The present study demonstrated that fatty acid synthesis in tubular cells lowers the cellular reduced form of NADP level and is associated with elevated mitochondrial ROS production and fibrotic gene expression. Importantly, DNL signature genes were highly expressed by proximal tubule cells. Deletion of ACSS2 suppressed FASN, and ACACA expression in tubular cells, lowered the cellular redox state, and prevented the expression of profibrotic genes, including Acta2, Col1a1, Col3a, and Fn1. The present study also show that scavenging mitochondrial ROS by MitoTempo diminished the expression of fibrotic genes. Inflammatory pathways, including the NLRP3 dependent pyroptosis, play a role in kidney, liver, and heart fibrosis. The present study found that expression of NLRP3, N-GSDMD, and cleaved caspase1 protein levels in UUO kidneys were lower in absence of ACSS2.

The present study identifies a new pharmacologically targetable mechanism for kidney fibrosis. The present study shows that not only genetic deletion of ACSS2, FASN, SCAP protects cells and mice from fibrosis development, but pharmacological targeting of these pathways is also protective. The present study show that small molecules, such as FASNall, which targets FASN, and ACSS2i, which targets ACSS2, protect mice from kidney disease development. It is reassuring that Acss2−/− mice does not have an observable phenotype at baseline, suggesting that pharmacological targeting of ACSS2 is likely to be safe. The studies would indicate that other compounds that are currently in clinical trials for NASH for example ACC1 (ACACA) inhibitors, could also ameliorate fibrosis development offering an entire class of new drugs for kidney dysfunction.

In summary, the present study identifies ACSS2 as a novel kidney disease gene. The present study demonstrates the key role of DNL and fatty acid synthesis in kidney fibrosis by regulating cellular redox status and inflammation (FIG. 65). Finally, the present study presents data indicating that pharmacological targeting of ACSS2 or DNL could be an important therapeutic strategy for CKD.

### Example 3-12: Material

Prioritization of Causal Genes for eGFR GWAS Loci

To prioritize target genes for eGFR GWAS loci on chr 20, the present study employed a priority scoring strategy by integrating eight different datasets: (1) microdisected kidney tubule and glomeruli eQTL data sets (https://susztaklab.com/Kidney_eQTL/index.php) (significant SNP˜gene associations, FDR<0.05); (2) mQTL (significant SNP-CpG-gene associations, FDR<0.05) and eQTM (CpG level FDR<0.05) analysis (https://susztaklab.com/Kidney_meQTL/index.php); (3) colocalization between eGFR GWAS and eQTL (H4>0.8); (4) multiple colocalization (moloc) analysis of SNP˜gene pairs between eGFRGWAS, eQTL and mQTL (PPA.abc>0.8); (5) summary mendelian randomization for the SNP˜gene pairs between eGFR GWAS and eQTL (PSMR<1.38×10-4); (6) SNP˜gene pairs passing HEIDI test between eGFR GWAS and eQTL (PHEIDI>0.01); (7) Cicero co-accessibility interactions data from 57,262 snATAC-seq cells (co-accessibility score>0.2)(https://susztaklab.com/Human_snATAC/index.php); and (8) Element-gene connections identified by Activity-by-Contact (ABC) model which predicts enhancers regulating genes based on estimating enhancer activity and enhancer-promoter contact frequency from epigenomic datasets (ABC scores>=0.015). Promoters were defined as ±2000 bp from the TSS of protein-coding transcripts from GENCODE v351ift3765 to annotate Cicero connections or Element-gene connections between gene promoters and eGFR GWAS variants. eGFRcrea GWAS meta-analysis, (n=1,508,659 individuals), DNA methylation data (n=506 human kidneys), cis-eQTL, kidney mQTL (n=686 individuals) and Bayesian colocalization, and summary Mendelian randomization analyses were performed.

### Human Kidney Single Nuclear ATAC-Sequencing

Adult human kidney single nuclear ATAC seq-data was used. The data can be viewed at the website (http://www.susztaklab.com/Human_snATAC/index.php).

### Generation and Maintenance of Acss2 Knockout and Tubule Specific Fasn Knockout Mice

ACSS2 knock-out mice were generated by at the CRISPR Cas9 Mouse Targeting Core facility at the University of Pennsylvania. Two sgRNAs were generated with Guide-it™ sgRNA In Vitro Transcription Kit (#632635, Takara). Knock-out mice were identified by standard tail genotyping PCR. The genetic deletion was confirmed by in situ hybridization, quantitative real time PCR and western blotting.

To generate tubule specific Fasn knockout (Fasn f/f Ksp Cre) mice, the present study procured mice harboring Cre recombinase under tubule gene cadherin16 promoter (Ksp Cre) from Jackson lab (stock #012237). These mice were mated with mice having FASN floxed allele. Scap f/f mice on C57BL/6 genetic background was generated earlier.

### Animal Studies

Animal studies were approved by the Institutional Animal Care and Use Committee (IACUC) of the University of Pennsylvania. Mice were housed in pathogen free conditions with 12 h dark and light cycle in a temperature- and humidity-controlled environment (23±1). The mice were fed ad libitum with water and rodent standard chow diet. 6- to 7-week-old male and female C57BL/6J mice were used in the study. Mice were randomly assigned to experimental groups for all experiments including drug efficacy studies. To induce kidney injury, the present study employed two widely used kidney disease models including unilateral ureteral obstruction (UUO) and folic acid nephropathy (FAN). UUO surgery experiments were conducted on male and female mice. Briefly, UUO was performed by ligating the right kidney ureter, and the left kidney served as a sham operated kidney. Post-surgical procedures were followed according to the IACUC guidelines. For FAN models, 300 mM sodium bicarbonate (NaHCO₃) solution was first made to solubilize folic acid (FA) and injected into male mice (FA 250 mg/kg i.p at single dose). Mice were sacrificed 7 days following injection or surgery. For the drug injection studies, mice were first injected one day before the UUO surgery.

### Adenine Induced Chronic Kidney Disease Model

Adenine (#A11500) was purchased from RPI (Saint Louis, MO, USA). Adenine was dissolved in water at a concentration 2.5 mg/ml. Male mice were given adenine by oral gavage at a dose of 50 mg/kg body weight daily for four weeks. Control male mice received 0.2 ml of vehicle every day for four weeks. Animals were sacrificed on day 30. BUN and serum creatinine was analyzed by BUN (#B7552-150, Pointe Scientific) and creatinine kits (#DZ072B-KY1, Diazyme). Daily body weights were recorded.

### Crispr SNP Deletion Experiments

Two Crispr guides were generated for the open chromatin of the prioritized SNP region. Guide RNAs were designed using Crispor software and cloned into pLKO5.SgRNA.EFS.GFP plasmid (#57822, Addgene) using Esp3I restriction enzyme (#FD0454, Thermo). Bacterial transformation was performed using OneShot-Stbl3 competent cells (#C737303, Thermo) and isolated plasmids were verified by Sanger sequencing. The guide RNA containing plasmids were transfected into HEK293 cells stably expressing Cas9 (gift from Dr. Liling Wan, University of Pennsylvania) using Lipofectamine 3000 (#L300015, Thermo). After 72 h of transfection, puromycin (4 μg/ml) was added for an additional 3 days. The cells were harvested, and RNA, and DNA were isolated. The DNA was cloned into TOPO-TA vector and TOP10 chemical competent cells (#K4500J10, Thermo). Genomic region deletion was further verified by Sanger sequencing. The isolated RNA was used to measure gene expression by quantitative real time PCR.

### Gene Expression Analysis

A total of 15 mg of kidney tissue was homogenized in 1 ml of Trizol (Ambion) with Qia Tissue Lyzer for 1 min 15 see at 4° C. After homogenization in Trizol, 200 ul of chloroform was directly dispensed into the Trizol lysate and vortexed for 15 see at RT. Lysates were then spun down at 12000 rpm for 15 min at 4° C., and the upper aqueous layer was collected into new, clean tubes. Next, 500 ul of isopropanol (100%) was slowly added through the wall of the tubes and mixed gently. The tubes were then spun down at 12500 rpm at 4° C. for 15 min, and the pellet was washed with 70% ethanol (made from clean 100% ethanol) at 10000 rpm for 10 min at 4° C. Finally, the RNA pellet was dried at RT for 15 min, resuspended in clean RNase free and Dnase free water. RNA was pretreated with Dnase before proceeding to cDNA conversion. A total of 2,000 ng RNA was converted into cDNA using the High-capacity cDNA Reverse Transcription Kit (#4368813, Applied Biosystems). Realtime quantitative PCR analysis was performed to measure the relative gene expression by normalizing the CT values of gene of interest with endogenous control gene (Gapdh was used). The data was calculated and presented as fold change by (2-ΔΔCT method).

### Western Blotting

Approximately 20-30 mg of kidney tissue was homogenized in SDS-blue loading buffer (#7722, CST) containing 42 mM DTT. Samples were loaded onto the SDS-PAGE gels and run at 100v for 1 h 40 min at RT in Tris-Glycine-SDS buffer. The proteins were transferred onto a PVDF membrane. Membranes were blocked with 3% non-fat dry milk powder in tris-buffer saline containing Tween-20 (TBST) for 30 min at RT. The blots were then incubated with primary antibody overnight at 4OC. After primary antibody incubation, the blots were washed three times with TBST, IRdye-conjugated secondary antibodies were probed for 1 h at RT. Finally, the blots were washed in TBST for three minutes each, 10 min at RT, and scanned at 600, 700, and 800 excitation wavelengths in Li-COR imager (Odysseyφ XF), Image Studio software. The images were quantified for relative abundance in Image J software.

### Immunofluorescence Staining

Immunofluorescence was performed. Briefly, 5 μm thin formalin-fixed paraffin-embedded kidney cortical sections were deparaffinized and rehydrated using ethanol gradients from 100%-70%. Slides were preheated in a 10 mM citrate buffer containing 0.10% triton X-100 to retrieve the target antigen. The slides were blocked with PBS containing 10% BSA and 0.1% Tween for 1 h at RT. The slides were incubated overnight at 4C with primary antibodies prepared in PBS. The following primary antibodies were used: anti-Ki67 1:50 dilution, anti-ACSS2 1:50 dilution, anti-FASN at 1:50 dilution, and anti-Perilipin2 at 1:50 dilution. The slides were then incubated at 37OC with anti-rabbit Alexa Fluor 488, and anti-mouse Alexa Fluor 594. Finally, the sections were stained with DAPI containing anti-fade mounting media (#P36941, Invitrogen).

### Histone Extraction and Western Blotting

To extract histones from kidneys, the present study first isolated nuclei and proceeded with acid-histone extraction method. Briefly, the present study washed nearly 40 mg of kidney tissue with ice-cold PBS and minced it in the nuclei isolation buffer NIB-250 is 15 mM Tris-HCl at pH 7.5, 60 mM KCl, 15 mM NaCl, 5 mM MgCl₂, 1 mM CaCl₂), and 250 mM sucrose, to which 0.1% Nonidet P-40, lx protease inhibitor cocktail, 1 mM DTT, and 10 mM sodium butyrate). The present study collected the kidney pieces into glass Dounce homogenizer on ice. After 5 min of incubation on ice, the present study spun down the homogenates, collected the nuclei pellet, washed it twice with NIB-250 without Nonidet P-40 and proceeded with histone extraction.

The present study incubated the nuclear pellet with 0.4 N H2SO4 at a 5:1 ratio for 2 h at 4OC. The present study then spun down the acidified nuclei at 11000rcf for 10 min at 4OC and collected the soluble fraction containing histones into a new tube and precipitated with 20% trichloroacetic acid at final concentration overnight at 4° C. The present study spun down the samples at 11000rcf for 10 min at 4OC to sediment the histone pellet at the bottom of the tube. The present study then washed the histone pellets with 1 ml of ice-cold acetone containing 0.1% 12N HCl, followed by two washes with ice-cold 100% acetone. The present study air-dried the pellet and dissolved them in RIPA buffer. Two micrograms of histone lysates were loaded onto 15% SDS-PAGE gels.

### H&E and Sirius Red Staining

The tissues were fixed in formalin, dehydrated by an ethanol gradient (30%, 50%, 75% and 95%) and then submitted to the histology Core in 100% ethanol. Once the tissue was sectioned, H&E and Sirius red staining was performed. Images were acquired in Olympus 5000 microscope with Cell Sense software. Percentage of relative fibrosis was quantified in image J.

### In Vivo Inhibitor Studies

For the inhibitor studies, littermate mice were randomly assigned to receive either FASNall or ACSS2i. A single dose of 1 mg/ml FASNall was given via i.p injection at 10 mg/kg body weight one day prior to the UUO surgery. Mice were sacrificed 3 days after UUO surgery, and their kidneys were harvested. ACSS2i was injected at 20 mg/kg body weight via i.p injection one day prior to UUO surgery, followed by daily injections for 5 days. Mice sacrificed on the 7th day after UUO surgery.

### Primary Tubular Epithelial Cell Isolation and In Vitro Experiments

Primary kidney tubular epithelial cells (TECs) were isolated from young pups (2.5-3 wk old) of WT, Acss2−/−, Fasnf/− and Scapf/f mice. Kidneys were collected on a petri dish on ice, minced in RPMI media (Corning), and then then digested with 200 ug/ml collagenase IV (1 mg/ml, calbiochem) at 37° C. for 30 min. Collagenase was inactivated by adding 100 μl of fetal bovine serum (100% FBS), and cells were passed through 100 μm, 70 μm and finally 40 μm strainers. Cells were then centrifuged at 1000 rpm for 5 min at 4OC. The cell pellet was resuspended in 1 ml of sterile ice-cold RBC lysis buffer (Hy-Clone) and incubated for 2 min on ice. Lysis was inhibited by adding ice cold PBS and then centrifuged at 1000 rpm for 10 min at 4° C. Finally, the cell pellet was resuspended in RPMI complete media (10% FBS with antibiotics 1×ITS and 50 ng/ml human EGF) and plated in 10 cm dishes. Cells were grown in the incubator at 5% CO₂ at 37° C., and the medium was changed every other day.

Cells were serum restricted in 0.5% FBS for 24 h. Cells were then treated with 20 ng/ml TGF-beta 1 for 48 h in the presence or absence of FASNall (4 μM/ml) or TVB-3664 (10 nM/ml) in 0.5% serum media for 48 h. Fasnf/− or Scapf/f cells were treated with adenovirus Ad5CMV-eGFP (Ad-GFP) or Ad5CMVCre-eGFP (Ad-Cre-GFP) (University of Iowa Gene Transfer Vector Core, Iowa City, IA) at a concentration of 0.5 μl/ml for 24 h in serum free media, and the infection efficiency was assessed by observing GFP signal under a fluorescence microscope before every experiment.

For, siRNA transfection experiments, a smart pool of non-target control siRNA and mouse siFasn were purchased from Dharmacon (Horizon Biosciences). Cells were seeded in 6-well plates, grown overnight at 80-90% confluent, and then transfected with 20 μM siFasn in RNAimax in OptiMEM for 48 h. After transfection, cells were treated with TGF-beta 1 (20 ng/ml) for 48 h in 0.5% serum containing media. RNA or protein was isolated from these cells, and knockdown efficiency was determined by quantifying the relative Fasn expression using real time qPCR.

### Cholesterol Measurement

Total kidney cholesterol was quantitatively estimated using established methods (#K603-100, BioVision). Approximately 10-15 mg of kidney tissue was homogenized in 300 μl of chloroform: isopropanol: NP-40 (7:11:0.1) in a microcentrifuge. The homogenate was centrifuged at 15000 g for 10 min, and the liquid layer was collected into a new microtube. The supernatant was air dried at 50° C. to remove chloroform and the samples kept under vacuum pressure (SpeedVac, Thermo Scientific) for 30 min to remove trace organic solvent. The dried lipids were dissolved in 200 μl of assay buffer and performed cholesterol measurements as per the manufacturer protocol.

### ³H Labeled FAO Measurements in Mice Kidneys

FAO measurements were performed by tracing tritium labeled water (³H₂O). Frozen whole kidney extracts prepared in Krebs-Ringer bicarbonate buffer containing HEPES (#K4002, Sigma). Nearly 500 μg of protein was used for FAO measurements. Briefly, the kidney homogenates were incubated with master cocktail (Krebs-Ringer bicarbonate buffer containing 100 mg/ml fatty acid free BSA, 2.5 mM palmitic acid, 10 mM carnitine, and 4 μCi of 9,10-³H-palmitoyl-CoA) for 2 h at 37° C. and 600 rpm in dark. The homogenates were then subjected to Folch's lipid extraction protocol (2:1 chloroform and methanol) and further precipitated with 10% trichloro acetic acid (#T6399, Sigma). After high-speed centrifugation at 4 C, 1 ml of supernatants were passed through activated AG 1-X8 resin formate columns (#7316221, BioRad) and eluted in roughly 1 ml volume (³H₂O) into glass vials. Nearly 500 ul of elutes were mixed into 3 ml of scintillation cocktail and read in a liquid scintillation counter (Beckman Coulter). The radioactive counts were subtracted from the sample containing no protein and from sample with cold palmitic acid (#P5585, Sigma). The final counts were normalized to the control samples and presented as relative FAO rate.

### In Vitro Palmitic Acid Oxidation Test by Seahorse Analyzer

Real-time fatty acid oxidation analysis was performed in renal tubule cells using an XF-96 Extracellular Flux Analyzer with the Palmitate Oxidation Stress Test Kit and FAO Substrate (Seahorse Bioscience). Briefly, primary tubule cells were isolated from WT and Acss2−/− mice and were cultured in a Seahorse 96-well plate at a density of 5×10\hat{ }3 cells per well. The day before performing the OCR analysis, the cell culture medium was replaced with substrate-limited medium (DMEM (#A14430-01), 0.5 mM glucose, 1 mM Glutamax, 0.5 mM carnitine, and 1% FBS) and maintained up to 18 hours. An hour before performing the OCR analysis, the substrate-limited medium was exchanged with FAO assay medium (lx potassium bicarbonate buffer with 2.5 mM glucose, 0.5 mM carnitine, and 5 mM HEPES). Etomoxir (40 μM) was added 15 minutes before the start of the OCR analysis to the specified wells. Control cells were supplemented with BSA (0.17 mM) while test cells were supplemented with 1 mM palmitic acid conjugated BSA (0.17 mM). The OCR analysis was performed by treating cells with 2 μM oligomycin, 1 μM fluorocarbonyl cyanide phenylhydrazone (FCCP), and 0.5 μM rotenone plus 1 μM antimycin A at final concentration.

In Vivo DNL Tracing with Deuterated Water

DNL tracing was performed at the GC-MS core at the University of Pennsylvania. To assess total lipogenesis, mice were subjected to UUO for seven days. On the 6th day, mice were fasted overnight at ˜7 μm. On the 7th day, the mice were refed for three hours and then injected with 400 ul of deuterated water (#151882, Sigma) prepared in 0.9% saline via i.p injection and continued feeding for three more hours. Systemic blood was collected by cardiac puncture, and livers and kidneys were harvested using clamps pre-cooled in liquid nitrogen. The blood was allowed to coagulate on ice for 15 min, and spun down at 10,000 g for 5 min at 4 C to collect serum. The frozen liver and kidney samples were ground at liquid nitrogen temperature with a Cryomill (Qiagen). Saponification of lipids and gas chromatography-mass spectrometry (GC-MS) analysis were performed at the GC-MS core. Briefly, 5 μl of serum, and 100 mg of liver or kidney powder was saponified, and fatty acids were extracted by adding 0.5 ml of hexane, vortexing, and transferring the top hexane layer to a new glass vial. Separation was performed by reversed phase ion-pairing chromatography on a C8 column coupled to negative-ion mode, full-scan GC-MS at 1-Hz scan time and 100,000 resolving power (Agilent 7890A Gas Chromatograph; 5975 Mass Spectrometer; Thermo Fischer Scientific). Palmitate was analyzed using GC-MS, and the absolute amount of newly made palmitate was assumed equivalent to the rate of DNL. Data analysis with MAVEN software and natural isotope correction were performed by the GC-MS core.

### Kidney Triglycerides Quantification

Kidney triglycerides were measured using Triglyceride Calorimetric Assay kit (#10010303, Cayman). Approximately 20 mg of kidney tissue was homogenized in NP40-substitute assay buffer containing protease and phosphatase inhibitors. Homogenates were collected after centrifuging at 10,000 g for 10 min at 4° C.

### Oil Red O Staining

For Oil Red O staining, the present study cut 5 μm thin frozen sections. Briefly, the sections were dried at RT for 15 min and fixed in prechilled 10% formalin buffered PBS for 10 min. Slides were washed three times with water for 5 min, and finally rinsed in 60% isopropanol for 5 min. Lipids were stained by incubating slides in fresh Oil Red ‘O’ working solution for 30-60 min at RT and rinsed in 60% isopropanol for five seconds. Slides were washed three times with water and counterstained with hematoxylin for 3 min. Finally, slides were washed in 70% ethanol and mounted with 90% glycerol, and immediately proceeded for microscopic analysis.

### NADPH/NADP+ Ratio Measurements

The NADPH/NADP+ ratio was calculated according to the manufacturer protocol (#G1009, Promega). Briefly, an equal number of cells were cultured in a 96-well format and starved overnight for TGF-beta 1 treatments. Next, the cells were lysed in 20% DTAB containing Basic solution for 10 min at RT. Samples were processed according to the protocol for measuring NADPH and NADP+ from the same well simultaneously. The absolute and relative NADPH/NADP+ values were calculated according to the manufacturer's formula.

### GSH/GSSH Ratio

Reduced and oxidized glutathione levels were measured according to the Glutathione Colorimetric Detection Kit (#EIAGSHC, Thermo Scientific) from the same well after completion of all treatments.

### GSH/GSSH Ratio

Reduced and oxidized glutathione levels were measured according to the Glutathione Colorimetric Detection Kit (#EIAGSHC, Thermo Scientific) from the same well after completion of all treatments.

### Mitochondrial Quality Assessments and Mitophagy

Primary cells were cultured on microscopic cover glass overnight until they reached 70% confluency. Cells were incubated in a 5 μM MitoSox (#M36008, Thermo Scientific) solution for 10 min in incubator and processed for imaging and quantifications. Cells were also incubated in 10 μM JC-1 (#T3168, Thermo Scientific) for 10 min and then proceeded with imaging and quantifications.

Mitophagy was assessed. Primary cells were transfected with Mito ‘Q’ mCherry-eGFP COX8 plasmid for 48 h. Cells were processed for various treatments and proceeded with imaging.

Cells cultured as above and pretreated with 100 μM MitoTempo (#SML0737, Sigma) for 2 h, followed by TGF-beta 1 treatments while MitoTempo was still present.

### In Situ Hybridization

In situ hybridization was performed on formalin-fixed paraffin-embedded tissue sections using the RNAscope 2.5 HD Duplex Detection Kit (#322436, ACD Bio) according to the manufacture protocol. Freshly cut kidney tissue sections were used in all in situ experiments. For the Gsdmd in situ hybridization, the antigen retrieval was performed for 30 min in heated water bath. The following probes were used for the RNAscope in situ assay: Mm Acss2, Hs-ACSS2, Mm-Gsdmd, Mm-Lrp2, Hs-LRP2 and Mm-Hnf4a.

Human kidney bulk RNA-seq, and single nuclear RNA sequencing data can be viewed at the http://www.susztaklab.com/hk_genemap/scRNA website.

### Statistical Methods

Statistical analysis was performed using the GraphPad Prism 9 software. One-way ANOVA and unpaired t-test performed on variables. No outliers were excluded in the in vivo study. Sample size estimation was not performed, and sample size was determined by the number of animals in the colony of a determined age and gender. The number of replicates (including number of animals used in each experiment) are indicated in the figures and/or figure legends. All data are expressed as mean±SEM. The statistical parameters can be found in the figures and the figure legends. P<0.05 was considered significant. P<0.05 (*), p<0.01 (**), p<0.001 (***) and p<0.0001 (****).

### Enumerated Embodiments

In some aspects, the present invention is directed to the following non-limiting embodiments:

Embodiment 1: A method for treating, ameliorating, and/or preventing a chronic kidney disease (CKD), and/or one or more complications thereof, in a subject in need thereof, the method comprising administering to the subject a therapeutically effective amount at least one selected from the group consisting of a DPEP1 inhibitor and a CASP9 inhibitor or a salt, solvate, tautomer, enantiomer, diastereoisomer, geometric isomer, and/or any combination thereof.

Embodiment 2: The method of Embodiment 1, wherein the administering prevents fibrosis in a subject.

Embodiment 3: The method of any of Embodiment 1, wherein the administering prevents ferroptosis in a subject.

Embodiment 4: The method of Embodiment 1, wherein the subject is a mammal.

Embodiment 5: The method of Embodiment 1, wherein the subject is a human subject.

Embodiment 6: A pharmaceutical composition for treating, ameliorating, and/or preventing a chronic kidney disease (CKD), and/or one or more complications thereof, in a subject in need thereof, the composition comprising a therapeutically effective amount of at least one selected from the group consisting of a DPEP1 inhibitor and a CASP9 inhibitor or a salt, solvate, tautomer, enantiomer, diastereoisomer, geometric isomer, and/or any combination thereof.

Embodiment 7: The composition of Embodiment 6, wherein the composition further comprises a pharmaceutically acceptable carrier or an adjuvant.

Embodiment 8: A kit comprising the pharmaceutical composition of Embodiment 6 and instruction material for use thereof, wherein the instructional material comprises instructions for treating CKD in a subject, wherein the treating includes administering the pharmaceutical composition to the subject.

Embodiment 9: A method for treating, ameliorating, and/or preventing a chronic kidney disease (CKD), and/or one or more complications thereof, in a subject in need thereof, the method comprising: identifying a target gene responsible for CKD by integrating evidence from omics datasets and analytical tools selected from the group consisting of eQTLs, meQTLs and eQTM, coloc (GWAS and eQTLs), moloc (GWASs, eQTLs and meQTLs), SMR, HEIDI, single-cell; co-accessibility and activity-by-contact (ABC) model; administering to the subject a therapeutically effective amount of an agent for modulating expression of the identified target gene.

Embodiment 10: The method of Embodiment 9, wherein the identified target gene is DPEP1 and the agent is a DPEP1 inhibitor.

Embodiment 11: The method of Embodiment 9, wherein the identified target gene is CASP9 and the agent is a CASP9 inhibitor.

Embodiment 12: A method for treating, ameliorating, and/or preventing a chronic kidney disease (CKD), and/or one or more complications thereof, in a subject in need thereof, the method comprising administering to the subject a therapeutically effective amount of a ACSS2 inhibitor.

Embodiment 13: The method of Embodiment 12, wherein the administering prevents fibrosis in the subject.

Embodiment 14: The method of Embodiment 12, wherein the subject is a mammal.

Embodiment 15: The method of Embodiment 12, wherein the subject is a human subject.

Embodiment 16: A pharmaceutical composition for treating, ameliorating, and/or preventing a chronic kidney disease (CKD), and/or one or more complications thereof, in a subject in need thereof, the composition comprising a therapeutically effective amount of an ACSS2 inhibitor.

Embodiment 17: The composition of Embodiment 16, wherein the composition further comprises a pharmaceutically acceptable carrier or an adjuvant.

Embodiment 18: A kit comprising the pharmaceutical composition of Embodiment 16 and instruction material for use thereof, wherein the instructional material comprises instructions for treating CKD in a subject, wherein the treating includes administering the pharmaceutical composition to the subject.

Embodiment 19: The kit of Embodiment 18, wherein the subject is a human subject.

Embodiment 20: A method for treating, ameliorating, and/or preventing a chronic kidney disease (CKD), and/or one or more complications thereof, in a subject in need thereof, the method comprising: identifying a target gene responsible for CKD by integrating evidence from omics datasets and analytical tools selected from the group consisting of eQTLs, meQTLs and eQTM, coloc (GWAS and eQTLs), moloc (GWASs, eQTLs and meQTLs), SMR, HEIDI, single-cell; co-accessibility and activity-by-contact (ABC) model; administering to the subject a therapeutically effective amount of an agent for modulating expression of the identified target gene.

Embodiment 21: The method of Embodiment 20, wherein the identified target gene is ACSS2 and the agent is a an ACSS2 inhibitor.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

What is claimed is:

1. A method for treating, ameliorating, and/or preventing a chronic kidney disease (CKD), and/or one or more complications thereof, in a subject in need thereof, the method comprising administering to the subject a therapeutically effective amount at least one selected from the group consisting of a DPEP1 inhibitor and a CASP9 inhibitor or a salt, solvate, tautomer, enantiomer, diastereoisomer, geometric isomer, and/or any combination thereof.

2. The method of claim 1, wherein the administering prevents fibrosis in a subject.

3. The method of any of claim 1, wherein the administering prevents ferroptosis in a subject.

4. The method of claim 1, wherein the subject is a mammal.

5. The method of claim 1, wherein the subject is a human subject.

6. A pharmaceutical composition for treating, ameliorating, and/or preventing a chronic kidney disease (CKD), and/or one or more complications thereof, in a subject in need thereof, the composition comprising a therapeutically effective amount of at least one selected from the group consisting of a DPEP1 inhibitor and a CASP9 inhibitor or a salt, solvate, tautomer, enantiomer, diastereoisomer, geometric isomer, and/or any combination thereof.

7. The composition of claim 6, wherein the composition further comprises a pharmaceutically acceptable carrier or an adjuvant.

8. A kit comprising the pharmaceutical composition of claim 6 and instruction material for use thereof, wherein the instructional material comprises instructions for treating CKD in a subject, wherein the treating includes administering the pharmaceutical composition to the subject.

9. A method for treating, ameliorating, and/or preventing a chronic kidney disease (CKD), and/or one or more complications thereof, in a subject in need thereof, the method comprising:
identifying a target gene responsible for CKD by integrating evidence from omics datasets and analytical tools selected from the group consisting of eQTLs, meQTLs and eQTM, coloc (GWAS and eQTLs), moloc (GWASs, eQTLs and meQTLs), SMR, HEIDI, single-cell; co-accessibility and activity-by-contact (ABC) model;
administering to the subject a therapeutically effective amount of an agent for modulating expression of the identified target gene.

10. The method of claim 9, wherein the identified target gene is DPEP1 and the agent is a DPEP1 inhibitor.

11. The method of claim 9, wherein the identified target gene is CASP9 and the agent is a CASP9 inhibitor.

12. A method for treating, ameliorating, and/or preventing a chronic kidney disease (CKD), and/or one or more complications thereof, in a subject in need thereof, the method comprising administering to the subject a therapeutically effective amount of a ACSS2 inhibitor.

13. The method of claim 12, wherein the administering prevents fibrosis in the subject.

14. The method of claim 12, wherein the subject is a mammal.

15. The method of claim 12, wherein the subject is a human subject.

16. A pharmaceutical composition for treating, ameliorating, and/or preventing a chronic kidney disease (CKD), and/or one or more complications thereof, in a subject in need thereof, the composition comprising a therapeutically effective amount of an ACSS2 inhibitor.

17. The composition of claim 16, wherein the composition further comprises a pharmaceutically acceptable carrier or an adjuvant.

18. A kit comprising the pharmaceutical composition of claim 16 and instruction material for use thereof, wherein the instructional material comprises instructions for treating CKD in a subject, wherein the treating includes administering the pharmaceutical composition to the subject.

19. The kit of claim 18, wherein the subject is a human subject.

20. A method for treating, ameliorating, and/or preventing a chronic kidney disease (CKD), and/or one or more complications thereof, in a subject in need thereof, the method comprising:
identifying a target gene responsible for CKD by integrating evidence from omics datasets and analytical tools selected from the group consisting of eQTLs, meQTLs and eQTM, coloc (GWAS and eQTLs), moloc (GWASs, eQTLs and meQTLs), SMR, HEIDI, single-cell; co-accessibility and activity-by-contact (ABC) model;
administering to the subject a therapeutically effective amount of an agent for modulating expression of the identified target gene.

21. The method of claim 20, wherein the identified target gene is ACSS2 and the agent is a an ACSS2 inhibitor.
